(19) United States
(12) Patent Application Publication
(10) Pub. No.: US 2003/0198955 A1
(43) Pub. Date: Oct. 23, 2003

(54) Novel proteins and nucleic acids encoding same

(76) Inventors: Li Li, Branford, CT (US); Muralidhara Padigaru, Branford, CT (US); Robert A. Ballinger, Newington, CT (US); Ramesh Kekuda, Danbury, CT (US); Steven D. Colman, Guilford, CT (US); Kimberly A. Spytek, New Haven, CT (US); Stacie J. Casman, North Haven, CT (US); Shlomit R. Edinger, New Haven, CT (US); Valerie Gerlach, Branford, CT (US); Paul Sciore, North Haven, CT (US); Glennda Smithson, Guilford, CT (US); John A. Peyman, New Haven, CT (US); John R. MacDougall, Hamden, CT (US); David J. Stone, Guilford, CT (US); Corine A.M. Vernet, Branford, CT (US); Suresh G. Shenoy, Branford, CT (US); Erik Gunther, Branford, CT (US); Isabelle Millet, Milford, CT (US); Velizar T. Tchernev, Branford, CT (US); David W. Anderson, Branford, CT (US); Vladimir Y. Gusev, Madison, CT (US); Uriel M. Malyankar, Branford, CT (US); Haihong Zhong, Guilford, CT (US); Karen Ellerman, Branford, CT (US); Adam R. Wolenc, New Haven, CT (US)

Correspondence Address: Ivor R. Elrifi, Mintz, Levin, Cohn, Ferris,, Glovsky and Popeo, P.C., One Financial Center, Boston, MA, 02111, US

(21) Appl. No.: 10/025,806

(22) Filed: Dec. 19, 2001

Related U.S. Application Data
(60) Provisional application No. 60/256,635, filed on Dec. 18, 2000.
(60) Provisional application No. 60/259,743, filed on Jan. 4, 2001.
(60) Provisional application No. 60/299,327, filed on Jun. 19, 2001.
(60) Provisional application No. 60/261,498, filed on Jan. 12, 2001.
(60) Provisional application No. 60/263,689, filed on Jan. 24, 2001.
(60) Provisional application No. 60/267,464, filed on Feb. 8, 2001.
(60) Provisional application No. 60/271,021, filed on Feb. 22, 2001.
(60) Provisional application No. 60/275,946, filed on Mar. 14, 2001.
(60) Provisional application No. 60/278,150, filed on Mar. 23, 2001.
(60) Provisional application No. 60/285,718, filed on Apr. 23, 2001.
(60) Provisional application No. 60/312,902, filed on Aug. 16, 2001.
(60) Provisional application No. 60/257,876, filed on Dec. 21, 2000.
(60) Provisional application No. 60/260,718, filed on Jan. 10, 2001.
(60) Provisional application No. 60/284,591, filed on Apr. 18, 2001.

(51) Int. Cl. C12Q 1/68; C07H 21/04; C12N 9/00; C12P 21/02; C12N 5/06; C07K 14/435

(52) U.S. Cl. 435/6; 435/69.1; 435/320.1; 435/183; 435/325; 530/350; 536/23.2

(57) ABSTRACT

Disclosed herein are nucleic acid sequences that encode G-coupled protein-receptor related polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S.S.No. 60/256,635 filed Dec. 18, 2000 (Cura-524); U.S.S.No. 60/259,743 filed Jan. 4, 2001 (Cura-524 A); U.S. Ser. No. 60/299,327 filed Jun. 19, 2001 (Cura-524 A1); U.S. Ser. No. 60/261,498 filed Jan. 12, 2001 (Cura-524 B); U.S.S.No. 60/263,689 filed Jan. 24, 2001 (Cura-524 C); U.S.S.No. 60/267,464 filed Feb. 8, 2001 (Cura-524 D); U.S.S.No. 60/271,021 filed Feb. 22, 2001 (Cura-524 E); U.S.S.No. 60/275,946 filed Mar. 14, 2001 (Cura-524 F); U.S.S.No. 60/278,150 filed Mar. 23, 2001 (Cura 524 G); U.S.S.No. 60/285,718 filed Apr. 23, 2001 (Cura-524H); U.S.S.No. 60/312,902 filed Aug. 16, 2001 (Cura-524 I); No. 60/257,876 filed Dec. 21, 2000 (Cura-527); U.S.S.No. 60/260,718 filed Jan. 10, 2001 (Cura-527 A); and U.S.S.No. 60/284,591 filed Apr. 18, 2001 (Cura-527 B), each of which is incorporated by reference in its entirety.

### BACKGROUND OF THE INVENTION

The invention generally relates to nucleic acids and polypeptides. More particularly, the invention relates to nucleic acids encoding novel G-protein coupled receptor (GPCR) polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as “GPCRX” nucleic acids and polypeptides. These nucleic acids and polypeptides, as well as derivatives, homologs, analogs and fragments thereof, will hereinafter be collectively designated as “GPCRX” nucleic acid or polypeptide sequences.

In one aspect, the invention provides an isolated GPCRX nucleic acid molecule encoding a GPCRX polypeptide that includes a nucleic acid sequence that has identity to the nucleic acids disclosed in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127. In some embodiments, the GPCRX nucleic acid molecule will hybridize under stringent conditions to a nucleic acid sequence complementary to a nucleic acid molecule that includes a protein-coding sequence of a GPCRX nucleic acid sequence. The invention also includes an isolated nucleic acid that encodes a GPCRX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127.

Also included in the invention is an oligonucleotide, e.g., an oligonucleotide which includes at least 6 contiguous nucleotides of a GPCRX nucleic acid (e.g., SEQ ID NOS:1, 3, 5, 7, 9, 11, 13, 15, 17, 19, 21, 23, 25, 27, 29, 31, 33, 35, 37, 39, 41, 43), 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127) or a complement of said oligonucleotide.

Also included in the invention are substantially purified GPCRX polypeptides (e.g., SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130). In certain embodiments, the GPCRX polypeptides include an amino acid sequence that is substantially identical to the amino acid sequence of a human GPCRX polypeptide.

The invention also features antibodies that immunoselectively bind to GPCRX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a GPCRX nucleic acid, a GPCRX polypeptide, or an antibody specific for a GPCRX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a GPCRX nucleic acid, under conditions allowing for expression of the GPCRX polypeptide encoded by the DNA. If desired, the GPCRX polypeptide can then be recovered.

In another aspect, the invention includes a method of detecting the presence of a GPCRX polypeptide in a sample. In the method, a sample is contacted with a compound that selectively binds to the polypeptide under conditions allowing for formation of a complex between the polypeptide and the compound. The complex is detected, if present, thereby identifying the GPCRX polypeptide within the sample.

The invention also includes methods to identify specific cell or tissue types based on their expression of a GPCRX.

Also included in the invention is a method of detecting the presence of a GPCRX nucleic acid molecule in a sample by contacting the sample with a GPCRX nucleic acid probe or primer, and detecting whether the nucleic acid probe or primer bound to a GPCRX nucleic acid molecule in the sample. In a further aspect, the invention provides a method for modulating the activity of a GPCRX polypeptide by contacting a cell sample that includes the GPCRX polypeptide with a compound that binds to the GPCRX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., developmental diseases; MHCII and III diseases (immune diseases); taste and scent detectability disorders; Burkitt's lymphoma; corticoneurogenic disease; signal transduction pathway disorders; metabolic pathway disorders; retinal diseases including those involving photoreception; cell growth rate disorders; cell shape disorders; metabolic disorders; feeding disorders; control of feeding; the metabolic syndrome X; wasting disorders associated with chronic diseases; obesity; potential obesity due to over-eating or metabolic disturbances; potential disorders due to starvation (lack of appetite); diabetes; noninsulin-dependent diabetes mellitus (NIDDM1); infectious disease; bacterial, fungal, protozoal and viral infections (particularly infections caused by HIV-1 or HIV-2); pain; cancer (including but not limited to neoplasm; adenocarcinoma; lymphoma; prostate cancer; uterus cancer); cancer-associated cachexia; anorexia; bulimia; asthma; Parkinson's disease; acute heart failure; hypotension; hypertension; urinary retention; osteoporosis; Crohn's disease; multiple sclerosis; Albright Hereditary Ostoeodystrophy; angina pectoris; myocardial infarction; ulcers; allergies; benign prostatic hypertrophy; and psychotic and neurological disorders; including anxiety; schizophrenia; manic depression; delirium; dementia; neurodegenerative disorders; Alzheimer's disease; severe mental retardation; Dentatorubro-pallidoluysian atrophy (DRPLA); Hypophosphatemic rickets; autosomal dominant (2) Acrocallosal syndrome and dyskinesias, such as Huntington's disease or Gilles de la Tourette syndrome; immune disorders; Adrenoleukodystrophy; Congenital Adrenal Hyperplasia; Hemophilia; Hypercoagulation; Idiopathic thrombocytopenic purpura; autoimmume disease; immunodeficiencies; transplantation; Von Hippel-Lindau (VHL) syndrome; Stroke; Tuberous sclerosis; hypercalceimia; Cerebral palsy; Epilepsy; Lesch-Nyhan syndrome; Ataxia-telangiectasia; Leukodystrophies; Behavioral disorders; Addiction; Neuroprotection; Cirrhosis; Transplantation; Systemic lupus erythematosus; Emphysema; Scleroderma; ARDS; Renal artery stenosis; Interstitial nephritis; Glomerulonephritis; Polycystic kidney disease; Systemic lupus erythematosus; Renal tubular acidosis; IgA nephropathy; Cardiomyopathy; Atherosclerosis; Congenital heart defects; Aortic stenosis; Atrial septal defect (ASD); Atrioventricular (A-V) canal defect; Ductus arteriosus; Pulmonary stenosis; Subaortic stenosis; Ventricular septal defect (VSD); valve diseases; Scleroderma; fertility; Pancreatitis; Endocrine dysfunctions; Growth and reproductive disorders; Inflammatory bowel disease; Diverticular disease; Leukodystrophies; Graft vesus host; Hyperthyroidism; Endometriosis; hematopoietic disorders and/or other pathologies and disorders of the like. The therapeutic can be, e.g., a GPCRX nucleic acid, a GPCRX polypeptide, or a GPCRX-specific antibody, or biologically-active derivatives or fragments thereof.

For example, the compositions of the present invention will have efficacy for treatment of patients suffering from the diseases and disorders listed above and/or other pathologies and disorders.

The polypeptides can be used as immunogens to produce antibodies specific for the invention, and as vaccines. They can also be used to screen for potential agonist and antagonist compounds. For example, a cDNA encoding GPCRX may be useful in gene therapy, and GPCRX may be useful when administered to a subject in need thereof. By way of nonlimiting example, the compositions of the present invention will have efficacy for treatment of patients suffering the diseases and disorders listed above and/or other pathologies and disorders.

The invention further includes a method for screening for a modulator of disorders or syndromes including, e.g., diseases and disorders listed above and/or other pathologies and disorders and those disorders related to cell signal processing and metabolic pathway modulation. The method includes contacting a test compound with a GPCRX polypeptide and determining if the test compound binds to said GPCRX polypeptide. Binding of the test compound to the GPCRX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including the diseases and disorders listed above and/or other pathologies and disorders or other disorders related to cell signal processing and metabolic pathway modulation by administering a test compound to a test animal at increased risk for the aforementioned disorders or syndromes. The test animal expresses a recombinant polypeptide encoded by a GPCRX nucleic acid. Expression or activity of GPCRX polypeptide is then measured in the test animal, as is expression or activity of the protein in a control animal which recombinantly-expresses GPCRX polypeptide and is not at increased risk for the disorder or syndrome. Next, the expression of GPCRX polypeptide in both the test animal and the control animal is compared. A change in the activity of GPCRX polypeptide in the test animal relative to the control animal indicates the test compound is a modulator of latency of the disorder or syndrome.

In yet another aspect, the invention includes a method for determining the presence of or predisposition to a disease associated with altered levels of a GPCRX polypeptide, a GPCRX nucleic acid, or both, in a subject (e.g., a human subject). The method includes measuring the amount of the GPCRX polypeptide in a test sample from the subject and comparing the amount of the polypeptide in the test sample to the amount of the GPCRX polypeptide present in a control sample. An alteration in the level of the GPCRX polypeptide in the test sample as compared to the control sample indicates the presence of or predisposition to a disease in the subject. Preferably, the predisposition includes diseases and disorders listed above and/or other pathologies and disorders. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a GPCRX polypeptide, a GPCRX nucleic acid, or a GPCRX-specific antibody to a subject (e.g., a human subject), in an amount sufficient to alleviate or prevent the pathological condition. In preferred embodiments, the disorder, includes the diseases and disorders listed above and/or other pathologies and disorders.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention is based, in part, upon the discovery of novel nucleic acid sequences that encode novel polypeptides. The novel nucleic acids and their encoded polypeptides are collectively designated herein as “GPCRX”.

The novel GPCRX nucleic acids of the invention include the nucleic acids whose sequences are provided in Table 1, inclusive, or a fragment, derivative, analog or homolog thereof. The novel GPCRX proteins of the invention include the protein fragments whose sequences are provided in Table 1, inclusive. The individual GPCRX nucleic acids and proteins are described below. Within the scope of this invention is a method of using these nucleic acids and peptides in the treatment or prevention of a disorder related to cell signaling or metabolic pathway modulation.

The GPCRX proteins of the invention have a high homology to the 7tm_{—}1 domain (PFam Acc. No. pfam00001). The 7tm_{—}1 domain is from the 7 transmembrane receptor family, which includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptamine receptors; (112821, 8488960, 112805, 231454, 1168221, 398971, 112806); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

Because of the close homology among the members of the GPCRX family, proteins that are homologous to any one member of the family are also largely homologous to the other members, except where the sequences are different as shown below.

The similarity information for the GPCRX proteins and nucleic acids disclosed herein suggest that GPCRX may have important structural and/or physiological functions characteristic of the Olfactory Receptor family and the GPCR family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

G-Protein Coupled Receptor proteins (“GPCRs”) have been identified as a large family of G protein-coupled receptors in a number of species. These receptors share a seven transmembrane domain structure with many neurotransmitter and hormone receptors, and are likely to underlie the recognition and G-protein-mediated transduction of various signals. Human GPCR generally do not contain introns and belong to four different gene subfamilies, displaying great sequence variability. These genes are dominantly expressed in olfactory epithelium. See, e.g., Ben-Arie et al., Hum. Mol. Genet. 1994 3:229-235; and, Online Mendelian Inheritance in Man (“OMIM”) entry # 164342 (http://www.ncbi.nlm.nih.gov/entrez/dispomim.cgi?).

The olfactory receptor (“OR”) gene family constitutes one of the largest GPCR multigene families and is distributed among many chromosomal sites in the human genome. See Rouquier et al., Hum. Mol. Genet. 7(9):1337-45 (1998); Malnic et al., Cell 96:713-23 (1999). Olfactory receptors constitute the largest family among G protein-coupled receptors, with up to 1000 members expected. See Vanderhaeghen et al., Genomics 39(3):239-46 (1997); Xie et al., Mamm. Genome 11(12):1070-78 (2000); Issel-Tarver et al., Proc. Natl. Acad. Sci. USA 93(20):10897-902 (1996). The recognition of odorants by olfactory receptors is the first stage in odor discrimination. See Krautwurst et al., Cell 95(7):917-26 (1998); Buck et al., Cell 65(1):175-87 (1991). Many ORs share some characteristic sequence motifs and have a central variable region corresponding to a putative ligand binding site. See Issel-Tarver et al., Proc. Natl. Acad. Sci. USA 93:10897-902 (1996).

Other examples of seven membrane spanning proteins that are related to GPCRs are chemoreceptors. See Thomas et al., Gene 178(1-2):1-5 (1996). Chemoreceptors have been identified in taste, olfactory, and male reproductive tissues. See id.; Walensky et al., J. Biol. Chem. 273(16):9378-87 (1998); Parmentier et al., Nature 355(6359):453-55 (1992); Asai et al., Biochem. Biophys. Res. Commun. 221(2):240-47 (1996).

The GPCRX nucleic acids of the invention encoding GPCR-like proteins include the nucleic acids whose sequences are provided herein, or fragments thereof. The invention also includes mutant or variant nucleic acids any of whose bases may be changed from the corresponding base shown herein while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

The GPCRX proteins of the invention include the GPCR-like proteins whose sequences are provided herein. The invention also includes mutant or variant proteins any of whose residues may be changed from the corresponding residue shown herein while still encoding a protein that maintains its GPCR-like activities and physiological functions, or a functional fragment thereof. The invention further encompasses antibodies and antibody fragments, such as F_{ab} or (F_{ab})₂, that bind immunospecifically to any of the proteins of the invention.

The GPCRX nucleic acids and proteins are useful in potential therapeutic applications implicated in various GPCR-related pathological disorders and/or OR-related pathological disorders, described further below. For example, a cDNA encoding the GPCR (or olfactory-receptor) like protein may be useful in gene therapy, and the receptor -like protein may be useful when administered to a subject in need thereof. The nucleic acids and proteins of the invention are also useful in potential therapeutic applications used in the treatment of developmental diseases; MHCII and III diseases (immune diseases); taste and scent detectability disorders; Burkitt's lymphoma; corticoneurogenic disease; signal transduction pathway disorders; metabolic pathway disorders; retinal diseases including those involving photoreception; cell growth rate disorders; cell shape disorders; metabolic disorders; feeding disorders; control of feeding; the metabolic syndrome X; wasting disorders associated with chronic diseases; obesity; potential obesity due to over-eating or metabolic disturbances; potential disorders due to starvation (lack of appetite); diabetes; noninsulin-dependent diabetes mellitus (NIDDM1); infectious disease; bacterial, fungal, protozoal and viral infections (particularly infections caused by HIV-1 or HIV-2); pain; cancer (including but not limited to neoplasm; adenocarcinoma; lymphoma; prostate cancer; uterus cancer); cancer-associated cachexia; anorexia; bulimia; asthma; Parkinson's disease; acute heart failure; hypotension; hypertension; urinary retention; osteoporosis; Crohn's disease; multiple sclerosis; Albright Hereditary Ostoeodystrophy; angina pectoris; myocardial infarction; ulcers; allergies; benign prostatic hypertrophy; and psychotic and neurological disorders; including anxiety; schizophrenia; manic depression; delirium; dementia; neurodegenerative disorders; Alzheimer's disease; severe mental retardation; Dentatorubro-pallidoluysian atrophy (DRPLA); Hypophosphatemic rickets; autosomal dominant (2) Acrocallosal syndrome and dyskinesias, such as Huntington's disease or Gilles de la Tourette syndrome; immune disorders; Adrenoleukodystrophy; Congenital Adrenal Hyperplasia; Hemophilia; Hypercoagulation; Idiopathic thrombocytopenic purpura; autoimmume disease; immunodeficiencies; transplantation; Von Hippel-Lindau (VHL) syndrome; Stroke; Tuberous sclerosis; hypercalceimia; Cerebral palsy; Epilepsy; Lesch-Nyhan syndrome; Ataxia-telangiectasia; Leukodystrophies; Behavioral disorders; Addiction; Neuroprotection; Cirrhosis; Transplantation; Systemic lupus erythematosus; Emphysema; Scleroderma; ARDS; Renal artery stenosis; Interstitial nephritis; Glomerulonephritis; Polycystic kidney disease; Systemic lupus erythematosus; Renal tubular acidosis; IgA nephropathy; Cardiomyopathy; Atherosclerosis; Congenital heart defects; Aortic stenosis ; Atrial septal defect (ASD); Atrioventricular (A-V) canal defect; Ductus arteriosus; Pulmonary stenosis; Subaortic stenosis; Ventricular septal defect (VSD); valve diseases; Scleroderma; fertility; Pancreatitis; Endocrine dysfunctions; Growth and reproductive disorders; Inflammatory bowel disease; Diverticular disease; Leukodystrophies; Graft vesus host; Hyperthyroidism; Endometriosis; hematopoietic disorders and/or other pathologies and disorders. Other GPCR-related diseases and disorders are contemplated.

The polypeptides can be used as immunogens to produce antibodies specific for the invention, and as vaccines. They can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the compositions of the present invention will have efficacy for treatment of patients suffering the diseases and disorders listed above and/or other pathologies and disorders. The novel nucleic acid encoding GPCR-like protein, and the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

All of the sequence listed in Table 1 have a high degree of homology to known GPCR sequences. Exemplary homology for the sequences is provided in the provisional applications from which the present application claims priority. This homology data are incorporated herein by reference in their entirety.

GPCRX Nucleic Acids and Polypeptides

One aspect of the invention pertains to isolated nucleic acid molecules that encode GPCRX polypeptides or biologically active portions thereof. Also included in the invention are nucleic acid fragments sufficient for use as hybridization probes to identify GPCRX-encoding nucleic acids (e.g., GPCRX mRNAs) and fragments for use as PCR primers for the amplification and/or mutation of GPCRX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An GPCRX nucleic acid can encode a mature GPCRX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated GPCRX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 as a hybridization probe, GPCRX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to GPCRX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127,or a portion of this nucleotide sequence (e.g., a fragment that can be used as a probe or primer or a fragment encoding a biologically-active portion of an GPCRX polypeptide). A nucleic acid molecule that is complementary to the nucleotide sequence shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 is one that is sufficiently complementary to the nucleotide sequence shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 that it can hydrogen bond with little or no mismatches to the nucleotide sequence shown SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of GPCRX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an GPCRX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human GPCRX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127, as well as a polypeptide possessing GPCRX biological activity. Various biological activities of the GPCRX proteins are described below.

An GPCRX polypeptide is encoded by the open reading frame (“ORF”) of an GPCRX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human GPCRX genes allows for the generation of probes and primers designed for use in identifying and/or cloning GPCRX homologues in other cell types, e.g. from other tissues, as well as GPCRX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127; or an anti-sense strand nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127; or of a naturally occurring mutant of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127.

Probes based on the human GPCRX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an GPCRX protein, such as by measuring a level of an GPCRX-encoding nucleic acid in a sample of cells from a subject e.g., detecting GPCRX mRNA levels or determining whether a genomic GPCRX gene has been mutated or deleted.

“A polypeptide having a biologically-active portion of an GPCRX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of GPCRX” can be prepared by isolating a portion SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 that encodes a polypeptide having an GPCRX biological activity (the biological activities of the GPCRX proteins are described below), expressing the encoded portion of GPCRX protein (e.g., by recombinant expression in vitro) and assessing the activity of the encoded portion of GPCRX.

GPCRX Nucleic Acid and Polypeptide Variants

The invention further encompasses nucleic acid molecules that differ from the nucleotide sequences shown SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 due to degeneracy of the genetic code and thus encode the same GPCRX proteins as that encoded by the nucleotide sequences shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130.

In addition to the human GPCRX nucleotide sequences shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 it will be appreciated by those skilled in the art that DNA sequence polymorphisms that lead to changes in the amino acid sequences of the GPCRX polypeptides may exist within a population (e.g., the human population). Such genetic polymorphism in the GPCRX genes may exist among individuals within a population due to natural allelic variation. As used herein, the terms “gene” and “recombinant gene” refer to nucleic acid molecules comprising an open reading frame (ORF) encoding an GPCRX protein, preferably a vertebrate GPCRX protein. Such natural allelic variations can typically result in 1-5% variance in the nucleotide sequence of the GPCRX genes. Any and all such nucleotide variations and resulting amino acid polymorphisms in the GPCRX polypeptides, which are the result of natural allelic variation and that do not alter the functional activity of the GPCRX polypeptides, are intended to be within the scope of the invention.

Moreover, nucleic acid molecules encoding GPCRX proteins from other species, and thus that have a nucleotide sequence that differs from the human sequence SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 are intended to be within the scope of the invention. Nucleic acid molecules corresponding to natural allelic variants and homologues of the GPCRX cDNAs of the invention can be isolated based on their homology to the human GPCRX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding GPCRX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at

pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2× SSC, 0.01% BSA at 50° C. An isolated nucleic acid molecule of the invention that hybridizes under stringent conditions to the sequences of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in 1× SSC, 0.1% SDS at 37° C. Other conditions of moderate stringency that may be used are well-known within the art. See, e.g., Ausubel, et al (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of GPCRX sequences that may exist in the population, the skilled artisan will further appreciate that changes can be introduced by mutation into the nucleotide sequences of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 thereby leading to changes in the amino acid sequences of the encoded GPCRX proteins, without altering the functional ability of said GPCRX proteins. For example, nucleotide substitutions leading to amino acid substitutions at “non-essential” amino acid residues can be made in the sequence of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130. A “non-essential” amino acid residue is a residue that can be altered from the wild-type sequences of the GPCRX proteins without altering their biological activity, whereas an “essential” amino acid residue is required for such biological activity. For example, amino acid residues that are conserved among the GPCRX proteins of the invention are predicted to be particularly non-amenable to alteration. Amino acids for which conservative substitutions can be made are well-known within the art.

Another aspect of the invention pertains to nucleic acid molecules encoding GPCRX proteins that contain changes in amino acid residues that are not essential for activity. Such GPCRX proteins differ in amino acid sequence from SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130 yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130. Preferably, the protein encoded by the nucleic acid molecule is at least about 60% homologous to SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130; more preferably at least about 70% homologous to SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130; still more preferably at least about 80% homologous to SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130; even more preferably at least about 90% homologous to SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130; and most preferably at least about 95% homologous to SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130.

An isolated nucleic acid molecule encoding an GPCRX protein homologous to the protein of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130 can be created by introducing one or more nucleotide substitutions, additions or deletions into the nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 such that one or more amino acid substitutions, additions or deletions are introduced into the encoded protein.

Mutations can be introduced into SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the GPCRX protein is replaced with another amino acid residue from the same side chain family. Alternatively, in another embodiment, mutations can be introduced randomly along all or part of an GPCRX coding sequence, such as by saturation mutagenesis, and the resultant mutants can be screened for GPCRX biological activity to identify mutants that retain activity. Following mutagenesis of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant GPCRX protein can be assayed for (i) the ability to form protein:protein interactions with other GPCRX proteins, other cell-surface proteins, or biologically-active portions thereof, (ii) complex formation between a mutant GPCRX protein and an GPCRX ligand; or (iii) the ability of a mutant GPCRX protein to bind to an intracellular target protein or biologically-active portion thereof, (e.g. avidin proteins).

In yet another embodiment, a mutant GPCRX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire GPCRX coding strand, or to only a portion thereof. Nucleic acid molecules encoding fragments, homologs, derivatives and analogs of an GPCRX protein of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130, or antisense nucleic acids complementary to an GPCRX nucleic acid sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127, are additionally provided.

In one embodiment, an antisense nucleic acid molecule is antisense to a “coding region” of the coding strand of a nucleotide sequence encoding an GPCRX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the GPCRX protein. The term “noncoding region” refers to 5′ and 3′ sequences which flank the coding region that are not translated into amino acids (ie., also referred to as 5′ and 3′ untranslated regions).

Given the coding strand sequences encoding the GPCRX protein disclosed herein, antisense nucleic acids of the invention can be designed according to the rules of Watson and Crick or Hoogsteen base pairing. The antisense nucleic acid molecule can be complementary to the entire coding region of GPCRX mRNA, but more preferably is an oligonucleotide that is antisense to only a portion of the coding or noncoding region of GPCRX mRNA. For example, the antisense oligonucleotide can be complementary to the region surrounding the translation start site of GPCRX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an GPCRX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter 4 are preferred.

In yet another embodiment, the antisense nucleic acid molecule of the invention is an α-anomeric nucleic acid molecule. An o-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave GPCRX mRNA transcripts to thereby inhibit translation of GPCRX mRNA. A ribozyme having specificity for an GPCRX-encoding nucleic acid can be designed based upon the nucleotide sequence of an GPCRX cDNA disclosed herein (i.e., SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127). For example, a derivative of a Tetrahymena L-19 IVS RNA can be constructed in which the nucleotide sequence of the active site is complementary to the nucleotide sequence to be cleaved in an GPCRX-encoding mRNA. See, e.g., U.S. Pat. No. 4,987,071 to Cech, et al. and U.S. Pat. No. 5,116,742 to Cech, et al. GPCRX mRNA can also be used to select a catalytic RNA having a h specific ribonuclease activity from a pool of RNA molecules. See, e.g., Bartel et al., (1993) Science 261:1411-1418.

Alternatively, GPCRX gene expression can be inhibited by targeting nucleotide sequences complementary to the regulatory region of the GPCRX nucleic acid (e.g., the GPCRX promoter and/or enhancers) to form triple helical structures that prevent transcription of the GPCRX gene in target cells. See, e.g., Helene, 1991. Anticancer Drug Des. 6: 569-84; Helene, et al. 1992. Ann. N. Y Acad. Sci. 660: 27-36; Maher, 1992. Bioassays 14: 807-15.

In various embodiments, the GPCRX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of GPCRX can be used in therapeutic and diagnostic applications. For example, PNAs can be used as antisense or antigene agents for sequence-specific modulation of gene expression by, e.g., inducing transcription or translation arrest or inhibiting replication. PNAs of GPCRX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of GPCRX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of GPCRX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

GPCRX Polypeptides

A polypeptide according to the invention includes a polypeptide including the amino acid sequence of GPCRX polypeptides whose sequences are provided in SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130. The invention also includes a mutant or variant protein any of whose residues may be changed from the corresponding residues shown in SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130 while still encoding a protein that maintains its GPCRX activities and physiological functions, or a functional fragment thereof.

In general, an GPCRX variant that preserves GPCRX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated GPCRX proteins, and biologically-active portions thereof, or derivatives, fragments, analogs or homologs thereof. Also provided are polypeptide fragments suitable for use as immunogens to raise anti-GPCRX antibodies. In one embodiment, native GPCRX proteins can be isolated from cells or tissue sources by an appropriate purification scheme using standard protein purification techniques. In another embodiment, GPCRX proteins are produced by recombinant DNA techniques. Alternative to recombinant expression, an GPCRX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the GPCRX protein is derived, or substantially free from chemical precursors or other chemicals when chemically synthesized. The language “substantially free of cellular material” includes preparations of GPCRX proteins in which the protein is separated from cellular components of the cells from which it is isolated or recombinantly-produced. In one embodiment, the language “substantially free of cellular material” includes preparations of GPCRX proteins having less than about 30% (by dry weight) of non-GPCRX proteins (also referred to herein as a “contaminating protein”), more preferably less than about 20% of non-GPCRX proteins, still more preferably less than about 10% of non-GPCRX proteins, and most preferably less than about 5% of non-GPCRX proteins. When the GPCRX protein or biologically-active portion thereof is recombinantly-produced, it is also preferably substantially free of culture medium, i.e., culture medium represents less than about 20%, more preferably less than about 10%, and most preferably less than about 5% of the volume of the GPCRX protein preparation.

The language “substantially free of chemical precursors or other chemicals” includes preparations of GPCRX proteins in which the protein is separated from chemical precursors or other chemicals that are involved in the synthesis of the protein. In one embodiment, the language “substantially free of chemical precursors or other chemicals” includes preparations of GPCRX proteins having less than about 30% (by dry weight) of chemical precursors or non-GPCRX chemicals, more preferably less than about 20% chemical precursors or non-GPCRX chemicals, still more preferably less than about 10% chemical precursors or non-GPCRX chemicals, and most preferably less than about 5% chemical precursors or non-GPCRX chemicals.

Biologically-active portions of GPCRX proteins include peptides comprising amino acid sequences sufficiently homologous to or derived from the amino acid sequences of the GPCRX proteins (e.g., the amino acid sequence shown in SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130) that include fewer amino acids than the full-length GPCRX proteins, and exhibit at least one activity of an GPCRX protein. Typically, biologically-active portions comprise a domain or motif with at least one activity of the GPCRX protein. A biologically-active portion of an GPCRX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native GPCRX protein.

In an embodiment, the GPCRX protein has an amino acid sequence shown in SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130. In other embodiments, the GPCRX protein is substantially homologous to SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130, andretains the functional activity of the protein of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130, yet differs in amino acid sequence due to natural allelic variation or mutagenesis, as described in detail, below. Accordingly, in another embodiment, the GPCRX protein is a protein that comprises an amino acid sequence at least about 45% homologous to the amino acid sequence SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130, and retains the functional activity of the GPCRX proteins of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130.

### Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 11 , 117, 119, 121, 123, 125 and 127.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

### Chimeric and Fusion Proteins

The invention also provides GPCRX chimeric or fusion proteins. As used herein, an GPCRX “chimeric protein” or “fusion protein” comprises an GPCRX polypeptide operatively-linked to a non-GPCRX polypeptide. An “GPCRX polypeptide” refers to a polypeptide having an amino acid sequence corresponding to an GPCRX protein (SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130), whereas a “non-GPCRX polypeptide” refers to a polypeptide having an amino acid sequence corresponding to a protein that is not substantially homologous to the GPCRX protein, e.g., a protein that is different from the GPCRX protein and that is derived from the same or a different organism. Within an GPCRX fusion protein the GPCRX polypeptide can correspond to all or a portion of an GPCRX protein. In one embodiment, an GPCRX fusion protein comprises at least one biologically-active portion of an GPCRX protein. In another embodiment, an GPCRX fusion protein comprises at least two biologically-active portions of an GPCRX protein. In yet another embodiment, an GPCRX fusion protein comprises at least three biologically-active portions of an GPCRX protein. Within the fusion protein, the term “operatively-linked” is intended to indicate that the GPCRX polypeptide and the non-GPCRX polypeptide are fused in-frame with one another. The non-GPCRX polypeptide can be fused to the N-terminus or C-terminus of the GPCRX polypeptide.

In one embodiment, the fusion protein is a GST-GPCRX fusion protein in which the GPCRX sequences are fused to the C-terminus of the GST (glutathione S-transferase) sequences. Such fusion proteins can facilitate the purification of recombinant GPCRX polypeptides.

In another embodiment, the fusion protein is an GPCRX protein containing a heterologous signal sequence at its N-terminus. In certain host cells (e.g., mammalian host cells), expression and/or secretion of GPCRX can be increased through use of a heterologous signal sequence.

In yet another embodiment, the fusion protein is an GPCRX-immunoglobulin fusion protein in which the GPCRX sequences are fused to sequences derived from a member of the immunoglobulin protein family. The GPCRX-immunoglobulin fusion proteins of the invention can be incorporated into pharmaceutical compositions and administered to a subject to inhibit an interaction between an GPCRX ligand and an GPCRX protein on the surface of a cell, to thereby suppress GPCRX-mediated signal transduction in vivo. The GPCRX-immunoglobulin fusion proteins can be used to affect the bioavailability of an GPCRX cognate ligand. Inhibition of the GPCRX ligand/GPCRX interaction may be useful therapeutically for both the treatment of proliferative and differentiative disorders, as well as modulating (e.g. promoting or inhibiting) cell survival. Moreover, the GPCRX-immunoglobulin fusion proteins of the invention can be used as immunogens to produce anti-GPCRX antibodies in a subject, to purify GPCRX ligands, and in screening assays to identify molecules that inhibit the interaction of GPCRX with an GPCRX ligand.

An GPCRX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An GPCRX-encoding nucleic acid can be cloned into such an expression vector such that the fusion moiety is linked in-frame to the GPCRX protein.

### GPCRX Agonists and Antagonists

The invention also pertains to variants of the GPCRX proteins that function as either GPCRX agonists (i.e., mimetics) or as GPCRX antagonists. Variants of the GPCRX protein can be generated by mutagenesis (e.g., discrete point mutation or truncation of the GPCRX protein). An agonist of the GPCRX protein can retain substantially the same, or a subset of, the biological activities of the naturally occurring form of the GPCRX protein. An antagonist of the GPCRX protein can inhibit one or more of the activities of the naturally occurring form of the GPCRX protein by, for example, competitively binding to a downstream or upstream member of a cellular signaling cascade which includes the GPCRX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the GPCRX proteins.

Variants of the GPCRX proteins that function as either GPCRX agonists (i.e., mimetics) or as GPCRX antagonists can be identified by screening combinatorial libraries of mutants (e.g., truncation mutants) of the GPCRX proteins for GPCRX protein agonist or antagonist activity. In one embodiment, a variegated library of GPCRX variants is generated by combinatorial mutagenesis at the nucleic acid level and is encoded by a variegated gene library. A variegated library of GPCRX variants can be produced by, for example, enzymatically ligating a mixture of synthetic oligonucleotides into gene sequences such that a degenerate set of potential GPCRX sequences is expressible as individual polypeptides, or alternatively, as a set of larger fusion proteins (e.g., for phage display) containing the set of GPCRX sequences therein. There are a variety of methods which can be used to produce libraries of potential GPCRX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential GPCRX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

### Polypeptide Libraries

In addition, libraries of fragments of the GPCRX protein coding sequences can be used to generate a variegated population of GPCRX fragments for screening and subsequent selection of variants of an GPCRX protein. In one embodiment, a library of coding sequence fragments can be generated by treating a double stranded PCR fragment of an GPCRX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the GPCRX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of GPCRX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify GPCRX variants. See, e.g., Arkin and Yourvan, 1992. Proc. Natl. Acad. Sci. USA 89:7811-7815; Delgrave, et al., 1993. Protein Engineering 6:327-331.

Anti-GPCRX Antibodies

Also included in the invention are antibodies to GPCRX proteins, or fragments of GPCRX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}′ and F(_{ab′})₂ fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as ace well, such as IgG₁, IgG₂, and others. Furthermore, in humans, the light chain may be a kappa chain or a lambda chain. Reference herein to antibodies includes a reference to all such classes, subclasses and types of human antibody species.

An isolated GPCRX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of GPCRX-related protein that is located on the surface of the protein, e.g., a hydrophilic region. A hydrophobicity analysis of the human GPCRX-related protein sequence will indicate which regions of a GPCRX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J JmmunoL, 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal . antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F(_{ab′})₂ fragment produced by pepsin digestion of an antibody molecule; (ii) an F_{ab} fragment generated by reducing the disulfide bridges of an F(_{ab′})₂ fragment; (iii) an F_{ab} fragment generated by the treatment of the antibody molecule with papain and a reducing agent and (iv) F, fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimi date HCL), active esters (such as disuccinimidyl suberate), aldehydes (such as glutareldehyde), bis-azido compounds (such as bis (p-azidobenzoyl) hexanediamrine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an GPCRX protein is facilitated by generation of hybridomas that bind to the fragment of an GPCRX protein possessing such a domain. Thus, antibodies that are specific for a desired domain within an GPCRX protein, or derivatives, fragments, analogs or homologs thereof, are also provided herein.

Anti-GPCRX antibodies may be used in methods known within the art relating to the localization and/or quantitation of an GPCRX protein (e.g., for use in measuring levels of the GPCRX protein within appropriate physiological samples, for use in diagnostic methods, for use in imaging the protein, and the like). In a given embodiment, antibodies for GPCRX proteins, or derivatives, fragments, analogs or homologs thereof, that contain the antibody derived binding domain, are utilized as pharmacologically-active compounds (hereinafter “Therapeutics”).

An anti-GPCRX antibody (e.g., monoclonal antibody) can be used to isolate an GPCRX polypeptide by standard techniques, such as affinity chromatography or immunoprecipitation. An anti-GPCRX antibody can facilitate the purification of natural GPCRX polypeptide from cells and of recombinantly-produced GPCRX polypeptide expressed in host cells. Moreover, an anti-GPCRX antibody can be used to detect GPCRX protein (e.g., in a cellular lysate or cell supernatant) in order to evaluate the abundance and pattern of expression of the GPCRX protein. Anti-GPCRX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

GPCRX Recombinant Expression Vectors and Host Cells

Another aspect of the invention pertains to vectors, preferably expression vectors, containing a nucleic acid encoding an GPCRX protein, or derivatives, fragments, analogs or Ihomologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., GPCRX proteins, mutant forms of GPCRX proteins, fusion proteins, etc.).

The recombinant expression vectors of the invention can be designed for expression of GPCRX proteins in prokaryotic or eukaryotic cells. For example, GPCRX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the GPCRX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, GPCRX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the a-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to GPCRX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, GPCRX protein can be expressed in bacterial cells such as E coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding GPCRX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) GPCRX protein. Accordingly, the invention further provides methods for producing GPCRX protein using the host cells of the invention. In one embodiment, the method comprises culturing the host cell of invention (into which a recombinant expression vector encoding GPCRX protein has been introduced) in a suitable medium such that GPCRX protein is produced. In another embodiment, the method further comprises isolating GPCRX protein from the medium or the host cell.

Transgenic GPCRX Animals

The host cells of the invention can also be used to produce non-human transgenic animals. For example, in one embodiment, a host cell of the invention is a fertilized oocyte or an embryonic stem cell into which GPCRX protein-coding sequences have been introduced. Such host cells can then be used to create non-human transgenic animals in which exogenous GPCRX sequences have been introduced into their genome or homologous recombinant animals in which endogenous GPCRX sequences have been altered. Such animals are useful for studying the function and/or activity of GPCRX protein and for identifying and/or evaluating modulators of GPCRX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous GPCRX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing GPCRX-encoding nucleic acid into the male pronuclei of a fertilized oocyte (e.g., by microinjection, retroviral infection) and allowing the oocyte to develop in a pseudopregnant female foster animal. The human GPCRX cDNA sequences of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 can be introduced as a transgene into the genome of a non-human animal. Alternatively, a non-human homologue of the human GPCRX gene, such as a mouse GPCRX gene, can be isolated based on hybridization to the human GPCRX cDNA (described further supra) and used as a transgene. Intronic sequences and polyadenylation signals can also be included in the transgene to increase the efficiency of expression of the transgene. A tissue-specific regulatory sequence(s) can be operably-linked to the GPCRX transgene to direct expression of GPCRX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the GPCRX transgene in its genome and/or expression of GPCRX mRNA in tissues or cells of the animals. A transgenic founder animal can then be used to breed additional animals carrying the transgene. Moreover, transgenic animals carrying a transgene-encoding GPCRX protein can further be bred to other transgenic animals carrying other transgenes.

To create a homologous recombinant animal, a vector is prepared which contains at least a portion of an GPCRX gene into which a deletion, addition or substitution has been introduced to thereby alter, e.g., functionally disrupt, the GPCRX gene. The GPCRX gene can be a human gene (e.g., the cDNA of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127), but more preferably, is a non-human homologue of a human GPCRX gene. For example, a mouse homologue of human GPCRX gene of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127 can be used to construct a homologous recombination vector suitable for altering an endogenous GPCRX gene in the mouse genome. In one embodiment, the vector is designed such that, upon homologous recombination, the endogenous GPCRX gene is functionally disrupted (i.e., no longer encodes a functional protein; also referred to as a “knock out” vector).

Alternatively, the vector can be designed such that, upon homologous recombination, the endogenous GPCRX gene is mutated or otherwise altered but still encodes functional protein (e.g., the upstream regulatory region can be altered to thereby alter the expression of the endogenous GPCRX protein). In the homologous recombination vector, the altered portion of the GPCRX gene is flanked at its 5′- and 3′-termini by additional nucleic acid of the GPCRX gene to allow for homologous recombination to occur between the exogenous GPCRX gene carried by the vector and an endogenous GPCRX gene in an embryonic stem cell. The additional flanking GPCRX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced GPCRX gene has homologously-recombined with the endogenous GPCRX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The GPCRX nucleic acid molecules, GPCRX proteins, and anti-GPCRX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transderrnal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an GPCRX protein or anti-GPCRX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g, Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express GPCRX protein (e.g., via a recombinant expression vector in a host cell in gene therapy applications), to detect GPCRX mRNA (e.g., in a biological sample) or a genetic lesion in an GPCRX gene, and to modulate GPCRX activity, as described further, below. In addition, the GPCRX proteins can be used to screen drugs or compounds that modulate the GPCRX protein activity or expression as well as to treat disorders characterized by insufficient or excessive production of GPCRX protein or production of GPCRX protein forms that have decreased or aberrant activity compared to GPCRX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-GPCRX antibodies of the invention can be used to detect and isolate GPCRX proteins and modulate GPCRX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to GPCRX proteins or have a stimulatory or inhibitory effect on, e.g., GPCRX protein expression or GPCRX protein activity. The invention also includes compounds identified in the screening assays described herein.

In one embodiment, the invention provides assays for screening candidate or test compounds which bind to or modulate the activity of the membrane-bound form of an GPCRX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. US.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of GPCRX protein, or a biologically-active portion thereof, on the cell surface is contacted with a test compound and the ability of the test compound to bind to an GPCRX protein determined. The cell, for example, can of mammalian origin or a yeast cell. Determining the ability of the test compound to bind to the GPCRX protein can be accomplished, for example, by coupling the test compound with a radioisotope or enzymatic label such that binding of the test compound to the GPCRX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of GPCRX protein, or a biologically-active portion thereof, on the cell surface with a known compound which binds GPCRX to form an assay mixture, contacting the assay mixture with a test compound, and determining the ability of the test compound to interact with an GPCRX protein, wherein determining the ability of the test compound to interact with an GPCRX protein comprises determining the ability of the test compound to preferentially bind to GPCRX protein or a biologically-active portion thereof as compared to the known compound.

In another embodiment, an assay is a cell-based assay comprising contacting a cell expressing a membrane-bound form of GPCRX protein, or a biologically-active portion thereof, on the cell surface with a test compound and determining the ability of the test compound to modulate (e.g., stimulate or inhibit) the activity of the GPCRX protein or biologically-active portion thereof. Determining the ability of the test compound to modulate the activity of GPCRX or a biologically-active portion thereof can be accomplished, for example, by determining the ability of the GPCRX protein to bind to or interact with an GPCRX target molecule. As used herein, a “target molecule” is a molecule with which an GPCRX protein binds or interacts in nature, for example, a molecule on the surface of a cell which expresses an GPCRX interacting protein, a molecule on the surface of a second cell, a molecule in the extracellular milieu, a molecule associated with the internal surface of a cell membrane or a cytoplasmic molecule. An GPCRX target molecule can be a non-GPCRX molecule or an GPCRX protein or polypeptide of the invention. In one embodiment, an GPCRX target molecule is a component of a signal transduction pathway that facilitates transduction of an extracellular signal (e.g. a signal generated by binding of a compound to a membrane-bound GPCRX molecule) through the cell membrane and into the cell. The target, for example, can be a second intercellular protein that has catalytic activity or a protein that facilitates the association of downstream signaling molecules with GPCRX.

Determining the ability of the GPCRX protein to bind to or interact with an GPCRX target molecule can be accomplished by one of the methods described above for determining direct binding. In one embodiment, determining the ability of the GPCRX protein to bind to or interact with an GPCRX target molecule can be accomplished by determining the activity of the target molecule. For example, the activity of the target molecule can be determined by detecting induction of a cellular second messenger of the target (i e. intracellular Ca²⁺, diacylglycerol, IP₃, etc.), detecting catalytic/enzymatic activity of the target an appropriate substrate, detecting the induction of a reporter gene (comprising an GPCRX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an GPCRX protein or biologically-active portion thereof with a test compound and determining the ability of the test compound to bind to the GPCRX protein or biologically-active portion thereof. Binding of the test compound to the GPCRX protein can be determined either directly or indirectly as described above. In one such embodiment, the assay comprises contacting the GPCRX protein or biologically-active portion thereof with a known compound which binds GPCRX to form an assay mixture, contacting the assay mixture with a test compound, and determining the ability of the test compound to interact with an GPCRX protein, wherein determining the ability of the test compound to interact with an GPCRX protein comprises determining the ability of the test compound to preferentially bind to GPCRX or biologically-active portion thereof as compared to the known compound.

In still another embodiment, an assay is a cell-free assay comprising contacting GPCRX protein or biologically-active portion thereof with a test compound and determining the ability of the test compound to modulate (e.g. stimulate or inhibit) the activity of the GPCRX protein or biologically-active portion thereof. Determining the ability of the test compound to modulate the activity of GPCRX can be accomplished, for example, by determining the ability of the GPCRX protein to bind to an GPCRX target molecule by one of the methods described above for determining direct binding. In an alternative embodiment, determining the ability of the test compound to modulate the activity of GPCRX protein can be accomplished by determining the ability of the GPCRX protein further modulate an GPCRX target molecule. For example, the catalytic/enzymatic activity of the target molecule on an appropriate substrate can be determined as described, supra.

In yet another embodiment, the cell-free assay comprises contacting the GPCRX protein or biologically-active portion thereof with a known compound which binds GPCRX protein to form an assay mixture, contacting the assay mixture with a test compound, and determining the ability of the test compound to interact with an GPCRX protein, wherein determining the ability of the test compound to interact with an GPCRX protein comprises determining the ability of the GPCRX protein to preferentially bind to or modulate the activity of an GPCRX target molecule.

The cell-free assays of the invention are amenable to use of both the soluble form or the membrane-bound form of GPCRX protein. In the case of cell-free assays comprising the membrane-bound form of GPCRX protein, it may be desirable to utilize a solubilizing agent such that the membrane-bound form of GPCRX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate,

3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either GPCRX protein or its target molecule to facilitate separation of complexed from uncomplexed forms of one or both of the proteins, as well as to accommodate automation of the assay. Binding of a test compound to GPCRX protein, or interaction of GPCRX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-GPCRX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or GPCRX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of GPCRX protein binding or activity determined using standard techniques.

Other techniques for immobilizing proteins on matrices can also be used in the screening assays of the invention. For example, either the GPCRX protein or its target molecule can be immobilized utilizing conjugation of biotin and streptavidin. Biotinylated GPCRX protein or target molecules can be prepared from biotin-NHS(N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with GPCRX protein or target molecules, but which do not interfere with binding of the GPCRX protein to its target molecule, can be derivatized to the wells of the plate, and unbound target or GPCRX protein trapped in the wells by antibody conjugation. Methods for detecting such complexes, in addition to those described above for the GST-immobilized complexes, include immunodetection of complexes using antibodies reactive with the GPCRX protein or target molecule, as well as enzyme-linked assays that rely on detecting an enzymatic activity associated with the GPCRX protein or target molecule.

In another embodiment, modulators of GPCRX protein expression are identified in a method wherein a cell is contacted with a candidate compound and the expression of GPCRX mRNA or protein in the cell is determined. The level of expression of GPCRX mRNA or protein in the presence of the candidate compound is compared to the level of expression of GPCRX mRNA or protein in the absence of the candidate compound. The candidate compound can then be identified as a modulator of GPCRX mRNA or protein expression based upon this comparison. For example, when expression of GPCRX mRNA or protein is greater (i.e., statistically significantly greater) in the presence of the candidate compound than in its absence, the candidate compound is identified as a stimulator of GPCRX mRNA or protein expression.

Alternatively, when expression of GPCRX mRNA or protein is less (statistically significantly less) in the presence of the candidate compound than in its absence, the candidate compound is identified as an inhibitor of GPCRX mRNA or protein expression. The level of GPCRX mRNA or protein expression in the cells can be determined by methods described herein for detecting GPCRX mRNA or protein.

In yet another aspect of the invention, the GPCRX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with GPCRX (“GPCRX-binding proteins” or “GPCRX-bp”) and modulate GPCRX activity. Such GPCRX-binding proteins are also likely to be involved in the propagation of signals by the GPCRX proteins as, for example, upstream or downstream elements of the GPCRX pathway.

The two-hybrid system is based on the modular nature of most transcription factors, which consist of separable DNA-binding and activation domains. Briefly, the assay utilizes two different DNA constructs. In one construct, the gene that codes for GPCRX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an GPCRX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with GPCRX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the GPCRX sequences, SEQ ID NOS:1 ,3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127, or fragments or derivatives thereof, can be used to map the location of the GPCRX genes, respectively, on a chromosome. The mapping of the GPCRX sequences to chromosomes is an important first step in correlating these sequences with genes associated with disease.

Briefly, GPCRX genes can be mapped to chromosomes by preparing PCR primers (preferably 15-25 bp in length) from the GPCRX sequences. Computer analysis of the GPCRX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the GPCRX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the GPCRX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the GPCRX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The GPCRX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the GPCRX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The GPCRX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 1113, 115, 117, 119, 121, 123, 125 and 127 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining GPCRX protein and/or nucleic acid expression as well as GPCRX activity, in the context of a biological sample (e.g., blood, serum, cells, tissue) to thereby determine whether an individual is afflicted with a disease or disorder, or is at risk of developing a disorder, associated with aberrant GPCRX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with GPCRX protein, nucleic acid expression or activity. For example, mutations in an GPCRX gene can be assayed in a biological sample. Such assays can be used for prognostic or predictive purpose to thereby prophylactically treat an individual prior to the onset of a disorder characterized by or associated with GPCRX protein, nucleic acid expression, or biological activity.

Another aspect of the invention provides methods for determining GPCRX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of GPCRX in clinical trials.

These and other agents are described in further detail in the following sections.

### Diagnostic Assays

An exemplary method for detecting the presence or absence of GPCRX in a biological sample involves obtaining a biological sample from a test subject and contacting the biological sample with a compound or an agent capable of detecting GPCRX protein or nucleic acid (e.g., mRNA, genomic DNA) that encodes GPCRX protein such that the presence of GPCRX is detected in the biological sample. An agent for detecting GPCRX mRNA or genomic DNA is a labeled nucleic acid probe capable of hybridizing to GPCRX mRNA or genomic DNA. The nucleic acid probe can be, for example, a full-length GPCRX nucleic acid, such as the nucleic acid of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127, or a portion thereof, such as an oligonucleotide of at least 15, 30, 50, 100, 250 or 500 nucleotides in length and sufficient to specifically hybridize under stringent conditions to GPCRX mRNA or genomic DNA. Other suitable probes for use in the diagnostic assays of the invention are described herein.

An agent for detecting GPCRX protein is an antibody capable of binding to GPCRX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect GPCRX mRNA, protein, or genomic DNA in a biological sample in vitro as well as in vivo. For example, in vitro techniques for detection of GPCRX mRNA include Northern hybridizations and in situ hybridizations. In vitro techniques for detection of GPCRX protein include enzyme linked immunosorbent assays (ELISAs), Western blots, immunoprecipitations, and immunofluorescence. In vitro techniques for detection of GPCRX genomic DNA include Southern hybridizations. Furthermore, in vivo techniques for detection of GPCRX protein include introducing into a subject a labeled anti-GPCRX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting GPCRX protein, mRNA, or genomic DNA, such that the presence of GPCRX protein, mRNA or genomic DNA is detected in the biological sample, and comparing the presence of GPCRX protein, mRNA or genomic DNA in the control sample with the presence of GPCRX protein, mRNA or genomic DNA in the test sample.

The invention also encompasses kits for detecting the presence of GPCRX in a biological sample. For example, the kit can comprise: a labeled compound or agent capable of detecting GPCRX protein or mRNA in a biological sample; means for determining the amount of GPCRX in the sample; and means for comparing the amount of GPCRX in the sample with a standard. The compound or agent can be packaged in a suitable container. The kit can further comprise instructions for using the kit to detect GPCRX protein or nucleic acid.

### Prognostic Assays

The diagnostic methods described herein can furthermore be utilized to identify subjects having or at risk of developing a disease or disorder associated with aberrant GPCRX expression or activity. For example, the assays described herein, such as the preceding diagnostic assays or the following assays, can be utilized to identify a subject having or at risk of developing a disorder associated with GPCRX protein, nucleic acid expression or activity. Alternatively, the prognostic assays can be utilized to identify a subject having or at risk for developing a disease or disorder. Thus, the invention provides a method for identifying a disease or disorder associated with aberrant GPCRX expression or activity in which a test sample is obtained from a subject and GPCRX protein or nucleic acid (e.g., mRNA, genomic DNA) is detected, wherein the presence of GPCRX protein or nucleic acid is diagnostic for a subject having or at risk of developing a disease or disorder associated with aberrant GPCRX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant GPCRX expression or activity. For example, such methods can be used to determine whether a subject can be effectively treated with an agent for a disorder. Thus, the invention provides methods for determining whether a subject can be effectively treated with an agent for a disorder associated with aberrant GPCRX expression or activity in which a test sample is obtained and GPCRX protein or nucleic acid is detected (e.g., wherein the presence of GPCRX protein or nucleic acid is diagnostic for a subject that can be administered the agent to treat a disorder associated with aberrant GPCRX expression or activity).

The methods of the invention can also be used to detect genetic lesions in an GPCRX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an GPCRX-protein, or the misexpression of the GPCRX gene. For example, such genetic lesions can be detected by ascertaining the existence of at least one of: (i) a deletion of one or more nucleotides from an GPCRX gene; (ii) an addition of one or more nucleotides to an GPCRX gene; (iii) a substitution of one or more nucleotides of an GPCRX gene, (iv) a chromosomal rearrangement of an GPCRX gene; (v) an alteration in the level of a messenger RNA transcript of an GPCRX gene, (vi) aberrant modification of an GPCRX gene, such as of the methylation pattern of the genomic DNA, (vii) the presence of a non-wild-type splicing pattern of a messenger RNA transcript of an GPCRX gene, (viii) a non-wild-type level of an GPCRX protein, (ix) allelic loss of an GPCRX gene, and (x) inappropriate post-translational modification of an GPCRX protein. As described herein, there are a large number of assay techniques known in the art which can be used for detecting lesions in an GPCRX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the GPCRX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an GPCRX gene under conditions such that hybridization and amplification of the GPCRX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an GPCRX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in GPCRX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in GPCRX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the GPCRX gene and detect mutations by comparing the sequence of the sample GPCRX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the GPCRX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type GPCRX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in GPCRX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an GPCRX sequence, e.g., a wild-type GPCRX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in GPCRX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control GPCRX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an GPCRX gene.

Furthermore, any cell type or tissue, preferably peripheral blood leukocytes, in which GPCRX is expressed may be utilized in the prognostic assays described herein. However, any biological sample containing nucleated cells may be used, including, for example, buccal mucosal cells.

### Pharmacogenomics

Agents, or modulators that have a stimulatory or inhibitory effect on GPCRX activity (e.g., GPCRX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of GPCRX protein, expression of GPCRX nucleic acid, or mutation content of GPCRX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of GPCRX protein, expression of GPCRX nucleic acid, or mutation content of GPCRX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an GPCRX modulator, such as a modulator identified by one of the exemplary screening assays described herein.

### Monitoring of Effects During Clinical Trials

Monitoring the influence of agents (e.g., drugs, compounds) on the expression or activity of GPCRX (e.g., the ability to modulate aberrant cell proliferation and/or differentiation) can be applied not only in basic drug screening, but also in clinical trials. For example, the effectiveness of an agent determined by a screening assay as described herein to increase GPCRX gene expression, protein levels, or upregulate GPCRX activity, can be monitored in clinical trails of subjects exhibiting decreased GPCRX gene expression, protein levels, or downregulated GPCRX activity. Alternatively, the effectiveness of an agent determined by a screening assay to decrease GPCRX gene expression, protein levels, or downregulate GPCRX activity, can be monitored in clinical trails of subjects exhibiting increased GPCRX gene expression, protein levels, or upregulated GPCRX activity. In such clinical trials, the expression or activity of GPCRX and, preferably, other genes that have been implicated in, for example, a cellular proliferation or immune disorder can be used as a “read out” or markers of the immune responsiveness of a particular cell.

By way of example, and not of limitation, genes, including GPCRX, that are modulated in cells by treatment with an agent (e.g., compound, drug or small molecule) that modulates GPCRX activity (e.g., identified in a screening assay as described herein) can be identified. Thus, to study the effect of agents on cellular proliferation disorders, for example, in a clinical trial, cells can be isolated and RNA prepared and analyzed for the levels of expression of GPCRX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of GPCRX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an GPCRX protein, mRNA, or genomic DNA in the preadministration sample; (iii) obtaining one or more post-administration samples from the subject; (iv) detecting the level of expression or activity of the GPCRX protein, mRNA, or genomic DNA in the post-administration samples; (v) comparing the level of expression or activity of the GPCRX protein, mRNA, or genomic DNA in the pre-administration sample with the GPCRX protein, mRNA, or genomic DNA in the post administration sample or samples; and (vi) altering the administration of the agent to the subject accordingly. For example, increased administration of the agent may be desirable to increase the expression or activity of GPCRX to higher levels than detected, i.e., to increase the effectiveness of the agent. Alternatively, decreased administration of the agent may be desirable to decrease expression or activity of GPCRX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant GPCRX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

### Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

### Prophylactic Methods

In one aspect, the invention provides a method for preventing, in a subject, a disease or condition associated with an aberrant GPCRX expression or activity, by administering to the subject an agent that modulates GPCRX expression or at least one GPCRX activity. Subjects at risk for a disease that is caused or contributed to by aberrant GPCRX expression or activity can be identified by, for example, any or a combination of diagnostic or prognostic assays as described herein. Administration of a prophylactic agent can occur prior to the manifestation of symptoms characteristic of the GPCRX aberrancy, such that a disease or disorder is prevented or, alternatively, delayed in its progression. Depending upon the type of GPCRX aberrancy, for example, an GPCRX agonist or GPCRX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating GPCRX expression or activity for therapeutic purposes. The modulatory method of the invention involves contacting a cell with an agent that modulates one or more of the activities of GPCRX protein activity associated with the cell. An agent that modulates GPCRX protein activity can be an agent as described herein, such as a nucleic acid or a protein, a naturally-occurring cognate ligand of an GPCRX protein, a peptide, an GPCRX peptidomimetic, or other small molecule. In one embodiment, the agent stimulates one or more GPCRX protein activity. Examples of such stimulatory agents include active GPCRX protein and a nucleic acid molecule encoding GPCRX that has been introduced into the cell. In another embodiment, the agent inhibits one or more GPCRX protein activity. Examples of such inhibitory agents include antisense GPCRX nucleic acid molecules and anti-GPCRX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an GPCRX protein or nucleic acid molecule. In one embodiment, the method involves administering an agent (e.g., an agent identified by a screening assay described herein), or combination of agents that modulates (e.g., up-regulates or down-regulates) GPCRX expression or activity. In another embodiment, the method involves administering an GPCRX protein or nucleic acid molecule as therapy to compensate for reduced or aberrant GPCRX expression or activity.

Stimulation of GPCRX activity is desirable in situations in which GPCRX is abnormally downregulated and/or in which increased GPCRX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The GPCRX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the GPCRX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the GPCRX protein, and the GPCRX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

All of the sequences listed in the attached Table 1 have a high degree of homology to known GPCR sequences, most within the range of 97-99%. Exemplary homology for the sequences is provided in the provisional applications from which the present application claims priority. This homology data are incorporated herein by reference in their entirety herein.

TABLE 1

| | | |
|---|---|---|
| SEQ ID | | SEQ ID |
| NO | PROTEIN | NO |

| | | | | |
|---|---|---|---|---|
| Acc. No. | DNA SEQUENCE | (NA) | SEQUENCE | (aa) |

| | | | | |
|---|---|---|---|---|
| GMAC | CCCTTCATATGCCAGGAAGAATGTCAGATTCCAACCTCAGTGATAACCA | 1 | MPGRMSDSNLSDNHLP | 2 |
| 011647_C | TCTTCCAGACACCTTCTTCTTAACAGGGATCCCAGGGCTGGAGGCTGCC | | DTFFLTGIPGLEAAHF | |
| | CACTTCTGGATTGCCATCCCTTTCTGTGCCATGTATCTTGTAGCACTGG | | WIAIPFCAMYLVALVG | |
| | TTGGAAATGCTGCCCTCATCCTGGTCATTGCCATGGACAATGCTCTTCA | | NAALILVIAMDNALHA | |
| | TGCACCTATGTACCTCTTCCTCTGCCTTCTCTCACTCACAGACCTGGCT | | PMYLFLCLLSLTDLAL | |
| | CTCAGTTCTACCACTGTGCCCAAGATGCTGGCCATTTTGTGGCTCCATG | | SSTTVPKMLAILWLHA | |
| | CTGGTGAGATTTCCTTTGGTGGATGCCTGGCCCAGATGTTTTGTGTCCA | | GEISFGGCLAQMFCVH | |
| | TTCTATCTATGCTCTGGAGTCCTCGATTCTACTTGCCATGGCCTTTGAT | | SIYALESSILLAMAFD | |
| | AGGTATGTGGCTATCTGTAACCCATTAAGGTATACAACCATTCTCAACC | | RYVAICNPLRYTTILN | |
| | ATGCTGTCATAGGCAGAATTGGCTTTGTTGGGCTATTCCGTAGTGTGGC | | HAVIGRIGFVGLFRSV | |
| | TATTGTCTCCCCCTTCATCTTCTTGCTGAGGCGACTCCCCTACTGTGGT | | AIVSPFIFLLRRLPYC | |
| | CACCGTGTCATGACACACACATACTGTGAGCATATGGGCATCGCCCGAC | | GHRVMTHTYCEHMGIA | |
| | TGGCCTGTGCCAACATCACTGTCAATATTGTCTATGGGCTAACTGTGGC | | RLACANITVNIVYGLT | |
| | TCTGCTGGCCATGGGACTGGATTCCATTCTCATTGCCATTTCCTATGGC | | VALLAMGLDSILIAIS | |
| | TTTATCCTCCATGCAGTCTTTCACCTTCCATCTCATGATGCCCAGCACA | | YGFILHAVFHLPSHDA | |
| | AAGCTCTGAGTACCTGTGGCTCCCACATTGGCATCATCCTGGTTTTCTA | | QHKALSTCGSHIGIIL | |
| | CATCCCTGCCTTCTTCTCCTTCCTCACCCACCGCTTTGGTCACCACGAA | | VFYIPAFFSFLTHRFG | |
| | GTCCCCAAGCATGTGCACATCTTTCTGGCTAATCTCTATGTGCTGGTGC | | HHEVPKHVHIFLANLY | |
| | CTCCTGTACTCAATCCTATTCTCTATGGAGCTAGAACCAAGGAGATTCG | | VLVPPVLNPILYGART | |
| | GAGTCGACTTCTAAAACTGCTTCACCTGGGGAAGACTTCAATATGAATG | | KEIRSRLLKLLHLGKT | |
| | CTGAGCAGAA | | SI | |
| CG | CTTCATATGCCAGGAAGAATGTCAGATTCCAACCTCAGTGATAACCATC | 3 | MPGRMSDSNLSDNHLP | 4 |
| 50299_01 | TTCCAGACACCTTCTTCTTAACAGGGATCCCAGGGCTGGAGGCTGCCCA | | DTFFLTGIPGLEAAHF | |
| | CTTCTGGATTGCCATCCCTTTCTGTGCCATGTATCTTGTAGCACTGGTT | | WIAIPFCAMYLVALVG | |
| | GGAAATGCTGCCCTCATCCTGGTCATTGCCATGGACAATGCTCTTCATG | | NAALILVIAMDNALHA | |
| | CACCTATGTACCTCTTCCTCTGCCTTCTCTCACTCACAGACCTGGCTCT | | PMYLFLCLLSLTDLAL | |
| | CAGTTCTACCACTGTGCCCAAGATGCTGGCCATTTTGTGGCTCCATGCT | | SSTTVPKMLAILWLHA | |
| | GGTGAGATTTCCTTTGGTGGATGCCTGGCCCAGATGTTTTGTGTCCATT | | GEISFGGCLAQMFCVH | |
| | CTATCTATGCTCTGGAGTCCTCGATTCTACTTGCCATGGCCTTTGATAG | | SIYALESSILLAMAFD | |
| | GTATGTGGCTATCTGTAACCCATTAAGGTATACAACCATTCTCAACCAT | | RYVAICNPLRYTTILN | |
| | GCTGTCATAGGCAGAATTGGCTTTGTTGGGCTATTCCGTAGTGTGGCTA | | HAVIGRIGFVGLFRSV | |
| | TTGTCTCCCCCTTCATCTTCTTGCTGAGGCGACTCCCCTACTGTGGTCA | | AIVSPFIFLLRRLPYC | |
| | CCGTGTCATGACACACACATACTGTGAGCATATGGGCATCGCCCGACTG | | GHRVMTHTYCEHMGIA | |
| | GCCTGTGCCAACATCACTGTCAATATTGTCTATGGGCTAACTGTGGCTC | | RLACANITVNIVYGLT | |
| | TGCTGGCCATGGGACTGGATTCCATTCTCATTGCCATTTCCTATGGCTT | | VALLAMGLDSILIAIS | |
| | TATCCTCCATGCAGTCTTTCACCTTCCATCTCATGATGCCCAGCACAAA | | YGFILHAVFHLPSHDA | |
| | GCTCTGAGTACCTGTGGCTCCCACATTGGCATCACCCTGGTTTTCTACA | | QHKALSTCGSHIGITL | |
| | TCCCTGCCTTCTTCTCCTTCCTCACCCACCGCTTTGGTCACCACGAAGT | | VFYIPAFFSFLTHRFG | |
| | CCCCAAGCATGTGCACATCTTTCTGGCTAATCTCTATGTGCTGGTGCCT | | HHEVPKHVHIFLANLY | |
| | CCTGTACTCAATCCTATTCTCTATGGAGTTAGAACCAAGGAGATTCGGA | | VLVPPVLNPILYGVRT | |
| | GTCGACTTCTAAAACTGCTTCACCTGGGGAAGACTTCAATATGAATGCT | | KEIRSRLLKLLHLGKT | |
| | | | SI | |
| GMAP | GAAATTATGAGAAGGAACTTCACGTTGGTGACTGAGTTCATTCTCCTGG | 5 | MRRNFTLVTEFILLGL | 6 |
| 001112_B | GACTGACGAATCACCAGGAATTACAGATTCTCCTCTTCATGCTGTTTCT | | TNHQELQILLFMLFLA | |
| | GGCCATTTACATGGTCACAGTGGCAGGGAATCTTAGCATGATTGCCCTC | | IYMVTVAGNLSMIALI | |
| | ATCCAGGCCAATGCCCGGCTCCACACGCCCATGTACTTTTTCCTGAGCC | | QANARLHTPMYFFLSH | |
| | ACTTATCCTTCCTGGATCTGTGCTTCTCTTCCAATGTGACCCCAAAGAT | | LSFLDLCFSSNVTPKM | |
| | GCTGGAGATTTTCCTTTCAGAGAAGAAAAGCATTTCCTATCCTGCCTGT | | LEIFLSEKKSISYPAC | |
| | CTTGTTCAGTGTTACCTTTATATCACCTTGGTACACGTTGAGATCTACA | | LVQCYLYITLVHVEIY | |
| | TCCTGGCTGTGATGGCCTTTGACCAGTACATGGCCATCCGAAACCCTCT | | ILAVMAFDQYMAIRNP | |
| | GCTTTATGGCAGCAAAATGTCCAAAAGTGTGTGTTCCTTCCTCATCACG | | LLYGSKMSKSVCSFLI | |
| | GTGCCTTATGTGTATGGAGCGCTCACTGGCCTGATGGAGACCATGTGGA | | TVPYVYGALTGLMETM | |
| | CCTACAACCTAGCCTTCTGTGGCCCCAACGAAATTAATCACTTCTACTG | | WTYNLAFCGPNEINHF | |
| | TGCAGACCCACCACTGATTAAGCTGGCTTGTTCTGACACCTACAACAAG | | YCADPPLIKLACSDTY | |
| | GAGTTGTCAATGTTTGTTGTGGCTGGCTGGAATCTTTCGTTTTCTCTCT | | NKELSMFVVAGWNLSF | |
| | TTATCATATTTATTTCCTACTTTTACATTTTTCCTGCTATCTTAAGGAT | | SLFIIFISYFYIFPAI | |
| | TCGCTCTACAGAGGGCAGGCAAAAAGCTTTTTCTACCTGTGGCTCCCAT | | LRIRSTEGRQKAFSTC | |
| | CTGACAGCTGTTACTATTTTCTATGCAACTCTGTTCTTCATGTGTCTCA | | GSHLTAVTIFYATLFF | |
| | GACCTCCATCAGAAGAGTCCATGGAGCAAGGACAAATGGTAGCTGTACT | | MCLRPPSEESMEQGQM | |
| | TTATACCACTGTGATCCCCATGTTAATCCCATGATCTACA | | VAVLYTTVIPMLIP | |
| GMAC | GGCGCTGCATAAAATCATGGCCCTTTTTTCTGCTAACAGCATAGGTGCT | 7 | MALFSANSIGAMNNSD | 8 |
| 011647_B | ATGAACAACTCTGACACTCGCATAGCAGGCTGCTTCCTCACTGGCATCC | | TRIAGCFLTGIPGLEQ | |
| | CTGGGCTGGAGCAACTACATATCTGGCTGTCCATCCCCTTCTGCATCAT | | LHIWLSIPFCIMYIAA | |
| | GTACATCGCTGCCCTGGAAGGCAATGGCATCCTAATTTGTGTCATCCTC | | LEGNGILICVILSQAI | |
| | TCCCAGGCAATCCTGCATGAGCCCATGTACATATTCTTATCTATGCTGG | | LHEPMYIFLSMLASAD | |
| | CCAGTGCTGATGTCTTGCTCTCTACCACCACCATGCCTAAGGCCCTGGC | | VLLSTTTMPKALANLW | |
| | CAATTTGTGGCTAGGTTATAGCCACATTTCCTTTGATGGCTGCCTCACT | | LGYSHISFDGCLTQMF | |
| | CAGATGTTCTTCATTCACTTCCTCTTCATTCACTCTGCTGTCCTGCTGG | | FIHFLFIHSAVLLAMA | |
| | CCATGGCCTTTGACCGCTATGTGGCCATCTGCTCCCCCCTGCGATATGT | | FDRYVAICSPLRYVTI | |
| | CACAATCCTCACAAGCAAGGTCATTGGGAAGATCGTCACTGCCACCCTG | | LTSKVIGKIVTATLSR | |
| | AGCCGCAGCTTCATCATTATGTTTCCATCCATCTTTCTCCTTGAGCACC | | SFIIMFPSIFLLEHLH | |
| | TGCACTATTGCCAGATCAACATCATTGCACACACATTTTGTGAGCACAT | | YCQINIIAHTFCEHMG | |
| | GGGCATTGCCCATCTGTCCTGTTCTGATATCTCCATCAATGTCTGGTAT | | IAHLSCSDISINVWYG | |
| | GGGTTGGCAGCTGCTCTTCTCTCCACAGGCCTGGACATCATGCTTATTA | | LAAALLSTGLDIMLIT | |
| | CTGTTTCCTACATCCACATCCTCCAAGCAGTCTTCCGCCTCCTTTCTCA | | VSYIHILQAVFRLLSQ | |
| | AGATGCCCGCTCCAAGGCCCTGAGTACCTGTGGATCCCATATCTGTGTC | | DARSKALSTCGSHICV | |
| | ATCCTACTCTTCTATGTCCCTGCCCTTTTTTCTGTCTTTGCCTACAGGT | | ILLFYVPALFSVFAYR | |
| | TTGGTGGGAGAAGCATCCCATGCTATGTCCATATTCTCCTGGCCAGCCT | | FGGRSIPCYVHILLAS | |
| | CTACGTTGTCATTCCTCCTATGCTCAATCCCGTTATTTATGGAGTGAGG | | LYVVIPPMLNPVIYGV | |
| | ACTAAGCCAATACTGGAAGGGGCTAAGCAGATGTTTTCAAATCTTGCCA | | RTKPILEGAKQMFSNL | |
| | AAGGATCTAAATAAATGCTTTCAACTTA | | AKGSK | |
| CG | GCTGCATAAAATCATGGCCCTTTTTTCTGCTAACAGCATAGGTGCTATG | 9 | MALFSANSIGAMNNSD | 10 |
| 104704-01 | AACAACTCTGACACTCGCATAGCAGGCTGCTTCCTCACTGGCATCCCTG | | TRIAGCFLTGIPGLEQ | |
| | GGCTGGAGCAACTACATATCTGGCTGTCCATCCCCTTCTGCATCATGTA | | LHIWLSIPFCIMYITA | |
| | CATCACTGCCCTGGAAGGCAATGGCATCCTAATTTGTGTCATCCTCTCC | | LEGNGILICVILSQAI | |
| | CAGGCAATCCTGCATGAGCCCATGTACATATTCTTATCTATGCTGGCCA | | LHEPMYIFLSMLASAD | |
| | GTGCTGATGTCTTGCTCTCTACCACCACCATGCCTAAGGCCCTGGCCAA | | VLLSTTTMPKALANLW | |
| | TTTGTGGCTAGGTTATAGCCTCATTTCCTTTGATGGCTGCCTCACTCAG | | LGYSLISFDGCLTQMF | |
| | ATGTTCTTCATTCACTTCCTCTTCATTCACTCTGCTGTCCTGCTGGCCA | | FIHFLFIHSAVLLAMA | |
| | TGGCCTTTGACCGCTATGTGGCCATCTGCTCCCCCCTGCGATATGTCAC | | FDRYVAICSPLRYVTI | |
| | AATCCTCACAAGCAAGGTCATTGGGAAGATCGTCACTGCCGCCCTGAGC | | LTSKVIGKIVTAALSH | |
| | CACAGCTTCATCATTATGTTTCCATCCATCTTTCTCCTTGAGCACCTGC | | SFIIMFPSIFLLEHLH | |
| | ACTATTGCCAGATCAATATCATTGCACACACATTTTGTGAGCACATGGG | | YCQINIIAHTFCEHMG | |
| | CATTGCCCATCTGTCCTGTTCTGATATCTCCATCAATGTCTGGTATGGG | | IAHLSCSDISINVWYG | |
| | TTGGCAGCTGCTCTTCTCTCCACAGGCCTAGACATCATGCTTATTACTG | | LAAALLSTGLDIMLIT | |
| | TTTCCTACATCCACATCCTCCAAGCAGTCTTCCGCCTCCTTTCTCAAGA | | VSYIHILQAVFRLLSQ | |
| | TGCCCGCTCCAAGGCCCTGAGTACCTGTGGATCCCATATCTGTGTCATC | | DARSKALSTCGSHICV | |
| | CTACTCTTCTATGTCCCTGCGCTTTTTTCTGTCTTTGCCTACAGGTTTG | | ILLFYVPALFSVFAYR | |
| | GTGGGAGAAGCGTCCCATGCTATGTCCATATTCTCCTGGCCAGCCTCTA | | FGGRSVPCYVHILLAS | |
| | CGTTGTCATTCCTCCTATGCTCAATCCCGTTATTTATGGAGTGAGGACT | | LYVVIPPMLNPVIYGV | |
| | AAGCCAATACTGGAAGGGGCTAAGCAGATGTTTTCAAATCTTGCCAAAG | | RTKPILEGAKQMFSNL | |
| | GATCTAAATAAATGCTT | | AKGSK | |
| GMAC | TTCCTTACCATAATTCTGGCATGTCAGCTCCCAACCACTCCACTGCCAA | 11 | MSAPNHSTANHDMFVL | 12 |
| 011647_A | TCATGATATGTTTGTCCTCATTGGCGTTCCTGGCCTGAAGGAGCTGCAC | | IGVPGLKELHVWISIP | |
| | GTGTGGATCTCCATCCCCTTCTGTCTGATGTACCTGGTGGCTGTGTCAG | | FCLMYLVAVSGNGLLV | |
| | GAAATGGTCTCCTTGTCTGTGTGGTGGCAGTGGAGCACAGTCTTCATGA | | CVVAVEHSLHEPMYLF | |
| | ACCTATGTACCTTTTCCTCTCCATGCTGGCATTTTGGGATCTGATTCTA | | LSMLAFWDLILSTSAV | |
| | TCCACATCTGCAGTACCCAAAGCCTTGAGCATTTTCTGGTTTGATGATG | | PKALSIFWFDDVDISF | |
| | TGGACATCTCCTTTGGTGGCTGTGTCACTCAGCTCTTTTTTATGCATTT | | GGCVTQLFFMHFAFVA | |
| | TGCCTTTGTAGCGGAGTCAGGCATTCTCTTGACCATGGCTTTCGACCGC | | ESGILLTMAFDRYVAI | |
| | TATGTGGCCATCTGCTACCCATTGAGGTATAGCACCATACTTAGCCACA | | CYPLRYSTILSHSVIG | |
| | GTGTTATTGGCAAAATTGGGGGTGTCGTGGTGTTCAGGAGTTTTGCAAC | | KIGGVVVFRSFATVFS | |
| | TGTCTTCTCCATCGTCTTCCTTGTGAAGCGTCTGCCCTTCTGCCGGACA | | IVFLVKRLPFCRTNII | |
| | AACATCATTGCCCACACCTTCTGTGAACACATGGGGCTGGCAAAGCTAG | | AHTFCEHMGLAKLGCS | |
| | GTTGTTCTGAAATCACCATCAATATTTGGTATGGAATCTCTGTACCACT | | EITINIWYGISVPLLS | |
| | ACTCAGTGTTACGTTAGATATGGTGACAATAGTCATCTCCCAGGGGCTC | | VTLDMVTIVISQGLIV | |
| | ATAGTTCAAGCAGTCTTCAGGCTGCCCTCCCTTGGTGCTTGGATGAAAG | | QAVFRLPSLGAWMKAL | |
| | CACTCAGCACCTGTGGTTCCCATGGCAGTGTCATCCTCATGTTCTGCCT | | STCGSHGSVILMFCLP | |
| | TCCAGGAATTTTCACTGTCATTGTTCAGCGCTTTGCCCGAAAATTTCCC | | GIFTVIVQRFARKFPK | |
| | AAGTATGTCCACATCCTGCTGGCCAATCTCTATGTTCTTGTTCCCCCCA | | YVHILLANLYVLVPPM | |
| | TGATGAACCCAATTATCTATGGAGTAAAGACTAAACAGATTCAGAAAGG | | MNPIIYGVKTKQIQKG | |
| | GGTTGCCCTTGTGTTTTCTCCAAAAGGAAAATGTTGCTGAGG | | VALVFSPKGKCC | |
| GMAP | TTAATGGCTGTGGAAAATGACTCTTCAGGGACAAGAGTTTATTCTTTTG | 13 | MTLQGQEFILLGLTDQ | 14 |
| 001804_I | GGATTAACAGACCAGCCTGAGATCCAATTGCCCCTGTTTTTCCTGTTCT | | PEIQLPLFFLFLVNYM | |
| | TGGTGAACTATATGACCACCATGGTGGGCAACTTGAGTTTAATTAATCT | | TTMVGNLSLINLICLN | |
| | AATTTGCCTGAATTCACACCTTCACACTCCCATGTATTTTTTCCTTTTC | | SHLHTPMYFFLFNLSF | |
| | AATCTGTCCTTCATTGATCTCTGTTATTCATTTGTCTTTACCCCCAAAA | | IDLCYSFVFTPKMLMS | |
| | TGCTGATGAGCTTTATTTCAGAGAGGAACATCATCTCCTTTCCAGGATG | | FISERNIISFPGCITQ | |
| | CATAACTCAGCTCTTTTTCTTCTGCTTTTTTGTCCACTCTGAGTGCTAT | | LFFFCFFVHSECYVLT | |
| | GTGCTGACAGCCATGGCCTATGATCGCTATGTGGCCATCTGCAAACCCC | | AMAYDRYVAICKPLLY | |
| | TTCTGTACATGGTCACCACGTCCCCTCAGATCTGTTCTCTACTGATGCT | | MVTTSPQICSLLMLGS | |
| | TGGTTCATATGTGATGGGGTTTGCTGGGGCCATGGTCCACACAGAGTGT | | YVMGFAGAMVHTECMM | |
| | ATGATGAAGCTCATCTTTTGTGACTCCAACGTCATCAACCATTACATGT | | KLIFCDSNVINHYMCD | |
| | GTGACATCTTCCCACTGCTCCAGCTCTCCTGCAGCAGCACCCAGGCCAA | | IFPLLQLSCSSTQANE | |
| | TGAGCTGGTGATGTCTGTTATTGTAGGCACAGTTGTTATAGTATCAAGC | | LVMSVIVGTVVIVSSL | |
| | CTCATTATCTTAATCTCTTATGCTTTGATTCTTTTCAATATCCTTCACA | | IILISYALILFNILHM | |
| | TGTCCTCAGCCGAGGGTTGGTTCAAAGCCATCGGTACCTGTGGCTCCCA | | SSAEGWFKAIGTCGSH | |
| | CATAATAACTGTTGGCCTATTCTATGAATTTGGGCTGATCACTCATGTT | | IITVGLFYEFGLITHV | |
| | AAGTTATCATCTGATTGGTATATGGGTCAGGGGAAGTTTCTCTCAGTGT | | KLSSDWYMGQGKFLSV | |
| | TTTACACGAATGTGGTACCCATGCTGAACCCCCTCATTTATAGCCTCAG | | FYTNVVPMLNPLIYSL | |
| | GAACAAGGATGTCAAACTTGCTCTAAAGGAAACCCTAAATAAAATTACA | | RNKDVKLALKETLNKI | |
| | AACTGA | | TN | |
| GMAP | ACAAAAAATGCTGGCTAGAAACAACTCCTTAGTGACTGAATTTATTCTT | 15 | MLARNNSLVTEFILAG | 16 |
| 001804_F | GCTGGATTAACAGATCATCCAGAGTTCCGGCAACCCCTCTTTTTCCTGT | | LTDHPEFRQPLFFLFL | |
| | TTCTAGTGATCTACATTGTCACCATGGTAGGCAACCTTGGCTTGATCAC | | VIYIVTMVGNLGLITL | |
| | TCTTTTCGGTCTAAATTCTCACCTCCACACACCAATGTACTATTTCCTC | | FGLNSHLHTPMYYFLF | |
| | TTCAATCTCTCCTTCATTGATCTCTGTTACTCCTCTGTTTTCACTCCCA | | NLSFIDLCYSSVFTPK | |
| | AAATGCTAATGAACTTTGTGTCAAAAAAGAATATTATCTCCAATGTTGG | | MLMNFVSKKNIISNVG | |
| | GTGCATGACTCGGCTGTTTTTCTTTCTCTTTTTCGTCATCTCTGAATGT | | CMTRLFFFLFFVISEC | |
| | TACATGTTGACTTCAATGGCATATGATCGCTATGTGGCCATCTGTAATC | | YMLTSMAYDRYVAICN | |
| | CATTGCTGTATAAGGTCACCATGTCCCATCAGGTCTGTTCTATGCTCAC | | PLLYKVTMSHQVCSML | |
| | TTTTGCTGCTTACATAATGGGATTGGCTGGAGCCACGGCCCACACCGGG | | TFAAYIMGLAGATAHT | |
| | TGCATGCTTAGACTCACCTTCTGCAGTGCTAATATCATCAACCATTACT | | GCMLRLTFCSANIINH | |
| | TGTGTGACATACTCCCCCTCCTCCAGCTTTCCTGCACCAGCACCTATGT | | YLCDILPLLQLSCTST | |
| | CAACGAGGTGGTTGTTCTCATTGTTGTGGGTACTAATATCACGGTACCC | | YVNEVVVLIVVGTNIT | |
| | AGTTGTACCATCCTCATTTCTTATGTTTTCATTGTCACTAGCATTCTTC | | VPSCTILISYVFIVTS | |
| | ATATCAAATCCACTCAAGGAAGATCAAAAGCCTTCAGTACTTGTAGCTC | | ILHIKSTQGRSKAFST | |
| | TCATGTCATTGCTCTGTCTCTGTTTTTTGGGTCAGCGGCATTCATGTAT | | CSSHVIALSLFFGSAA | |
| | ATTAAATATTCTTCTGGATCTATGGAGCAGGGAAAAGTTTCTTCTGTTT | | FMYIKYSSGSMEQGKV | |
| | TCTACACTAATGTGGTGCCCATGCTCAATCCCCTCATCTACAGTTTGAG | | SSVFYTNVVPMLNPLI | |
| | GAACAAGGATGTCAAAGTTGCACTGAGGAAAGCTCTGATTAAAATTCAG | | YSLRNKDVKVALRKAL | |
| | AGGAGAAATATATTCTAATTAGAAGCA | | IKIQRRNIF | |
| GMAP | GATGGACTCAAGCTTATGTACCTTCCCCGAGGTCGCTCATGAGTTTATC | 17 | MDSSLCTFPEVAHEFI | 18 |
| 001804_D | CTGGCAGGCTTGACACAACGCCCAGAACTTCAACTGCCACTCTTCCTCC | | LAGLTQRPELQLPLFL | |
| | TGTTCCTTGGAATATATGTGGTCACAGTGGTGGGGAACCTGGGCATGAT | | LFLGIYVVTVVGNLGM | |
| | CTTCTTAATTGCTCTCAGTTCTCAACTTTACCCTCCAGTGTATTATTTT | | IFLIALSSQLYPPVYY | |
| | CTCAGTCATTTGTCTTTCATTGATCTCTGCTACTCCTCTGTCATTACCC | | FLSHLSFIDLCYSSVI | |
| | CTAAGATGCTGGTGAACTTTGTTCCAGAGGAGAACATTATCTCCTTTCT | | TPKMLVNFVPEENIIS | |
| | GGAATGCATTACTCAACTTTATTTCTTCCTTATTTTTGTAATTGCAGAA | | FLECITQLYFFLIFVI | |
| | GGCTACCTTCTGACAGCCATGGAATATGACCGTTATGTTGCTATCTGTC | | AEGYLLTAMEYDRYVA | |
| | GCCCACTGCTTTACAATATTGTCATGTCCCACAGGGTCTGTTCCATAAT | | ICRPLLYNIVMSHRVC | |
| | GATGGCTGTGGTATACTCACTGGGTTTTCTGTGGGCCACAGTCCATACT | | SIMMAVVYSLGFLWAT | |
| | ACCCGCATGTCAGTGTTGTCATTCTGTAGGTCTCATACGGTCAGTCATT | | VHTTRMSVLSFCRSHT | |
| | ATTTTTGTGATATTCTCCCCTTATTGACTCTGTCTTGCTCCAGCACCCA | | VSHYFCDILPLLTLSC | |
| | CATCAATGAGATTCTGCTGTTCATTATTGGAGGAGTTAATACCTTAGCA | | SSTHINEILLFIIGGV | |
| | ACTACACTGGCGGTCCTTATCTCTTATGCTTTCATTTTCTCTAGTATCC | | NTLATTLAVLISYAFI | |
| | TTGGTATTCATTCCACTGAGGGGCAATCCAAAGCCTTTGGCACTTGTAG | | FSSILGIHSTEGQSKA | |
| | CTCCCATCTCTTGGCTGTGGGCATCTTTTTTGGGTCTATAACATTCATG | | FGTCSSHLLAVGIFFG | |
| | TATTTCAAGCCCCCTTCCAGCACTACTATGGAAAAAGAGAAGGTGTCTT | | SITFMYFKPPSSTTME | |
| | CTGTGTTCTACATCACAATAATCCCCATGCTGAATCCTCTAATCTATAG | | KEKVSSVFYITIIPML | |
| | CCTGAGGAACAAGGATGTGAAAAATGCACTGAAGAAGATGACTAGGGGA | | NPLIYSLRNKDVKNAL | |
| | AGGCAGTCATCCTGACAAAGAGG | | KKMTRGRQSS | |
| GMAC | TGTCTTCAACCAACCATGGCAATATTCAATAACACCACTTCGTCTTCCT | 19 | MAIFNNTTSSSSNFLL | 20 |
| 011711_H | CAAACTTCCTCCTCACTGCATTCCCTGGGCTGGAATGTGCTCATGTCTG | | TAFPGLECAHVMISIP | |
| | GATCTCCATTCCAGTCTGCTGTCTCTACACCATTGCCCTCTTGGGAAAC | | VCCLYTIALLGNSMIF | |
| | AGTATGATCTTTCTTGTCATCATTACTAAGCGGAGACTCCACAAACCCA | | LVIITKRRLHKPMYYF | |
| | TGTATTATTTCCTCTCCATGCTGGCAGCTGTTGATCTATGTCTGACCAT | | LSMLAAVDLCLTITTL | |
| | TACGACCCTTCCCACTGTGCTTGGTGTTCTCTGGTTTCATGCCCGGGAG | | PTVLGVLWFHAREISF | |
| | ATCAGCTTTAAAGCTTGCTTCATTCAAATGTTCTTTGTGCATGCTTTCT | | KACFIQMFFVHAFSLL | |
| | CCTTGCTGGAGTCCTCGGTGCTGGTAGCCATGGCCTTTGACCGCTTCGT | | ESSVLVAMAFDRFVAI | |
| | GGCTATCTGTAACCCACTGAACTATGCTACTATCCTCACAGACAGGATG | | CNPLNYATILTDRMVL | |
| | GTCCTGGTGATAGGGCTGGTCATCTGCATTAGACCAGCAGTTTTCTTAC | | VIGLVICIRPAVFLLP | |
| | TTCCCCTTCTTGTAGCCATAAACACTGTGTCTTTTCATGGGGGTCACGA | | LLVAINTVSFHGGHEL | |
| | GCTTTCCCATCCATTTTGCTACCACCCAGAAGTGATCAAATACACATAT | | SHPFCYHPEVIKYTYS | |
| | TCCAAACCTTGGATCAGCAGTTTTTGGGGACTGTTTCTTCAGCTCTACC | | KPWISSFWGLFLQLYL | |
| | TGAATGGCACTGACGTATTGTTTATTCTTTTCTCCTATGTCCTGATCCT | | NGTDVLFILFSYVLIL | |
| | CCGTACTGTTCTGGGCATTGTGGCCCGAAAGAAGCAACAAAAAGCTCTC | | RTVLGIVARKKQQKAL | |
| | AGCACTTGTGTCTGTCACATCTGTGCAGTCACTATTTTCTATGTGCCAC | | STCVCHICAVTIFYVP | |
| | TGATCAGCCTCTCTTTGGCACACCGCCTCTTCCACTCCACCCCAAGGGT | | LISLSLAHRLFHSTPR | |
| | GCTCTGTAGCACTTTGGCCAATATTTATCTGCTCTTACCACCTGTGCTG | | VLCSTLANIYLLLPPV | |
| | AACCCTATCATTTACAGCTTGAAGACCAAGACAATCCGCCAGGCTATGT | | LNPIIYSLKTKTIRQA | |
| | TCCAGCTGCTCCAATCCAAGGGTTCATGGGGTTTTAATGTGAGGGGTCT | | MFQLLQSKGSWGFNVR | |
| | TAGGGGAAGATGGGATTGAAGGTAGGAAATT | | GLRGRWD | |
| CG | TGTCTTCAACCAACCATGGCAATATTCAATAACACCACTTCGTCTTCCT | 21 | MAIFNNTTSSSSNFLL | 22 |
| 55972-01 | CAAACTTCCTCCTCACTGCATTCCCTGGGCTGGAATGTGCTCATGTCTG | | TAFPGLECAHVWISIP | |
| | GATCTCCATTCCAGTCTGCTGTCTCTACACCATTGCCCTCTTGGGAAAC | | VCCLYTIALLGNSMIF | |
| | AGTATGATCTTTCTTGTCATCATTACTAAGCGGAGACTCCACAAACCCA | | LVIITKRRLHKPMYYF | |
| | TGTATTATTTCCTCTCCATGCTGGCAGCTGTTGATCTATGTCTGACCAT | | LSMLAAVDLCLTITTL | |
| | TACGACCCTTCCCACTGTGCTTGGTGTTCTCTGGTTTCATGCCCGGGAG | | PTVLGVLWFHAREISF | |
| | ATCAGCTTTAAAGCTTGCTTCATTCAAATGTTCTTTGTGCATGCTTTCT | | KACFIQMFFVHAFSLL | |
| | CCTTGCTGGAGTCCTCGGTGCTGGTAGCCATGGCCTTTGACCGCTTCGT | | ESSVLVAMAFDRFVAI | |
| | GGCTATCTGTAACCCACTGAACTATGCTACTATCCTCACAGACAGGATG | | CNPLNYATILTDRMVL | |
| | GTCCTGGTGATAGGGCTGGTCATCTGCATTAGACCAGCAGTTTTCTTAC | | VIGLVICIRPAVFLLP | |
| | TTCCCCTTCTTGTAGCCATAAACACTGTGTCTTTTCATGGGGGTCACGA | | LLVAINTVSFHGGHEL | |
| | GCTTTCCCATCCATTTTGCTACCACCCAGAAGTGATCAAATACACATAT | | SHPFCYHPEVIKYTYS | |
| | TCCAAACCTTGGATCAGCAGTTTTTGGGGACTGTTTCTTCAGCTCTACC | | KPWISSFWGLFLQLYL | |
| | TGAATGGCACTGACGTATTGTTTATTCTTTTCTCCTATGTCCTGATCCT | | NGTDVLFILFSYVLIL | |
| | CCGTACTGTTCTGGGCATTGTGGCCCGAAAGAAGCAACAAAAAGCTCTC | | RTVLGIVARKKQQKAL | |
| | AGCACTTGTGTCTGTCACATCTGTGCAGTCACTATTTTCTATGTGCCAC | | STCVCHICAVTIFYVP | |
| | TGATCAGCCTCTCTTTGGCACACCGCCTCTTCCACTCCACCCCAAGGGT | | LISLSLAHRLFHSTPR | |
| | GCTCTGTAGCACTTTGGCCAATATTTATCTGCTCTTACCACCTGTGCTG | | VLCSTLANIYLLLPPV | |
| | AACCCTATCATTTACAGCTTGAAGACCAAGACAATCCGCCAGGCTATGT | | LNPIIYSLKTKTIRQA | |
| | TCCAGCTGCTCCAATCCAAGGGTTCATGGGGTTTTAATGTGAGGGGTCT | | MFQLLQSKGSWGFNVR | |
| | TAGGGGAAGATGGGATTGAAGGTAGGAAATT | | GLRGRWD | |
| GMAC | AACAACATGACGAACTTGAATGCATCACAGGCCAACCACCGTAACTTCA | 23 | MTNLNASQANHRNFIL | 24 |
| 011711_A | TTCTGACAGGTATCCCAGGAACGCCAGACAAGAACCCATGGTTGGCCTT | | TGIPGTPDKNPWLAFP | |
| | TCCCCTGGGATTTCTCTACACACTCACACTCCTGGGAAATGGTACCATC | | LGFLYTLTLLGNGTIL | |
| | CTAGCTGTCATCAAGGTGGAGCCAAGTCTCCATGAGCCCACGTATTACT | | AVIKVEPSLHEPTYYF | |
| | TCCTTTCTATCTTGGCTCTCACTGACGTTAGTCTCTCCATGTCCACCTT | | LSILALTDVSLSMSTL | |
| | GCCCTCCATGCTCAGCATCTACTGGTTTAATGCCCCTCAGATTGTTTTT | | PSMLSIYWFNAPQIVF | |
| | GATGCATGCATCATGCAGATGTTCTTCATCCATGTATTTGGAATAGTAG | | DACIMQMFFIHVFGIV | |
| | AATCAGGAGTCCTAGTGTCCATGGCCTTTGACAGATTTGTGGCCATCCG | | ESGVLVSMAFDRFVAI | |
| | AAACCCATTACACTATGTTTCCATCCTCACTCACGATGTTATTCGAAAG | | RNPLHYVSILTHDVIR | |
| | ACTGGAATAGCTGTCCTCACCCGGGCAGTCTGTGTGGTATTCCCTGTGC | | KTGIAVLTRAVCVVFP | |
| | CCTTCCTTATAAAGTGCCTACCCTTCTGCCATTCCAATGTCTTGTCTCA | | VPFLIKCLPFCHSNVL | |
| | TTCATACTGTCTTCACCAAAACATGATGCGGCTAGCTTGTGCCAGCACC | | SHSYCLHQNMMRLACA | |
| | CGCATCAACAGCCTCTACGGCCTCATCGTCGTCATCTTCACACTGGGGC | | STRINSLYGLIVVIFT | |
| | TCGATGTTCTCCTCACTCTACTGTCTTATGTACTCACCCTGAAGACTGT | | LGLDVLLTLLSYVLTL | |
| | GCTGGGCATTGTCTCCAGAGGTGAAAGGCTGAAAACCCTCAGCACATGC | | KTVLGIVSRGERLKTL | |
| | CTCTCTCACATGTCTACCGTGCTCCTCTTCTATGTTCCTTTTATGGGTG | | STCLSHMSTVLLFYVP | |
| | CTGCCTCCATGATCCACAGATTTTGGGAGCATTTATCACCAGTAGTGCA | | FMGAASMIHRFWEHLS | |
| | CATGGTCATGGCTGATATATACCTACTGCTCCCGCCTGTGCTAAACCCC | | PVVHMVMADIYLLLPP | |
| | ATTGTCTACAGTGTGAAGACCAAGCAAATTGGAAGAATGATCTTTCAAG | | VLNPIVYSVKTKQIGR | |
| | TGTTCCAGAGGCAAAAAAAATAGG | | MIFQVFQRQKK | |
| GMAC | AAATCATGAATTTGGATTCTTTTTTCTCTTTCCTCCTCAAGTCATTGAT | 25 | MALSNSSWRLPQPSFF | 26 |
| 009642_D | AATGGCACTTAGCAATTCCAGCTGGAGGCTACCCCAGCCTTCTTTTTTC | | LVGIPGLEESQHWIAL | |
| | CTGGTAGGAATTCCGGGTTTAGAGGAAAGCCAGCACTGGATCGCACTGC | | PLGILYLLALVGNVTI | |
| | CCCTGGGCATCCTTTACCTCCTTGCTCTAGTGGGCAATGTTACCATTCT | | LFIIWMDPSLHQSMYL | |
| | CTTCATCATCTGGATGGACCCATCCTTGCACCAATCTATGTACCTCTTC | | FLSMLAAIDLVVASST | |
| | CTGTCCATGCTAGCTGCCATCGACCTGGTTGTGGCCTCCTCCACTGCAC | | APKALAVLLVRAQEIG | |
| | CCAAAGCCCTTGCAGTGCTCCTGGTTCGTGCCCAAGAGATTGGTTACAC | | YTVCLIQMFFTHAFSS | |
| | TGTCTGCCTGATCCAGATGTTCTTCACCCATGCATTCTCCTCCATGGAG | | MESGVLVAMALDRYVA | |
| | TCAGGGGTACTTGTGGCCATGGCTCTGGATCGCTATGTAGCCATTTGTC | | ICHPLHHSTILHPGVI | |
| | ACCCCTTGCACCATTCCACAATCCTGCATCCAGGGGTCATAGGGCACAT | | GHIGMVVLVRGLLLLI | |
| | CGGAATGGTGGTGCTGGTGCGGGGATTACTACTCCTCATCCCCTTCCTC | | PFLILLRKLIFCQATI | |
| | ATTCTGTTGCGAAAACTTATCTTCTGCCAAGCCACCATCATAGGCCATG | | IGHAYCEHMAVVKLAC | |
| | CCTATTGTGAACATATGGCTGTTGTGAAACTTGCCTGCTCAGAAACCAC | | SETTVNRAYGLTVALL | |
| | AGTCAATCGAGCTTATGGGCTGACTGTGGCCTTGCTTGTGGTTGGGCTG | | VVGLDVLAIGVSYAHI | |
| | GATGTCCTGGCCATTGGTGTTTCCTATGCCCACATTCTCCAGGCAGTGC | | LQAVLKVPGNEARLKA | |
| | TGAAGGTACCAGGAAATGAGGCCCGACTTAAGGCCTTTAGCACATGTGG | | FSTCGSHVCVILVFYI | |
| | CTCTCATGTTTGTGTCATCCTGGTCTTCTATATCCCGGGAATGTTCTCC | | PGMFSFLTHRFGHHVP | |
| | TTCCTCACTCACCGCTTTGGTCATCATGTACCCCATCACGTCCATGTTC | | HHVHVLLAILYRLVPP | |
| | TTCTGGCCATACTGTATCGCCTTGTGCCACCTGCACTCAATCCTCTTGT | | ALNPLVYRVKTQKIHQ | |
| | CTATAGGGTGAAGACCCAGAAGATCCACCAGGGAGTGCTCAGGGTGTTT | | GVLRVFTLKD | |
| | ACACTAAAGGATTGACCTGAATATATTCTCACT | | | |
| GMAP | AATAAAACAAATTCATTTACTTTCCTTTTGAAACAATTTCTCCATGGGC | 27 | MGNTDLAYTKAMPNFT | 28 |
| 002512_F | AACACAGACTTGGCCTATACTAAGGCAATGCCTAATTTCACGGATGTGA | | DVTEFTLLGLTCRQEL | |
| | CAGAATTTACTCTCCTGGGGCTGACCTGTCGTCAGGAGCTACAGGTTCT | | QVLFFVVFLAVYMITL | |
| | CTTTTTTGTGGTGTTCCTAGCGGTTTACATGATCACTCTGTTGGGAAAT | | LGNIGMIILISISPQL | |
| | ATTGGTATGATCATTTTGATTAGCATCAGTCCTCAGCTTCAGAGTCCCA | | QSPMYFFLSHLSFADV | |
| | TGTACTTTTTCCTGAGTCATCTGTCTTTTGCGGACGTGTGCTTCTCCTC | | CFSSNVTPKMLENLLS | |
| | CAACGTTACCCCCAAAATGCTGGAAAACTTATTATCAGAGACAAAAACC | | ETKTISYVGCLVQCYF | |
| | ATTTCCTATGTGGGATGCTTGGTGCAGTGCTACTTTTTCATTGCCGTTG | | FIAVVHVEVYILAVMA | |
| | TCCACGTGGAGGTCTATATCCTGGCTGTGATGGCCTTTGACAGGTACAT | | FDRYMAGCNPLLYGSK | |
| | GGCCGGCTGCAACCCTCTGCTTTATGGCAGTAAAATGTCTAGGACTGTG | | MSRTVCVRLISVPYVY | |
| | TGTGTTCGGCTCATCTCTGTGCCTTATGTCTATGGATTCTCTGTCAGCC | | GFSVSLICTLWTYGLY | |
| | TAATATGCACACTATGGACTTATGGCTTATACTTCTGTGGAAACTTTGA | | FCGNFEINHFYCADPP | |
| | AATCAATCACTTCTATTGTGCAGATCCCCCTCTCATCCAGATTGCCTGT | | LIQIACGRVHIKEITM | |
| | GGGAGAGTGCACATCAAAGAAATCACAATGATTGTTATTGCTGGAATTA | | IVIAGINFTYSLSVVL | |
| | ACTTCACATATTCCCTCTCGGTGGTCCTCATCTCCTACACTCTCATTGT | | ISYTLIVVAVLRMRSA | |
| | AGTAGCTGTGCTACGCATGCGCTCTGCCGATGGCAGGAGGAAGGCGTTC | | DGRRKAFSTCGSHLTA | |
| | TCCACCTGTGGGTCCCACTTGACGGCTGTTTCTATGTTTTATGGGACCC | | VSMFYGTPIFMYLRRP | |
| | CCATCTTCATGTATCTCAGGAGACCCACTGAGGAATCCGTAGAGCAGGG | | TEESVEQGKMVAVFYT | |
| | CAAAATGGTGGCTGTGTTTTACACCACAGTAATTCCTATGTTGAATCCC | | TVIPMLNPMIYSLRNK | |
| | ATGATCTACAGTCTGAGAAATAAGGATGTAAAAGAAGCAGTCAACAAAG | | DVKEAVNKAITKTYVR | |
| | CAATCACCAAGACATATGTGAGGCAGTAAAACT | | Q | |
| CG | AATAAAACAAATTCATTTACTTTCCTTTTGAAACAATTTCTCCATGGGC | 29 | MGNTDLAYTKAMPNFT | 30 |
| 55958-01 | AACACAGACTTGGCCTATACTAAGGCAATGCCTAATTTCACGGATGTGA | | DVTEFTLLGLTCRQEL | |
| | CAGAATTTACTCTCCTGGGGCTGACCTGTCGTCAGGAGCTACAGGTTCT | | QVLFFVVFLAVYMITL | |
| | CTTTTTTGTGGTGTTCCTAGCGGTTTACATGATCACTCTGTTGGGAAAT | | LGNIGMIILISISPQL | |
| | ATTGGTATGATCATTTTGATTAGCATCAGTCCTCAGCTTCAGAGTCCCA | | QSPMYFFLSHLSFADV | |
| | TGTACTTTTTCCTGAGTCATCTGTCTTTTGCGGACGTGTGCTTCTCCTC | | CFSSNVTPKMLENLLS | |
| | CAACGTTACCCCCAAAATGCTGGAAAACTTATTATCAGAGACAAAAACC | | ETKTISYVGCLVQCYF | |
| | ATTTCCTATGTGGGATGCTTGGTGCAGTGCTACTTTTTCATTGCCGTTG | | FIAVVHVEVYILAVMA | |
| | TCCACGTGGAGGTCTATATCCTGGCTGTGATGGCCTTTGACAGGTACAT | | FDRYMAGCNPLLYGSK | |
| | GGCCGGCTGCAACCCTCTGCTTTATGGCAGTAAAATGTCTAGGACTGTG | | MSRTVCVRLISVPYVY | |
| | TGTGTTCGGCTCATCTCTGTGCCTTATGTCTATGGATTCTCTGTCAGCC | | GFSVSLICTLWTYGLY | |
| | TAATATGCACACTATGGACTTATGGCTTATACTTCTGTGGAAACTTTGA | | FCGNFEINHFYCADPP | |
| | AATCAATCACTTCTATTGTGCAGATCCCCCTCTCATCCAGATTGCCTGT | | LIQIACGRVHIKEITM | |
| | GGGAGAGTGCACATCAAAGAAATCACAATGATTGTTATTGCTGGAATTA | | IVIAGINFTYSLSVVL | |
| | ACTTCACATATTCCCTCTCGGTGGTCCTCATCTCCTACACTCTCATTGT | | ISYTLIVVAVLRMRSA | |
| | AGTAGCTGTGCTACGCATGCGCTCTGCCGATGGCAGGAGGAAGGCGTTC | | DGRRKAFSTCGSHLTA | |
| | TCCACCTGTGGGTCCCACTTGACGGCTGTTTCTATGTTTTATGGGACCC | | VSMFYGTPIFMYLRRP | |
| | CCATCTTCATGTATCTCAGGAGACCCACTGAGGAATCCGTAGAGCAGGG | | TEESVEQGKMVAVFYT | |
| | CAAAATGGTGGCTGTGTTTTACACCACAGTAATTCCTATGTTGAATCCC | | TVIPMLNPMIYSLRNK | |
| | ATGATCTACAGTCTGAGAAATAAGGATGTAAAAGAAGCAGTCAACAAAG | | DVKEAVNKAITKTYVR | |
| | CAATCACCAAGACATATGTGAGGCAGTAAAACT | | Q | |
| GMAC | TGAACTGATACCTCCCCTGCTGGGACATGTCCTTACAGAAACTCATGGA | 31 | MSLQKLMEPEAGTNRT | 32 |
| 023106_A | GCCAGAAGCTGGGACCAATAGGACCGCTGTTGCTGAGTTCATTCTACTG | | AVAEFILLGLVQTEEM | |
| | GGCCTAGTGCAAACAGAAGAGATGCAGCCAGTTGTCTTTGTGCTCCTCC | | QPVVFVLLLFAYLVTT | |
| | TCTTTGCCTATCTGGTCACAACTGGGGGCAACCTCAGCATCCTGGCAGC | | GGNLSILAAVLVEPKL | |
| | CGTCTTGGTGGAGCCCAAACTCCACGCCCCCATGTACTTCTTCCTGGGG | | HAPMYFFLGNLSVLDV | |
| | AACCTGTCAGTGCTGGATGTCGGATGTATCACTGTCACTGTTCCTGCAA | | GCITVTVPAMLGRLLS | |
| | TGTTGGGTCGTCTCTTGTCCCACAAGTCCACAATTTCCTATGACGCCTG | | HKSTISYDACLSQLFF | |
| | CCTCTCCCAGCTCTTCTTCTTCCACCTTCTGGCTGGGATGGACTGCTTC | | FHLLAGMDCFLLTAMA | |
| | CTGCTGACCGCCATGGCCTATGACCGACTCCTGGCCATCTGCCAGCCCC | | YDRLLAICQPLTYSTR | |
| | TCACCTACAGCACCCGCATGAGTCAGACAGTCCAGAGGATGTTGGTGGC | | MSQTVQRMLVAASLAC | |
| | TGCGTCCTTGGCTTGTGCCTTCACCAACGCACTGACCCACACTGTGGCC | | AFTNALTHTVAMSTLN | |
| | ATGTCCACGCTCAACTTCTGTGGCCCCAATGAGGTCAATCACTTCTACT | | FCGPNEVNHFYCDLPQ | |
| | GTGACCTCCCACAGCTCTTCCAGCTCTCCTGCTCCAGCACCCAACTCAA | | LFQLSCSSTQLNELLL | |
| | TGAGCTGCTGCTCTTTGCTGTGGGTTTCATCATGGCAGGCACACCTTTG | | FAVGFIMAGTPLVLII | |
| | GTTCTCATCATCACTGCCTACAGCCACGTGGCAGCTGCAGTTCTACGAA | | TAYSHVAAAVLRIRSV | |
| | TCCGTTCAGTGGAGGGCCGAAAGAAGGCCTTCTCCACGTGTGGCTCCCA | | EGRKKAFSTCGSHLTV | |
| | CCTCACCGTGGTTTGTCTTTTCTTTGGAAGAGGTATCTTCAACTACATG | | VCLFFGRGIFNYMRLG | |
| | AGACTGGGTTCAGAGGAGGCTTCAGACAAGGATAAAGGGGTTGGAGTTT | | SEEASDKDKGVGVFNT | |
| | TCAACACTGTTATCAACCCTATGCTGAACCCTCTTATCTACAGCCTCAG | | VINPMLNPLIYSLRNP | |
| | AAACCCTGATGTTCAGGGTGCTCTGTGGCAAATATTTTTGGGGAGGAGA | | DVQGALWQIFLGRRSL | |
| | TCACTGACCTGAGAG | | T | |
| GMAC | CTATGAGTTCCTGCAACTTCACACATGCCACCTTTGTGCTTATTGGTAT | 33 | MSSCNFTHATFVLIGI | 34 |
| 027367_A | CCCAGGATTAGAGAAAGCCCATTTCTGGGTTGGCTTCCCCCTCCTTTCC | | PGLEKAHFWVGFPLLS | |
| | ATGTATGTAGTGGCAATGTTTGGAAACTGCATCGTGGTCTTCATCGTAA | | MYVVAMFGNCIVVFIV | |
| | GGACGGAACGCAGCCTGCACGCTCCGATGTACCTCTTTCTCTGCATGCT | | RTERSLHAPMYLFLCM | |
| | TGCAGCCATTGACCTGGCCTTATCCACATCCACCATGCCTAAGATCCTT | | LAAIDLALSTSTMPKI | |
| | GCCCTTTTCTGGTTTGATTCCCGAGAGATTAGCTTTGAGGCCTGTCTTA | | LALFWFDSREISFEAC | |
| | CCCAGATGTTCTTTATTCATGCCCTCTCAGCCATTGAATCCACCATCCT | | LTQMFFIHALSAIEST | |
| | GCTGGCCATGGCCTTTGACCGTTATGTGGCCATCTGCCACCCACTGCGC | | ILLAMAFDRYVAICHP | |
| | CATGCTGCAGTGCTCAACAATACAGTAACAGCCCAGATTGGCATCGTGG | | LRHAAVLNNTVTAQIG | |
| | CTGTGGTCCGCGGATCCCTCTTTTTTTTCCCACTGCCTCTGCTGATCAA | | IVAVVRGSLFFFPLPL | |
| | GCGGCTGGCCTTCTGCCACTCCAATGTCCTCTCGCACTCCTATTGTGTC | | LIKRLAFCHSNVLSHS | |
| | CACCAGGATGTAATGAAGTTGGCCTATGCAGACACTTTGCCCAATGTGG | | YCVHQDVMKLAYADTL | |
| | TATATGGTCTTACTGCCATTCTGCTGGTCATGGGCGTGGACGTAATGTT | | PNVVYGLTAILLVMGV | |
| | CATCTCCTTGTCCTATTTTCTGATAATACGAACGGTTCTGCAACTGCCT | | DVMFISLSYFLIIRTV | |
| | TCCAAGTCAGAGCGGGCCAAGGCCTTTGGAACCTGTGTGTCACACATTG | | LQLPSKSERAKAFGTC | |
| | GTGTGGTACTCGCCTTCTATGTGCCACTTATTGGCCTCTCAGTGGTACA | | VSHIGVVLAFYVPLIG | |
| | CCGCTTTGGAAACAGCCTTCATCCCATTGTGCGTGTTGTCATGGGTGAC | | LSVVHRFGNSLHPIVR | |
| | ATCTACCTGCTGCTGCCTCCTGTCATCAATCCCATCATCTATGGTGCCA | | VVMGDIYLLLPPVINP | |
| | AAACCAAACAGATCAGAACACGGGTGCTGGCTATGTTCAAGATCAGCTG | | IIYGAKTKQIRTRVLA | |
| | TGACAAGGACTTGCAGGCTGTGGGAGGCAAGTGACCCTTAACACTACAC | | MFKISCDKDLQAVGGK | |
| | TTCTCCTTATCTTTATTGGCTTGATAAACATAATTATTTCTAAC | | | |
| GMAC | AATGTCCAGCACTCTTGGCCACAACATGGAATCTCCTAATCACACTGAT | 35 | MSSTLGHNMESPNHTD | 36 |
| 026090_D | GTTGACCCTTCTGTCTTCTTCCTCCTGGGCATCCCAGGTCTGGAACAAT | | VDPSVFFLLGIPGLEQ | |
| | TTCATTTGTGGCTCTCACTCCCTGTGTGTGGCTTAGGCACAGCCACAAT | | FHLWLSLPVCGLGTAT | |
| | TGTGGGCAATATAACTATTCTGGTTGTTGTTGCCACTGAACCAGTCTTG | | IVGNITILVVVATEPV | |
| | CACAAGCCTGTGTACCTTTTTCTGTGCATGCTCTCAACCATCGACTTGG | | LHKPVYLFLCMLSTID | |
| | CTGCCTCTGTCTCCACAGTTCCCAAGCTACTGGCTATCTTCTGGTGTGG | | LAASVSTVPKLLAIFW | |
| | AGCCGGACATATATCTGCCTCTGCCTGCCTGGCACAGATGTTCTTCATT | | CGAGHISASACLAQMF | |
| | CATGCCTTCTGCATGATGGAGTCCACTGTGCTACTGGCCATGGCCTTTG | | FIHAFCMMESTVLLAM | |
| | ATCGCTACGTGGCCATCTGCCACCCACTCCGCTATGCCACAATCCTCAC | | AFDRYVAICHPLRYAT | |
| | TGACACCATCATTGCCCACATAGGGGTGGCAGCTGTAGTGCGAGGCTCC | | ILTDTIIAHIGVAAVV | |
| | CTGCTCATGCTCCCATGTCCCTTCCTTATTGGGCGTTTGAACTTCTGCC | | RGSLLMLPCPFLIGRL | |
| | AAAGCCATGTGATCCTACACACGTACTGTGAGCACATGGCTGTGGTGAA | | NFCQSHVILHTYCEHM | |
| | GCTGGCCTGTGGAGACACCAGGCCTAACCGTGTGTATGGGCTGACAGCT | | AVVKLACGDTRPNRVY | |
| | GCACTGTTGGTCATTGGGGTTGACTTGTTTTGCATTGGTCTCTCCTATG | | GLTAALLVIGVDLFCI | |
| | CCCTAAGTGCACAAGCTGTCCTTCGCCTCTCATCCCATGAAGCTCGGTC | | GLSYALSAQAVLRLSS | |
| | CAAGGCCCTAGGGACCTGTGGTTCCCATGTCTGTGTCATCCTCATCTCT | | HEARSKALGTCGSHVC | |
| | TATACACCAGCCCTCTTCTCCTTTTTTACACACCGCTTTGGCCATCACG | | VILISYTPALFSFFTH | |
| | TTCCAGTCCATATTCACATTCTTTTGGCCAATGTTTATCTGCTTTTGCC | | RFGHHVPVHIHILLAN | |
| | ACCTGCTCTTAATCCTGTGGTATATGGAGTTAAGACCAAACAGATCCGT | | VYLLLPPALNPVVYGV | |
| | AAAAGAGTTGTCAGGGTGTTTCAAAGTGGGCAGGGAATGGGCATCAAGG | | KTKQIRKRVVRVFQSG | |
| | CATCTGAGTGACTCTGGA | | QGMGIKASE | |
| CG | AATGTCCAGCACTCTTGGCCACAACATGGAATCTCCTAATCACACTGAT | 37 | MSSTLGHNMESPNHTD | 38 |
| 56826-01 | GTTGACCCTTCTGTCTTCTTCCTCCTGGGCATCCCAGGTCTGGAACAAT | | VDPSVFFLLGIPGLEQ | |
| | TTCATTTGTGGCTCTCACTCCCTGTGTGTGGCTTAGGCACAGCCACAAT | | FHLWLSLPVCGLGTAT | |
| | TGTGGGCAATATAACTATTCTGGTTGTTGTTGCCACTGAACCAGTCTTG | | IVGNITILVVVATEPV | |
| | CACAAGCCTGTGTACCTTTTTCTGTGCATGCTCTCAACCATCGACTTGG | | LHKPVYLFLCMLSTID | |
| | CTGCCTCTGTCTCCACAGTTCCCAAGCTACTGGCTATCTTCTGGTGTGG | | LAASVSTVPKLLAIFW | |
| | AGCCGGACATATATCTGCCTCTGCCTGCCTGGCACAGATGTTCTTCATT | | CGAGHISASACLAQMF | |
| | CATGCCTTCTGCATGATGGAGTCCACTGTGCTACTGGCCATGGCCTTTG | | FIHAFCMMESTVLLAM | |
| | ATCGCTACGTGGCCATCTGCCACCCACTCCGCTATGCCACAATCCTCAC | | AFDRYVAICHPLRYAT | |
| | TGACACCATCATTGCCCACATAGGGGTGGCAGCTGTAGTGCGAGGCTCC | | ILTDTIIAHIGVAAVV | |
| | CTGCTCATGCTCCCATGTCCCTTCCTTATTGGGCGTTTGAACTTCTGCC | | RGSLLMLPCPFLIGRL | |
| | AAAGCCATGTGATCCTACACACGTACTGTGAGCACATGGCTGTGGTGAA | | NFCQSHVILHTYCEHM | |
| | GCTGGCCTGTGGAGACACCAGGCCTAACCGTGTGTATGGGCTGACAGCT | | AVVKLACGDTRPNRVY | |
| | GCACTGTTGGTCATTGGGGTTGACTTGTTTTGCATTGGTCTCTCCTATG | | GLTAALLVIGVDLFCI | |
| | CCCTAAGTGCACAAGCTGTCCTTCGCCTCTCATCCCATGAAGCTCGGTC | | GLSYALSAQAVLRLSS | |
| | CAAGGCCCTAGGGACCTGTGGTTCCCATGTCTGTGTCATCCTCATCTCT | | HEARSKALGTCGSHVC | |
| | TATACACCAGCCCTCTTCTCCTTTTTTACACACCGCTTTGGCCATCACG | | VILISYTPALFSFFTH | |
| | TTCCAGTCCATATTCACATTCTTTTGGCCAATGTTTATCTGCTTTTGCC | | RFGHHVPVHIHILLAN | |
| | ACCTGCTCTTAATCCTGTGGTATATGGAGTTAAGACCAAACAGATCCGT | | VYLLLPPALNPVVYGV | |
| | AAAAGAGTTGTCAGGGTGTTTCAAAGTGGGCAGGGAATGGGCATCAAGG | | KTKQIRKRVVRVFQSG | |
| | CATCTGAGTGACTCTGGA | | QGMGIKASE | |
| GMAP | CCATGCAGAGGAGCAATCACACAGTGACTGAGTTCATCCTGCTGGGCTT | 39 | MQRSNHTVTEFILLGF | 40 |
| 002418_D | CACCACAGATCCAGGGATGCAACTGGGCCTCTTTGTGGTGTTCCTGGGT | | TTDPGMQLGLFVVFLG | |
| | GTGTACTGTCTGACTGTGGTAGGAAGTAGCACCCTCATCGTGTTGATCT | | VYCLTVVGSSTLIVLI | |
| | GTAATGACTCCCGCCTACACACACCCATGTATTTTGTCATTGGAAATCT | | CNDSRLHTPMYFVIGN | |
| | GTCATTTCTGGATCTCTGGTATTCTTCTGTCCACACCCCAAAGATCCTA | | LSFLDLWYSSVHTPKI | |
| | GTGACCTGCATCTCTGAAGACAAAAGCATCTCCTTTGCTGGCTGCCTGT | | LVTCISEDKSISFAGC | |
| | GTCAGTTCTTCTCTGCCAGGCTGGCCTATAGTGAGTGCTACCTACTGGC | | LCQFFSARLAYSECYL | |
| | TGCCATGGCTTATGACCACTACGTGGCCATCTCCAAGCCCCTGCTTTAT | | LAAMAYDHYVAISKPL | |
| | GCTCAGACCATGCCAAGGAGATTGTGCATCTGTTTGGTTTTATATTCCT | | LYAQTMPRRLCICLVL | |
| | ATACTGGGGGTTTTGTCAATGCAATAATATTAACCAGCAACACATTCAC | | YSYTGGFVNAIILTSN | |
| | ATTGGATTTTTGTGGTGACAATGTCATTGATGACTTTTTCTGTGATGTT | | TFTLDFCGDNVIDDFF | |
| | CCACCCCTCGTGAAGCTGGCATGCAGTGTGAGAGAGAGCTACCAGGCTG | | CDVPPLVKLACSVRES | |
| | TGCTGCACTTCCTTCTGGCCTCCAATGTCATCTCCCCTACTGTGCTCAT | | YQAVLHFLLASNVISP | |
| | CCTTGCCTCTTACCTCTCCATCATCACCACCATCCTGAGGATCCACTCT | | TVLILASYLSIITTIL | |
| | ACCCAGGGCCGCATCAAAGTCTTCTCCACATGCTCCTCCCACCTGATCT | | RIHSTQGRIKVFSTCS | |
| | CCGTTACCTTATACTATGGCTCCATTCTCTACAACTACTCCCGGCCAAG | | SHLISVTLYYGSILYN | |
| | TTCCAGCTACTCCCTCAAGAGGGACAAAATGGTTTCTACCTTTTATACT | | YSRPSSSYSLKRDKMV | |
| | ATGCTGTTCCCCATGTTGAATCCCATGATCTACAGTCTGAGGAGTAAAG | | STFYTMLFPMLNPMIY | |
| | ACATGAAAGACGCTCTGAAAAAATTCTTCAAGTCAGCATAATCCAAA | | SLRSKDMKDALKKFFK | |
| | | | SA | |
| GMAL | CCATGGGGAACCACACCACCGTCACCGAGTTTGTCCTGCTGGGGCTCTC | 41 | MGNHTTVTEFVLLGLS | 42 |
| 160314_B | AGAGACCTGTGAGCTGCAGATGCTCATCTTCCTGGGGCTCCTCCTGACC | | ETCELQMLIFLGLLLT | |
| | TACCTCCTCACACTGCTGGGGAATCTGGTCATCGTGGTCATCACCCTCA | | YLLTLLGNLVIVVITL | |
| | TGGACAGGCGCCTCCACACCACCATGTACTACTTCCTCCGCAACTTTGC | | MDRRLHTTMYYFLRNF | |
| | TGTCCCGGAGATCTGGTTCACCTCGGTCATCTTTCCCAAGGTGCTGGCC | | AVPEIWFTSVIFPKVL | |
| | AACATCCTCACAGGATACAAGCAAGACCATTCCCTCCCAGGCTGCTTCC | | ANILTGYKQDHSLPGC | |
| | TGCAAAGTTTGCTCTATTTTTTCTTGGGCACCACAGAGTTCTTCCTCCT | | FLQSLLYFFLGTTEFF | |
| | GGCGGTGATGTCCTTTGACAGGTACGTGGCCGTATGTAACCCTTTGCAT | | LLAVMSFDRYVAVCNP | |
| | TATGCCACCATCATGAGCAAAAGGGTCTGTGTCCAGCTAGTCCTCTGTT | | LHYATIMSKRVCVQLV | |
| | GGTGGATGACAGGATTCCTTCTCATCATTATTCCAAGTTTTCTTGTCCT | | LCWWMTGFLLIIIPSF | |
| | TCAGCAGCCATTCTGTGGCCCCAACATCATTAACCATTTCTTCTGTGAC | | LVLQQPFCGPNIINHF | |
| | AACTTTCCCCTCTTGAAACTCATTTGTGCAGACATGACTCTGATAGAGC | | FCDNFPLLKLICADMT | |
| | TCCTGGGTTTTGTTATAGCCAACGTCAGCTTACTGGGCACTCTGTCTAT | | LIELLGFVIANVSLLG | |
| | GACGGCCACTTGCTATGGCCACATCCTCCACGCCATTCTGCACATCCCC | | TLSMTATCYGHILHAI | |
| | TCAGCCAAAGAGAAGCAGAAAGCCTTCTCCGCCTGCTCCTCCCACATCA | | LHIPSAKEKQKAFSAC | |
| | TTGTCGTGTCTCTCTTCTATGGCAGCTGCATCTTCATGTACATTCAGTC | | SSHIIVVSLFYGSCIF | |
| | AGGCAAGAGTGACCAGAAGGAAGACAGGAACAAGGTGGCGGCATTGCTT | | MYIQSGKSDQKEDRNK | |
| | AACACCGTGGTGACCCTGATGCTCAACCCCTTCATCTACACCCTGAGGA | | VAALLNTVVTLMLNPF | |
| | ACAAACAGGTGAAACAGGTGTTTAGGCAGCAGGTGAGCAAACTCCTCAT | | IYTLRNKQVKQVFRQQ | |
| | ATAAAGCT | | VSKLLI | |
| GMAP | ATGAAGAATAAAAGGAATGTGACTGAATTCGTTTTAACAGGTCTTACAC | 43 | MKNKRNVTEFVLTGLT | 44 |
| 002509_A | AGAACCCTAAAATGGAGAAAGTCATGTTTGCAGTATTTTTGGTTCTTTA | | QNPKMEKVMFAVFLVL | |
| | CATGATAACACTTTCAGGCAACCTGCTCCTTGTGGTTACAATTACCACC | | YMITLSGNLLLVVTIT | |
| | AGCCAGGCTCTTAGCTCCCCCATGTACTTCTTCCTGAGCCACCTTTCTT | | TSQALSSPMYFFLSHL | |
| | TGATAGACACAGTTTATTCTTCTTCTTCAGCTCCTAAGTTGATTGTCGA | | SLIDTVYSSSSAPKLI | |
| | TTCCCTTCATGAGAAGAAAATCATCTCCTTTAATGGGTGTATGGCTCAA | | VDSLHEKKIISFNGCM | |
| | GCCTATGAAGAACACATTTTTGGTGCTACTGAGATCATCCTGCTGACAG | | AQAYEEHIFGATEIIL | |
| | TGATGGCCTGTGACAACTATGTGGCCATCTGCAAACCTCTGCACTACAC | | LTVMACDNYVAICKPL | |
| | AACCATCATGAGCCACAGCCTGTGCATTCTCCTAGTGGTAGTGGCCTGG | | HYTTIMSHSLCILLVV | |
| | ATAGGAGGATTTCTCCATGCAAATATTCAGATTCTATTTACAGTATGGC | | VAWIGGFLHANIQILF | |
| | TGCCCTTCTGTGGCCCCAATGTCATAGACCACTTCATGTGTGACTTGTG | | TVWLPFCGPNVIDHFM | |
| | CCCTTTGTTAAAACTTGTTTGCCTGGACACTCATACCCTTGGTCTCTTT | | CDLCPLLKLVCLDTHT | |
| | GTTGCTGCCAACAGTGGGTTCATCTGCTTATTAAACTTCCTTCTCTGGG | | LGLFVAANSGFICLLN | |
| | TGGTATCCTATGTGATCATCTTGAGATGTTTAAAGAACTATATCTTGGA | | FLLWVVSYVIILRCLK | |
| | GGGGAGGGGTAAAGCCCTCTCCACCTGTATTTCTCACATCATAATAGTT | | NYILEGRGKALSTCIS | |
| | GTCTTATTCTTTGTGCCTTGTATATTTGTGTATCTGCACCCAGTGACAA | | HIIIVVLFFVPCIFVY | |
| | ACTCTGCCGATAAAGCTGCTGCTGTATTTTATACTATGGTGGTCCCAAT | | LHPVTNSADKAAAVFY | |
| | GTTAAATCCTTTGATCTACACACTCAGAAATGCTGAGGTAAAAAGTGCA | | TMVVPMLNPLIYTLRN | |
| | ATAAGGAAGCTTTGGAGAAAAAAAGTTATTTCAGATAATGACTAAATAA | | AEVKSAIRKLWRKKVI | |
| | | | SDND | |
| GMAL | AAAACAAAACTGTTTGTATAAATGGTGTTTTCTGTCATCTCTACAGCCC | 45 | MVFSVISTALEFTNNS | 46 |
| 356019_F | TGGAATTCACAAACAATTCAGAGACAAGCACTATGACGGAATTTGTTCT | | ETSTMTEFVLLGFPGC | |
| | CCTTGGCTTTCCTGGTTGTCAGGAGATGCAAAGTTTCCTCTTCTCCCTG | | QEMQSFLFSLFFVIYV | |
| | TTCTTTGTGATCTATGTATTTACCATAATAGGAAATGGGACCATTGTCT | | FTIIGNGTIVCAVRLD | |
| | GTGCTGTGAGATTGGACAAACGGCTTCATACCCCAATGTATATTCTCCT | | KRLHTPMYILLGNFAF | |
| | AGGGAACTTTGCTTTCCTTGAAATCCGGGAAGTTACTTCCACTGTACCC | | LEIREVTSTVPNMLVN | |
| | AACATGCTAGTCAACTTCCTCTCAGAGACAAAAACCATCTCTTTTGTTG | | FLSETKTISFVGCFLQ | |
| | GCTGTTTCCTCCAGTTCTACTTTTTTACTTCCCTTGGTACAATAGAAGC | | FYFFTSLGTIEAYFLC | |
| | ATACTTCCTCTGCATCATGGCATATGATCGGTACCTTGCTATCTGCCGC | | IMAYDRYLAICRPLHY | |
| | CCATTGCACTACCCAACCATCATGACCCCACAACTCTGCTACATATTGA | | PTIMTPQLCYILMSFC | |
| | TGTCTTTTTGCTGGGTGTTTGGATTCCTCAGTTACTCTGTCTCCACTGT | | WVFGFLSYSVSTVQLS | |
| | GCAACTGTCTCAACTGCCTTTCTGTGGGCCCAACATCATCAATCACTTT | | QLPFCGPNIINHFLCD | |
| | TTGTGTGACATGGACCCACTGATGGCTCTGTCCTGTGCCTCAGCTCCTA | | MDPLMALSCASAPITE | |
| | TCACTGAGATTATCTTCTATATCCTGAGCTCCCTCATTATCATTCTCAC | | IIFYILSSLIIILTLL | |
| | TCTTCTGTACATCTGTGGCTCCTATATGCTTTACCTGATAGCTGTATTA | | YICGSYMLYLIAVLKV | |
| | AAAGTCCCTTCAGCAGCTGGCCAGCAGAAGGCCTTTTCCACCTGTGGAT | | PSAAGQQKAFSTCGSH | |
| | CTCATCTGACAGTGGTGTGTTTATTCTTTGGGGCCCTACTGGCAATGTA | | LTVVCLFFGALLAMYV | |
| | TGTGAGCCCCACAACTGATAACCCAGCTGCAATTTAGAAGATTATAACT | | SPTTDNPAAI | |
| | TTGTTCTATTCTGTGGTGACCCCCTTCTTAAACCCCCTGATTTACAGCT | | | |
| | TACGAAACAAAGAGATGAAGGCTGCGTTGAAGAAAGTCCTGAGGATAGA | | | |
| | ATGAGAATAAA | | | |
| GMAC | ATGGAGACTGAAAACAATACAACAGTGACAGAGTTCATTATTTTGGGAT | 47 | METENNTTVTEFIILG | 48 |
| 022998_A | TAACAGACAATCCTATGCTATGTGCCATTTTCTTCGTGTTTTTTCTAGC | | LTDNPMLCAIFFVFFL | |
| | AGTTTATATAGTTACTATACCGGGAAATATTAGCATAATCCTCTTAATC | | AVYIVTIPGNISIILL | |
| | CAAAGCAGCCCACAGCTTCACACGCTAATGTACCTTTTTCTCAGCCATT | | IQSSPQLHTLMYLFLS | |
| | TGGCTTCTGTGGACATTGGGTATTCCATATCAGTTACGCCAATCATTCT | | HLASVDIGYSISVTPI | |
| | CATCAATTTCTTAAGAGAGAAAACGACTATTCCTGTCACAGGCTGTATA | | ILINFLREKTTIPVTG | |
| | GCACAGCTTGGCTCTGATGTCATGTTTGGAACCACAGAGTGCTTCCTGC | | CIAQLGSDVMFGTTEC | |
| | TGGTCACTATGATGGCTATCTGCTCTCCCCTGCTTTACTCCATCCAAAT | | FLLVTMMAICSPLLYS | |
| | GCCCCCAGTCGTCTGCTTCCTCCTACTGGGAGCCTCCTACCTGGGTGGA | | IQMPPVVCFLLLGASY | |
| | TGCCTGAACGCTTCGTCTTTTACAGGCTGTTTGATGAACCTGTCCTTCT | | LGGCLNASSFTGCLMN | |
| | GCGGTCCAAATAAAATCAACCACTTTTTCTGTGACCTCTTCCCACTCTT | | LSFCGPNKINHFFCDL | |
| | GAAGCTTTCTTGTGGCCATGTTTACATTGCTGAAATATCCCCTGCCATC | | FPLLKLSCGHVYIAEI | |
| | TCCTGTGCATCTGTCCTTATCAGCACGCTGTTTACCATAATCGTGTCCT | | SPAISCASVLISTLFT | |
| | ACATCTACATCCTTCACTCCATCCTGAAGGTGTGCTCTACTGAGGGAAG | | IIVSYIYILHSILKVC | |
| | GAAGAAGGCTTTCTCCACCTGCGCTTCCCACCTCACTGCAGTCACTTTG | | STEGRKKAFSTCASHL | |
| | TTCTATGGGACCATTTTGTTTGTTTATGTGATGCCCAAGTCAAGCTATT | | TAVTLFYGTILFVYVM | |
| | CAGCGGATCAGGTCAAGGTGGCATTTGTGATCTACACGGTGGTGATTCC | | PKSSYSADQVKVAFVI | |
| | CATGCTGAACCCCCTCATCTACAGTCTCAGGAATAAGGAGGTGAAAGAG | | YTVVIPMLNPLIYSLR | |
| | GCCATGAGAAAATTGATGGCAAGAACACATTGGTTTTCCTGAATTAAAT | | NKEVKEAMRKLMARTH | |
| | CA | | WFS | |
| GMAC | ATGAATTTCCAAACTCTGACATGGCTCCTGAAAATTTCACCAGGGTCAC | 49 | MAPENFTRVTEFILTG | 50 |
| 022882_G | TGAGTTTATTCTTACAGGTGTCTCTAGCTGTCCAGAGCTCCAGATTCCC | | VSSCPELQIPLFLVFL | |
| | CTCTTCCTGGTCTTTCTGGTGCTCTATGGGCTGACCATGGCAGGGAACC | | VLYGLTMAGNLGIITL | |
| | TGGGCATCATCACCCTCACCAGTGTTGACTCTCGACTTCAAACCCCCAT | | TSVDSRLQTPMYFFLQ | |
| | GTACTTTTTCCTGCAACATCTGGCTCTCATTAATCTTGGTAACTCTACT | | HLALINLGNSTVIAPK | |
| | GTCATTGCCCCTAAAATGCTGATTAACTTTTTAGTAAAGAAGAAAACTA | | MLINFLVKKKTTSFYE | |
| | CCTCATTCTATGAATGTGCCACCCAACTGGGAGGGTTCTTGTTCTTTAT | | CATQLGGFLFFIVSEV | |
| | TGTATCGGAGGTAATCATGCTGGCTTTGATGGCCTGTGACCGCTATGTG | | IMLALMACDRYVAICN | |
| | GCTATTTGTAACCCTCTGCTGTACATGGTGGTGGTGTCTCGGCGGCTCT | | PLLYMVVVSRRLCLLL | |
| | GCCTCCTGCTGGTCTCCCTCACATACCTCTATGGCTTTTCTACAGCTAT | | VSLTYLYGFSTAIVVS | |
| | TGTGGTTTCATCTTATGTATTCTCTGTGTCTTATTGCTCTTCTAATATA | | SYVFSVSYCSSNIINH | |
| | ATCAATCATTTTTACTGTGATAATGTTCCTCTGTTAGCATTATCTTGCT | | FYCDNVPLLALSCSDT | |
| | CTGATACTTACTTACCAGAAACAGTTGTCTTTATATCTGCAGCAACAAA | | YLPETVVFISAATNVV | |
| | TGTGGTTGGTTCCTTGATTATAGTTCTAGTATCTTATTTCAATATTGTT | | GSLIIVLVSYFNIVLS | |
| | TTGTCTATTTTAAAAATATGTTCATCAGAAGGAAGGAAAAAAGCCTTTT | | ILKICSSEGRKKAFST | |
| | CTACCTGTGCTTCACATATGATGGCAGTCACAATTTTTTATGGGACATT | | CASHMMAVTIFYGTLL | |
| | GCTATTCATGTATGTGCAGCCCCGAAGTAACCATTCATTGGATACTGAT | | FMYVQPRSNHSLDTDD | |
| | GATAAGATGGCTTCTGTGTTTTACACGTTGGTAATTCCTATGCTGAATC | | KMASVFYTLVIPMLNP | |
| | CCTTGATCTACAGCCTGAGGAATAAGGATGTGAAGACTGCTCTACAGAG | | LIYSLRNKDVKTALQR | |
| | ATTCATGACAAATCTGTGCTATTCCTTTAAAACAATGTAATTTTAAACA | | FMTNLCYSFKTM | |
| CG | ATGAATTTCCAAACTCTGACATGGCTCCTGAAAATTTCACCAGGGTCAC | 51 | MAPENFTRVTEFILTG | 52 |
| 56113-01 | TGAGTTTATTCTTACAGGTGTCTCTAGCTGTCCAGAGCTCCAGATTCCC | | VSSCPELQIPLFLVFL | |
| | CTCTTCCTGGTCTTTCTGGTGCTCTATGGGCTGACCATGGCAGGGAACC | | VLYGLTMAGNLGIITL | |
| | TGGGCATCATCACCCTCACCAGTGTTGACTCTCGACTTCAAACCCCCAT | | TSVDSRLQTPMYFFLQ | |
| | GTACTTTTTCCTGCAACATCTGGCTCTCATTAATCTTGGTAACTCTACT | | HLALINLGNSTVIAPK | |
| | GTCATTGCCCCTAAAATGCTGATTAACTTTTTAGTAAAGAAGAAAACTA | | MLINFLVKKKTTSFYE | |
| | CCTCATTCTATGAATGTGCCACCCAACTGGGAGGGTTCTTGTTCTTTAT | | CATQLGGFLFFIVSEV | |
| | TGTATCGGAGGTAATCATGCTGGCTTTGATGGCCTGTGACCGCTATGTG | | IMLALMACDRYVAICN | |
| | GCTATTTGTAACCCTCTGCTGTACATGGTGGTGGTGTCTCGGCGGCTCT | | PLLYMVVVSRRLCLLL | |
| | GCCTCCTGCTGGTCTCCCTCACATACCTCTATGGCTTTTCTACAGCTAT | | VSLTYLYGFSTAIVVS | |
| | TGTGGTTTCATCTTATGTATTCTCTGTGTCTTATTGCTCTTCTAATATA | | SYVFSVSYCSSNIINH | |
| | ATCAATCATTTTTACTGTGATAATGTTCCTCTGTTAGCATTATCTTGCT | | FYCDNVPLLALSCSDT | |
| | CTGATACTTACTTACCAGAAACAGTTGTCTTTATATCTGCAGCAACAAA | | YLPETVVFISAATNVV | |
| | TGTGGTTGGTTCCTTGATTATAGTTCTAGTATCTTATTTCAATATTGTT | | GSLIIVLVSYFNIVLS | |
| | TTGTCTATTTTAAAAATATGTTCATCAGAAGGAAGGAAAAAAGCCTTTT | | ILKICSSEGRKKAFST | |
| | CTACCTGTGCTTCACATATGATGGCAGTCACAATTTTTTATGGGACATT | | CASHMMAVTIFYGTLL | |
| | GCTATTCATGTATGTGCAGCCCCGAAGTAACCATTCATTGGATACTGAT | | FMYVQPRSNHSLDTDD | |
| | GATAAGATGGCTTCTGTGTTTTACACGTTGGTAATTCCTATGCTGAATC | | KMASVFYTLVIPMLNP | |
| | CCTTGATCTACAGCCTGAGGAATAAGGATGTGAAGACTGCTCTACAGAG | | LIYSLRNKDVKTALQR | |
| | ATTCATGACAAATCTGTGCTATTCCTTTAAAACAATGTAATTTTAAACA | | FMTNLCYSFKTM | |
| SC | CTATGGAGCAGAGCAATTATTCCGTGTATGCCGACTTTATCCTTCTGGG | 53 | MEQSNYSVYADFILLG | 54 |
| 13491216 | TTTGTTCAGCAACGCCCGTTTCCCCTGGCTTCTCTTTGCCCTCATTCTC | | LFSNARFPWLLFALIL | |
| 7_A | CTGGTCTTTGTGACCTCCATAGCCAGCAACGTGGTCAAGATCATTCTCA | | LVFVTSIASNVVKIIL | |
| | TCCACATAGACTCCCGCCTCCACACCCCCATGTACTTCCTGCTCAGCCA | | IHIDSRLHTPMYFLLS | |
| | GCTCTCCCTCAGGGACATCTTGTATATTTCCACCATTGTGCCCAAAATG | | QLSLRDILYISTIVPK | |
| | CTGGTCGACCAGGTGATGAGCCAGAGAGCCATTTCCTTTGCAGGATGCA | | MLVDQVMSQRAISFAG | |
| | CTGCCCAACACTTCCTCTACTTGACCTTAGCAGGGGCTGAGTTCTTCCT | | CTAQHFLYLTLAGAEF | |
| | CCTAGGACTCATGTCCTGTGATCGCTACGTAGCCATCTGCAACCCTCTG | | FLLGLMSCDRYVAICN | |
| | CACTATCCTGACCTCATGAGCCGCAAGATCTGCTGGTTGATTGTGGCGG | | PLHYPDLMSRKICWLI | |
| | CAGCCTGGCTGGGAGGGTCTATCGATGGTTTCTTGCTCACCCCCGTCAC | | VAAAWLGGSIDGFLLT | |
| | CATGCAGTTCCCCTTCTGTGCCTCTCGGGAGATCAACCACTTCTTCTGC | | PVTMQFPFCASREINH | |
| | GAGGTGCCTGCCCTTCTGAAGCTCTCCTGCACGGACACATCAGCCTACG | | FFCEVPALLKLSCTDT | |
| | AGACAGCCATGTATGTCTGCTGTATTATGATGCTCCTCATCCCTTTCTC | | SAYETAMYVCCIMMLL | |
| | TGTGATCTCGGGCTCTTACACAAGAATTCTCATTACTGTTTATAGGATG | | IPFSVISGSYTRILIT | |
| | AGCGAGGCAGAGGGGAGGCGAAAGGCTGTGGCCACCTGCTCCTCACACA | | VYRMSEAEGRRKAVAT | |
| | TGGTGGTTGTCAGCCTCTTCTATGGGGCTGCCATGTACACATACGTGCT | | CSSHMVVVSLFYGAAM | |
| | GCCTCATTCTTACCACACCCCTGAGCAGGACAAAGCTGTATCTGCCTTC | | YTYVLPHSYHTPEQDK | |
| | TACACCATCCTCACTCCCATGCTCAATCCACTCATTTACAGCCTTAGGA | | AVSAFYTILTPMLNPL | |
| | ACAAGGATGTCACGGGGGCCCTACAGAAGGTTGTTGGGAGGTGTGTGTC | | IYSLRNKDVTGALQKV | |
| | CTCAGGAAAGGTAACCACTTTCTAAAC | | VGRCVSSGKVTTF | |
| SC | GTGAAACCTCATGGCCAACATCACCTGGATGGCCAACCACACTGGAAAG | 55 | MANITWMANHTGKLDF | 56 |
| 13501109 | TTGGATTTCATCCTCATGGGACTCTTCAGACGATCCAAACATCCAGCTC | | ILMGLFRRSKHPALLS | |
| 8_A_ | TACTTAGTGTGGTCATCTTTGTGGTTTTCCTGATGGCGTTGTCTGAAAA | | VVIFVVFLMALSENAV | |
| | TGCTGTCCTGATCCTTCTGATACACTGTGACACCTACCTCCACACCCCC | | LILLIHCDTYLHTPMY | |
| | ATGTACTTTTTCATCAGTCAATTGTCTCTCATGGACATGGCGTACATTT | | FFISQLSLMDMAYISV | |
| | CTGTCACTGTGCCCAAGATGCTCCTGGACCAGGTCATGGGTGTGAATAA | | TVPKMLLDQVMGVNKI | |
| | GATCTCAGCCCCTGAGTGTGGGATGCAGATGTTCCTCTATCTGACACTA | | SAPECGMQMFLYLTLA | |
| | GCAGGTTCGGAATTTTTCCTTCTAGCCACCATGGCCTATGACCGCTACG | | GSEFFLLATMAYDRYV | |
| | TGGCCATCTGCCATCCTCTCCGTTACCCTGTCCTCATGAACCATAGGGT | | AICHPLRYPVLMNHRV | |
| | CTGTCTTTTCCTGGCATCGGGCTGCTGGTTCCTGGGCTCAGTGGATGGC | | CLFLASGCWFLGSVDG | |
| | TTCATGCTCACTCCCATCACCATGAGCTTCCCCTTCTGCAGATCCTCGG | | FMLTPITMSFPFCRSW | |
| | AGATTCATCATTTCTTCTGTGAAGTCCCTGCTGTAACGATCCTGTCCTG | | EIHHFFCEVPAVTILS | |
| | CTCAGACACCTCACTCTATGAGACCCTCATGTACCTATGCTGTGTCCTC | | CSDTSLYETLMYLCCV | |
| | ATGCTCCTCATCCCTGTGACGATCATTTCAAGCTCCTATTTACTCATCC | | LMLLIPVTIISSSYLL | |
| | TCCTCACCATCCACAGGATGAACTCAGCAGAGGGCCGGAAAAAGGCCTT | | ILLTIHRMNSAEGRKK | |
| | TGCCACCTGCTCCTCCCACCTGACTGTGGTCATCCTCTTCTATGGGGCT | | AFATCSSHLTVVILFY | |
| | GCCGTCTACACCTACATGCTCCCCAGCTCCTACCACACCCCTGAGAAGG | | GAAVYTYMLPSSYHTP | |
| | ACATGATGGTATCTGTCTTCTATACCATCCTCACTCCGGTGCTGAACCC | | EKDMMVSVFYTILTPV | |
| | TTTAATCTATAGTCTTAGGAATAAGGATGTCATGGGGGCTCTGAAGAAA | | LNPLIYSLRNKDVMGA | |
| | ATGTTAACTGTGAGATTCGTCCTTTAGGAAAT | | LKKMLTVRFVL | |
| GMAC | CTACATGGAAGCAGAAAACCTTACAGAATTATCAAAATTTCTCCTCCTG | 57 | MEAENLTELSKFLLLG | 58 |
| 006271_A | GGACTCTCAGATGATCCTGAACTGCAGCCCGTCCTCTTTGGGCTGTTCC | | LSDDPELQPVLFGLFL | |
| | TGTCCATGTACCTGGTCACGGTGCTGGGGAACCTGCTCATCATTCTGGC | | SMYLVTVLGNLLIILA | |
| | CGTCAGCTCTGACTCCCACCTCCACACCCCCATGTACTTCTTCCTCTCC | | VSSDSHLHTPMYFFLS | |
| | AACCTGTCCTTTGTTGACATCTGTTTCATCTCCACCACAGTCCCCAAGA | | NLSFVDICFISTTVPK | |
| | TGCTAGTGAGCATCCAGGCACGGAGCAAAGACATCTCCTACATGGGGTG | | MLVSIQARSKDISYMG | |
| | CCTCACTCAGGTGTATTTTTTAATGATGTTTGCTGGAATGGATACTTTC | | CLTQVYFLMMFAGMDT | |
| | CTACTGGCCGTGATGGCCTATGACCGGTTTGTGGCCATCTGCCACCCAC | | FLLAVMAYDRFVAICH | |
| | TGCACTACACGGTCATCATGAACCCCTGCCTCTGTGGCCTCCTGGTTCT | | PLHYTVIMNPCLCGLL | |
| | GGCATCTTGGTTCATCATTTTCTGGTTCTCCCTGGTTCATATTCTACTG | | VLASWFIIFWFSLVHI | |
| | ATGAAGAGGTTGACCTTCTCCACAGGCACTGAGATTCCGCATTTCTTCT | | LLMKRLTFSTGTEIPH | |
| | GTGAACCGGCTCAGGTCCTCAAGGTGGCCTGCTCTAACACCCTCCTCAA | | FFCEPAQVLKVACSNT | |
| | TAACATTGTCTTGTATGTGGCCACGGCACTGCTGGGTGTGTTTCCTGTA | | LLNNIVLYVATALLGV | |
| | GCTGGGATCCTCTTCTCCTACTCTCAGATTGTCTCCTCCTTAATGGGAA | | FPVAGILFSYSQIVSS | |
| | TGTCCTCCACCAAGGGCAAGTACAAAGCCTTTTCCACCTGTGGATCTCA | | LMGMSSTKGKYKAFST | |
| | CCTCTGTGTGGTCTCCTTGTTCTATGGAACAGGACTTGGGGTCTATCTG | | CGSHLCVVSLFYGTGL | |
| | AGTTCTGCTGTGACCCATTCTTCCCAGAGCAGCTCCACCGCCTCACTGA | | GVYLSSAVTHSSQSSS | |
| | TGTACGCCATGGTCACCCCCATGCTGAACCCCTTCATCTACAGCCTGAG | | TASVMYAMVTPMLNPF | |
| | GAACAAGGATGTGAAGGGGGCCCTGGAAAGACTCCTCAGCAGGGCCGAC | | IYSLRNKDVKGALERL | |
| | TCTTGTCCATGACAAATCAGGGCCTCAGAACTAAGAGGACACACTGCGT | | LSRADSCP | |
| | ACCCCTAAGGCAAAT | | | |
| GMAL | TTGATGATATGGAAAGAGCAAACCATTCAGTGGTATCGGAATTTATTTT | 59 | MERANHSVVSEFILLG | 60 |
| 163152_A | GTTGGGACTTTCCAAATCTCAAAATCTTCAGATTTTATTCTTCTTGGGA | | LSKSQNLQILFFLGFS | |
| | TTCTCTGTGGTCTTCGTGGGGATTGTGTTAGGAAACCTGCTCATCTTGG | | VVFVGIVLGNLLILVT | |
| | TGACTGTGACCTTTGATTCGCTCCTTCACACACCAATGTATTTTCTGCT | | VTFDSLLHTPMYFLLS | |
| | TAGCAACCTCTCCTGCATTGATATGATCCTGGCTTCTTTTGCTACCCCT | | NLSCIDMILASFATPK | |
| | AAGATGATTGTAGATTTCCTCCGAGAACGTAAGACCATCTCATGGTGGG | | MIVDFLRERKTISWWG | |
| | GATGTTATTCCCAGATGTTCTTTATGCACCTCCTGGGTGGGAGTGAGAT | | CYSQMFFMHLLGGSEM | |
| | GATGTTGCTTGTAGCCATGGCAATAGACAGGTATGTTGCCATATGCAAA | | MLLVAMAIDRYVAICK | |
| | CCCCTCCATTACATGACCATCATGAGCCCACGGGTGCTCACTGGGCTAC | | PLHYMTIMSPRVLTGL | |
| | TGTTATCCTCCTATGCAGTTGGATTTGTGCACTCATCTAGTCAAATGGC | | LLSSYAVGFVHSSSQM | |
| | TTTCATGTTGACTTTGCCCTTCTGTGGTCCCAATGTTATAGACAGCTTT | | AFMLTLPFCGPNVIDS | |
| | TTCTGTGACCTTCCCCTTGTGATTAAACTTGCCTGCAAGGACACCTACA | | FFCDLPLVIKLACKDT | |
| | TCCTACAGCTCCTGGTCATTGCTGACAGTGGGCTCCTGTCACTGGTCTG | | YILQLLVIADSGLLSL | |
| | CTTCCTCCTCTTGCTTGTCTCCTATGGAGTCATAATATTCTCAGTTAGG | | VCFLLLLVSYGVIIFS | |
| | TACCGTGCTGCTAGTCGATCCTCTAAGGCTTTCTCCACTCTCTCAGCTC | | VRYRAASRSSKAFSTL | |
| | ACATCACAGTTGTGACTCTGTTCTTTGCTCCGTGTGTCTTTATCTACGT | | SAHITVVTLFFAPCVF | |
| | CTGGCCCTTCAGCAGATACTCGGTAGATAAAATTCTTTCTGTGTTTTAC | | IYVWPFSRYSVDKILS | |
| | ACAATTTTCACACCTCTCTTAAATCCTATTATTTATACATTAAGAAATC | | VFYTIFTPLLNPIIYT | |
| | AAGAGGTAAAAGCAGCCATTAAAAAAAGACTCTGCATATAAATTTAAAG | | LRNQEVKAAIKKRLCI | |
| | CATACTTTTTAGATGAGACTTTTGAAGAGACA | | | |
| GMAL | TTGACGATATGGAAAGAGCAAACCATTCAGTGGTATCGGAATTTATTTT | 61 | MERANHSVVSEFILLG | 62 |
| 359218_C | GTTGGGACTTTCCAAATCTCAAAATCTTCAGATTTTATTCTTCTTGGGA | | LSKSQNLQILFFLGFS | |
| | TTCTCTGTGGTCTTCGTGGGGATTGTGTTAGGAAACCTGCTCATCTTGG | | VVFVGIVLGNLLILVT | |
| | TGACTGTGACCTTTGATTCGCTCCTTCACACACCAATGTATTTTCTGCT | | VTFDSLLHTPMYFLLS | |
| | TAGCAACCTCTCCTGCATTGATATGATCCTGGCTTCTTTTGCTACCCCT | | NLSCIDMILASFATPK | |
| | AAGATGATTGTAGATTTCCTCCGAGAACGTAAGACCATCTCATGGTGGG | | MIVDFLRERKTISWWG | |
| | GATGTTATTCCCAGATGTTCTTTATGCACCTCCTGGGTGGGAGTGAGAT | | CYSQMFFMHLLGGSEM | |
| | GATGTTGCTTGTAGCCATGGCAATAGACAGGTATGTTGCCATATGCAAA | | MLLVAMAIDRYVAICK | |
| | CCCCTCCATTACATGACCATCATGAGCCCACGGGTGCTCACTGGGCTAC | | PLHYMTIMSPRVLTGL | |
| | TGTTATCCTCCTATGCAGTTGGATTTGTGCACTCATCTAGTCAAATGGC | | LLSSYAVGFVHSSSQM | |
| | TTTCATGTTGACTTTGCCCTTCTGTGGTCCCAATGTTATAGACAGCTTT | | AFMLTLPFCGPNVIDS | |
| | TTCTGTGACCTTCCCCTTGTGATTAAACTTGCCTGCAAGGACACCTACA | | FFCDLPLVIKLACKDT | |
| | TCCTACAGCTCCTGGTCATTGCTGACAGTGGGCTCCTGTCACTGGTCTG | | YILQLLVIADSGLLSL | |
| | CTTCCTCCTCTTGCTTGTCTCCTATGGAGTCATAATATTCTCAGTTAGG | | VCFLLLLVSYGVIIFS | |
| | TACCGTGCTGCTAGTCGATCCTCTAAGGCTTTCTCCACTCTCTCAGCTC | | VRYRAASRSSKAFSTL | |
| | ACATCACAGTTGTGACTCTCTTCTTTGCTCCGTGTGTCTTTATCTACGT | | SAHITVVTLFFAPCVF | |
| | CTGGCCCTTCAGCAGATACTCGGTAGATAAAATTCTTTCTGTGTTTTAC | | IYVWPFSRYSVDKILS | |
| | ACAATTTTCACACCTCTCTTAAATCCTATTATTTATACATTAAGAAATC | | VFYTIFTPLLNPIIYT | |
| | AAGAGGTAAAAGCAGCCATTAAAAAAAGACTCTGCATATAAATTTAAAG | | LRNQEVKAAIKKRLCI | |
| | CATACTTTTTAGATGAGACTTTTGAAGACACTCTCTT | | | |
| GMAP | TCCCTTCCAAATTCAATGAATGAGACAAATCATTCTTGGGTGACAGAA | 63 | MNETNHSWVTEFVLLG | 64 |
| 001465_A | TTTGTGTTGCTGGGACTGTCTAGTTCAAGGGAGCTCCAACCTTTCTTGT | | LSSSRELQPFLFLIFS | |
| | TTCTTATATTTTCACTACTTTATCTAGCAATTCTGTTGGGCAACTTTCT | | LLYLAILLGNFLIILT | |
| | CATCATCCTCACTGTGACCTCAGATTCCCGCCTTCACACCCCCATGTAC | | VTSDSRLHTPMYFLLA | |
| | TTTCTGCTTGCCAACCTGTCATTTATAGACGTATGTGTTGCCTCTTCTG | | NLSFIDVCVASSATPK | |
| | CTACCCCTAAAATGATTGCAGACTTTCTGGTTGAGCACAAGACTATTTC | | MIADFLVEHKTISFDA | |
| | TTTTGATGCCCGCCTGGCCCAGATTTTCTTTGTTCATCTCTTCACTGGC | | RLAQIFFVLFTGSEM | |
| | AGTGAAATGGTGCTCCTAGTTTCCATGGCCTATGACCGTTATGTTGCTA | | VLLVSMAYDRYVAICK | |
| | TATGCAAACCTCCCCACTACATGACAATCATGAGCTGCTGTGTATGTGT | | PPHYMTIMSCCVCVVL | |
| | TGTGCTCTTCCTCATTTCCTGGTTTGTGGGCTTCATCCATACCACCAGC | | FLISWFVGFIHTTSQL | |
| | CAGTTGGCATTCACTGTTAATCTGCCATTTTGTGGTCCTAATAAGGTAG | | AFTVNLPFCGPNKVDS | |
| | ATAGTTTTTTCTGTGACCTTCCTCTAGTGACCAAGTTAGCCTGCATAGA | | FFCDLPLVTKLACIDT | |
| | CACTTATGTTGTCAGCCTACTAATAGTTGCAGATAGTGGCTTTCTTTCT | | YVVSLLIVADSGFLSL | |
| | CTGAGTTCCTTTCTCCTCTTGGTTGTCTCCTACACTGTAATACTTGTTA | | SSFLLLVVSYTVILVT | |
| | CAGTTAGGAATAGCTCCTCTGTAAGCATGGTGAAGGCCTGCTCCACATT | | VRNSSSVSMVKACSTL | |
| | GACTGCTCACATCACTGTGGTCACTTTATTCTTTGGACCGTGTATTTTC | | TAHITVVTLFFGPCIF | |
| | ATCTATGTGTGGCCCTTCAGCAGTTACTCAGTTGACAAAGTCCTTGCTG | | IYVWPFSSYSVDKVLA | |
| | TATTCTACACCATCTTCACGTCTATTTTAAACCCTGTAATCTACATGCT | | VFYTIFTSILNPVIYM | |
| | AAGAAACAAAGAAGTGAAGGCAGCTATGTCAAAACTGAAGAGTCGGTAT | | LRNKEVKAAMSKLKSR | |
| | CAGAAGCTTGGTCAGGTTTCTGTAGTCATAAGAAACGTTCTTTTCCTAG | | YQKLGQVSVVIRNVLF | |
| | AAACAAAGTAAACTTATGAGACT | | LETK | |
| GMAC | TTGGCTGGACCAATGGATGGAGAGAATCACTCAGTGGTATCTGAGTTTT | 65 | MDGENHSVVSEFLFLG | 66 |
| 004908_A | TGTTTCTGGGACTCACTCATTCATGGGAGATCCAGCTCCTCCTCCTAGT | | LTHSWEIQLLLLVFSS | |
| | GTTTTCCTCTGTGCTCTATGTGGCAAGCATTACTGGAAACATCCTCATT | | VLYVASITGNILIVFS | |
| | GTGTTTTCTGTGACCACTGACCCTCACTTACACTCCCCCATGTACTTTC | | VTTDPHLHSPMYFLLA | |
| | TACTGGCCAGTCTCTCCTTCATTGACTTAGGAGCCTGCTCTGTCACTTC | | SLSFIDLGACSVTSPK | |
| | TCCCAAGATGATTTATGACCTGTTCAGAAAGCGCAAAGTCATCTCCTTT | | MIYDLFRKRKVISFGG | |
| | GGAGGCTGCATCGCTCAAATCTTCTTCATCCACGTCATTGGTGGTGTGG | | CIAQIFFIHVIGGVEM | |
| | AGATGGTGCTGCTCATAGCCATGGCCTTTGACAGATATGTGGCCCTATG | | VLLIAMAFDRYVALCK | |
| | TAAGCCCCTCCACTATCTGACCATTATGAGCCCAAGAATGTGCCTTTCA | | PLHYLTIMSPRMCLSF | |
| | TTTCTGGCTGTTGCCTGGACCCTTGGTGTCAGTCACTCCCTGTTCCAAC | | LAVAWTLGVSHSLFQL | |
| | TGGCATTTCTTGTTAATTTAGCCTTCTGTGGCCCTAATGTGTTGGACAG | | AFLVNLAFCGPNVLDS | |
| | CTTCTACTGTGACCTTCCTCGGCTTCTCAGACTAGCCTGTACCGACACC | | FYCDLPRLLRLACTDT | |
| | TACAGATTGCAGTTCATGGTCACTGTTAACAGTGGGTTTATCTGTGTGG | | YRLQFMVTVNSGFICV | |
| | GTACTTTCTTCATACTTCTAATCTCCTACGTCTTCATCCTGTTTACTGT | | GTFFILLISYVFILFT | |
| | TTGGAAACATTCCTCAGGTGGTTCATCCAAGGCCCTTTCCACTCTTTCA | | VWKHSSGGSSKALSTL | |
| | GCTCACAGCACAGTGGTCCTTTTGTTCTTTGGTCCACCCATGTTTGTGT | | SAHSTVVLLFFGPPMF | |
| | ATACACGGCCACACCCTAATTCACAGATGGACAAGTTTCTGGCTATTTT | | VYTRPHPNSQMDKFLA | |
| | TGATGCAGTTCTCACTCCTTTTCTGAATCCAGTTGTCTATACATTCAGG | | IFDAVLTPFLNPVVYT | |
| | AATAAGGAGATGAAGGCAGCAATAAAGAGAGTATGCAAACAGCTAGTGA | | FRNKEMKAAIKRVCKQ | |
| | TTTACAAGAGGATCTCATAAATGATATAATAAGCCCTTCTC | | LVIYKRIS | |
| GMAC | ATCCTATGGAACCACAGAACACCACACAGGTATCAATGTTTGTCCTCTT | 67 | PMEPQNTTQVSMFVLL | 68 |
| 005962_A | AGGGTTTTCACAGACCCAAGAGCTCCAGAAATTCCTGTTCCTTCTGTTC | | GFSQTQELQKFLFLLF | |
| | CTGTTAGTCTATGTTACCACCATTGTGGGAAACCTCCTTATCATGGTCA | | LLVYVTTIVGNLLIMV | |
| | CAGTGACTTTTGACTGCCGGCTCCACACACCCATGTATTTTCTGCTCCG | | TVTFDCRLHTPMYFLL | |
| | AAATCTAGCTCTCATAGACCTCTGCTATTCCACAGTCACCTCTCCAAAG | | RNLALIDLCYSTVTSP | |
| | ATGCTGGTGGACTTCCTCCATGAGACCAAGACGATCTCCTACCAGGGCT | | KMLVDFLHETKTISYQ | |
| | GCATGGCCCAGATCTTCTTCTTCCACCTTTTGGGAGGTGGGACTGTCTT | | GCMAQIFFFHLLGGGT | |
| | TTTTCTCTCAGTCATGGCCTATGACCGCTACATAGCCATCTCCCAGCCC | | VFFLSVMAYDRYIAIS | |
| | CTCCGGTATGTCACCATCATGAACACTCAATTGTGTGTGGGCCTGGTAG | | QPLRYVTIMNTQLCVG | |
| | TAGCCGCCTGGGTGGGGGGCTTTGTCCACTCCATTGTCCAACTGGCTCT | | LVVAAWVGGFVHSIVQ | |
| | GATACTTCCACTGCCCTTCTGTGGCCCCAATATCCTAGATAACTTCTAC | | LALILPLPFCGPNILD | |
| | TGTGATGTTCCCCAAGTACTGAGACTTGCCTGCACTGATACCTCCCTCC | | NFYCDVPQVLRLACTD | |
| | TGGAGTTCCTCATGATCTCCAACAGTGGGCTGCTAGTTATCATCTGGTT | | TSLLEFLMISNSGLLV | |
| | CCTCCTCCTTCTGATCTCTTATACTGTCATCCTGGTGATGCTGAGGTCC | | IIWFLLLLISYTVILV | |
| | CACTCGGGAAAGGCAAGGAGGAAGGCAGCTTCCACCTGCACCACCCACA | | MLRSHSGKARRKAAST | |
| | TCATCGTGGTGTCCATGATCTTCATTCCCTGTATCTATATCTATACCTG | | CTTHIIVVSMIFIPCI | |
| | GCCCTTCACCCCATTCCTCATGGACAAGGCTGTGTCCATCAGCTACACA | | YIYTWPFTPFLMDKAV | |
| | GTCATGACCCCCATGCTCAACCCCATGATCTACACCCTGAGAAACCAGG | | SISYTVMTPMLNPMIY | |
| | ACATGAAAGCAGCCATGAGGAGATTAGGCAAGTGCCTAGTAATTTGCAG | | TLRNQDMKAAMRRLGK | |
| | GGAGTAAACTTTAA | | CLVICRE | |
| CG | TGGGAAAACCATACTACACTGCCTGAATTCCTCCTTCTGGGATTCTCTG | 69 | WENHTTLPEFLLLGFS | 70 |
| 50275_01 | ACCTTAAGGCCCTGCAGGACCCCCTGTTCTGGGTGGTGCTTCTGGTCTA | | DLKALQDPLFWVVLLV | |
| | CCTGGTCACCTTGCTGGGTAACTCCCTGATCATCCTCCTCACACAGGTC | | YLVTLLGNSLIILLTQ | |
| | AGCCCTGCCCTGCACTCCCCCATGTACTTCTTCCTGCGCCAACTCTCAG | | VSPALHSPMYFFLRQL | |
| | TGGTGGAGCTCTTCTACACCACTGACATCGTGCCCAGGACCCTGGCCAA | | SVVELFYTTDIVPRTL | |
| | TCTGGGCTCCCCGCATCCCCAGGCCATCTCTTTCCAGGGCTGTGCAGCC | | ANLGSPHPQAISFQGC | |
| | CAGATGTACGTCTTCATTGTCCTGGGCATCTCGGAGTGCTGCCTGCTCA | | AAQMYVFIVLGISECC | |
| | CGGCCATGGCCTATGACCGATATGTTGCCATCTGCCAGCCCCTACGCTA | | LLTAMAYDRYVAICQP | |
| | TTCCACCCTCTTGAGCCCACGGGCCTGCATGGCCATGGTGGGTACCTCC | | LRYSTLLSPRACMAMV | |
| | TGGCTCACAGGCATCATCACGGCCACCACCCATGCCTCCCTCATCTTCT | | GTSWLTGIITATTHAS | |
| | CTCTACCTTTTCGCAGCCACCCGATCATCCCGCACTTTCTCTGTGACAT | | LIFSLPFRSHPIIPHF | |
| | CCTGCCAGTACTGAGGCTGGCAAGTGCTGGGAAGCACAGGAGCGAGATC | | LCDILPVLRLASAGKH | |
| | TCCGTGATGACAGCCACCATAGTCTTCATTATGATCCCCTTCTCTCTGA | | RSEISVMTATIVFIMI | |
| | TTGTCACCTCTCACATCCGCATCCTGGGTGCCATCCTAGCAATGGCCTC | | PFSLIVTSHIRILGAI | |
| | CACCCAGAGCCGCCGCAAGGTCTTCTCCACCTGCTCCTCCCATCTGCTC | | LAMASTQSRRKVFSTC | |
| | GTGGTCTCTCTCTTCTTTGGAGCAGCCAGCATCACCTACATCCGGCCGC | | SSHLLVVSLFFGAASI | |
| | AGGCAGGCTCCTCTGTTACCACAGACCGCGTCCTCAGTCTCTTCTACAC | | TYIRPQAGSSVTTDRV | |
| | AGTCATCACACCCATGCTCAACCCCATCATCTACACCCTTCGGAACAAG | | LSLFYTVITPMLNPII | |
| | GACGTGAGGAGGGCCCTGCGACACTTGGTGAAGAGGCAGCGCCCCTCAC | | YTLRNKDVRRALRHLV | |
| | CCTGAAGGGACTCGGAT | | KRQRPSP | |
| CG | GGCTTTGGGACTAACATCTCAAGTACTACCAGCTTCACTCTAACAGGCT | 71 | GFGTNISSTTSFTLTG | 72 |
| 55970-02 | TCCCTGAGATGAAGGGTCTGGAGCACTGGCTGGCTGCCCTTCTGCTGCT | | FPEMKGLEHWLAALLL | |
| | GCTTTATGCTATTTCCTTCCTGGGCAACATCCTCATCCTCTTTATCATA | | LLYAISFLGNILILFI | |
| | AAGGAAGAGCAGAGCTTGCACCAGCCAATGTACTACTTCCTGTCTCTTT | | IKEEQSLHQPMYYFLS | |
| | TTTCTGTTAATGACCTGGGTGTGTCCTTTTCTACATTGCCCACTGTACT | | LFSVNDLGVSFSTLPT | |
| | GGCTGCTGTGTGTTTTCATGCCCCAGAGACAACTTTTGATGCCTGCCTG | | VLAAVCFHAPETTFDA | |
| | GCCCAGATGTTCTTCATCCACTTTTCCTCCTGGACAGAGTTTGGCATCC | | CLAQMFFIHFSSWTEF | |
| | TACTGGCCATGAGTTTTGACCACTATGTGGCCATCTGTAACCCGCTGCG | | GILLAMSFDHYVAICN | |
| | CTATGCCACAGTGCTCACTGATGTCCGTGTGGCCCACAATGGCATATCC | | PLRYATVLTDVRVAHN | |
| | ATTGTCATCCGCAGCTTCTGCATGGTATTCCCACTTCCCTTCCTCCTGA | | GISIVIRSFCMVFPLP | |
| | AGAGACTGTCTTTCTGTAAGGCCAGTGTGGTACTGGCCCATTCCTACTG | | FLLKRLSFCKASVVLA | |
| | TCTGCATGCAGACCTGATTCGGCTGCCCTGGGGAGACACTACCATCAAC | | HSYCLHADLIRLPWGD | |
| | AGCATGTATGGCCTGTTCATTGTCATCTCTGCCTTTGGTGTAGATTCAC | | TTINSMYGLFIVISAF | |
| | TGCTCATCCTCCTCTCCTATGTGCTCATTCTGCGTTCTGTGCTGGCCAT | | GVDSLLILLSYVLILR | |
| | TGCCTCCAGGGGTGAGAGGCTTAAGACACTCAACACATGTGTGTCACAT | | SVLAIASRGERLKTLN | |
| | ATCTATGCAGTGCTGATCTTCTATGTGCCTATGGTTGGTGTGTCCATGG | | TCVSHIYAVLIFYVPM | |
| | TTCATCGATTTGGGAGGCATGCTCCTGAATATGTGCACAAGTTCATGTC | | VGVSMVHRFGRHAPEY | |
| | TCTTTGTACCTCCAATGCTCTACCCAATTATCTATTCCATCAAG | | VHKFMSLCTSNALPNY | |
| | | | LFHQ | |
| CG | CCAATGACTGGGGGAGGAAATATTACAGAAATCACCTATTTCATCCTGC | 73 | MTGGGNITEITYFILL | 74 |
| 56119-01 | TGGGATTCTCAGATTTTCCCAGGATCATAAAAGTGCTCTTCACTATATT | | GFSDFPRIIKVLFTIF | |
| | CCTGGTGATCTACATTACATCTCTGGCCTGGAACCTCTCCCTCATTGTT | | LVIYITSLAWNLSLIV | |
| | TTAATAAGGATGGATTCCCACCTCCATACACCCATGTATTTCTTCCTCA | | LIRMDSHLHTPMYFFL | |
| | GTAACCTGTCCTTCATAGATGTCTGCTATATCAGCTCCACAGTCCCCAA | | SNLSFIDVCYISSTVP | |
| | GATGCTCTCCAACCTCTTACAGGAACAGCAAACTATCACTTTTGTTGGT | | KMLSNLLQEQQTITFV | |
| | TGTATTATTCAGTACTTTATCTTTTCAACGATGGGACTGAGTGAGTCTT | | GCIIQYFIFSTMGLSE | |
| | GTCTCATGACAGCCATGGCTTATGATCGTTATGCTGCCATTTGTAACCC | | SCLMTAMAYDRYAAIC | |
| | CCTGCTCTATTCATCCATCATGTCACCCACCCTCTGTGTTTGGATGGTA | | NPLLYSSIMSPTLCVW | |
| | CTGGGAGCCTACATGACTGGCCTCACTGCTTCTTTATTCCAAATTGGTG | | MVLGAYMTGLTASLFQ | |
| | CTTTGCTTCAACTCCACTTCTGTGGGTCTAATGTCATCAGACATTTCTT | | IGALLQLHFCGSNVIR | |
| | CTGTGACATGCCCCAACTGTTAATCTTGTCCTGTACTGACACTTTCTTT | | HFFCDMPQLLILSCTD | |
| | GTACAGGTCATGACTGCTATATTAACCATGTTCTTTGGGATAGTAAGTG | | TFFVQVMTAILTMFFG | |
| | CCCTAGTTATCATGATATCCTATGGCTATATTGGCATCTCCATCATGAA | | IVSALVIMISYGYIGI | |
| | GATCACTTCAGCTAAAGGCAGGTCCAAGGCATTCAACACCTGTGCTTCT | | SIMKITSAKGRSKAFN | |
| | CATCTAACAGCTGTTTCCCTCTTCTATACATCAGGAATCTTTGTCTATT | | TCASHLTAVSLFYTSG | |
| | TGAGTTCCAGCTCTGGAGGTTCTTCAAGCTTTGACAGATTTGCATCTGT | | IFVYLSSSSGGSSSFD | |
| | TTTCTACACTGTGGTCATTCCCATGTTAAATCCCTTCATTTACAGTTTG | | RFASVFYTVVIPMLNP | |
| | AGGAACAAAGAAATTAAAGATGCCTTAAAGAGGTTGCAAAAGAGAAAGT | | FIYSLRNKEIKDALKR | |
| | GCTGCTGA | | LQKRKCC | |
| GMAL | AAACCTGAGGCAATGGACCCACAGAACTATTCCTTGGTGTCAGAATTTG | 75 | MDPQNYSLVSEFVLHG | 76 |
| 359218_D_da1 | TGTTGCATGGACTCTGCACTTCACGACATCTTCAAAATTTTTTCTTTAT | | LCTSRHLQNFFFIFFF | |
| | ATTTTTCTTTGGGGTCTATGTGGCCATTATGCTGGGTAACCTTCTCATT | | GVYVAIMLGNLLILVT | |
| | TTGGTCACTGTAATTTCTGATCCCTGCCTGCACTCCTCCCCTATGTACT | | VISDPCLHSSPMYFLL | |
| | TCCTGCTGGGGAACCTAGCTTTCCTGGACATGTGGCTGGCCTCATTTGC | | GNLAFLDMWLASFATP | |
| | CACTCCCAAGATGATCAGGGATTTCCTTAGTGATCAAAAACTCATCTCC | | KMIRDFLSDQKLISFG | |
| | TTTGGAGGATGTATGGCTCAAATCTTCTTCTTGCACTTTACTGGTGGGG | | GCMAQIFFLHFTGGAE | |
| | CTGAGATGGTGCTCCTGGTTTCCATGGCCTATGACAGATATGTGGCCAT | | MVLLVSMAYDRYVAIC | |
| | ATGCAAACCCTTGCATTACATGACTTTGATGAGTTGGCAGACTTGCATC | | KPLHYMTLMSWQTCIR | |
| | AGGCGGGTGCTGGCTTCATGGGTCGTTGGATTTGTGCACTCCATCAGTC | | RVLASWVVGFVHSISQ | |
| | AAGTGGCTTTCACTGTAAATTTGCCTTACTGTGGCCCCAATGAGGTAGA | | VAFTVNLPYCGPNEVD | |
| | CAGCTTCTTCTGTGACCTCCCTCTGGTGATCAAACTTGCCTGCATGGAC | | SFFCDLPLVIKLACMD | |
| | ACCTATGTCTTGGGTATAATTATGATCTCAGACAGTGGGTTGCTTTCCT | | TYVLGIIMISDSGLLS | |
| | TGAGCTGTTTTCTGCTCCTCCTGATCTCCTACACCGTGATCCTCCTCGC | | LSCFLLLLISYTVILL | |
| | TATCAGACAGCGTGCTGCCGGTAGCACATCCAAAGCACTCTCCACTTGC | | AIRQRAAGSTSKALST | |
| | TCTGCACATATCATGGTAGTGACGCTGTTCTTTGGCCCTTGCATTTTTG | | CSAHIMVVTLFFGPCI | |
| | CTTATGTGCGGCCTTTCAGTAGGTTCTCTGTGGACAAGCTGCTGTCTGT | | FAYVRPFSRFSVDKLL | |
| | GTTTTATACCATTTTTACTCCACTCCTGAACCCCATTATCTACACATTG | | SVFYTIFTPLLNPIIY | |
| | AGAAATGAGGAAATGAAAGCAGCTATGAAGAAACTGCAAAACCGACGGG | | TLRNEEMKAAMKKLQN | |
| | TGACTTTTCAATGAAATCCAGCCTTCCA | | RRVTFQ | |
| CG | AACTAAATGTTGATGAATTACTCTAGTGCCACTGAATTTTATCTCCTTG | 77 | MLMNYSSATEFYLLGF | 78 |
| 56880-01 | GCTTCCCTGGCTCTGAAGAACTACATCATATCCTTTTTGCTATATTCTT | | PGSEELHHILFAIFFF | |
| | CTTTTTCTACTTGGTGACATTAATGGGAAACACAGTCATCATCATGATT | | FYLVTLMGNTVIIMIV | |
| | GTCTGTGTGGATAAACGTCTGCAGTCCCCCATGTATTTCTTCCTCGGCC | | CVDKRLQSPMYFFLGH | |
| | ACCTCTCTGCCCTGGAGATCCTGGTCACAACCATAATCGTCCCCGTGAT | | LSALEILVTTIIVPVM | |
| | GCTTTGGGGATTGCTGCTCCCTGGGATGCAGACAATATATTTGTCTGCC | | LWGLLLPGMQTIYLSA | |
| | TGTGTTGTCCAGCTCTTCTTGTACCTTGCTGTGGGGACAACAGAGTTCG | | CVVQLFLYLAVGTTEF | |
| | CATTACTTGGAGCAATGGCTGTGGACCGTTATGTGGCTGTCTGTAACCC | | ALLGAMAVDRYVAVCN | |
| | TCTGAGGTACAACATCATTATGAACAGACACACCTGCAACTTTGTGGTT | | PLRYNIIMNRHTCNFV | |
| | CTTGTGTCATGGGTGTTTGGGTTTCTTTTTCAAATCTGGCCGGTCTATG | | VLVSWVFGFLFQIWPV | |
| | TCATGTTTCAGCTTACTTACTGCAAATCAAATGTGGTGAACAATTTTTT | | YVMFQLTYCKSNVVNN | |
| | TTGTGACCGAGGGCAATTGCTCAAACTATCCTGCAATAATACTCTTTTC | | FFCDRGQLLKLSCNNT | |
| | ACGGAGTTTATCCTCTTCTTAATGGCTGTTTTTGTTCTCTTTGGTTCTT | | LFTEFILFLMAVFVLF | |
| | TGATCCCTACAATTGTCTCCAACGCCTACATCATCTCCACCATTCTCAA | | GSLIPTIVSNAYIIST | |
| | GATCCCGTCATCCTCTGGCCGGAGGAAATCCTTCTCCACTTGTGCCTCC | | ILKIPSSSGRRKSFST | |
| | CACTTCACCTGTGTTGTGATTGGCTACGGCAGCTGCTTGTTTCTCTACG | | CASHFTCVVIGYGSCL | |
| | TGAAACCCAAGCAAACGCAGGCAGCTGATTACAATTGGGCAGTTTCCCC | | FLYVKPKQTQAADYNW | |
| | GATGGTTTCAGTAGTAACTCCTTTCCTCAATCCTTTCATCTTCACCCTC | | AVSPMVSVVTPFLNPF | |
| | CGGAATGATAAAGTCATAGAGGCCCTTCGGATGGGGTGAAACGCTGCT | | IFTLRNDKVIEALRMG | |
| CG | ATGAATCATGTGGTAAAACACAATCACACGGCAGTGACCAAGGTGACTG | 79 | MNHVVKHNHTAVTKVT | 80 |
| 57423-01 | AATTTATTCTCATGGGGATTACAGACAACACTGGGCTGCAGGCTCCACT | | EFILMGITDNTGLQAP | |
| | GTTTGGACTCTTCCTCATCATATATCTGGTCACAGTGATAGGCAATCTG | | LFGLFLIIYLVTVIGN | |
| | GGCATGGTTATCTTGACCTACTTGGACTCCAAGCTACACACCCCCATGT | | LGMVILTYLDSKLHTP | |
| | ACTTTTTCCTTAGACATTTGTCAATCACTGATCTTGGTTACTCCACTGT | | MYFFLRHLSITDLGYS | |
| | CATTGCCCCGAAGATGTTAGTAAACTTCATAGTGCACAAAAACACAATT | | TVIAPKMLVNFIVHKN | |
| | TCTTACAATTGGTATGCCACTCAGCTAGCATTCTTTGAGATTTTCATCA | | TISYNWYATQLAFFEI | |
| | TCTCTGAGCTCTTTATTCTATCAGCAATGGCCTATGATCGCTACGTAGC | | FIISELFILSAMAYDR | |
| | CATCTGTAAACCTCTTCTGTACGTGATCATCATGGCAGAGAAAGTACTT | | YVAICKPLLYVIIMAE | |
| | TGGGTGCTGGTAATTGTTCCCTATCTCTATAGCACGTTTGTGTCACTAT | | KVLWVLVIVPYLYSTF | |
| | TTCTCACAATTAAGTTATTTAAACTGTCCTTCTGTGGCTCAAACATAAT | | VSLFLTIKLFKLSFCG | |
| | CAGCTATTTTTACTGTGACTGTATCCCTCTGATGTCCATACTCTGTTCT | | SNIISYFYCDCIPLMS | |
| | GACACAAATGAATTAGAATTAATAATTTTGATCTTCTCAGGCTGTAATT | | ILCSDTNELELIILIF | |
| | TGCTCTTCTCCCTCTCAATTGTTCTCATATCCTACATGTTTATTCTAGT | | SGCNLLFSLSIVLISY | |
| | GGCCATTCTCAGAATGAACTCAAGGAAAGGGAGGTACAAAGCCTTCTCC | | MFILVAILRMNSRKGR | |
| | ACCTGTAGCTCTCATCTGACAGTGGTGATCATGTTCTATGGGACATTGT | | YKAFSTCSSHLTVVIM | |
| | TATTTATTTACTTGCAACCCGAGTCCAGTCATACTTTGGCTATTGATAA | | FYGTLLFIYLQPESSH | |
| | AATGGCCTCAGTGTTTTATACCCTGTTGATTCCTATGCTGAATCCGTTG | | TLAIDKMASVFYTLLI | |
| | ATCTACAGCCTAAGGAACAAAGAAGTAAAAGATGCTCTAAAGAGAACTT | | PMLNPLIYSLRNKEVK | |
| | TAACCAATCGATTCAAAATTCCCATTTAATATCTTAATACTCA | | DALKRTLTNRFKIPI | |
| CG | GTCCTCAATTCATGCTGCTATCCAACATTACTCAGTTTAGCCCCATATT | 81 | MLLSNITQFSPIFYLT | 82 |
| 57564-01 | CTATCTCACCAGCTTTCCTGGATTGGAAGGCATCAAACACTGGATTTTC | | SFPGLEGIKHWIFIPF | |
| | ATCCCCTTTTTCTTTATGTACATGGTTGCCATCTCAGGCAATTGTTTCA | | FFMYMVAISGNCFILI | |
| | TTCTGATCATTATTAAGACCAACCCTCGTCTGCACACACCCATGTACTA | | IIKTNPRLHTPMYYLL | |
| | TCTACTATCCTTGCTGGCCCTCACTGACCTGGGGCTGTGTGTGTCCACG | | SLLALTDLGLCVSTLP | |
| | TTGCCCACCACTATGGGGATCTTCTGGTTTAACTCCCATAGTATCTACT | | TTMGIFWFNSHSIYFG | |
| | TTGGAGCGTGTCAAATCCAGATGTTCTGCATCCACTCTTTTTCCTTCAT | | ACQIQMFCIHSFSFME | |
| | GGAGTCCTCAGTGCTCCTCATGATGTCCTTTGACCGCTTTGTGGCCATC | | SSVLLMMSFDRFVAIC | |
| | TGCCACCCTCTGAGGTATTCGGTCATTATCACTGGCCAGCAAGTGGTCA | | HPLRYSVIITGQQVVR | |
| | GAGCAGGCCTAATTGTCATCTTCCGGGGACCTGTGGCCACTATCCCTAT | | AGLIVIFRGPVATIPI | |
| | TGTCCTCCTCCTGAAGGCTTTTCCCTACTGTGGATCTGTGGTCCTCTCC | | VLLLKAFPYCGSVVLS | |
| | CACTCATTTTGCCTGCACCAGGAAGTGATACAGCTGGCCTGCACAGATA | | HSFCLHQEVIQLACTD | |
| | TCACCTTCAATAATCTGTATGGACTGATGGTGGTAGTTTTCACTGTGAT | | ITFNNLYGLMVVVFTV | |
| | GCTGGACCTGGTGCTCATCGCACTGTCCTATGGACTCATCCTGCACACA | | MLDLVLIALSYGLILH | |
| | GTAGCAGGCCTGGCCTCCCAAGAGGAGCAGCGCCGTGCCTTTCAGACAT | | TVAGLASQEEQRRAFQ | |
| | GCACCGCTCATCTCTGTGCTGTGCTAGTATTCTTTGTGCCCATGATGGG | | TCTAHLCAVLVFFVPM | |
| | GCTGTCCCCGGTGCACCGTTTTGGGAAGCATGCCCCACCTGCTATTCAT | | MGLSPVHRFGKHAPPA | |
| | CTTCTTATGGCCAATGTCTACCTTTTTGTGCCTCCCATGCTTAACCCAA | | IHLLMANVYLFVPPML | |
| | TCATATACAGCATTAAGACCAAGGAGATCCACCGTGCCATTATCAAGTT | | NPIIYSIKTKEIHRAI | |
| | CCTAGGTCTTAAAAAGGCCAGTAAATGAGTCCTGGGGCTAAAACTAACC | | IKFLGLKKASK | |
| | CATAGAGGCCTATCCAACAGTAAATGAGCCCCAAATGGGACTGA | | | |
| CG | CAGTGGTGTCTGAATTTGTACTGTTGGGACTCTGTAGTTCTCAAAAACT | 83 | VVSEFVLLGLCSSQKL | 84 |
| 57691-01 | CCAGCTTTTCTATTTTTGTTTCTTCTCTGTGTTGTATACAGTCATTGTG | | QLFYFCFFSVLYTVIV | |
| | CTGGGAAATCTTCTCATTATCCTCACAGTGACTTCTGATACCAGCCTGC | | LGNLLIILTVTSDTSL | |
| | ACTCCCCTATGTACTTTCTCTTGGGAAACCTTTCCTTTGTTGACATTTG | | HSPMYFLLGNLSFVDI | |
| | TCAGGCTTCTTTTGCTACCCCTAAAATGATTGCAGATTTTCTGAGTGCA | | CQASFATPKMIADFLS | |
| | CACGAGACCATATCTTTCAGTGGCTGCATAGCCCAAATTTTCTTTATTC | | AHETISFSGCIAQIFF | |
| | ACCTTTTTACTGGAGGGGAGATGGTGCTACTTGTTTCGATGGCCTATGA | | IHLFTGGEMVLLVSMA | |
| | CAGGTATGTAGCCATATGCAAACCCTTATACTATGTGGTCATCATGAGC | | YDRYVAICKPLYYVVI | |
| | CGAAGGACATGCACTGTCTTGGTAATGATCTCCTGGGCTGTGAGCTTGG | | MSRRTCTVLVMISWAV | |
| | TGCACACATTAAGCCAGTTATCATTTACTGTGAACCTGCCTTTTTGTGG | | SLVHTLSQLSFTVNLP | |
| | ACCTAATGTAGTAGACAGCTTTTTTTGTGATCTTCCTCGAGTCACCAAA | | FCGPNVVDSFFCDLPR | |
| | CTTGCCTGCCTGGACTCTTACATCATTGAAATACTAATTGTGGTCAATA | | VTKLACLDSYIIEILI | |
| | GTGGAATTCTTTCCCTAAGCACTTTCTCTCTCTTGGTCAGCTCCTACAT | | VVNSGILSLSTFSLLV | |
| | CATTATTCTTGTTACAGTTTGGCTCAAGTCTTCAGCTGCAATGGCAAAG | | SSYIIILVTVWLKSSA | |
| | GCATTTTCTACGCTGGCTTCCCATATTGCAGTAGTAATATTATTCTTTG | | AMAKAFSTLASHIAVV | |
| | GACCTTGCATCTTCATCTATGTGTGGCCCTTTACCATCTCTCCTTTGGA | | ILFFGPCIFIYVWPFT | |
| | TAAATTTCTTGCCATATTTTACACTGTTTTCACCCCCGTCCTAAACCCC | | ISPLDKFLAIFYTVFT | |
| | ATTATTTATACACTAAGGAATAGGGATATGAAGGCTGCCGTAAGGAAAA | | PVLNPIIYTLRNRDMK | |
| | TTGTGAACCATTACCTGAGGCCAAGGAGAATTTCTGAAATGTCACTAGT | | AAVRKIVNHYLRPRRI | |
| | AGTGAGAACTTCCTTTCATTAAGACAAAACTCCTTCAA | | SEMSLVVRTSFH | |
| CG | AAAACACCATGGAAACAGGGAACCTCACGTGGGTATCAGACTTTGTCTT | 85 | METGNLTWVSDFVFLG | 86 |
| 59408-01 | CCTGGGGCTCTCGCAGACTCGGGAGCTCCAGCGTTTCCTGTTTCTAATG | | LSQTRELQRFLFLMFL | |
| | TTCCTGTTTGTCTACATCACCACTGTTATGGGAAACATCCTTATCATCA | | FVYITTVMGNILIIIT | |
| | TCACAGTGACCTCTGATTCCCAGCTCCACACACCCATGTACTTTCTGCT | | VTSDSQLHTPMYFLLR | |
| | CCGAAACCTGGCTGTCCTAGACCTCTGTTTCTCTTCAGTCACTGCTCCC | | NLAVLDLCFSSVTAPK | |
| | AAAATGCTAGTGGACCTCCTCTCTGAGAAGAAAACCATCTCTTACCAGG | | MLVDLLSEKKTISYQG | |
| | GCTGCATGGGTCAGATCTTCTTCTTCCACTTTTTGGGAGGTGCCATGGT | | CMGQIFFFHFLGGAMV | |
| | CTTCTTCCTCTCAGTGATGGCCTTTGACCGCCTCATTGCCATCTCCCGG | | FFLSVMAFDRLIAISR | |
| | CCCCTCCGCTATGTCACCGTCATGAACACTCAGCTCTGGGTGGGGCTGG | | PLRYVTVMNTQLWVGL | |
| | TGGTAGCCACCTGGGTGGGAGGCTTTGTCCACTCTATTGTCCAGCTGGC | | VVATWVGGFVHSIVQL | |
| | TCTGATGCTCCCACTGCCCTTCTGTGGCCCCAACATTTTGGATAACTTC | | ALMLPLPFCGPNILDN | |
| | TACTGTGATGTTCCCCAAGTACTGAGACTTGCCTGCACTGACACCTCAC | | FYCDVPQVLRLACTDT | |
| | TGCTGGAGTTCCTCAAGATCTCCAACAGTGGGCTGCTGGATGTCGTCTG | | SLLEFLKISNSGLLDV | |
| | GTTCTTCCTCCTCCTGATGTCCTACTTATTCATCCTGGTGATGCTGAGG | | VWFFLLLMSYLFILVM | |
| | TCACATCCAGGGGAGGCAAGAAGGAAGGCAGCTTCCACCTGCACCACCC | | LRSHPGEARRKAASTC | |
| | ACATCATCGTGGTTTCCATGATCTTCGTTCCAAGCATTTACCTCTATGC | | TTHIIVVSMIFVPSIY | |
| | CCGGCCCTTCACTCCATTCCCTATGGACAAGCTTGTGTCCATCGGCCAC | | LYARPFTPFPMDKLVS | |
| | ACAGTCATGACCCCCATGCTCAACCCCATGATCTATAACCTGAGGAACC | | IGHTVMTPMLNPMIYN | |
| | CGGACATGCAGGCAGCAGTGAGAAGATTAGGGAGACACCGGCTGGTTTG | | LRNPDMQAAVRRLGRH | |
| | AGA | | RLV | |
| CG | ACCCTGAGGGGCAAAAGGTTTTATTTGTCACATTCTTACTAATCTACAT | 87 | PEGQKVLFVTFLLIYM | 88 |
| 90352-01 | GGTGACGATAATGGGCAACCTGCTTATCATAGTGACCATCATGGCCAGC | | VTIMGNLLIIVTIMAS | |
| | CAGTCCCTGGGTTCCCCCATGTACTTTTTTCTGGCTTCTTTATCATTCA | | QSLGSPMYFFLASLSF | |
| | TAGATACCGTCTATTCTACTGCATTTGCTCCCAAAATGATTGTTGACTT | | IDTVYSTAFAPKMIVD | |
| | GCTCTCTGAGAAAAAGACCATTTCCTTTCAGGGTTGTATGGCTCAACTT | | LLSEKKTISFQGCMAQ | |
| | TTTATGGATCATTTATTTGCTGGTGCTGAAGTCATTCTTCTGGTGGTAA | | LFMDHLFAGAEVILLV | |
| | TGGCCTATGATCGATACATGGCCATCTGTAAGCCTCTTCATGAATTGAT | | VMAYDRYMAICKPLHE | |
| | CACCATGAATCGTCGAGTCTGTGTTCTTATGCTGTTGGCGGCCTGGATT | | LITMNRRVCVLMLLAA | |
| | GGAGGCTTTCTTCACTCATTGGTTCAATTTCTCTTTATTTATCAGCTCC | | WIGGFLHSLVQFLFIY | |
| | CTTTCTGTGGACCCAATGTCATTGACAACTTCCTGTGTGATTTGTATCC | | QLPFCGPNVIDNFLCD | |
| | CTTATTGAAACTTGCTTGCACCAATACCTATGTCACTGGGCTTTCTATG | | LYPLLKLACTNTYVTG | |
| | ATAGCTAATGGAGGAGCGATTTGTGCTGTCACCTTCTTCACTATCCTGC | | LSMIANGGAICAVTFF | |
| | TTTCCTATGGGGCCATATTACACTCTCTTAAGACTCAGAGTTTGGAAGG | | TILLSYGAILHSLKTQ | |
| | GAAACGAAAAGCTTTCTACACCTGTGCATCCCACGTCACTGTGGTCATT | | SLEGKRKAFYTCASHV | |
| | TTATTCTTTGTCCCCTGTATCTTCTTGTATGCAAGGCCCAATTCTACTT | | TVVILFFVPCIFLYAR | |
| | TTCCCATTGATAAATCCATGACTGTAGTTCTAACTTTTATAACTCCCAT | | PNSTFPIDKSMTVVLT | |
| | GCTGAACCCACTAATCTATACCCTGAAGAATGCAGAAATGAAAAGTGCC | | FITPMLNPLIYTLKNA | |
| | ATGAGGAAACTTTGGAGTAAAAAAGTAAGCTTAGCTGGGAAATGGCTGT | | EMKSAMRKLWSKKVSL | |
| | ATCACTCATGAGAAT | | AGKWLYHS | |
| CG | TGAAGATAGCAAACAACACAGTAGTGACAGAATTTATCCTCCTTGGTCT | 89 | KIANNTVVTEFILLGL | 90 |
| 92727_01 | GACTCAGTCTCAAGATATTCAGCTCTTGGTCTTTGTGCTGATCTTAATT | | TQSQDIQLLVFVLILI | |
| | TTCTACCTTATCATCCTCCCTGGAAATTTTCTCATTATTTTCACCATAA | | FYLIILPGNFLIIFTI | |
| | GGTCAGACCCTGGGCTCACAGCCCCCCTCTATTTATTTCTGGGCAACTT | | RSDPGLTAPLYLFLGN | |
| | GGCCTTCCTGGATGCATCCTACTCCTTCATTGTGGCTCCCAGGATGTTG | | LAFLDASYSFIVAPRM | |
| | GTGGACTTCCTCTCTGAGAAGAAGGCAATCTCCTACAGAGGCTGCATCA | | LVDFLSEKKAISYRGC | |
| | CTCAGCTCTTTTTCTTGCACTTCCTTGGAGGAGGGGAGGGATTACTCCT | | ITQLFFLHFLGGGEGL | |
| | TGTTGTGATGGCCTTTGACCGCTACATCGCCATCTGCCGGCCTCTGCAC | | LLVVMAFDRYIAICRP | |
| | TGTTCAACTGTCATGAACCCTAGAGCCTGCTATGCAATGATGTTGGCTC | | LHCSTVMNPRACYAMM | |
| | TGTGGCTTGGGGGTTTTGTCCACTCCATTATCCAGGTGGTCCTCATCCT | | LALWLGGFVHSIIQVV | |
| | CCGCTTGCCTTTTTGTGGCCCAAACCAGCTGGACAACTTCTTCTGTGAT | | LILRLPFCGPNQLDNF | |
| | GTCCGACAGGTCATCAAGCTGGCTTGCACCGACATGTTTGTGGTGGAGC | | FCDVRQVIKLACTDMF | |
| | TTCTGATGGTCTTCAACAGTGGCCTGATGACACTCCTGTGCTTTCTGGG | | VVELLMVFNSGLMTLL | |
| | GCTTCTGGCTTCCTATGCAGTCATCCTCTGCCATGTTCGTAGGGCAGCT | | CFLGLLASYAVILCHV | |
| | TCTGAAGGGAAGAACAAGGCCATGTCCACATGCACCACTCGTGTCATTA | | RRAASEGKNKAMSTCT | |
| | TTATACTTCTTATGTTTGGACCTGCTATCTTCATCTACATGTGCCCTTT | | TRVIIILLMFGPAIFI | |
| | CAGGGCCTTACCAGCTGACAAGATGGTTTCTCTCTTTCACACAGTGATC | | YMCPFRALPADKMVSL | |
| | TTTCCATTGATGAATCCTATGATTTATACCCTTCGCAACCAGGAAGTGA | | FHTVIFPLMNPMIYTL | |
| | AAACTTCCATGAAGAGGTTATTGAGTCGACATGTAGTCTGTCAAGTGGA | | RNQEVKTSMKRLLSRH | |
| | TTTTATAATAAGAAACTGAGAAGGAGGAATTCTGGCTGGAATTCATATC | | VVCQVDFIIRN | |
| | ATTCATTTA | | | |
| GMAC | CATGGAAGGCAACCAGACATGGATCACAGACATCACCCTGCTGGGATTC | 91 | MEGNQTWITDITLLGF | 92 |
| 076959_B | CAGGTTGGTCCAGCACTGGCGATTCTCCTCTGTGGACTCTTCTCTGTCT | | QVGPALAILLCGLFSV | |
| | TCTATACACTCACCCTGCTGGGGAATGGGGTCATCTTTGGGATTATCTG | | FYTLTLLGNGVIFGII | |
| | CCTGGACTCTAAGCTTCACACACCCATGTACTTCTTCCTCTCACACCTG | | CLDSKLHTPMYFFLSH | |
| | GCCATCATTGACATGTCCTATGCTTCCAACAATGTTCCCAAGATGTTGG | | LAIIDMSYASNNVPKM | |
| | CAAACCTAATGAACCAGAAAAGAACCATCTCCTTTGTTCCATGCATAAT | | LANLMNQKRTISFVPC | |
| | GCAGACTTTTTTGTATTTGGCTTTTGCTGTTACAGAGTGCCTGATTTTG | | IMQTFLYLAFAVTECL | |
| | GTGGTGATGTCCTATGATAGGTATGTGGCCATCTGCCACCCTTTCCAGT | | ILVVMSYDRYVAICHP | |
| | ACACTGTCATCATGAGCTGGAGAGTGTGCACGATCCTGGTTCTCACGTC | | FQYTVIMSWRVCTILV | |
| | CTGGTCATGTGGGTTTGCCCTGTCCCTGGTACATGAAATTCTCCTTCTA | | LTSWSCGFALSLVHEI | |
| | AGGTTGCCCTTCTGTGGGCCCCGGGATGTGAACCACCTCTTCTGTGAAA | | LLLRLPFCGPRDVNHL | |
| | TTCTGTCTGTCCTCAAGCTGGCCTGTGCTGACACCTGGGTTAACCAAGT | | FCEILSVLKLACADTW | |
| | GGTCATATTTGCTACCTGTGTGTTTGTCTTAGTCGGGCCTCTTTCCTTG | | VNQVVIFATCVFVLVG | |
| | ATTCTGGTCTCCTACATGCACATCCTCGGGGCCATCCTGAAGATCCAGA | | PLSLILVSYMHILGAI | |
| | CAAAGGAGGGCCGCATAAAGGCCTTCTCCACCTGCTCCTCCCACCTGTG | | LKIQTKEGRIKAFSTC | |
| | TGTGGTTGGACTATTCTTTGGCATAGCCATGGTGGTTTACATGGTCCCA | | SSHLCVVGLFFGIAMV | |
| | GACTCTAATCAACGAGAGGAGCAGGAGAAAATGCTGTCCCTGTTTCACA | | VYMVPDSNQREEQEKM | |
| | GTGTCTTGAACCCAATGCTGAACCCCCTGATCTACAGCCTGAGGAATGC | | LSLFHSVLNPMLNPLI | |
| | TCAGTTGAAGGGCGCCCTCCACAGAGCACTCCAGAGGAAGAGGTCCATG | | YSLRNAQLKGALHRAL | |
| | AGAACGGTGTATGGGCTTTGCCTTTAAAACAT | | QRKRSMRTVYGLCL | |
| GMAC | GAATGGGGGACAACCAATCACGGGTCACAGAATTCATCCTGGTTGGATT | 93 | MGDNQSRVTEFILVGF | 94 |
| 076959_E | CCAGCTCAGTGTGGAGATGGAAGTGCTCCTCTTCTGGATCTTCTCCCTG | | QLSVEMEVLLFWIFSL | |
| | TTATATCTCTTCAGCCTGCTGGCAAATGGCATGATCTTGGGGCTCATCT | | LYLFSLLANGMILGLI | |
| | GTCTGGATCCCAGACTGCGCACCCCCATGTACTTCTTCCTGTCACACTT | | CLDPRLRTPMYFFLSH | |
| | GGCCGTCATTGACATATACTATGCTTCCAGCAATTTGCTCAACATGCTG | | LAVIDIYYASSNLLNM | |
| | GAAAACCTAGTGAAACACAAAAAAACTATCTCGTTCATCTCTTGCATTA | | LENLVKHKKTISFISC | |
| | TGCAGATGGCTTTGTATTTGACTTTTGCTGCTGCAGTGTGCATGATTTT | | IMQMALYLTFAAAVCM | |
| | GGTGGTGATGTCCTATGACAGATTTGTGGCGATCTGCCATCCCCTGCAT | | ILVVMSYDRFVAICHP | |
| | TACACTGTCATCATGAACTGGAGAGTGTGCACAGTACTGGCTATTACTT | | LHYTVIMNWRVCTVLA | |
| | CCTGGGCATGTGGATTTTCCCTGGCCCTCATAAATCTAATTCTCCTTCT | | ITSWACGFSLALINLI | |
| | AAGGCTGCCCTTCTGTGGGCCCCAGGAGGTGAACCACTTCTTCGGTGAA | | LLLRLPFCGPQEVNHF | |
| | ATTCTGTCTGTCCTCAAACTGGCCTGTGCAGACACCTGGATTAATGAAA | | FGEILSVLKLACADTW | |
| | TTTTTGTCTTTGCTGGTGGTGTGTTTGTCTTAGTCGGGCCCCTTTCCTT | | INEIFVFAGGVFVLVG | |
| | GATGCTGATCTCCTACATGCGCATCCTCTTGGCCATCCTGAAGATCCAG | | PLSLMLISYMRILLAI | |
| | TCAAAGGAGGGCCGCAAAAAAGCCTTTTCCACCTGCTCCTCCCACCTCT | | LKIQSKEGRKKAFSTC | |
| | GTGTGGTTGGGCTTTACTTTGGCATGGCCATGGTGGTTTACCTGGTCCC | | SSHLCVVGLYFGMAMV | |
| | AGACAACAGTCAACGACAGAAGCAGCAGAAAATTCTCACCCTGTTTTAC | | VYLVPDNSQRQKQQKI | |
| | AGCCTTTTCAACCCATTGCTGAACCCCCTCATCTACAGCCTGCGGAATG | | LTLFYSLFNPLLNPLI | |
| | CTCAAGTGAAGGGTGCCTTATACAGAGCACTGCAGAAAAAGAGGACCAT | | YSLRNAQVKGALYRAL | |
| | GTGAATGAGGGGAGAATTTTG | | QKKRTM | |
| SC | TGATGTATGCTCCATGGAGCGGGTCAATGAGACTGTGGTGAGAGAG | 95 | DCGERGHLPRLSSLAR | 96 |
| 122737711_ | GTCATCTTCCTCGGCTTTCATCCCTGGCCAGGCTGCAGCAGCTGCT | | LQQLLFVIFLLLYLFT | |
| A_2 | CTTTGTTATCTTCCTGCTCCTCTACCTGTTCACTCTGGGCACCAAT | | LGTNAIIISTIVLDRA | |
| | GCAATCATCATTTCCACCATTGTCCTGGACAGGGCCCTTCATATCC | | LHIPMYFFLAILSCSE | |
| | CCATGTACTTCTTCCTTGCCATCCTCTCTTGCTCTGAGATTTGCTA | | ICYTFIIVPKMLVDLL | |
| | CACCTTCATCATTGTACCCAAGATGCTGGTTGACCTGCTGTCCCAG | | SQKKTISFLGCAIQMF | |
| | AAGAAGACCATTTCTTTCCTGGGCTGTGCCATCCAAATGTTTTCCT | | SFLFLGCSHSFLLAVM | |
| | TCCTCTTCCTTGGCTGCTCTCACTCCTTTCTGCTGGCAGTCATGGG | | GYDRYIAICNPLRYSV | |
| | TTATGATCGTTACATAGCCATCTGTAACCCACTGCGCTACTCAGTG | | LMGHGVCMGLVAAACA | |
| | CTAATGGGACATGGGGTGTGTATGGGACTAGTGGCTGCTGCCTGTG | | CGFTVAQIITSLVFHL | |
| | CCTGTGGCTTCACTGTTGCACAGATCATCACATCCTTGGTATTTCA | | PFYSSNQLHHFFCDIA | |
| | CCTGCCTTTTTATTCCTCCAATCAACTACATCACTTCTTCTGTGAC | | PVLKLASHHNHFSQIV | |
| | ATTGCTCCTGTCCTCAAGCTGGCATCTCACCATAACCACTTTAGTC | | IFMLCTLVLAIPLLLI | |
| | AGATTGTCATCTTCATGCTCTGTACATTGGTCCTGGCTATCCCCTT | | LVSYVHILSAILQFPS | |
| | ATTGTTGATCTTGGTGTCCTATGTTCACATCCTCTCTGCCATACTT | | TLGRCKAFSTCVSHLI | |
| | CAGTTTCCTTCCACACTGGGTAGGTGCAAAGCTTTTTCTACCTGTG | | IVTVHYGCASFIYLRP | |
| | TATCTCACCTCATTATTGTCACTGTCCACTATGGCTGTGCCTCCTT | | QSNYSSSQDALISVSY | |
| | TATCTACTTAAGGCCTCAGTCCAACTACTCCTCAAGCCAGGATGCT | | TIITPLFNPMIYSLRN | |
| | CTAATATCAGTATCCTACACTATTATAACTCCATTGTTCAACCCAA | | KEFKSALCKIVRRTIS | |
| | TGATTTATAGCTTGAGAAATAAAGAGTTCAAATCAGCTCTTTGTAA | | LL | |
| | AATTGTGAGAAGAACAATTTCCCTGTTGTAAAATTAATCTTGATTC | | | |
| | TGGCCAG | | | |
| GMbA | TTCTCTCCCTAATTGTCTCCTGATGATGTATGCTCCATGGAGCGGGT | 97 | MERVNETVVREVIFLG | 98 |
| 144L1_B | CAATGAGACTGTGGTGAGAGAGGTCATCTTCCTCGGCTTCTCATCCC | | FSSLARLQQLLFVIFL | |
| | TGGCCAGGCTGCAGCAGCTGCTCTTTGTTATCTTCCTGCTCCTCTAC | | LLYLFTLGTNAIIIST | |
| | CTGTTCACTCTGGGCACCAATGCAATCATCATTTCCACCATTGTCCT | | IVLDRALHIPMYFFLA | |
| | GGACAGGGCCCTTCATATCCCCATGTACTTCTTCCTTGCCATCCTCT | | ILSCSEICYTFIIVPK | |
| | CTTGCTCTGAGATTTGCTACACCTTCATCATTGTACCCAAGATGCTG | | MLVDLLSQKKTISFLG | |
| | GTTGACCTGCTGTCCCAGAAGAAGACCATTTCTTTCCTGGGCTGTGC | | CAIQMFSFLFLGCSHS | |
| | CATCCAAATGTTTTCCTTCCTCTTCCTTGGCTGCTCTCACTCCTTTC | | FLLAVMGYDRYIAICN | |
| | TGCTGGCAGTCATGGGTTATGATCGTTACATAGCCATCTGTAACCCA | | PLRYSVLMGHGVCMGL | |
| | CTGCGCTACTCAGTGCTAATGGGACATGGGGTGTGTATGGGACTAGT | | VAAACACGFTVAQIIT | |
| | GGCTGCTGCCTGTGCCTGTGGCTTCACTGTTGCACAGATCATCACAT | | SLVFHLPFYSSNQLHH | |
| | CCTTGGTATTTCACCTGCCTTTTTATTCCTCCAATCAACTACATCAC | | FFCDIAPVLKLASHHN | |
| | TTCTTCTGTGACATTGCTCCTGTCCTCAAGCTGGCATCTCACCATAA | | HFSQIVIFMLCTLVLA | |
| | CCACTTTAGTCAGATTGTCATCTTCATGCTCTGTACATTGGTCCTGG | | IPLLLILVSYVHILSA | |
| | CTATCCCCTTATTGTTGATCTTGGTGTCCTATGTTCACATCCTCTCT | | ILQFPSTLGRCKAFST | |
| | GCCATACTTCAGTTTCCTTCCACACTGGGTAGGTGCAAAGCTTTTTC | | CVSHLIIVTVHYGCAS | |
| | TACCTGTGTATCTCACCTCATTATTGTCACTGTCCACTATGGCTGTG | | FIYLRPQSNYSSSQDA | |
| | CCTCCTTTATCTACTTAAGGCCTCAGTCCAACTACTCCTCAAGCCAG | | LISVSYTIITPLFNPM | |
| | GATGCTCTAATATCAGTATCCTACACTATTATAACTCCATTGTTCAA | | IYSLRNKEFKSALCKI | |
| | CCCAATGATTTATAGCTTGAGAAATAAAGAGTTCAAATCAGCTCTTT | | VRRTISLL | |
| | GTAAAATTGTGAGAAGAACAATTTCCCTGTTGTAAAATTAATCTTGA | | | |
| | TTCTGGCCAG | | | |
| SC | ATGCCCCAAATTCTTATATTCACATACCTGAATATGTTTTACTTCTT | 99 | MFYFFPPLQILAENLT | 100 |
| 128993196_A | TCCCCCTTTGCAGATCTTGGCAGAAAACCTCACCATGGTCACCGAAT | | MVTEFLLLGFSSLGEI | |
| | TCCTGTTGCTGGGTTTTTCCAGCCTTGGTGAAATTCAGCTGGCCCTC | | QLALFVVFLFLYLVIL | |
| | TTTGTAGTTTTTCTTTTTCTGTATCTAGTCATTCTTAGTGGCAATGT | | SGNVTIISVIHLDKSL | |
| | CACCATTATCAGTGTCATCCACCTGGATAAAAGCCTCCACACACCAA | | HTPMYFFLGILSTSET | |
| | TGTACTTCTTCCTTGGCATTCTCTCAACATCTGAGACCTTCTACACC | | FYTFVILPKMLINLLS | |
| | TTTGTCATTCTACCCAAGATGCTCATCAATCTACTTTCTGTGGCCAG | | VARTISFNCCALQMFF | |
| | GACAATCTCCTTCAACTGTTGTGCTCTTCAAATGTTCTTCTTCCTTG | | FLGFAITNCLLLGVMG | |
| | GTTTTGCCATTACCAACTGCCTGCTATTGGGTGTGATGGGTTATGAT | | YDRYAAICHPLHYPTL | |
| | CGCTATGCTGCCATTTGTCACCCTCTGCATTACCCCACTCTTATGAG | | MSWQVCGKLAAACAIG | |
| | CTGGCAGGTGTGTGGAAAACTGGCAGCTGCCTGTGCAATTGGTGGCT | | GFLASLTVVNLVFSLP | |
| | TCTTGGCCTCTCTTACAGTAGTAAATTTAGTTTTCAGCCTCCCTTTT | | FCSANKVNHYFCDISA | |
| | TGTAGCGCCAACAAAGTCAATCATTACTTCTGTGACATCTCAGCAGT | | VILLACTNTDVNEFVI | |
| | CATTCTTCTGGCTTGTACCAACACAGATGTTAACGAATTTGTGATAT | | FICGVLVLVVPFLFIC | |
| | TCATTTGTGGAGTTCTTGTACTTGTGGTTCCCTTTCTGTTTATCTGT | | VSYLCILRTILKIPSA | |
| | GTTTCTTATCTCTGCATTCTGAGGACTATCCTGAAGATTCCCTCAGC | | EGRRKAFSTCASHLSV | |
| | TGAGGGCAGACGGAAAGCGTTTTCCACCTGCGCCTCTCACCTCAGTG | | VIVHYGCASFIYLRPT | |
| | TTGTTATTGTTCATTATGGCTGTGCTTCCTTCATCTACCTGAGGCCT | | ANYVSNKDRLVTVTYT | |
| | ACAGCAAACTATGTGTCCAACAAAGACAGGCTGGTGACGGTGACATA | | IVTPLLNPMVYSLRNK | |
| | CACGATTGTCACTCCATTACTAAACCCCATGGTTTATAGCCTCAGAA | | DVQLAIRKVLGKKGSL | |
| | ACAAGGATGTCCAACTTGCTATCAGAAAAGTGTTGGGCAAGAAAGGT | | KLYN | |
| | TCTCTAAAACTATATAATTGAAATATTATTACATTTTAG | | | |
| SC | ATTTATGCCATCTGCCTCTGCCATGATCATTTTCAACCTGAGCAGTT | 101 | MPSASAMIIFNLSSYN | 102 |
| 35113271_A | ACAATCCAGGACCCTTCATTCTGGTAGGGATCCCAGGCCTGGAGCAA | | PGPFILVGIPGLEQFH | |
| | TTCCATGTGTGGATTGGAATTCCCTTCTGTATCATCTACATTGTAGC | | VWIGIPFCIIYIVAIV | |
| | TATTGTGGGAAACTGCATCCTTCTCTACCTCATTGTGGTGGAGCATA | | GNCILLYLIVVEHSLH | |
| | GTCTTCATGAACCCATGTTCTTCTTTCTCTCCATGCTGGCCATGACT | | EPMFFFLSMLAMTDLI | |
| | GACCTCATCTTGTCCACAGCTGGTGTGCCTAAAGCACTCAGTATCTT | | LSTAGVPKALSIFWLG | |
| | TTGGCTAGGGGCTCGCGAAATCACATTCCCAGGATGCCTTACACAAA | | AREITFPGCLTQMFFL | |
| | TGTTCTTCCTTCACTATAACTTTGTCCTGGATTCAGCCATTCTGATG | | HYNFVLDSAILMAMAF | |
| | GCCATGGCATTTGATCACTATGTAGCTATCTGTTCTCCCTTGAGATA | | DHYVAICSPLRYTTIL | |
| | TACCACCATCTTGACTCCCAAGACCATCATCAAGAGTGCTATGGGCA | | TPKTIIKSAMGISFRS | |
| | TCTCCTTTCGAAGCTTCTGCATCATCCTGCCAGATGTATTCTTGCTG | | FCIILPDVFLLTCLPF | |
| | ACATGCCTGCCTTTCTGCAGGACACGCATCATACCCCACACATACTG | | CRTRIIPHTYCEHIGV | |
| | TGAGCATATAGGTGTTGCCCAGCTCGCCTGTGCTGATATCTCCATCA | | AQLACADISINFWYGF | |
| | ACTTCTGGTATGGCTTTTGTGTTCCCATCATGACGGTCATCTCAGAT | | CVPIMTVISDVILIAV | |
| | GTGATTCTCATTGCTGTTTCCTACGCACACATCCTCTGTGCTGTCTT | | SYAHILCAVFGLPSQD | |
| | TGGCCTTCCCTCCCAAGATGCCTGCCAGAAAGCCCTCGGCACTTGTG | | ACQKALGTCGSHVCVI | |
| | GTTCTCATGTCTGTGTCATCCTCATGTTTTATACACCTGCCTTTTTC | | LMFYTPAFFSILAHRF | |
| | TCCATCCTCGCCCATCGCTTTGCACACAATGTCTCTCGCACCTTCCA | | GHNVSRTFHIMFANLY | |
| | CATCATGTTTGCCAATCTCTACATTGTTATCCCACCTGCACTCAACC | | IVIPPALNPMVYGVKT | |
| | CCATGGTTTACGGAGTGAAGACCAAGCAGATCAGAGATAAGGTTATA | | KQIRDKVILLFSKGTG | |
| | CTTTTGTTTTCTAAGGGTACAGGATGAT | | | |
| CG 55956- | GAATCAACGAGTGAAACGAATAACTCTATGGTGACTGAATTCATTTT | 103 | MVTEFIFLGLSDSQEL | 104 |
| 02 | TCTGGGTCTCTCTGATTCTCAGGAACTCCAGACCTTCCTATTTATGT | | QTFLFMLFFVFYGGIV | |
| | TGTTTTTTGTATTCTATGGAGGAATCGTGTTTGGAAACCTTCTTATT | | FGNLLIVITVVSDSHL | |
| | GTCATAACAGTGGTATCTGACTCCCACCTTCACTCTCCCATGTACTC | | HSPMYSLLANLSLIDL | |
| | CCTGCTAGCCAACCTCTCACTCATTGATCTGTCTCTGTCTTCAGTCA | | SLSSVTAPKMITDFFS | |
| | CAGCCCCCAAGATGATTACTGACTTTTTCAGCCAGCGCAAAGTCATC | | QRKVISFKGCLVQIFL | |
| | TCTTTCAAGGGCTGCCTTGTTCAGATATTTCTCCTTCACTTCTTTGG | | LHFFGGGEMVILIAMG | |
| | TGGGGGTGAGATGGTGATCCTCATAGCCATGGGCTTTGACAGATATA | | FDRYIAICKPLHYTTI | |
| | TAGCAATATGCAAGCCCCTACACTACACTACAATTATGTGTGGCAAC | | MCGNACVGIMAVAWGI | |
| | GCATGTGTCGGCATTATGGCTGTCGCATGGGGAATTGGCTTTCTCCA | | GFLHSVSQLAFAVHLL | |
| | TTCGGTGAGCCAGTTGGCCTTTGCCGTGCACTTACTCTTCTGTGGTC | | FCGPNEVDSFYCDLPR | |
| | CCAATGAGGTCGATAGTTTTTATTGTGACCTTCCTAGGGTAATCAAA | | VIKLACTDTYRLDIMV | |
| | CTTGCCTGTACAGATACCTACAGGCTAGATATTATGGTCATTGCTAA | | IANSGVLTVCSFVLLI | |
| | CAGTGGTGTGCTCACTGTGTGTTCTTTTGTTCTTCTAATCATCTCAT | | ISYTIILMTIQHRPLD | |
| | ACACTATCATCCTAATGACCATCCAGCATCGCCCTTTAGATAAGTCG | | KSSKALSTLTAHITVV | |
| | TCCAAAGCTCTGTCCACTTTGACTGCTCACATTACAGTAGTTCTTTT | | LLFFGPCVFIYAWPFP | |
| | GTTCTTTGGACCATGTGTCTTTATTTATGCCTGGCCATTCCCCATCA | | IKSLDKFLAVFYSVIT | |
| | AGTCATTAGATAAATTCCTTGCTGTATTTTATTCCGTGATCACCCCT | | PLLNPIIYTLRNKDMK | |
| | CTCTTGAACCCAATTATATACACACTGAGGAACAAAGACATGAAGAC | | TAIRQLRKWDAHSSVK | |
| | GGCAATAAGACAGCTGAGAAAATGGGATGCACATTCTAGTGTAAAGT | | F | |
| | TTTAGATCTTATATAACTGTGAGATTAATCTCAGATAATGACACAAA | | | |
| | ATATAGTGAAGTTG | | | |
| CG 56103- | GGGTGGCCGCCATGCAGGGGCTAAACCACACCTCCGTGTCTGAATTC | 105 | MQGLNHTSVSEFILVG | 106 |
| 02 | ATCCTCGTTGGCTTCTCTGCCTTCCCCCACCTCCAGCTGATGCTCTT | | FSAFPHLQLMLFLLFL | |
| | CCTGCTGTTCCTGCTGATGTACCTGTTCACGCTGCTGGGCAACCTGC | | LMYLFTLLGNLLIMAT | |
| | TCATCATGGCCACTGTCTGGAGCGAGCGCAGCCTCCACATGCCCATG | | VWSERSLHMPMYLFLC | |
| | TACCTCTTCCTGTGTGCCCTCTCCATCACCGAGATCCTCTACACCGT | | ALSITEILYTVAIIPR | |
| | GGCCATCATCCCGCGCATGCTGGCCGACCTGCTGTCCACCCAGCGCT | | MLADLLSTQRSIAFLA | |
| | CCATCGCCTTCCTGGCCTGTGCCAGTCAGATGTTCTTCTCCTTCAGC | | CASQMFFSFSFGFTHS | |
| | TTCGGCTTCACCCACTCCTTCCTGCCCACTGTCATGGGCTACGACCG | | FLPTVMGYDRYVAICH | |
| | CTACGTGGCCATCTGCCACCCCCTGCGTTACAACGTGCTCATGAGCC | | PLRYNVLMSLRGCTCR | |
| | TGCGGGGCTGCACCTGCCGGGTGGGCTGCTCCTGGGCTGGTGGCTTG | | VGCSWAGGLVMGMVVT | |
| | GTCATGGGGATGGTGGTGACCTCGGCCATTTTCCACCTCGCCTTCTG | | SAIFHLAFCGHKEIHH | |
| | TGGACACAAGGAGATCCACCATTTCTTCTGCCACGTGCCACCTCTGT | | FFCHVPPLLKLACGDD | |
| | TGAAGTTGGCCTGTGGAGATGATGTGCTGGTGGTGGCCAAAGGCGTG | | VLVVAKGVGLVCITAL | |
| | GGCTTGGTGTGTATCACGGCCCTGCTGGGCTGTTTTCTCCTCATCCT | | LGCFLLILLSYAFIVA | |
| | CCTCTCCTATGCCTTCATCGTGGCCGCCATCTTGAAGATCCCTTCTG | | AILKIPSAEGRNKAFS | |
| | CTGAAGGTCGGAACAAGGCCTTCTCCACCTGTGCCTCTCACCTCACT | | TCASHLTVVVVHYGFA | |
| | GTGGTGGTCGTGCACTATGGCTTTGCCTCCGTCATTTACCTGAAGCC | | SVIYLKPKGPQYPEGD | |
| | CAAAGGTCCCCAGTATCCGGAAGGAGACACCTTGATGGGCATCACCT | | TLMGITYTVLTPFLSP | |
| | ACACGGTCCTCACACCCTTCCTCAGCCCCATCATCTTCAGCCTCAGG | | IIFSLRNKELKVAMKK | |
| | AACAAGGAGCTGAAGGTCGCCATGAAGAAGACTTGCTTCACCAAACT | | TCFTKLFPQNC | |
| | CTTTCCACAGAACTGCTGAAATGGCTGACTTTCTCTCAAGAGAT | | | |
| CG 55773- | CAATGCATTTTGTGACTGAGTTTGTCCTCCTGGGTTTCCATGGTCAA | 107 | MHFVTEFVLLGFHGQR | 108 |
| 02 | AGGGAGATGCAGAGCTGCTTCTTCTCATTCATCCTGGTTCTCTATCT | | EMQSCFFSFILVLYLL | |
| | CCTGACACTGCTAGGGAATGGAGCTATTGTCTGTGCAGTGAAATTGG | | TLLGNGAIVCAVKLDR | |
| | ACAGGCGGCTCCACACACCCATGTACATCCTTCTGGGAAACTTTGCC | | RLHTPMYILLGNFAFL | |
| | TTTCTAGAGATCTGGTACATTTCCTCCACTGTCCCAAACATGCTAGT | | EIWYISSTVPNMLVNI | |
| | CAATATCCTCTCTGAGACTAAAACCATCTCCTTCTCTGGTTGCTTCC | | LSETKTISFSGCFLQF | |
| | TGCAATTCTATTTCTTTTTTTCACTGGGTACAACAGAGTGTTTCTTT | | YFFFSLGTTECFFLSV | |
| | TTATCAGTTATGGCTTATGATCGGTATCTGGCCATCTGTCGTCCATT | | MAYDRYLAICRPLHYP | |
| | ACACTACCCCTCCATCATGACTGGGAAGTTCTGTATAATTCTGGTCT | | SIMTGKFCIILVCVCW | |
| | GTGTATGCTGGGTAGGCGGATTTCTCTGCTATCCAGTCCCTATTGTT | | VGGFLCYPVPIVLISQ | |
| | CTTATCTCCCAACTTCCCTTCTGTGGGCCCAACATCATTGACCACTT | | LPFCGPNIIDHFVCDP | |
| | TGTGTGTGACCCAGGCCCATTGTTTGCACTGGCCTGCATCTCTGCTC | | GPLFALACISAPSTEL | |
| | CTTCCACTGAGCTTATCTGTTACACCTTCAACTCGATGATTATCTTT | | ICYTFNSMIIFGPFLS | |
| | GGGCCCTTCCTCTCCATCTTGGGATCTCACACTCTGGTCATCAGAGC | | ILGSHTLVIRAVLCIP | |
| | TGTGCTTTGTATTCCCTCTGGTGCTGGTCGAACTAAAGCTTTCTCCA | | SGAGRTKAFSTRGSHL | |
| | CACGTGGGTCCCACCTAATGGTGGTGTCTCTATTCTATGGAACCCTT | | MVVSLFYGTLMVMYVS | |
| | ATGGTGATGTATGTGAGCCCAACATCAGGGAACCCAGCAGGAATGCA | | PTSGNPAGMQKIITLV | |
| | GAAGATCATCACTCTGGTATACACAGCAATGACTCCATTCTTAAATC | | YTAMTPFLNPLIYSLR | |
| | CCCTTATCTATAGTCTTCGAAACAAAGACATGAAAGATGCTTTAAAG | | NKDMKDALKRVLGLTV | |
| | AGAGTCCTGGGGTTAACAGTTAGCCAAAACTGAGA | | SQN | |
| CG 50285- | ATTTGTTGGGAATATGGAAAAAAGAAATCTAACAGTTGTCAGGGAAT | 109 | MEKRNLTVVREFVLLG | 110 |
| 02 | TCGTCCTTCTGGGACTTCCTAGCTCAGCAGAGCAGCAGCACCTCCTG | | LPSSAEQQHLLSVLFL | |
| | TCTGTGCTCTTTCTCTGTATGTATTTAGCCACCACCTTGGGGAACAT | | CMYLATTLGNMLIIAT | |
| | GCTCATCATTGCGACGATTGGCTTTGACTCTCACCTCCATTCCCCTA | | IGFDSHLHSPMYFFLS | |
| | TGTACTTCTTCCTTAGTAACTTGGCCTTTGTTGACATCTGCTTTACG | | NLAFVDICFTSTTVPQ | |
| | TCGACTACAGTCCCCCAAATGGTAGTGAATATCTTGACTGGCACCAA | | MVVNILTGTKTISFAG | |
| | GACTATCTCTTTTGCAGGCTGCCTCACCCAGCTCTTCTTCTTCGTTT | | CLTQLFFFVSFVNMDS | |
| | CTTTTGTGAATATGGACAGCCTCCTTCTGTGTGTGATGGCGTATGAT | | LLLCVMAYDRYVAICH | |
| | AGATATGTGGCGATTTGCCACCCCTTACATTACACCGCCAGAATGAA | | PLHYTARMNLCLCVQL | |
| | CCTGTGCCTTTGTGTCCAGCTAGTGGCTGGACTGTGGCTTGTTACTT | | VAGLWLVTYLHALLHT | |
| | ACCTCCACGCCCTCCTGCATACTGTCCTAATAGCACAGCTGTCCTTC | | VLIAQLSFCASNIIHH | |
| | TGTGCCTCCAATATCATCCATCATTTCTTCTGTGATCTCAATCCTCT | | FFCDLNPLLQLSCSDV | |
| | CCTGCAGCTCTCTTGCTCTGACGTCTCCTTCAATGTAATGATCATTT | | SFNVMIIFAVGGLLAL | |
| | TTGCAGTAGGAGGTCTATTGGCTCTCACGCCCCTTGTCTGTATCCTC | | TPLVCILVSYGLIFST | |
| | GTATCTTATGGACTTATCTTCTCCACTGTTCTGAAGATCACCTCTAC | | VLKITSTQGKQRAVST | |
| | TCAGGGCAAGCAGAGAGCTGTTTCCACCTGCAGCTGCCACCTGTCAG | | CSCHLSVVVLFYGTAI | |
| | TGGTGGTGTTGTTTTACGGCACAGCCATCGCCGTCTATTTCAGCCCT | | AVYFSPSSPHMPESDT | |
| | TCATCCCCCCATATGCCTGAGAGCGACACTCTGTCAACCATCATGTA | | LSTIMYSMVAPMLNPF | |
| | TTCAATGGTGGCTCCGATGCTGAATCCTTTCATCTATACCCTAAGGA | | IYTLRNRDMKRGLQKM | |
| | ACAGGGATATGAAGAGGGGACTTCAGAAAATGCTTCTCAAGTGCACA | | LLKCTVFQQQ | |
| | GTCTTTCAGCAGCAATAATGACCTCA | | | |
| SC | ATTTGTTGGGAATATGGAAAAAAGAAATCTAACAGTTGTCAGGGAAT | 111 | MEKRNLTVVREFVLLG | 112 |
| 88066237_A | TCGTCCTTCTGGGACTTCCTAGCTCAGCAGAGCAGCAGCACCTCCTG | | LPSSAEQQHLLSVLFL | |
| | TCTGTGCTCTTTCTCTGTATGTATTTAGCCACCACCTTGGGGAACAT | | CMYLATTLGNMLIIAT | |
| | GCTCATCATTGCGACGATTGGCTTTGACTCTCACCTCCATTCCCCTA | | IGFDSHLHSPMYFFLS | |
| | TGTACTTCTTCCTTAGTAACTTGGCCTTTGTTGACATCTGCTTTACG | | NLAFVDICFTSTTVPQ | |
| | TCGACTACAGTCCCCCAAATGGTAGTGAATATCTTGACTGGCACCAA | | MVVNILTGTKTISFAG | |
| | GACTATCTCTTTTGCAGGCTGCCTCACCCAGCTCTTCTTCTTCGTTT | | CLTQLFFFVSFVNMDS | |
| | CTTTTGTGAATATGGACAGCCTCCTTCTGTGTGTGATGGCGTATGAT | | LLLCVMAYDRYVAICH | |
| | AGATATGTGGCGATTTGCCACCCCTTACATTACACCGCCAGAATGAA | | PLHYTARMNLCLCVQL | |
| | CCTGTGCCTTTGTGTCCAGCTAGTGGCTGGACTGTGGCTTGTTACTT | | VAGLWLVTYLHALLHT | |
| | ACCTCCACGCCCTCCTGCATACTGTCCTAATAGCACAGCTGTCCTTC | | VLIAQLSFCASNIIHH | |
| | TGTGCCTCCAATATCATCCATCATTTCTTCTGTGATCTCAATCCTCT | | FFCDLNPLLQLSCSDV | |
| | CCTGCAGCTCTCTTGCTCTGACGTCTCCTTCAATGTAATGATCATTT | | SFNVMIIFAVGGLLAL | |
| | TTGCAGTAGGAGGTCTATTGGCTCTCACGCCCCTTGTCTGTATCCTC | | TPLVCILVSYGLIFST | |
| | GTATCTTATGGACTTATCTTCTCCACTGTTCTGAAGATCACCTCTAC | | VLKITSTQGKQRAVST | |
| | TCAGGGCAAGCAGAGAGCTGTTTCCACCTGCAGCTGCCACCTGTCAG | | CSCHLSVVVLFYGTAI | |
| | TGGTGGTGTTGTTTTACGGCACAGCCATCGCCGTCTATTTCAGCCCT | | AVYFSPSSPHMPESDT | |
| | TCATCCCCCCATATGCCTGAGAGCGACACTCTGTCAACCATCATGTA | | LSTIMYSMVAPMLNPF | |
| | TTCAATGGTGGCTCCGATGCTGAATCCTTTCATCTATACCCTAAGGA | | IYTLRNRDMKRGLQKM | |
| | ACAGGGATATGAAGAGGGGACTTCAGAAAATGCTTCTCAAGTGCACA | | LLKCTVFQQQ | |
| | GTCTTTCAGCAGCAATAATGACCTCA | | | |
| CG 55766- | ATGTCCATAAAATCAATGCACGACTTCATTACTGAAAATGGAAAGAC | 113 | MERQNQSCVVEFILLG | 114 |
| 01 | AAAATCAAAGCTGTGTGGTTGAATTCATCCTCTTGGGCTTTTCTAAC | | FSNYPELQGQLFVAFL | |
| | TATCCTGAGCTCCAGGGGCAGCTCTTTGTGGCTTTCCTGGTTATTTA | | VIYLVTLIGNAIIIVI | |
| | TCTGGTGACCCTGATAGGAAATGCCATTATTATAGTCATCGTCTCCC | | VSLDQSLHVPMYLFLL | |
| | TAGACCAGAGCCTCCACGTTCCCATGTACCTGTTTCTCCTGAACTTA | | NLSVVDLSFSAVIMPE | |
| | TCTGTGGTGGACCTGAGTTTCAGTGCAGTTATTATGCCTGAAATGCT | | MLVVLSTEKTTISFGG | |
| | GGTGGTCCTCTCTACTGAAAAAACTACAATTTCTTTTGGGGGCTGTT | | CFAQMYFILLFGGAEC | |
| | TTGCACAGATGTATTTCATCCTTCTTTTTGGTGGGGCTGAATGTTTT | | FLLGVMAYDRFAAICH | |
| | CTTCTGGGAGTAATGGCTTATGACCGATTTGCTGCAATTTGCCATCC | | PLNYQMIMNKGGFMKL | |
| | TCTCAACTACCAAATGATTATGAATAAAGGAGGTTTTATGAAATTAA | | IIFSWALGFMLGTVQT | |
| | TTATATTTTCATGGGCCTTAGGTTTTATGTTAGGTACTGTTCAAACA | | SWVSSFPFCGLNEINH | |
| | TCATGGGTATCTAGTTTTCCCTTTTGTGGCCTTAATGAAATTAACCA | | ISCETPAVLELACADT | |
| | TATATCTTGTGAAACCCCAGCAGTGTTAGAACTTGCATGTGCAGACA | | FLFEIYAFTGTFLIIL | |
| | CGTTTTTGTTTGAAATCTATGCATTCACAGGCACCTTTTTGATTATT | | VPFLLILLSYIRVLFA | |
| | TTGGTTCCTTTCTTGTTGATACTCTTGTCTTACATTCGAGTTCTGTT | | ILKMPSTTGRQKAFST | |
| | TGCCATCCTGAAGATGCCATCAACCACTGGGAGACAAAAGGCCTTTT | | CAAHLTSVTLFYGTAS | |
| | CCACCTGTGCCGCTCACCTCACATCTGTGACCCTATTCTATGGCACA | | MTYLQPKSGYSPETKK | |
| | GCCAGTATGACTTATTTACAACCCAAATCTGGCTACTCACCGGAAAC | | VMSLSYSLLTPLLNPL | |
| | CAAGAAAGTGATGTCATTGTCTTACTCACTTCTGACACCACTGCTGA | | IYSLRNSEMKRALMKL | |
| | ATCCGCTTATCTACAGTTTGCGAAATAGTGAGATGAAGAGGGCTTTG | | WRRRVVLHTI | |
| | ATGAAATTATGGCGAAGGCGAGTGGTTTTACACAATCTGACT | | | |
| CG 56968- | CTATCAGGTGCACCCTCCTGGTGGACATTGCCCCTCATTGCTGTCTA | 115 | LSGAPSWWTLPLIAVY | 116 |
| 01 | CCTTCTCTCTGCCCTGGGAAATGGCACCATCCTCTGGATCATTGCCC | | LLSALGNGTILWIIAL | |
| | TGGAGCCCGCCCTGCACCGCCCAATGCACTTCTTCCTCTTCTTGCTT | | EPALHRPMHFFLFLLS | |
| | AGTGTGTCTGATATTGGATTGGTCACTGCCCTGATGCCCACACTGCT | | VSDIGLVTALMPTLLG | |
| | GGGCATCGCCCTTGCTGGTGCTCACACTGTTCCTGCCTCAGCCTGCC | | IALAGAHTVPASACLL | |
| | TTCTACAGATGGTTTTTATCCATGTCTTTTCTGTCATGGAGTCCTCT | | QMVFIHVFSVMESSVL | |
| | GTCTTGCTCGCCATGTCCATTGATCGGGCACTGGCCATCTGCCGACC | | LAMSIDRALAICRPLH | |
| | TCTCCACTACCCAGCGCTCCTCACCAATGGTGTAATTAGCAAAATCA | | YPALLTNGVISKISLA | |
| | GCCTGGCCATTTCTTTTCGATGCCTGGGTCTCCATCTGCCCCTGCCA | | ISFRCLGLHLPLPFLL | |
| | TTCCTGCTGGCCTACATGCCCTACTGCCGCCCACAGGTCCTAACCCA | | AYMPYCRPQVLTHSYC | |
| | TTCTTATTGCTTGCATCCAGATGTGGCTCGTTTGGCCTGCCCAGAAG | | LHPDVARLACPEAWGA | |
| | CTTGGGGTGCAGCCTACAGCCTATTTGTGGTTCTTTCAGCCATGGGT | | AYSLFVVLSAMGLDPL | |
| | TTGGACCCCCTGCTTATTTTCTTCTCCTATGGCCTGATTGGCAAGGT | | LIFFSYGLIGKVLQGV | |
| | GTTGCAAGGTGTGGAGTCCAGAGAGGATCGCTGGAAGGCTGGTCAAA | | ESREDRWKAGQTCAAH | |
| | CCTGTGCTGCCCACCTCTCTGCAGTGCTCCTCTTCTATATCCCTATG | | LSAVLLFYIPMIFPAL | |
| | ATCTTCCCGGCACTGATTAACCATCCTGAGCTGCCAATCACTCAGCA | | INHPELPITQHTHTLL | |
| | TACCCATACTCTTCTATCCTATGTCCATTTCCTTCTTCCTCCATTGA | | SYVHFLLPPLINPILY | |
| | TAAACCCTATTCTCTATAGTGTCAAGATGAAGGAGATTAGAAAGAGA | | SVKMKEIRKRILNRLQ | |
| | ATACTCAACAGGTTGCAGCCCAGGAAGGTGGGTGGTGCTCAGTGA | | PRKVGGAQ | |
| CG 57860- | ATTACTCCTGCAATAATGGCAAATCTCACAATCGTGACTGAATTTAT | 117 | MANLTIVTEFILMGFS | 118 |
| 01 | CCTCATGGGGTTTTCTACCAATAAAAATATGTGCATTTTGCATTCGA | | TNKNMCILHSILFLLI | |
| | TTCTCTTCTTGTTGATTTATTTGTGTGCCCTGATGGGGAATGTCCTC | | YLCALMGNVLIIMITT | |
| | ATTATCATGATCACAACTTTGGACCATCATCTCCACACCCCCGTGTA | | LDHHLHTPVYFFLKNL | |
| | TTTCTTCTTGAAGAATCTATCTTTCTTGGATCTCTGCCTTATTTCAG | | SFLDLCLISVTAPKSI | |
| | TCACGGCTCCCAAATCTATCGCCAATTCTTTGATACACAACAACTCC | | ANSLIHNNSISFLGCV | |
| | ATTTCATTCCTTGGCTGTGTTTCCCAGGTCTTTTTGTTGCTTTCTTC | | SQVFLLLSSASAELLL | |
| | AGCATCTGCAGAGCTGCTCCTCCTCACGGTGATGTCCTTTGACCGCT | | LTVMSFDRYTAICHPL | |
| | ATACTGCTATATGTCACCCTCTGCACTATGATGTCATCATGGACAGG | | HYDVIMDRSTCVQRAT | |
| | AGCACCTGTGTCCAAAGAGCCACTGTGTCTTGGCTGTATGGGGGTCT | | VSWLYGGLIAVMHTAG | |
| | GATTGCTGTGATGCACACAGCTGGCACCTTCTTATCCTACTGTGGGT | | TFLSYCGSNMVHQFFC | |
| | CCAACATGGTCCATCAGTTCTTCTGTGACATTCCCCAGTTATTAGCT | | DIPQLLAISCSENLIR | |
| | ATTTCTTGCTCAGAAAATTTAATAAGAGAAATTGCACTCATCCTTAT | | EIALILINVVLDFCCF | |
| | TAATGTAGTTTTGGATTTCTGCTGTTTTATTGTCATCATCATTACCT | | IVIIITYVHVFSTVKK | |
| | ATGTCCACGTCTTCTCTACAGTCAAGAAGATCCCTTCCACAGAAGGC | | IPSTEGQSKAYSTCLP | |
| | CAGTCAAAAGCCTACTCTACTTGCCTTCCACACTTGCTGGTTGTGTT | | HLLVVLFLSTGFIAYL | |
| | ATTTCTTTCCACTGGATTCATTGCTTATCTGAAGCCAGCTTCAGAGT | | KPASESPSILDAVISV | |
| | CTCCTTCTATTTTGGATGCTGTAATTTCTGTGTTCTACACTATGCTG | | FYTMLPPTFNPIIYSL | |
| | CCCCCAACCTTTAATCCCATTATATACAGTTTGAGAAACAAGGCCAT | | RNKAIKVALGMLIKGK | |
| | AAAGGTGGCTCTGGGGATGTTGATAAAGGGAAAGCTCACCAAAAAGT | | LTKK | |
| | AAAAGCT | | | |
| CG 57372- | CTACAGGTACTATTGTTTGCCCTTTTGCTGCTGGCCTATGTGTTGGT | 119 | LQVLLFALLLLAYVLV | 120 |
| 01 | GCTGACTGAGAACACACTCATCATTATGGCAATTAGGAACCATTCCA | | LTENTLIIMAIRNHST | |
| | CCCTCCACAAACCCATGTACTTTTTTCTAGCTAATATGTCCTTTCTG | | LHKPMYFFLANMSFLE | |
| | GAGATCTGGTATGTCACTGTCACTATTCCCAAGATGCTTGCTGGCTT | | IWYVTVTIPKMLAGFV | |
| | TGTTGGATCCAAACAGGATCATGGACAGCTAATCTCCTTTGGGGGAT | | GSKQDHGQLISFGGCM | |
| | GCATGACACAGCTCTACTTTTTCCTTGGCTTGGGCTGCACTGAGTGT | | TQLYFFLGLGCTECVL | |
| | GTCCTTCTCGCTGTTATGGCCTATGATCGCTATATGGCCATCTGCTA | | LAVMAYDRYMAICYPL | |
| | TCCTCTCCACTACCCAGTCATTGTCAGTGGCCGGCTGTGTGTGCAGA | | HYPVIVSGRLCVQMAA | |
| | TGGCTGCTGGCTCTTGGGCTGGAGGTTTTGGCATCTCCATGGTCAAA | | GSWAGGFGISMVKVFL | |
| | GTTTTTCTTATTTCTGGCCTCTCTTACTGTGGCCCCAACATCATCAA | | ISGLSYCGPNIINHFF | |
| | CCACTTTTTCTGTGATGTCTCTCCATTGCTCAACCTCTCATGCACTG | | CDVSPLLNLSCTDMST | |
| | ATATGTCCACAGCAGAGCTTACAGATTTCATCCTGGCCATTTTTATT | | AELTDFILAIFILLGP | |
| | CTTCTAGGGCCACTCTCTGTCACTGGGGCCTCCTATGTGGCCATTAC | | LSVTGASYVAITGAVM | |
| | TGGTGCTGTGATGCACATTCCTTCGGCTGCTGGACGCTATAAGGCCT | | HIPSAAGRYKAFSTCA | |
| | TTTCCACCTGTGCCTCTCATCTCACTGTTGTGATAATCTTCTATGCA | | SHLTVVIIFYAASIFI | |
| | GCCAGTATCTTCATCTATGCTCGGCCAAAGGCACTCTCAGCTTTTGA | | YARPKALSAFDTNKLV | |
| | CACCAACAAGTTGGTCTCTGTACTGTATGCTGTCATTGTACCATTGC | | SVLYAVIVPLLNPIIY | |
| | TCAATCCCATCATTTACTGCCTGAGCAATCAAGAGGTCAAGAGAGCC | | CLSNQEVKRALCCTLQ | |
| | CTATGCTGTACTCTGCAACCTGTACCAGCACCAGGATCCTGACCCCA | | PVPAPGS | |
| | AGAAAGCTAGCAGAAATGTATAGAAGGGAT | | | |
| CG 56081- | ATCCTATGGAACCACAGAACACCACACAGGTATCAATGTTTGTCCTC | 121 | MEPQNTTQVSMFVLLG | 122 |
| 01 | TTAGGGTTTTCACAGACCCAAGAGCTCCAGAAATTCCTGTTCCTTCT | | FSQTQELQKFLFLLFL | |
| | GTTCCTGTTAGTCTATGTTACCACCATTGTGGGAAACCTCCTTATCA | | LVYVTTIVGNLLIMVT | |
| | TGGTCACAGTGACTTTTGACTGCCGGCTCCACACACCCATGTATTTT | | VTFDCRLHTPMYFLLR | |
| | CTGCTCCGAAATCTAGCTCTCATAGACCTCTGCTATTCCACAGTCAC | | NLALIDLCYSTVTSPK | |
| | CTCTCCAAAGATGCTGGTGGACTTCCTCCATGAGACCAAGACGATCT | | MLVDFLHETKTISYQG | |
| | CCTACCAGGGCTGCATGGCCCAGATCTTCTTCTTCCACCTTTTGGGA | | CMAQIFFFHLLGGGTV | |
| | GGTGGGACTGTCTTTTTTCTCTCAGTCATGGCCTATGACCGCTACAT | | FFLSVMAYDRYIAISQ | |
| | AGCCATCTCCCAGCCCCTCCGGTATGTCACCATCATGAACACTCAAT | | PLRYVTIMNTQLCVGL | |
| | TGTGTGTGGGCCTGGTAGTAGCCGCCTGGGTGGGGGGCTTTGTCCAC | | VVAAWVGGFVHSIVQL | |
| | TCCATTGTCCAACTGGCTCTGATACTTCCACTGCCCTTCTGTGGCCC | | ALILPLPFCGPNIIDN | |
| | CAATATCATAGATAACTTCTACTGTGATGTTCCCCAAGTACTGAGAC | | FYCDVPQVLRLACTDT | |
| | TTGCCTGCACTGATACCTCCCTCCTGGAGTTCCTCATGATCTCCAAC | | SLLEFLMISNSGLLVI | |
| | AGTGGGCTGCTAGTTATCATCTGGTTCCTCCTCCTTCTGATCTCTTA | | IWFLLLLISYTVILVM | |
| | TACTGTCATCCTGGTGATGCTGAGGTCCCACTCGGGAAAGGCAAGGA | | LRSHSGKARRKAASTC | |
| | GGAAGGCAGCTTCCACCTGCACCACCCACATCATCGTGGTGTCCATG | | TTHIIVVSMIFIPCIY | |
| | ATCTTCATTCCCTGTATCTATATCTATACCTGGCCCTTCACCCCATT | | IYTWPFTPFLIDKAVS | |
| | CCTCATAGACAAGGCTGTGTCCATCAGCTACACAGTCATGACCCCCA | | ISYTVMTPMLNPMIYN | |
| | TGCTCAACCCCATGATCTACAACCTGAGAAACCAGGACATGAAAGCA | | LRNQDMKAAMRRLGKC | |
| | GCCATGAGGAGATTAGGCAAGTGCCTAGTAATTTGCAGGGAGTAAAC | | LVICRE | |
| | TTTAA | | | |
| GMAP | ATGTTCTCCCCAAACCACACCATAGTGACAGAATTCATTCTCTTGGG | 123 | MFSPNHTIVTEFILLG | 124 |
| 002517_A | ACTGACAGACGACCCAGTGCTAGAGAAGATCCTGTTTGGGGTATTCC | | LTDDPVLEKILFGVFL | |
| | TTGCGATCTACCTAATCACACTGGCAGGCAACCTGTGCATGATCCTG | | AIYLITLAGNLCMILL | |
| | CTGATCAGGACCAATTCCCACCTGCAAACACCCATGTATTTCTTCCT | | IRTNSHLQTPMYFFLG | |
| | TGGCCACCTCTCCTTTGTAGACATTTGCTATTCTTCCAATGTTACTC | | HLSFVDICYSSNVTPN | |
| | CAAATATGCTGCACAATTTCCTCTCAGAACAGAAGACCATCTCCTAC | | MLHNFLSEQKTISYAG | |
| | GCTGGATGCTTCACACAGTGTCTTCTCTTCATCGCCCTGGTGATCAC | | CFTQCLLFIALVITEF | |
| | TGAGTTTTACATCCTTGCTTCAATGGCATTGGATCGCTATGTAGCCA | | YILASMALDRYVAICS | |
| | TTTGCAGCCCTTTGCATTACAGTTCCAGGATGTCCAAGAACATCTGT | | PLHYSSRMSKNICVCL | |
| | GTCTGTCTGGTCACTATCCCTTACATGTATGGGTTTCTTAGTGGGTT | | VTIPYMYGFLSGFSQS | |
| | CTCTCAGTCACTGCTAACCTTTCACTTATCCTTCTGTGGCTCCCTTG | | LLTFHLSFCGSLEINH | |
| | AAATCAATCATTTCTACTGCGCTGATCCTCCTCTTATCATGCTGGCC | | FYCADPPLIMLACSDT | |
| | TGCTCTGACACCCGTGTCAAAAAGATGGCAATGTTTGTAGTTGCAGG | | RVKKMAMFVVAGFNLS | |
| | CTTTAATCTCTCAAGCTCTCTCTTCATCATTCTTCTGTCCTATCTTT | | SSLFIILLSYLFIFAA | |
| | TCATTTTTGCAGCGATCTTCAGGATCCGTTCTGCTGAAGGCAGGCAC | | IFRIRSAEGRHKAFST | |
| | AAAGCCTTTTCTACGTGTGCTTCCCACCTGACAATAGTCACTTTGTT | | CASHLTIVTLFYGTLF | |
| | TTATGGAACCCTCTTCTGCATGTACGTAAGGCCTCCATCAGAGAAGT | | CMYVRPPSEKSVEESK | |
| | CTGTAGAGGAGTCCAAAATAACTGCAGTCTTTTATACTTTTTTGAGC | | ITAVFYTFLSPMLNPL | |
| | CCAATGCTGAACCCATTGATCTATAGCCTACGGAACACAGATGTAAT | | IYSLRNTDVILAMQQM | |
| | CCTTGCCATGCAACAAATGATTAGGGGAAAATCCTTTCATAAAATTG | | IRGKSFHKIAV | |
| | CAGTTTAGGCTT | | | |
| CG 56117- | ATGTTCTCCCCAAACCACACCATAGTGACAGAATTCATTCTCTTGGG | 125 | MFSPNHTIVTEFILLG | 126 |
| 01 | ACTGACAGACGACCCAGTGCTAGAGAAGATCCTGTTTGGGGTATTCC | | LTDDPVLEKILFGVFL | |
| | TTGCGATCTACCTAATCACACTGGCAGGCAACCTGTGCATGATCCTG | | AIYLITLAGNLCMILL | |
| | CTGATCAGGACCAATTCCCACCTGCAAACACCCATGTATTTCTTCCT | | IRTNSHLQTPMYFFLG | |
| | TGGCCACCTCTCCTTTGTAGACATTTGCTATTCTTCCAATGTTACTC | | HLSFVDICYSSNVTPN | |
| | CAAATATGCTGCACAATTTCCTCTCAGAACAGAAGACCATCTCCTAC | | MLHNFLSEQKTISYAG | |
| | GCTGGATGCTTCACACAGTGTCTTCTCTTCATCGCCCTGGTGATCAC | | CFTQCLLFIALVITEF | |
| | TGAGTTTTACATCCTTGCTTCAATGGCATTGGATCGCTATGTAGCCA | | YILASMALDRYVAICS | |
| | TTTGCAGCCCTTTGCATTACAGTTCCAGGATGTCCAAGAACATCTGT | | PLHYSSRMSKNICVCL | |
| | GTCTGTCTGGTCACTATCCCTTACATGTATGGGTTTCTTAGTGGGTT | | VTIPYMYGFLSGFSQS | |
| | CTCTCAGTCACTGCTAACCTTTCACTTATCCTTCTGTGGCTCCCTTG | | LLTFHLSFCGSLEINH | |
| | AAATCAATCATTTCTACTGCGCTGATCCTCCTCTTATCATGCTGGCC | | FYCADPPLIMLACSDT | |
| | TGCTCTGACACCCGTGTCAAAAAGATGGCAATGTTTGTAGTTGCAGG | | RVKKMAMFVVAGFNLS | |
| | CTTTAATCTCTCAAGCTCTCTCTTCATCATTCTTCTGTCCTATCTTT | | SSLFIILLSYLFIFAA | |
| | TCATTTTTGCAGCGATCTTCAGGATCCGTTCTGCTGAAGGCAGGCAC | | IFRIRSAEGRHKAFST | |
| | AAAGCCTTTTCTACGTGTGCTTCCCACCTGACAATAGTCACTTTGTT | | CASHLTIVTLFYGTLF | |
| | TTATGGAACCCTCTTCTGCATGTACGTAAGGCCTCCATCAGAGAAGT | | CMYVRPPSEKSVEESK | |
| | CTGTAGAGGAGTCCAAAATAACTGCAGTCTTTTATACTTTTTTGAGC | | ITAVFYTFLSPMLNPL | |
| | CCAATGCTGAACCCATTGATCTATACCCTACGGAACACAGATGTAAT | | IYSLRNTDVILAMQQM | |
| | CCTTGCCATGCAACAAATGATTAGGGGAAAATCCTTTCATAAAATTG | | IRGKSFHKIAV | |
| | CAGTTTAGGCTT | | | |
| CG 55708- | TGGAATCCATGGCCAGTACAAGTAATGTGACTGAGTTGATTTTCACT | 127 | MASTSNVTELIFTGLF | 128 |
| 01 | GGCCTTTTCCAGGATCCAGCGGTGCAGAGTGTATGCTTTGTGGTGTT | | QDPAVQSVCFVVFLPV | |
| | TCTCCCCGTGTACCTTGCCGCGGTGGTGGGCAATGGCCTCATCGTTC | | YLAAVVGNGLIVLTVS | |
| | TGACGGTCAGTATCAGCAAGAGTCTGGATTCTCCCATGTACTTCTTC | | ISKSLDSPMYFFLSYL | |
| | CTTAGCTACCTGTCCTTGGTGGAGATCAGTTATTCCTCCACTATCGC | | SLVEISYSSTIAPKFI | |
| | CCCTAAATTCATCATAGACTTACTTGCCAAGATTAAAACCATCTCTC | | IDLLAKIKTISLEGCL | |
| | TGGAAGGCTGTCTGACTCAGATATTCTTCTTCCACTTCTTTGGGGTT | | TQIFFFHFFGVAEILL | |
| | GCTGAGATCCTTTTGATTGTGGTGATGGCCTATGATTGCTACGTGGC | | IVVMAYDCYVAICKPL | |
| | CATTTGCAAGCCTCTTCATTATATGAACATTATCAGTCGTCAACTGT | | HYMNIISRQLCHLLVA | |
| | GTCACCTTCTGGTGGCTGGTTCCTGGCTGGGGGGCTTTTGTCACTCC | | GSWLGGFCHSIIQILV | |
| | ATAATTCAGATTCTCGTTATCATCCAATTGCCCTTCTGTGGTCCCAA | | IIQLPFCGPNVIDHYF | |
| | TGTGATTGACCACTATTTCTGTGACCTCCAGCCTTTATTCAAGCTTG | | CDLQPLFKLACTDTFM | |
| | CCTGCACTGACACCTTCATGGAGGGGGTTATTGTGTTGGCCAACAGT | | EGVIVLANSGLFSVFS | |
| | GGATTATTCTCTGTCTTCTCCTTCCTCATCTTGGTGTCCTCTTATAT | | FLILVSSYIVILVNLR | |
| | TGTCATTCTGGTCAACTTGAGGAACCATTCTGCAGAGGGGAGGCACA | | NHSAEGRHKALSTCAS | |
| | AAGCCCTCTCCACCTGTGCTTCTCACATCACAGTGGTCATCTTGTTT | | HITVVILFFGPAIFLY | |
| | TTTGGACCTGCTATCTTCCTCTACATGCGACCTTCTTCCACTTTCAC | | MRPSSTFTEDKLVAVF | |
| | TGAAGATAAACTTGTGGCTGTATTCTACATGGTCATCACCCCCATGC | | YMVITPMLNPIIYTLR | |
| | TGAACCCCATCATTTACACACTCAGGAATGCAGAGGTGAAAATCGCC | | NAEVKIAIRRLWSKKE | |
| | ATAAGAAGATTGTGGAGCAAAAAGGAGAATCCAGGGAGGGAGTGA | | NPGRE | |
| SC1227377 | 000 | 129 | MERVNETVVREVIFLG | 130 |
| 11_A_2 | | | FSSLARLQQLLFVIFL | |
| | | | LLYLFTLGTNAIIIST | |
| | | | IVLDRALHIPMYFFLA | |
| | | | ILSCSEICYTFIIVPK | |
| | | | MLVDLLSQKKTISFLG | |
| | | | CAIQMFSFLFLGCSHS | |
| | | | FLLAVMGYDRYIAICN | |
| | | | PLRYSVLMGHGVCMGL | |
| | | | VAAACACGFTVAQIIT | |
| | | | SLVFHLPFYSSNQLHH | |
| | | | FFCDIAPVLKLASHHN | |
| | | | HFSQIVIFMLCTLVLA | |
| | | | IPLLLILVSYVHILSA | |
| | | | ILQFPSTLGRCKAFST | |
| | | | CVSHLIIVTVHYGCAS | |
| | | | FIYLRPQSNYSSSQDA | |
| | | | LISVSYTIITPLFNPM | |
| | | | IYSLRNKEFKSALCKI | |
| | | | VRRTISLL | |

The invention will be further described in the following examples, which do not limit the scope of the invention described in the claims.

### EXAMPLES

### Example 1

### Identification of GPCRX Nucleic Acids

TblastN using CuraGen Corporation's sequence file for polypeptides or homologs was run against the Genomic Daily Files made available by GenBank or from files downloaded from the individual sequencing centers. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The novel GPCRX target sequences identified in the present invention were subjected to the exon linking process to confirm the sequence. PCR primers were designed by starting at the most upstream sequence available, for the forward primer, and at the most downstream sequence available for the reverse primer. PCR primer sequences were used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### Example 2

### Identification of Single Nucleotide Polymorphisms in GPCRX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderbom et al., Determination of Single Nucleotide Polymorphisms by Real-time Pyrophosphate DNA Sequencing. Genome Research. 10 (8) 1249-1265, 2000).

### Example 3

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive panel (containing normal tissue and samples from autoimmune diseases), Panel CNSD.01 (containing samples from normal and diseased brains) and CNS_neurodegeneration_panel (containing central nervous system samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tₘ=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqManOR Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1 .3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effision,

glio=glioma,

astro=astrocytoma, and

neuro=neuroblastoma.

General_screening_panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1 .3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5xl I⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 kg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, UT), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶cells/ml in DMEM 5% FCS (Hyclone), 100 M non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD 106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷cells/ml using Trizol (Gibco BRL). Briefly, 1/10 volume of bromochloropropane (Molecular Research Corporation) was added to the RNA samnple, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_comprehensive panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample.

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supemuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. CG50299-02/GMAC011647_C and CG50299-01: Olfactory Receptor

Expression of gene CG50299-02 and variant CG50299-01 was assessed using the primer-probe set Ag2214, described in Table AA. Results of the RTQ-PCR runs are shown in Tables AB, AC, AD, and AE.

TABLE AA
Probe Name Ag2214

| | | | | |
|---|---|---|---|---|
| | | | Start | SEQ ID |
| Primers | Sequences | Length | Position | NO |

| | | | | |
|---|---|---|---|---|
| Forward | 5′-atgccaggaagaatgtcagatt-3′ | 22 | 9 | 131 |
| Probe | TET-5′-ccaacctcagtgataaccatcttcca-3′-TAMRA | 26 | 31 | 132 |
| Reverse | 5′-tgggatccctgttaagaagaag-3′ | 22 | 62 | 133 |

TABLE AB
General_screening_panel_v1.5

| | Rel. Exp.(%) | | |
|---|---|---|---|
| | Ag2214, Run | | Rel. Exp.(%) |
| Tissue Name | 228633525 | Tissue Name | Ag2214, Run |
| Adipose | 0.1 | Renal ca. TK-10 | 0.0 |
| Melanoma* Hs688(A).T | 0.0 | Bladder | 0.3 |
| Melanoma* Hs688(B).T | 0.0 | Gastric ca. (liver met.) NCI-N87 | 0.1 |
| Melanoma* M14 | 0.1 | Gastric ca. KATO III | 0.3 |
| Melanoma* LOXIMVI | 1.8 | Colon ca. SW-948 | 0.0 |
| Melanoma* SK-MEL-5 | 16.5 | Colon ca. SW480 | 0.1 |
| Squamous cell carcinoma | 0.0 | Colon ca.* (SW480 met) | 1.2 |
| SCC-4 | | SW620 | |
| Testis Pool | 0.4 | Colon ca. HT29 | 0.0 |
| Prostate ca.* (bone met) PC-3 | 0.0 | Colon ca. HCT-116 | 100.0 |
| Prostate Pool | 0.3 | Colon ca. CaCo-2 | 0.1 |
| Placenta | 0.0 | Colon cancer tissue | 0.0 |
| Uterus Pool | | Colon ca. SW1116 | 0.0 |
| Ovarian ca. OVCAR-3 | 0.1 | Colon ca. Colo-205 | 0.0 |
| Ovarian ca. SK-OV-3 | 0.7 | Colon ca. SW-48 | 0.0 |
| Ovarian ca. OVCAR-4 | 0.0 | Colon Pool | 0.2 |
| Ovarian ca. OVCAR-5 | 0.1 | Small Intestine Pool | 0.1 |
| Ovarian ca. IGROV-1 | 0.0 | Stomach Pool | 0.3 |
| Ovarian ca. OVCAR-8 | 0.1 | Bone Marrow Pool | 0.2 |
| Ovary | 0.2 | Fetal Heart | 0.2 |
| Breast ca. MCF-7 | 0.0 | Heart Pool | 0.2 |
| Breast ca. MDA-MB-231 | 0.0 | Lymph Node Pool | 0.0 |
| Breast ca. BT 549 | 0.0 | Fetal Skeletal Muscle | 0.0 |
| Breast ca. T47D | 0.0 | Skeletal Muscle Pool | 0.1 |
| Breast ca. MDA-N | 1.1 | Spleen Pool | 0.1 |
| Breast Pool | 0.7 | Thymus Pool | 0.1 |
| Trachea | 0.2 | CNS cancer (glio/astro) U87- | 0.0 |
| | | MG | |
| Lung | 0.3 | CNS cancer (glio/astro) U-118- | 0.2 |
| | | MG | |
| Fetal Lung | 0.6 | CNS cancer (neuro;met) SK-N- | 0.0 |
| | | AS | |
| Lung ca. NCI-N417 | 0.0 | CNS cancer (astro) SF-539 | 0.0 |
| Lung ca. LX-1 | 14.8 | CNS cancer (astro) SNB-75 | 0.0 |
| Lung ca. NCI-H146 | 0.0 | CNS cancer (glio) SNB-19 | 0.0 |
| Lung ca. SHP-77 | 0.4 | CNS cancer (glio) SF-295 | 0.5 |
| Lung ca. A549 | 0.0 | Brain (Amygdala) Pool | 0.1 |
| Lung ca. NCI-H526 | 0.0 | Brain (cerebellum) | 0.0 |
| Lung ca. NCI-H23 | 0.0 | Brain (fetal) | 0.1 |
| Lung ca. NCI-H460 | 0.3 | Brain (Hippocampus) Pool | 0.0 |
| Lung ca. HOP-62 | 40.0 | Cerebral Cortex Pool | 0.0 |
| Lung ca. NCI-H522 | 0.0 | Brain (Substantia nigra) Pool | 0.0 |
| Liver | 0. | Brain (Thalamus) Pool | 0.1 |
| Fetal Liver | 0.0 | Brain (whole) | 0.0 |
| Liver ca. HepG2 | 0.0 | Spinal Cord Pool | 0.0 |
| Kidney Pool | 0.5 | Adrenal Gland | 0.0 |
| Fetal Kidney | 0.5 | Pituitary gland Pool | 0.1 |
| Renal ca. 786-0 | 0.0 | Salivary Gland | 0.0 |
| Renal ca. A498 | 0.0 | Thyroid (female) | 0.1 |
| Renal ca. ACHN | 0.0 | Pancreatic ca. CAPAN2 | 0.0 |
| Renal ca. UO-31 | 0.0 | Pancreas Pool | 0.4 |

TABLE AC
Panel 1.3D

| | Rel. Exp.(%) | | Rel. Exp.(%) |
|---|---|---|---|
| | Ag2214, Run | | Ag2214, Run |
| Tissue Name | 165974837 | Tissue Name | 165974837 |
| Liver adenocarcinoma | 0.0 | Kidney (fetal) | 1.7 |
| Pancreas | 0.6 | Renal ca. 786-0 | 0.0 |
| Pancreatic ca. CAPAN 2 | 0.0 | Renal ca. A498 | 0.0 |
| Adrenal gland | 1.2 | Renal ca. RXF 393 | 0.0 |
| Thyroid | 0.0 | Renal ca. ACHN | 0.7 |
| Salivary gland | 1.0 | Renal ca. UO-31 | 0.0 |
| Pituitary gland | 0.0 | Renal ca. TK-10 | 0.0 |
| Brain (fetal) | 0.7 | Liver | 1.1 |
| Brain (whole) | 0.6 | Liver (fetal) | 0.0 |
| Brain (amygdala) | 0.6 | Liver ca. (hepatoblast) | 0.5 |
| | | HepG2 | |
| Brain (cerebellum) | 0. | Lung | 0.0 |
| Brain (hippocampus) | 0.0 | Lung (fetal) | 0.0 |
| Brain (substantia nigra) | 0.0 | Lung ca. (small cell) | 53.2 |
| | | LX-1 | |
| Brain (thalamus) | 0.0 | Lung ca. (small cell) | 0.5 |
| | | NCI-H69 | |
| Cerebral Cortex | 0.0 | Lung ca. (s. cell var.) | 1.5 |
| | | SHP-77 | |
| Spinal cord | 0.0 | Lung ca. (large | 0.0 |
| | | cell)NCI-H460 | |
| glio/astro U87-MG | 0.0 | Lung ca. (non-sm. cell) | 0.0 |
| | | A549 | |
| glio/astro U-118-MG | 0.5 | Lung ca. (non-s. cell) | 0.7 |
| | | NCI-H23 | |
| astrocytoma SW1783 | 0.0 | Lung ca. (non-s. cell) | 0.0 |
| | | HOP-62 | |
| neuro*;met SK-N-AS | 0.0 | Lung ca. (non-s. cl) | 0.0 |
| | | NCI-H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.9 |
| | | H596 | |
| glioma SNB-19 | 0.3 | Mammary gland | 0.5 |
| glioma U251 | 0.6 | Breast ca.* (pl. ef) | 0.5 |
| | | MCF-7 | |
| glioma SF-295 | 1.0 | Breast ca.* (pl. ef) | 0.0 |
| | | MDA-MB-231 | |
| Heart (Fetal) | 0.0 | Breast ca.* (pl. ef) | 0.0 |
| | | T47D | |
| Heart | 0.0 | Breast ca. BT-549 | 0.0 |
| Skeletal muscle (Fetal) | 0.0 | Breast ca. MDA-N | 6.5 |
| Skeletal muscle | 0.0 | Ovary | 0.0 |
| Bone marrow | 0.0 | Ovarian ca. OVCAR-3 | 0.6 |
| Thymus | 0.0 | Ovarian ca. OVCAR-4 | 0.0 |
| Spleen | 0.0 | Ovarian ca. OVCAR-5 | 0.0 |
| Lymph node | 0.3 | Ovarian ca. OVCAR-8 | 0.0 |
| Colorectal | 0.3 | Ovarian ca. IGROV-1 | 0.0 |
| Stomach | 0.0 | Ovarian ca. (ascites) | 3.2 |
| | | SK-OV-3 | |
| Small intestine | 0.0 | Uterus | 0.0 |
| Colon ca. SW480 | 0.9 | Placenta | 1.5 |
| Colon ca.* SW620 | 2.8 | Prostate | 0.0 |
| (SW480 met) | | | |
| Colon ca. HT29 | 0.5 | Prostate ca.* (bone | 0.0 |
| | | met) PC-3 | |
| Colon ca. HCT-116 | 100.0 | Testis | 4.5 |
| Colon ca. CaCo-2 | 0.8 | Melanoma Hs688(A).T | 0.0 |
| CC Well to Mod Diff | 1.0 | Melanoma* (met) | 0.4 |
| (ODO3866) | | Hs688(B).T | |
| Colon ca. HCC-2998 | 1.5 | Melanoma UACC-62 | 0.0 |
| Gastric ca. (liver met) | 0.5 | Melanoma M14 | 0.0 |
| NCI-N87 | | | |
| Bladder | 2.5 | Melanoma LOX IMVI | 6.6 |
| Trachea | 0.0 | Melanoma* (met) SK- | 31.9 |
| | | MEL-5 | |
| Kidney | 0.0 | Adipose | 0.4 |

TABLE AD
Panel 4.1D

| | Rel. Exp.(%) Ag2214, | | Rel. Exp.(%) Ag2214, |
|---|---|---|---|
| Tissue Name | Run 224781630 | Tissue Name | Run 224781630 |
| Secondary Th1 act | 0.0 | HUVEC IL-1 beta | 0.5 |
| Secondary Th2 act | 0.0 | HUVEC IFN gamma | 0.0 |
| Secondary Tr1 act | 0.5 | HUVEC TNF alpha + IFN | 0.0 |
| | | gamma | |
| Secondary Th1 rest | 0.7 | HUVEC TNF alpha + IL4 | 0.0 |
| Secondary Th2 rest | 0.6 | HUVEC IL-11 | 0.6 |
| Secondary Tr1 rest | 0.6 | Lung Microvascular EC none | 1.5 |
| Primary Th1 act | 0.7 | Lung Microvascular EC | 1.1 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.5 |
| | | TNFalpha + IL-1beta | |
| Primary Th2 rest | 0.4 | Small airway epithelium none | 0.0 |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.5 | Coronery artery SMC | 0.0 |
| lymphocyte act | | TNFalpha + IL-1beta | |
| CD8 lymphocyte act | 0.5 | Astrocytes rest | 0.5 |
| Secondary CD8 | 0.0 | Astrocytes TNFalpha + IL- | 0.5 |
| lymphocyte rest | | 1beta | |
| Secondary CD8 | 0.0 | KU-812 (Basophil) rest | 0.8 |
| lymphocyte act | | | |
| CD4 lymphocyte none | 0.0 | KU-812 (Basophil) | 0.6 |
| | | PMA/ionomycin | |
| 2ry Th1/Th2/Tr1_anti- | 0.9 | CCD1106 (Keratinocytes) | 0.0 |
| CD95 CH11 | | none | |
| LAK cells rest | 2.8 | CCD1106 (Keratinocytes) | 0.0 |
| | | TNFalpha + IL-1beta | |
| LAK cells IL-2 | 1.7 | Liver cirrhosis | 0.0 |
| LAK cells IL-2 + IL-12 | 1.6 | NCI-H292 none | 0.0 |
| LAK cells IL-2 + IFN | 3.5 | NCI-H292 IL-4 | 0.0 |
| gamma | | | |
| LAK cells IL-2 + IL-18 | 3.9 | NCI-H292 IL-9 | 1.1 |
| LAK cells | 0.0 | NCI-H292 IL-13 | 0.8 |
| PMA/ionomycin | | | |
| NK Cells IL-2 rest | 1.1 | NCI-H292 IFN gamma | 0.0 |
| Two Way MLR 3 day | 2.7 | HPAEC none | 0.0 |
| Two Way MLR 5 day | 1.8 | HPAEC TNF alpha + IL-1 | 0.0 |
| | | beta | |
| Two Way MLR 7 day | 0.0 | Lung fibroblast none | 0.9 |
| PBMC rest | 0.0 | Lung fibroblast TNF alpha + | 1.6 |
| | | IL-1 beta | |
| PBMC PWM | 0.0 | Lung fibroblast IL-4 | 0.0 |
| PBMC PHA-L | 0.0 | Lung fibroblast IL-9 | 0.9 |
| Ramos (B cell) none | 21.3 | Lung fibroblast IL-13 | 0.5 |
| Ramos (B cell) | 24.8 | Lung fibroblast IFN gamma | 0.2 |
| ionomycin | | | |
| B lymphocytes PWM | 0.0 | Dermal fibroblast CCD1070 | 0.6 |
| | | rest | |
| B lymphocytes CD40L | 0.0 | Dermal fibroblast CCD1070 | 0.0 |
| and IL-4 | | TNF alpha | |
| EOL-1 dbcAMP | 0.9 | Dermal fibroblast CCD1070 | 0.0 |
| | | IL-1 beta | |
| EOL-1 dbcAMP | 1.8 | Dermal fibroblast IFN gamma | 0.0 |
| PMA/ionomycin | | | |
| Dendritic cells none | 1.1 | Dermal fibroblast IL-4 | 1.9 |
| Dendritic cells LPS | 0.0 | Dermal Fibroblasts rest | 2.6 |
| Dendritic cells anti-CD40 | 0.0 | Neutrophils TNFa + LPS | 0.0 |
| Monocytes rest | 0.0 | Neutrophils rest | 1.8 |
| Monocytes LPS | 0.5 | Colon | 0.5 |
| Macrophages rest | 0.7 | Lung | 1.8 |
| Macrophages LPS | 0.0 | Thymus | 19.3 |
| HUVEC none | 0.0 | Kidney | 100.0 |
| HUVEC starved | 0.3 | | |

TABLE AE
Panel 4D

| | Rel. Exp.(%) Ag2214, | | Rel. Exp.(%) Ag2214, |
|---|---|---|---|
| Tissue Name | Run 163724300 | Tissue Name | Run 163724300 |
| Secondary Th1 act | 0.0 | HUVEC IL-1beta | 0.0 |
| Secondary Th2 act | 0.0 | HUVEC IFN gamma | 0.3 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.5 |
| Primary Th1 act | 0.0 | Lung Microvascular EC | 0.0 |
| | | TNFalpha + IL-1beta | |
| Primary Th2 act | 0.0 | Microvascular | 0.2 |
| | | Dermal EC none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.3 |
| | | TNFalpha + IL1beta | |
| Primary Th2 rest | 0.8 | Small airway epithelium none | 0.0 |
| Primary Tr1 rest | 1.1 | Small airway epithelium | 1.0 |
| | | TNFalpha + IL-1beta | |
| CD45RA CD4 | 0.3 | Coronery artery SMC rest | 0.3 |
| lymphocyte act | | | |
| CD45RO CD4 | 0.5 | Coronery artery SMC | 0.3 |
| lymphocyte act | | TNFalpha + IL-1beta | |
| CD8 lymphocyte act | 0.0 | Astrocytes rest | 0.0 |
| Secondary CD8 | 0.2 | Astrocytes TNFalpha + IL- | 0.0 |
| lymphocyte rest | | 1beta | |
| Secondary CD8 | 0.2 | KU-812 (Basophil) rest | 0.4 |
| lymphocyte act | | | |
| CD4 lymphocyte none | 0.0 | KU-812 (Basophil) | 0.3 |
| | | PMA/ionomycin | |
| 2ry Th1/Th2/ Tr1_anti- | 0.7 | CCD1106 (Keratinocytes) | 0.0 |
| CD95 CH11 | | none | |
| LAK cells rest | 0.5 | CCD1106 (Keratinocytes) | 0.0 |
| | | TNFalpha + IL-1beta | |
| LAK cells IL-2 | 0.2 | Liver cirrhosis | 2.3 |
| LAK cells IL-2 + IL-12 | 0.8 | Lupus kidney | 0.3 |
| LAK cells IL-2 + IFN | 0.9 | NCI-H292 none | 0.3 |
| gamma | | | |
| LAK cells IL-2 + IL-18 | 0.9 | NCI-H292 IL-4 | 0.3 |
| LAK cells | 0.0 | NCI-H292 IL-9 | 0.4 |
| PMA/ionomycin | | | |
| NK Cells IL-2 rest | 0.5 | NCI-H292 IL-13 | 0.6 |
| Two Way MLR 3 day | 0.3 | NCI-H292 IFN gamma | 0.0 |
| Two Way MLR 5 day | 0.8 | HPAEC none | 0.1 |
| Two Way MLR 7 day | 0.0 | HPAEC TNF alpha + IL-1 | 0.0 |
| | | beta | |
| PBMC rest | 0.0 | Lung fibroblast none | 0.9 |
| PBMC PWM | 1.1 | Lung fibroblast TNF alpha + | 1.5 |
| | | IL-1 beta | |
| PBMC PHA-L | 0.0 | Lung fibroblast IL-4 | 1.3 |
| Ramos (B cell) none | 23.5 | Lung fibroblast IL-9 | 0.3 |
| Ramos (B cell) | 100.0 | Lung fibroblast IL-13 | 0.0 |
| ionomycin | | | |
| B lymphocytes PWM | 0.3 | Lung fibroblast IFN gamma | 0.0 |
| B lymphocytes CD40L | 0.3 | Dermal fibroblast CCD1070 | 0.0 |
| and IL-4 | | rest | |
| EOL-1 dbcAMP | 0.0 | Dermal fibroblast CCD1070 | 0.4 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.3 |
| Dendritic cells LPS | 0.0 | Dermal fibroblast IL-4 | 0.0 |
| Dendritic cells anti-CD40 | 0.9 | IBD Colitis 2 | 0.1 |
| Monocytes rest | 0.0 | IBD Crohn's | 0.0 |
| Monocytes LPS | 0.0 | Colon | 0.3 |
| Macrophages rest | 0.3 | Lung | 1.2 |
| Macrophages LPS | 0.0 | Thymus | 11.3 |
| HUVEC none | 0.0 | Kidney | 3.6 |
| HUVEC starved | 0.0 | | |

General_screening panel_v1.5 Summary: Ag2214 The expression of this gene is highest in a colon cancer cell line (HCT 116). In addition there appears to be substantial expression in a lung cancer cell line (LX-1) and a melanoma cell line (SK-MEL-5). Thus, the expression of this gene could be used to distinguish these samples from others in the panel. Moreover, therapeutic modulation of this gene, through the use of small molecule drugs, antibodies or protein therapeutics might be of benefit to the treatment of melanoma, colon cancer or lung cancer.

Among tissues with metabolic activity, this gene is expressed at low levels in pancreas and heart. Low expression is also seen in a number of other normal tissues including testis, prostate, kidney, lung, breast, uterus and ovary (CTs=31-35).

Panel 1.3D Summary: Ag2214 The expression of this gene is highest in a colon cancer cell line (HCT 1 16)(CT=30.6). In addition there appears to be substantial expression in a lung cancer cell line (LX-1) and two melanoma cell lines (SK-MEL-5 LOX IMVI). Thus, the expression of this gene could be used to distinguish these samples from others in the panel. Moreover, therapeutic modulation of this gene, through the use of small molecule drugs, antibodies or protein therapeutics might be of benefit to the treatment of melanoma, colon cancer or lung cancer.

Panel 2.2 Summary: Ag2214 Expression is low/undetectable across the samples in this panel. (Data not shown.)

Panel 4.1D Summary: Ag2214 Highest expression is seen in a sample from normal kidney (CT=30.5). This gene is also expressed at low levels in Ramos B cells (treated and untreated) and thymus. The expression of this transcript in B cells suggests that this gene may be involved in rheumatoid arthritis, osteoarthritis, rheumatic disease including lupus, and hyperproliferative B cell disorders and may represent a target for antibody or small molecule therapeutics.

Panel 4D Summary: Ag2214 Highest expression in this panel is seen in Ramos B cells. Lower expression levels are also detected in thymus and kidney. Please see Panel 4.1D for discussion of potential utility in the context of autoimmune disease.

B. CG142324-01/GMAP001112_B: Olfactory Receptor

Expression of gene CG142324-01 was assessed using the primer-probe set Ag2223, described in Table BA. Results of the RTQ-PCR runs are shown in Tables BB, and BC.

TABLE BA

Probe Name Ag2223

| | | | Start | SEQ ID |
|---|---|---|---|---|
| Primers | Sequences | Length | Position | NO |
| Forward | 5′-ctacagagggcaggcaaaa-3′ | 19 | 692 | 134 |
| Probe | TET-5′-tttctacctgtggctcccatctgaca-3′-TAMRA | 26 | 716 | 135 |
| Reverse | 5′-ggaggtctgagacacatgaaga-3′ | 22 | 770 | 136 |

TABLE BB
Panel 2D

| | Rel. Exp.(%) Ag2223, | | Rel. Exp.(%) Ag2223, |
|---|---|---|---|
| Tissue Name | Run 153731708 | Tissue Name | Run 153731708 |
| Normal Colon | 0.0 | Kidney Margin 8120608 | 0.0 |
| CC Well to Mod Diff | 0.0 | Kidney Cancer 8120613 | 0.0 |
| (ODO3866) | | | |
| CC Margin (ODO3866) | 0.0 | Kidney Margin 8120614 | 0.0 |
| CC Gr.2 rectosigmoid | 0.0 | Kidney Cancer 9010320 | 0.0 |
| (ODO3868) | | | |
| CC Margin (ODO3868) | 0.0 | Kidney Margin 9010321 | 0.0 |
| CC Mod Diff (ODO3920) | 0.0 | Normal Uterus | 0.0 |
| CC Margin (ODO3920) | 0.0 | Uterine Cancer 064011 | 0.0 |
| CC Gr.2 ascend colon | 0.0 | Normal Thyroid | 0.0 |
| (ODO3921) | | | |
| CC Margin (ODO3921) | 0.0 | Thyroid Cancer | 0.0 |
| CC from Partial | 0.0 | Thyroid Cancer | 0.0 |
| Hepatectomy (ODO4309) | | A302152 | |
| Mets | | | |
| Liver Margin (ODO4309) | 0.0 | Thyroid Margin | 8.8 |
| | | A302153 | |
| Colon mets to lung | 0.0 | Normal Breast | 18.0 |
| (OD04451-01) | | | |
| Lung Margin (OD04451-02) | 0.0 | Breast Cancer | 0.0 |
| Normal Prostate 6546-1 | 0.0 | Breast Cancer | 0.0 |
| | | (OD04590-01) | |
| Prostate Cancer (OD04410) | 0.0 | Breast Cancer Mets | 0.0 |
| | | (OD04590-03) | |
| Prostate Margin (OD04410) | 0.0 | Breast Cancer Metastasis | |
| Prostate Cancer (OD04720- | 0.0 | Breast Cancer | 0.0 |
| 01) | | | |
| Prostate Margin (OD04720- | 0.0 | Breast Cancer | 0.0 |
| 02) | | | |
| Normal Lung | 0.0 | Breast Cancer 9100266 | 0.0 |
| Lung Met to Muscle | 20.0 | Breast Margin 9100265 | 0.0 |
| (ODO4286) | | | |
| Muscle Margin (ODO4286) | 0.0 | Breast Cancer A209073 | 0.0 |
| Lung Malignant Cancer | 0.0 | Breast Margin | 0.0 |
| (OD03126) | | A2090734 | |
| Lung Margin (OD03126) | 0.0 | Normal Liver | 0.0 |
| Lung Cancer (OD04404) | 0.0 | Liver Cancer | 17.2 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237-01) | 0.0 | Liver Tissue 6004-N | 0.0 |
| Lung Margin (OD04237-02 | 0.0 | Liver Cancer 6005-T | 0.0 |
| Ocular Mel Met to Liver | 0.0 | Liver Tissue 6005-N | 0.0 |
| (ODO4310) | | | |
| Liver Margin (ODO4310) | 0.0 | Normal Bladder | 0.0 |
| Melanoma Metastasis | 0.0 | Bladder Cancer | 0.0 |
| Lung Margin (OD04321) | 0.0 | Bladder Cancer | 100.0 |
| Normal Kidney | 0.0 | Bladder Cancer | 0.0 |
| | | (OD04718-01) | |
| Kidney Ca, Nuclear grade 2 | 0.0 | Bladder Normal | 0.0 |
| (OD04338) | | Adjacent (OD04718-03) | |
| Kidney Margin (OD04338) | 0.0 | Normal Ovary | 0.0 |
| Kidney Ca Nuclear grade 1/2 | 0.0 | Ovarian Cancer | 0.0 |
| (OD04339) | | | |
| Kidney Margin (OD04339) | 0.0 | Ovarian Cancer | 0.0 |
| | | (OD04768-07) | |
| Kidney Ca, Clear cell type | 0.0 | Ovary Margin | 0.0 |
| (OD04340) | | (OD04768-08) | |
| Kidney Margin (OD04340) | 0.0 | Normal Stomach | 0.0 |
| Kidney Ca, Nuclear grade 3 | 0.0 | Gastric Cancer 9060358 | 0.0 |
| (OD04348) | | | |
| Kidney Margin (OD04348) | 0.0 | Stomach Margin | 0.0 |
| | | 9060359 | |
| Kidney Cancer (OD04622- | 0.0 | Gastric Cancer 9060395 | 0.0 |
| 01) | | | |
| Kidney Margin (OD04622- | 0.0 | Stomach Margin | 0.0 |
| 03) | | 9060394 | |
| Kidney Cancer (OD04450- | 0.0 | Gastric Cancer 90603917 | 0.0 |
| 01) | | | |
| Kidney Margin (OD04450- | 0.0 | Stomach Margin | 0.0 |
| 03) | | 9060396 | |
| Kidney Cancer 8120607 | 0.0 | Gastric Cancer 064005 | 0.0 |

TABLE BC
Panel 4D

| | Rel. Exp.(%) | | Rel. Exp.(%) |
|---|---|---|---|
| | Ag2223, Run | | Ag2223, Run |
| Tissue Name | 153731723 | Tissue Name | 153731723 |
| Secondary Th1 act | 0.0 | HUVEC IL-1 beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 lymphocyte | 0.0 | Coronery artery SMC rest | 11.5 |
| act | | | |
| CD45RO CD4 lymphocyte | 0.0 | Coronery artery SMC | 0.0 |
| act | | TNFalpha + IL-1beta | |
| CD8 lymphocyte act | 0.0 | Astrocytes rest | |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_ anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 19.8 |
| B lymphocytes CD40L and | 0.0 | Dermal fibroblast CCD1070 | 0.0 |
| IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 59.5 |
| Macrophages rest | 0.0 | Lung | 24.1 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag2223 Data from one experiment with this probe and primer is not included because the amp plot indicates that there were experimental difficulties with this run.

Panel 1.3D Summary: Ag2223 Expression low/undetectable (CTs>35) in all of the samples on this panel. (Data not shown.)

Panel 2D Summary: Ag2223 Low but significant expression of this gene is limited to a single bladder cancer sample (CT=34.4). Therefore, expression of this gene may be used to distinguish bladder cancers from the other samples on this panel. Furthermore, therapeutic modulation of the activity of the GPCR encoded by this gene may be beneficial in the treatment of bladder cancer.

Panel 4D Summary: Ag2223 Significant expression of this gene is detected in a liver cirrhosis sample (CT=34.8). Furthermore, expression of this gene is not detected in normal liver in Panel 1.3D, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

C. CG104704-02/GMAC011647_B and CG104704-01: Olfactory Receptor

Expression of gene CG104704-02 and variant CG104704-01 was assessed using the primer-probe set Ag2213, described in Table CA. Results of the RTQ-PCR runs are shown in Tables CB, and CC.

TABLE CA
Probe Name Ag2213

| | | | Start | SEQ ID |
|---|---|---|---|---|
| Primers | Sequences | Length | Position | NO |
| Forward | 5′-aatggcatcctaatttgtgtca-3′ | 22 | 170 | 137 |
| Probe | TET-5′-caatcctgcatgagcccatgtacata-3′-TAMRA | 26 | 204 | 138 |
| Reverse 5′-cactggccagcatagataagaa-3′ | 22 | 230 | 139 | |

TABLE CB
Panel 1.3D

| | Rel. Exp.(%) | | Rel. Exp.(%) |
|---|---|---|---|
| | Ag2213, Run | | Ag2213, Run |
| Tissue Name | 165974918 | Tissue Name | 165974918 |
| Liver adenocarcinoma | 0.0 | Kidney (fetal) | 0.0 |
| Pancreas | 4.2 | Renal ca. 786-0 | 14.1 |
| Pancreatic ca. CAPAN 2 | 0.0 | Renal ca. A498 | 5.1 |
| Adrenal gland | 23.0 | Renal ca. RXF 393 | 0.0 |
| Thyroid | 9.5 | Renal ca. ACHN | 0.0 |
| Salivary gland | 5.5 | Renal ca. UO-31 | 7.4 |
| Pituitary gland | 6.7 | Renal ca. TK-10 | 0.0 |
| Brain (fetal) | 5.7 | Liver | 0.0 |
| Brain (whole) | 11.3 | Liver (fetal) | |
| Brain (amygdala) | 20.0 | Liver ca. (hepatoblast) | 0.0 |
| | | HepG2 | |
| Brain (cerebellum) | 0.0 | Lung | 0.0 |
| Brain (hippocampus) | 21.3 | Lung (fetal) | 6.7 |
| Brain (substantia nigra) | 0.0 | Lung ca. (small cell) | 0.0 |
| | | LX-1 | |
| Brain (thalamus) | 31.0 | Lung ca. (small cell) | 0.0 |
| | | NCI-H69 | |
| Cerebral Cortex | 0.0 | Lung ca. (s.cell var.) | 0.0 |
| | | SHP-77 | |
| Spinal cord | 26.8 | Lung ca. (large | 0.0 |
| | | cell)NCI-H460 | |
| glio/astro U87-MG | 0.0 | Lung ca. (non-sm. cell) | 8.8 |
| | | A549 | |
| glio/astro U-118-MG | 0.0 | Lung ca. (non-s. cell) | 14.2 |
| | | NCI-H23 | |
| astrocytoma SW1783 | 3.4 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 24.7 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 5.1 | Mammary gland | 0.0 |
| glioma U251 | 0.0 | Breast ca.* (pl.ef) MCF- | 0.0 |
| | | 7 | |
| glioma SF-295 | 6.6 | Breast ca.* (pl.ef) MDA- | 0.0 |
| | | MB-231 | |
| Heart (Fetal) | 0.0 | Breast ca.* (pl.ef) T47D | 0.0 |
| Heart | 17.7 | Breast ca. | 0.0 |
| | | BT-549 | |
| Skeletal muscle (Fetal) | 0.0 | Breast ca. MDA-N | 0.0 |
| Skeletal muscle | 5.9 | Ovary | 0.0 |
| Bone marrow | 7.8 | Ovarian ca. OVCAR-3 | 7.3 |
| Thymus | 17.9 | Ovarian ca. OVCAR-4 | 0.0 |
| Spleen | 14.6 | Ovarian ca. OVCAR-5 | 12.5 |
| Lymph node | 0.0 | Ovarian ca. OVCAR-8 | 3.5 |
| Colorectal | 0.0 | Ovarian ca. IGROV-1 | 0.0 |
| Stomach | 11.9 | Ovarian ca. (ascites) SK- | 100.0 |
| | | OV-3 | |
| Small intestine | 0.0 | Uterus | 0.0 |
| Colon ca. SW480 | 0.0 | Placenta | 4.4 |
| Colon ca.* SW620 | 0.0 | Prostate | 7.7 |
| (SW480 met) | | | |
| Colon ca. HT29 | 0.0 | Prostate ca.* (bone met) | 17.2 |
| | | PC-3 | |
| Colon ca. HCT-116 | 0.0 | Testis | 48.6 |
| Colon ca. CaCo-2 | 0.0 | Melanoma Hs688(A).T | 0.0 |
| CC Well to Mod | 0.0 | Melanoma* (met) | 0.0 |
| Diff (ODO3866) | | Hs688(B).T | |
| Colon ca. HCC-2998 | 0.0 | Melanoma UACC-62 | 10.3 |
| Gastric ca. (liver met) | 21.9 | Melanoma M14 | 0.0 |
| NCI-N87 | | | |
| Bladder | 6.4 | Melanoma LOX IMVI | 0.0 |
| Trachea | 1.7 | Melanoma* (met) SK- | 0.0 |
| | | MEL-5 | |
| Kidney | 14.9 | Adipose | 13.6 |

TABLE CC
Panel 4D

| | Rel. Exp.(%) | | Rel. Exp.(%) |
|---|---|---|---|
| | Ag2213, Run | | Ag2213, Run |
| Tissue Name | 163633529 | Tissue Name | 163633529 |
| Secondary Th1 act | 0.0 | HUVEC IL-1beta | 2.4 |
| Secondary Th2 act | 5.3 | HUVEC IFN gamma | 7.5 |
| Secondary Tr1 act | 2.1 | HUVEC TNF alpha + IFN | 4.8 |
| | | gamma | |
| Secondary Th1rest | 4.1 | HUVEC TNF alpha + IL4 | 0.0 |
| Secondary Th2rest | 3.9 | HUVEC IL-11 | 0.0 |
| Secondary Tr1rest | 15.9 | Lung Microvascular EC none | 8.8 |
| Primary Th1 act | 2.8 | Lung Microvascular EC | 9.9 |
| | | TNFalpha + IL-1beta | |
| Primary Th2 act | 2.4 | Microvascular Dermal EC | 4.2 |
| | | none | |
| Primary Tr1 act | 1.8 | Microsvasular Dermal EC | 0.0 |
| | | TNFalpha + IL-1beta | |
| Primary Th1 rest | 100.0 | Bronchial epithelium | 14.9 |
| | | TNFalpha + IL1beta | |
| Primary Th2 rest | 19.1 | Small airway epithelium none | 3.0 |
| Primary Tr1 rest | 14.6 | Small airway epithelium | 40.6 |
| | | TNFalpha + IL-1beta | |
| CD45RA CD4 lymphocyte | 4.5 | Coronery artery SMC rest | 1.1 |
| act | | | |
| CD45RO CD lymphocyte | 18.9 | Coronery artery SMC | 4.4 |
| act | | TNFalpha + IL-1beta | |
| CD8 lymphocyte act | 13.6 | Astrocytes rest | 5.3 |
| Secondary CD8 | 2.8 | Astrocytes TNFalpha + IL- | 2.5 |
| lymphocyte rest | | 1beta | |
| Secondary CD8 | 0.0 | KU-812 (Basophil) rest | 12.9 |
| lymphocyte act | | | |
| CD4 lymphocyte none | 10.1 | KU-812 (Basophil) | 36.6 |
| | | PMA/ionomycin | |
| 2ry Th1/Th2/Tr1_anti- | 12.1 | CCD1106 (Keratinocytes) | 2.9 |
| CD95 CH11 | | none | |
| LAK cells rest | 17.6 | CCD1106 (Keratinocytes) | 0.0 |
| | | TNFalpha + IL-1beta | |
| LAK cells IL-2 | 16.6 | Liver cirrhosis | 38.4 |
| LAK cells IL-2 + IL-12 | 26.8 | Lupus kidney | 2.1 |
| LAK cells IL-2 + IFN | 25.7 | NCI-H292 none | 26.2 |
| gamma | | | |
| LAK cells IL-2 + IL-18 | 12.0 | NCI-H292 IL-4 | 25.7 |
| LAK cells | 4.1 | NCI-H292 IL-9 | 22.5 |
| PMA/ionomycin | | | |
| NK Cells IL-2 rest | 11.3 | NCI-H292 IL-13 | 9.5 |
| Two Way MLR 3 day | 31.4 | NCI-H292 IFN gamma | 26.1 |
| Two Way MLR 5 day | 5.6 | HPAEC none | 4.1 |
| Two Way MLR 7 day | 5.8 | HPAEC TNF alpha + IL-1 beta | 2.7 |
| PBMC rest | 3.1 | Lung fibroblast none | 9.2 |
| PBMC PWM | 5.3 | Lung fibroblas TNF alpha + | 28.1 |
| | | IL-1beta | |
| PBMC PHA-L | 12.8 | Lung fibroblast IL-4 | 7.1 |
| Ramos (B cell) none | 0.0 | Lung fibroblast IL-9 | 19.6 |
| Ramos (B cell) ionomycin | 4.9 | Lung fibroblast IL-13 | 5.7 |
| B lymphocytes PWM | 16.3 | Lung fibroblast IFN gamma | 10.6 |
| B lymphocytes CD40L and | 5.6 | Dermal fibroblast CCD1070 | 4.4 |
| IL-4 | | rest | |
| EOL-1 dbcAMP | 2.2 | Dermal fibroblast CCD1070 | 16.0 |
| | | TNF alpha | |
| EOL-1 dbcAMP | 2.5 | Dermal fibroblast CCD1070 | 3.7 |
| PMA/ionomycin | | IL-1 beta | |
| Dendritic cells none | 5.2 | Dermal fibroblast IFN gamma | 4.0 |
| Dendritic cells LPS | 18.3 | Dermal fibroblast IL-4 | 6.4 |
| Dendritic cells anti-CD40 | 3.1 | IBD Colitis 2 | 0.0 |
| Monocytes rest | 2.9 | IBD Crohn's | 0.0 |
| Monocytes LPS | 1.3 | Colon | 22.8 |
| Macrophages rest | 3.1 | Lung | 5.6 |
| Macrophages LPS | 0.0 | Thymus | 89.5 |
| HUVEC none | 0.0 | Kidney | 67.8 |
| HUVEC starved | 7.3 | | |

CNS_neurodegeneration_v1.0 Summary: Ag2213 Data from one run with this probe and primer set is not included because the amp plot suggests that there were experimental difficulties with this run.

Panel 1.3D Summary: Ag2213 Significant expression of this gene is seen exclusively in a single ovarian cancer cell line (CT=33.5). Therefore, expression of this gene may be used to distinguish ovarian cancer cell lines from the other samples on this panel. Furthermore, therapeutic modulation of the activity of the GPCR encoded by this gene may be beneficial in the treatment of ovarian cancer.

Panel 2.2 Summary: Ag2213 Data from one run with this probe and primer set is not included because the data indicates that there was a high probability of a chemistry failure.

Panel 4D Summary: Ag2213 This gene is expressed at moderate to low levels in a wide range of cell types of significance to the immune response and tissue response in normal and disease states, with the highest expression in resting primary Th1 cells (CT=30.9). This expression suggests that targeting of the protein encoded by this gene with a small molecule drug or antibody therapeutic may modulate the functions of B cells, T cells, and/or other cells of the immune system as well as resident tissue cells (eg. lung fibroblasts) and lead to improvement of the symptoms of patients suffering from autoimmune and inflammatory diseases such as asthma, allergies, inflammatory bowel disease, lupus erythematosus, and arthritis.

D. CG143529-01/CG143529-01: Olfactory Receptor

Expression of gene CG143529-01 was assessed using the primer-probe sets Ag2372 and Ag2212, described in Tables DA and DB. Results of the RTQ-PCR runs are shown in Table DC.

TABLE DA
Probe Name Ag2372

| | | | Start | SEQ ID |
|---|---|---|---|---|
| Primers | Sequences | Length | Position | NO |
| Forward | 5′-ctggccaatctctatgttcttg-3′ | 22 | 852 | 140 |
| Probe | TET-5′-ccccatgatgaacccaattatctatgga-3′-TAMRA | 28 | 878 | 141 |
| Reverse | 5′-caacccctttctgaatctgttt-3′ | 22 | 915 | 142 |

TABLE DB
Probe Name Ag2212

| | | | Start | SEQ ID |
|---|---|---|---|---|
| Primers | Sequences | Length | Position | NO |
| Forward | 5′-ctggccaatctctatgttcttg-3′ | 22 | 852 | 143 |
| Probe | TET-5′-ccccatgatgaacccaattatctatgga-3′-TAMRA | 28 | 878 | 144 |
| Reverse | 5′-caacccctttctgaatctggttt-3′ | 22 | 915 | 145 |

TABLE DC
Panel 4D

| | Rel. Exp.(%) | | Rel. Exp.(%) |
|---|---|---|---|
| | Ag2372, Run | | Ag2372, Run |
| Tissue Name | 163724374 | Tissue Name | 163724374 |
| Secondary Th1 act | 0.0 | HUVEC IL-1beta | 0.0 |
| Secondary Th2 act | 0.1 | HUVEC IFN gamma | 0.0 |
| Secondary Tr1 act | 0.1 | HUVEC TNF alpha + IFN | 0.0 |
| | | gamma | |
| Secondary Th1 rest | 0.2 | HUVEC TNF alpha + IL4 | 0.0 |
| Secondary Th2 rest | 0.1 | HUVEC IL-11 | 0.1 |
| Secondary Tr1 rest | 0.1 | Lung Microvascular EC none | 0.1 |
| Primary Th1 act | 0.0 | Lung Microvascular EC | 0.1 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.4 | Bronchial epithelium | 0.1 |
| | | TNFalpha + IL1beta | |
| Primary Th2 rest | 0.1 | Small airway epithelium none | 0.0 |
| Primary Tr1 rest | 0.2 | Small airway epithelium | 0.1 |
| | | TNFalpha + IL-1beta | |
| CD45RA CD4 lymphocyte | 0.1 | Coronery artery SMC rest | 0.0 |
| act | | | |
| CD45RO CD4 lymphocyte | 0.1 | Coronery artery SMC | 0.0 |
| act | | TNFalpha + IL-1beta | |
| CD8 lymphocyte act | 0.2 | Astrocytes rest | 0.1 |
| Secondary CD8 | 0.1 | Astrocytes TNFalpha + IL- | 0.0 |
| lymphocyte rest | | 1beta | |
| Secondary CD8 | 100.0 | KU-812 (Basophil) rest | 0.2 |
| lymphocyte act | | | |
| CD4 lymphocyte none | 0.1 | KU-812 (Basophil) | 0.3 |
| | | PMA/ionomycin | |
| 2ry Th1/Th2/Tr1_anti- | 0.1 | CCD1106 (Keratinocytes) | 0.0 |
| CD95 CH11 | | none | |
| LAK cells rest | 0.2 | CCD1106 (Keratinocytes) | 0.0 |
| | | TNFalpha + IL-1beta | |
| LAK cells IL-2 | 0.3 | Liver cirrhosis | 0.2 |
| LAK cells IL-2 + IL-12 | 0.1 | Lupus kidney | 0.1 |
| LAK cells IL-2 + IFN | 0.1 | NCI-H292 none | 0.3 |
| gamma | | | |
| LAK cells IL-2 + IL-18 | 0.2 | NCI-H292 IL-4 | 0.3 |
| LAK cells | 0.1 | NCI-H292 IL-9 | 0.2 |
| PMA/ionomycin | | | |
| NK Cells IL-2 rest | 0.1 | NCI-H292 IL-13 | 0.1 |
| Two Way MLR 3 day | 0.5 | NCI-H292 IFN gamma | 0.3 |
| Two Way MLR 5 day | 0.2 | HPAEC none | 0.0 |
| Two Way MLR 7 day | 0.1 | HPAEC TNF alpha + IL-1 beta | 0.0 |
| PBMC rest | 0.0 | Lung fibroblast none | 0.1 |
| PBMC PWM | 0.1 | Lung fibroblast TNF alpha + | 0.2 |
| | | IL-1 beta | |
| PBMC PHA-L | 0.0 | Lung fibroblast IL-4 | 0.1 |
| Ramos (B cell) none | 0.0 | Lung fibroblast IL-9 | 0.1 |
| Ramos (B cell) ionomycin | 0.1 | Lung fibroblast IL-13 | 0.1 |
| B lymphocytes PWM | 0.0 | Lung fibroblast IFN gamma | 0.2 |
| B lymphocytes CD40L and | 0.0 | Dermal fibroblast CCD1070 | 0.1 |
| IL-4 | | rest | |
| EOL-1 dbcAMP | 0.1 | Dermal fibroblast CCD1070 | 0.2 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.2 |
| Dendritic cells LPS | 0.1 | Dermal fibroblast IL-4 | 0.2 |
| Dendritic cells anti-CD40 | 0.0 | IBD Colitis 2 | 0.0 |
| Monocytes rest | 0.1 | IBD Crohn's | 0.1 |
| Monocytes LPS | 0.1 | Colon | 0.1 |
| Macrophages rest | 0.0 | Lung | 0.1 |
| Macrophages LPS | 0.0 | Thymus | 0.6 |
| HUVEC none | 0.0 | Kidney | 0.6 |
| HUVEC starved | 0.1 | | |

CNS_neurodegeneration_v1.0 Summary: Ag2372 Expression is low/undetectable in all samples in this panel (CTs>34.5). (Data not shown.)

Panel 1.3D Summary: Ag2212 Expression is low/undetectable in all samples in this panel (CTs>35). (Data not shown.)

Panel 4D Summary: Ag2372 Expression of this transcript is detected at the highest level in activated CD8+ cytotoxic T cells (CT=25.4). Low but significant levels of expression are detected in other cells of the immune system. Thus, targeting of the protein encoded by this gene through the application of a small molecule drug or antibody therapeutic may modulate its function and lead to improvement of the symptoms of patients suffering from autoimmune and infectious diseases.

E. CG144264-01/GMAP001804_I: Olfactory Receptor

Expression of gene CG144264-01 was assessed using the primer-probe set Ag2217, described in Table EA. Results of the RTQ-PCR runs are shown in Table EB.

TABLE EA
Probe Name Ag2217

| | | | Start | SEQ ID |
|---|---|---|---|---|
| Primers | Sequences | Length | Position | NO |
| Forward | 5′-gctgtggaaaatgactcttcag-3′ | 22 | 7 | 146 |
| Probe | TET-5′-ttcttttgggattaacagaccagcct-3′-TAMRA | 26 | 42 | 147 |
| Reverse | 5′-acaggggcaattggatctc-3′ | 19 | 68 | 148 |

TABLE EB
Panel 1.3D

| | Rel. Exp.(%) Ag2217, | | Rel. Exp.(%) Ag2217, |
|---|---|---|---|
| Tissue Name | Run 165974924 | Tissue Name | Run 165974924 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 15.3 | Lung | 0.0 |
| Brain (hippocampus) | 0.0 | Lung (fetal) | 0.0 |
| Brain (substantia nigra) | 0.0 | Lung ca. (small cell) | 7.0 |
| | | LX-1 | |
| Brain (thalamus) | 13.5 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 0.0 | Lung ca. (large | 0.0 |
| | | cell)NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met | 0.0 | Lung ca. (non-s. cl) NCI- | 0.0 |
| SK-N-AS | | H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl. ef) MCF- | 100.0 |
| | | 7 | |
| glioma SF-295 | 0.0 | Breast ca.* (pl.ef) MDA- | 0.0 |
| | | MB-231 | |
| Heart (Fetal) | 0.0 | Breast ca.* (pl. ef) T47D | 0.0 |
| Heart | 0.0 | Breast ca. BT-549 | 0.0 |
| Skeletal muscle (Fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 13.2 | Ovarian ca. IGROV-1 | 0.0 |
| Stomach | 0.0 | Ovarian ca. (ascites) SK- | 0.0 |
| | | OV-3 | |
| Small intestine | 0.0 | Uterus | 0.0 |
| Colon ca SW480 | 0.0 | Placenta | 42.6 |
| Colon ca.* SW620 | 7.1 | Prostate | 0.0 |
| (SW480 met) | | | |
| Colon ca. HT29 | 6.6 | Prostate ca.* (bone met) | 61.6 |
| | | PC-3 | |
| Colon ca. HCT-116 | 0.0 | Testis | 8.6 |
| Colon ca. CaCo-2 | 0.0 | Melanoma Hs688(A).T | 0.0 |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 62.0 |
| Gastric ca. (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

Panel 1.3D Summary: Ag2217 The expression of this gene is highest in and exclusive to one breast cancer cell line (MCF-7). Thus, the expression of this gene could be used to distinguish samples derived from this cell line from other samples in the panel. Moreover, therapeutic modulation of this gene, through the use of small molecule drugs, antibodies or protein therapeutics might be of benefit in the treatment of breast cancer.

Panel 2.2 Summary: Ag2217 Data from one experiment with this probe and primer set is not included because of the high probability of a chemistry failure.

Panel 4D Summary: Ag2217 Expression low/undetectable across all of the samples on this panel. (Data not shown.)

F. CG54353-04/GMAP001804_F: Olfactory Receptor

Expression of gene CG54353-04 was assessed using the primer-probe set Ag2549, described in Table FA. Results of the RTQ-PCR runs are shown in Table FB.

TABLE FA
Probe Name Ag2549

| | | | Start | SEQ ID |
|---|---|---|---|---|
| Primers | Sequences | Length | Position | NO |
| Forward | 5′-tctcacctccacacaccaat-3′ | 20 | 164 | 149 |
| Probe | TET-5′-ttcctcttcaatctctccttcattga-3′-TAMRA | 26 | 191 | 150 |
| Reverse | 5′-gcattttggggagtgaaaaca-3′ | 20 | 232 | 151 |

TABLE FB
Panel 4.1

| | Rel. Exp.(%) | | Rel. Exp.(%) |
|---|---|---|---|
| | Ag2549, Run | | Ag2549, Run |
| Tissue Name | 224781632 | Tissue Name | 224781632 |
| Secondary Th1 act | 0.0 | HUVEC IL-1beta | 0.0 |
| Secondary Th2 act | 0.0 | HUVEC IFN gamma | 0.2 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.2 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC | 0.0 |
| act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.2 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 0.3 |
| LAK cells IL-2 + IL-12 | 0.0 | NCI-H292 none | 0.2 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1 beta | 0.0 |
| Two Way MLR 7 day | 0.0 | Lung fibroblast none | 0.0 |
| PBMC rest | 0.0 | Lung fibroblast TNF alpha + | 0.2 |
| | | IL-1 beta | |
| PBMC PWM | 0.0 | Lung fibroblast IL-4 | 0.0 |
| PBMC PHA-L | 0.0 | Lung fibroblast IL-9 | 0.7 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 | 0.0 |
| | | rest | |
| B lymphocytes CD40L and | 0.0 | Dermal fibroblast CCD1070 | 0.0 |
| IL-4 | | TNF alpha | |
| EOL-1 dbcAMP | 0.2 | Dermal fibroblast CCD1070 | 0.0 |
| | | IL-1 beta | |
| EOL-1 dbcAMP | 0.0 | Dermal fibroblast IFN gamma | 0.4 |
| PMA/ionomycin | | | |
| Dendritic cells none | 0.9 | Dermal fibroblast IL-4 | 0.2 |
| Dendritic cells LPS | 0.2 | Dermal Fibroblasts rest | 0.6 |
| Dendritic cells anti-CD40 | 0.4 | Neutrophils TNFa + LPS | 0.4 |
| Monocytes rest | 0.0 | Neutrophils rest | 1.1 |
| Monocytes LPS | 0.0 | Colon | 1.3 |
| Macrophages rest | 0.7 | Lung | 2.6 |
| Macrophages LPS | 0.0 | Thymus | 14.3 |
| HUVEC none | 0.0 | Kidney | 100.0 |
| HUVEC starved | 0.0 | | |

CNS_neurodegeneration_v1.0 Summary: Ag2549 Expression low/undetectable across all of the samples on this panel. (Data not shown.)

Panel 1.3D Summary: Ag2549 Expression low/undetectable across all of the samples on this panel. (Data not shown.)

Panel 2.2 Summary: Ag2549 Expression low/undetectable across all of the samples on this panel. (Data not shown.)

Panel 4.1D Summary: Ag2549 This gene is expressed at detectable levels in the kidney with lower expression in the thymus. The putative GPCR encoded for by this gene could allow cells within the kidney to respond to specific microenvironmental signals. Therefore, antibody or small molecule therapies designed with the protein encoded for by this gene could modulate kidney function and be important in the treatment of inflammatory or autoimmune diseases that affect the kidney, including lupus and glomerulonephritis. Please note that data from a second experiment with the same probe and primer set showed low/undetectable levels of expression in all the samples in this panel. (Data not shown.)

G. CG54326-03/GMAP001804_D: Olfactory Receptor

Expression of gene CG54326-03 was assessed using the primer-probe sets Ag2357 and Ag1634, described in Tables GA and GB. Results of the RTQ-PCR runs are shown in Tables GC and GD.

TABLE GA
Probe Name Ag2357

| | | | Start | SEQ ID |
|---|---|---|---|---|
| Primers | Sequences | Length | Position | NO |
| Forward | 5′-tgaactttgttccagaggagaa-3′ | 22 | 258 | 151 |
| Probe | TET-5′-tctcctttctggaatgcattactcaa-3′-TAMRA | 26 | 285 | 152 |
| Reverse | 5′-ggtagccttctgcaatacaaa-3′ | 22 | 329 | 153 |

TABLE GB
Probe Name Ag1634

| | | | Start | SEQ ID |
|---|---|---|---|---|
| Primers | Sequences | Length | Position | NO |
| Forward | 5′-tgaactttgttccagaggagaa-3′ | 22 | 258 | 154 |
| Probe | TET-5′-tctcctttctggaatgcattactcaa-3′-TAMRA | 26 | 285 | 155 |
| Reverse | 5′-ggtagccttctgcaattacaaa-3′ | 22 | 329 | 156 |

TABLE GC
Panel 1.3D

| | Rel. Exp.(%) Ag1634, | | Rel. Exp.(%) Ag1634, |
|---|---|---|---|
| Tissue Name | Run 165924466 | Tissue Name | Run 165924466 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 3.9 | Liver ca. (hepatoblast) | 0.0 |
| | | HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) LX-1 | 6.5 |
| Brain (thalamus) | 6.0 | Lung ca. (small cell) NCI- | 0.0 |
| | | H69 | |
| Cerebral Cortex | 4.7 | Lung ca. (s. cell var.) SHP- | 0.0 |
| | | 77 | |
| Spinal cord | 0.0 | Lung ca. (large cell) NCI- | 9.3 |
| | | H460 | |
| glio/astro U87-MG | 0.0 | Lung ca. (non-sm. cell) | 0.0 |
| | | A549 | |
| glio/astro U-118-MG | 0.0 | Lung ca. (non-s. cell) NCI- | 0.0 |
| | | H23 | |
| astrocytoma SW1783 | 0.0 | Lung ca. (non-s. cell) | 4.9 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW 900 | 0.0 |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl. ef) MCF-7 | 0.0 |
| glioma SF-295 | 0.0 | Breast ca.* (pl. ef) MDA- | 0.0 |
| | | MB-231 | |
| Heart (Fetal) | 0.0 | Breast ca.* (pl. ef) T47D | 137.1 |
| Heart | 0.0 | Breast ca. BT-549 | 0.0 |
| Skeletal muscle (Fetal) | 0.0 | Breast ca. MDA-N | 0.0 |
| Skeletal muscle | 3.7 | Ovary | 0.0 |
| Bone marrow | 0.0 | Ovarian ca. OVCAR-3 | 4.0 |
| Thymus | 0.0 | Ovarian ca. OVCAR-4 | 0.0 |
| Spleen | 100.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca. (ascites) SK- | 5.1 |
| | | OV-3 | |
| Small intestine | 0.0 | Uterus | 0.0 |
| Colon ca. SW480 | 0.0 | Placenta | 4.7 |
| Colon ca.* SW620 | 0.0 | Prostate | 0.0 |
| (SW480 met) | | | |
| Colon ca. HT29 | 4.5 | Prostate ca.* (bone met) | 4.2 |
| | | PC-3 | |
| Colon ca. HCT-116 | 0.0 | Testis | 9.3 |
| Colon ca. CaCo-2 | 0.0 | Melanoma Hs688(A).T | 0.0 |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 23.7 |
| Gastric ca. (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE GD
Panel 4D

| | Rel. Exp.(%) Ag1634, | | Rel. Exp.(%) Ag1634, |
|---|---|---|---|
| Tissue Name | Run 165762887 | Tissue Name | Run 165762887 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular | 0.0 |
| | | Dermal EC none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 3.4 |
| lymphocyte act | | | |
| CD4 lymphocyte none | 0.0 | KU-812 (Basophil) | 4.8 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 72.7 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 6.7 |
| | | beta | |
| PBMC rest | 0.0 | Lung fibroblast none | 2.9 |
| PBMC PWM | 0.0 | Lung fibroblast TNF alpha + | 0.0 |
| | | IL-1 beta | |
| PBMC PHA-L | 0.0 | Lung fibroblast IL-4 | 5.3 |
| Ramos (B cell) none | 0.0 | Lung fibroblast IL-9 | 0.0 |
| Ramos (B cell) | 0.0 | Lung fibroblast IL-13 | 6.9 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 3.0 | Dermal fibroblast IFN gamma | 0.0 |
| Dendritic cells LPS | 0.0 | Dermal fibroblast IL-4 | 0.0 |
| Dendritic cells anti-CD40 | 57.8 | IBD Colitis 2 | 11.5 |
| Monocytes rest | 0.0 | IBD Crohn's | 0.0 |
| Monocytes LPS | 0.0 | Colon | 100.0 |
| Macrophages rest | 36.6 | Lung | 6.0 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag2537 Data from one experiment with this probe and primer set is not included due to the high probability of a probe failure.

Panel 1.3D Summary: Ag1634 Expression of this gene is low/undetectable (CT values>35) in all cell lines and tissues except for spleen. Therefore, this gene may be used to distinguish spleen from other tissues. A second experiment with the same probe and primer set shows low/undetectable (CT values 40) in all samples on this panel.

Panel 2D Summary: Ag2537 Data from one experiment with this probe and primer set is not included due to the high probability of a probe failure.

Panel 4D Summary: Ag1634 Expression of this transcript is detected in colitis 1 and in dendritic cells treated with anti-CD40. The protein encoded for by this antigen may be important in the inflammatory process and particularly in the function of activated dendritic cells. Antagonistic antibodies or small molecule therapeutics that inhibit the function of the protein encoded by this gene may therefore reduce or inhibit inflammation in the bowel due to inflammatory bowl disease (IBD). Ag2357 Expression was low/undetectable (CT values 40) in all samples on this panel and chemistry control did not work well (CT=35).

H. CG55972-01/GMAC011711_H: OLFACTORY RECEPTOR

Expression of gene CG55972-01 was assessed using the primer-probe set Ag2351, described in Table HA. Results of the RTQ-PCR runs are shown in Tables HB.

TABLE HA
Probe Name Ag2351

| | | | Start | SEQ ID |
|---|---|---|---|---|
| Primers | Sequences | Length | Position | NO |
| Forward | 5′-ctaccacccagaagtgatcaaa-3′ | 22 | 558 | 157 |
| Probe | TET-5′-cacatattccaaaccttggatcagca-3′-TAMRA | 26 | 582 | 158 |
| Reverse | 5′-ccattcaggtagagctgaagaa-3′ | 22 | 623 | 159 |

TABLE HB
Panel 4D

| | Rel. Exp.(%) | | Rel. Exp.(%) |
|---|---|---|---|
| | Ag2351, Run | | Ag2351, Run |
| Tissue Name | 164023402 | Tissue Name | 164023402 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC | 0.0 |
| act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L and | 0.0 | Dermal fibroblast CCD1070 | 0.0 |
| IL-4 | | rest | |
| EOL-1 dbcAMP | 0.0 | Dermal fibroblast CCD1070 | 17.4 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 13.9 |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

Panel 1.3D Summary: Ag2351 Data from one experiment with this probe and primer set is not included because the amp plot suggests that there were experimental difficulties with this run.

Panel 2.2 Summary: Ag2351 Expression is low/undetectable (CTs>35) across all of the samples on this panel. (Data not shown.)

Panel 4D Summary: Ag2351 This transcript is only detected in liver cirrhosis. Furthermore, this transcript is not detected in normal liver in Panel 1.3D, suggesting that this gene's expression is unique to liver cirrhosis. The gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

I. CG152475-01/GMAC011711_A: Olfactory Receptor

Expression of gene CG152475-01 was assessed using the primer-probe set Ag2344, described in Table IA. Results of the RTQ-PCR runs are shown in Tables IB, IC and ID.

TABLE IA
Probe Name Ag2344

| | | | Start | SEQ ID |
|---|---|---|---|---|
| Primers | Sequences | Length | Position NO | |
| Forward | 5′-tgggagcattatcaccagtag-3′ | 22 | 808 | 160 |
| Probe | TET-5′-atatacctactgctcccgcctgtgct-3′-TAMRA | 26 | 850 | 161 |
| Reverse | 5′-cacactgtagacaatggggttt-3′ | 22 | 876 | 162 |

TABLE IB
Panel 1.3D

| | Rel. Exp.(%) Ag2344, | | Rel. Exp.(%) Ag2344, |
|---|---|---|---|
| Tissue Name | Run 165975025 | Tissue Name | Run 165975025 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 5.9 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 26.6 |
| | | NCI-H69 | |
| Cerebral Cortex | 0.0 | Lung ca. (s. cell var.) | 100.0 |
| | | SHP-77 | |
| Spinal cord | 0.0 | Lung ca. (large | 0.0 |
| | | cell)NCI-H460 | |
| glio/astro U87-MG | 0.0 | Lung ca. (non-sm. cell) | 0.0 |
| | | A549 | |
| glio/astro U-118-MG | 0.0 | Lung ca. non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 12.4 |
| | | H596 | |
| glioma SNB-19 | 0.0 | Mammary gland | 0.0 |
| glioma U251 | 0.0 | Breast ca.* (pl. ef) MCF- | 0.0 |
| | | 7 | |
| glioma SF-295 | 0.0 | Breast ca.* (pl. ef) MDA- | 0.0 |
| | | MB-231 | |
| Heart (Fetal) | 0.0 | Breast ca.* (pl. ef) T47D | 0.0 |
| Heart | 0.0 | Breast ca. BT-549 | 0.0 |
| Skeletal muscle (Fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 24.0 | Ovarian ca. OVCAR-5 | 0.0 |
| Lymph node | 0.0 | Ovarian ca. OVCAR-8 | 0.0 |
| Colorectal | 2.6 | Ovarian ca. IGROV-1 | 0.0 |
| Stomach | 0.0 | Ovarian ca. (ascites) SK- | 9.5 |
| | | OV-3 | |
| Small intestine | 3.5 | Uterus | 0.0 |
| Colon ca. SW480 | 0.0 | Placenta | 0.0 |
| Colon ca.* SW620 | 0.0 | Prostate | 0.0 |
| (SW480 met) | | | |
| Colon ca. HT29 | 0.0 | Prostate ca.* (bone met) | 0.0 |
| | | PC-3 | |
| Colon ca. HCT-116 | 0.0 | Testis | 8.5 |
| Colon ca. CaCo-2 | 0.0 | Melanoma Hs688(A).T | 0.0 |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE IC
Panel 2.2

| | Rel. Exp.(%) Ag2344, | | Rel. Exp.(%) Ag2344, |
|---|---|---|---|
| Tissue Name | Run 1745553624 | Tissue Name | Run 1745553624 |
| Normal Colon | 0.0 | Kidney Margin (OD04348) | 24.8 |
| Colon cancer (OD06064) | 0.0 | Kidney malignant cancer | 0.0 |
| | | (OD06204B) | |
| Colon Margin (OD06064) | 0.0 | Kidney normal adjacent | 0.0 |
| | | tissue (OD06204E) | |
| Colon cancer (OD06159) | 0.0 | Kidney Cancer (OD04450- | 0.0 |
| | | 01) | |
| Colon Margin (OD06159) | 0.0 | Kidney Margin (OD04450- | 0.0 |
| | | 03) | |
| Colon cancer (OD06297-04) | 0.0 | Kidney Cancer 8120613 | 0.0 |
| Colon Margin (OD06297- | 0.0 | Kidney Margin 8120614 | 0.0 |
| 015) | | | |
| CC Gr.2 ascend colon | 0.0 | Kidney Cancer 9010320 | 0.0 |
| (ODO3921) | | | |
| CC Margin (ODO3921) | 0.0 | Kidney Margin 9010321 | 0.0 |
| Colon cancer metastasis | 0.0 | Kidney Cancer 8120607 | 0.0 |
| (OD06104) | | | |
| Lung Margin (OD06104) | 0.0 | Kidney Margin 8120608 | 0.0 |
| Colon mets to lung | 0.0 | Normal Uterus | 0.0 |
| (OD04451-01) | | | |
| Lung Margin (OD04451-02) | 0.0 | Uterine Cancer 064011 | 0.0 |
| Normal Prostate | 34.9 | Normal Thyroid | 0.0 |
| Prostate Cancer (OD04410) | 17.3 | Thyroid Cancer | 0.0 |
| Prostate Margin (OD04410) | 42.6 | Thyroid Cancer A302152 | 0.0 |
| Normal Ovary | 0.0 | Thyroid Margin A302153 | 0.0 |
| Ovarian cancer (OD06283- | 0.0 | Normal Breast | 0.0 |
| 03) | | | |
| Ovarian Margin (OD06283- | 0.0 | Breast Cancer | 0.0 |
| 07) | | | |
| Ovarian Cancer | 100.0 | Breast Cancer | 0.0 |
| Ovarian cancer (OD06145) | 0.0 | Breast Cancer (OD04590- | 0.0 |
| | | 01) | |
| Ovarian Margin (OD06145) | 0.0 | Breast Cancer Mets | 0.0 |
| | | (OD04590-03) | |
| Ovarian cancer (OD06455- | 0.0 | Breast Cancer Metastasis | 0.0 |
| 03) | | | |
| Ovarian Margin (OD06455- | 0.0 | Breast Cancer | 0.0 |
| 07) | | | |
| Normal Lung | 0.0 | Breast Cancer 9100266 | 0.0 |
| Invasive poor diff. lung | 0.0 | Breast Margin 9100265 | 0.0 |
| adeno (ODO4945-01 | | | |
| Lung Margin (ODO4945- | 18.3 | Breast Cancer A209073 | 0.0 |
| 03) | | | |
| Lung Malignant Cancer | 39.5 | Breast Margin A2090734 | 0.0 |
| (OD03126) | | | |
| Lung Margin (OD03126) | 0.0 | Breast cancer (OD06083) | 0.0 |
| Lung Cancer (OD05014A) | 0.0 | Breast cancer node | 0.0 |
| | | metastasis (OD06083) | |
| Lung Margin (OD05014B) | 0.0 | Normal Liver | 0.0 |
| Lung cancer (OD06081) | 0.0 | Liver Cancer 1026 | 18.8 |
| Lung Margin (OD06081) | 0.0 | Liver Cancer 1025 | 17.6 |
| Lung Cancer (OD04237-01) | 0.0 | Liver Cancer 6004-T | 0.0 |
| Lung Margin (OD04237-02) | 0.0 | Liver Tissue 6004-N | 0.0 |
| Ocular Mel Met to Liver | 0.0 | Liver Cancer 6005-T | 0.0 |
| (OD04310) | | | |
| Liver Margin (ODO4310) | 0.0 | Liver Tissue 6005-N | 0.0 |
| Melanoma Metastasis | 0.0 | Liver Cancer | 0.0 |
| Lung Margin (OD04321) | 0.0 | Normal Bladder | 0.0 |
| Normal Kidney | 0.0 | Bladder Cancer | 0.0 |
| Kidney Ca, Nuclear grade 2 | 0.0 | Bladder Cancer | 0.0 |
| (OD04338) | | | |
| Kidney Margin (OD04338) | 0.0 | Normal Stomach | 0.0 |
| Kidney Ca Nuclear grade | 0.0 | Gastric Cancer 9060397 | 0.0 |
| 1/2 (OD04339) | | | |
| Kidney Margin (OD04339) | 0.0 | Stomach Margin 9060396 | 0.0 |
| Kidney Ca, Clear cell type | 0.0 | Gastric Cancer 9060395 | 0.0 |
| (OD04340) | | | |
| Kidney Margin (OD04340) | 0.0 | Stomach Margin 9060394 | 0.0 |
| Kidney Ca, Nuclear grade 3 | 0.0 | Gastric Cancer 064005 | 0.0 |
| (OD04348) | | | |

TABLE ID
Panel 4D

| | Rel. Exp.(%) | | Rel. Exp.(%) |
|---|---|---|---|
| | Ag2344, Run | | Ag2344, Run |
| Tissue Name | 163921378 | Tissue Name | 163921378 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC | 0.0 |
| act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | |
| LAK cells | 13.3 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 0.0 | Dermal fibroblast CCD1070 | 0.0 |
| IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 51.8 |
| Dendritic cells anti-CD40 | 0.0 | IBD Colitis 2 | 24.1 |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

Panel 1.3D Summary: Ag2344 Significant restriction of this gene is restricted to samples derived from lung cancer cell lines. Thus, expression of this gene could be used to differentiate between these samples and the other samples on this panel and as a marker for lung cancer. Furthermore, therapeutic modulation of the expression or function of this gene product may be effective in the treatment of lung cancer.

Panel 2.2 Summary: Ag2344 Significant restriction of this gene is restricted to a sample derived from ovarian cancer. Thus, expression of this gene could be used to differentiate between these samples and the other samples on this panel and as a marker for ovarian cancer. Furthermore, therapeutic modulation of the expression or function of this gene product may be effective in the treatment of ovarian cancer.

Panel 4D Summary: Ag2344 Low but significant expression of this gene is detected in a liver cirrhosis sample (CT=33.36). Furthermore, expression of this gene is not detected in normal liver in Panel 11.3D, suggesting that its expression is unique to liver cirrhosis. Low levels of expression in IL-4 stimulated dermal fibroblasts are also detected. This gene encodes a putative GPCR; therefore, antibodies or small molecule therapeutics may potentially reduce or inhibit fibrosis that occurs in liver cirrhosis and scleroderma. In addition, antibodies to this putative GPCR could potentially be used for the diagnosis of liver cirrhosis.

J. CG152485-01/GMAC009642_D: Olfactory Receptor

Expression of gene CG152485-01 was assessed using the primer-probe set Ag2342, described in Table JA. Results of the RTQ-PCR runs are shown in Tables JB, JC and JD.

TABLE JA
Probe Name Ag2342

| | | | Start | SEQ ID |
|---|---|---|---|---|
| Primers | Sequences | Length | Position | NO |
| Forward | 5′-catgttcttctggccatactgt-3′ | 22 | 876 | 163 |
| Probe | TET-5′-cttgtgccacctgcactcaatcctct-3′-TAMRA | 26 | 903 | 164 |
| Reverse | 5′-ctgggtcttcaccctatagaca-3′ | 22 | 929 | 165 |

TABLE JB
Panel 1.3D

| | Rel. Exp.(%) Ag2342, | | Rel. Exp.(%) Ag2342, |
|---|---|---|---|
| Tissue Name | Run 165975017 | Tissue Name | Run 165975017 |
| Liver adenocarcinoma | 6.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 12.3 |
| Thyroid | 0.0 | Renal ca. ACHN | 0.0 |
| Salivary gland | 0.0 | Renal ca. UO-31 | 0.0 |
| Pituitary gland | | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) LX-1 | 0.0 |
| Brain (thalamus) | 0.0 | Lung ca. (small cell) | 0.0 |
| | | NCI-H69 | |
| Cerebral Cortex | 0.0 | Lung ca. (s. cell var.) | 5.6 |
| | | SHP-77 | |
| Spinal cord | 0.0 | Lung ca. (large | 0.0 |
| | | cell)NCI-H460 | |
| glio/astro U87-MG | 0.0 | Lung ca. (non-sm. cell) | 0.0 |
| | | A549 | |
| glio/astro U-118-MG | 0.0 | Lung ca. (non-s.cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-194 | 5.2 | Mammary gland | 0.0 |
| glioma U251 | 0.0 | Breast ca.* (pl. ef) MCF- | 0.0 |
| | | 7 | |
| glioma SF-295 | 0.0 | Breast ca.* (pl. ef) MDA- | 3.7 |
| | | MB-231 | |
| Heart (Fetal) | 0.0 | Breast ca.* (pl. ef) T47D | 0.0 |
| Heart | 0.0 | Breast ca. BT-549 | 0.0 |
| Skeletal muscle (Fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 3.3 |
| Thymus | 0.0 | Ovarian ca. OVCAR-4 | 0.0 |
| Spleen | 100.0 | Ovarian ca. OVCAR-5 | 0.0 |
| Lymph node | 0.0 | Ovarian ca. OVCAR-8 | 0.0 |
| Colorectal | 20.2 | Ovarian ca. IGROV-1 | 0.0 |
| Stomach | 0.0 | Ovarian ca. (ascites) SK- | 17.8 |
| | | OV-3 | |
| Small intestine | 0.0 | Uterus | 0.0 |
| Colon ca. SW480 | 0.0 | Placenta | 0.0 |
| Colon ca.* SW620 | 0.0 | Prostate | 9.3 |
| (SW480 met) | | | |
| Colon ca. HT29 | 0.0 | Prostate ca.* (bone met) | 4.5 |
| | | PC-3 | |
| Colon ca. HCT-116 | 0.0 | Testis | 0.0 |
| Colon ca. CaCo-2 | 0.0 | Melanoma Hs688(A).T | 0.0 |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE JC
Panel 2.2

| | Rel. Exp.(%) Ag2342, | | Rel. Exp.(%) Ag2342, |
|---|---|---|---|
| Tissue Name | Run 174553623 | Tissue Name | Run 174553623 |
| Normal Colon | 65.5 | Kidney Margin (OD04348) | 0.0 |
| Colon cancer (OD06064) | 0.0 | Kidney malignant cancer | 0.0 |
| | | (OD06204B) | |
| Colon Margin (OD06064) | 19.5 | Kidney normal adjacent | 33.9 |
| | | tissue (OD06204E) | |
| Colon cancer (OD06159) | 0.0 | Kidney Cancer (OD04450- | 0.0 |
| | | 01) | |
| Colon Margin (OD06159) | 0.0 | Kidney Margin (OD04450- | 0.0 |
| | | 03) | |
| Colon cancer (OD06297-04) | 0.0 | Kidney Cancer 8120613 | 0.0 |
| Colon Margin (OD06297- | 0.0 | Kidney Margin 8120614 | 0.0 |
| 015) | | | |
| CC Gr.2 ascend colon | 0.0 | Kidney Cancer 9010320 | 27.7 |
| (ODO3921) | | | |
| CC Margin (ODO3921) | 0.0 | Kidney Margin 9010321 | 0.0 |
| Colon cancer metastasis | 0.0 | Kidney Cancer 8120607 | 0.0 |
| (OD06104) | | | |
| Lung Margin (OD06104) | 0.0 | Kidney Margin 8120608 | 0.0 |
| Colon mets to lung | 28.1 | Normal Uterus | 36.1 |
| (OD04451-01) | | | |
| Lung Margin (OD04451-02) | 0.0 | Uterine Cancer 064011 | 84.7 |
| Normal Prostate | 0.0 | Normal Thyroid | 0.0 |
| Prostate Cancer (OD04410) | 0.0 | Thyroid Cancer | 0.0 |
| Prostate Margin (OD04410) | 0.0 | Thyroid Cancer A302152 | 0.0 |
| Normal Ovary | 0.0 | Thyroid Margin A302153 | 0.0 |
| Ovarian cancer (OD06283- | 0.0 | Normal Breast | 0.0 |
| 03) | | | |
| Ovarian Margin (OD06283- | 30.4 | Breast Cancer | 0.0 |
| 07) | | | |
| Ovarian Cancer | 52.9 | Breast Cancer | 0.0 |
| Ovarian cancer (OD06145) | 0.0 | Breast Cancer (OD04590- | 0.0 |
| | | 01) | |
| Ovarian Margin (OD06145) | 0.0 | Breast Cancer Mets | 0.0 |
| | | (OD04590-03) | |
| Ovarian cancer (OD06455- | 0.0 | Breast Cancer Metastasis | 0.0 |
| 03) | | | |
| Ovarian Margin (OD06455- | 0.0 | Breast Cancer | 0.0 |
| 07) | | | |
| Normal Lung | 0.0 | Breast Cancer 9100266 | 0.0 |
| Invasive poor diff. lung | 0.0 | Breast Margin 9100265 | 0.0 |
| adeno (OD04945-01 | | | |
| Lung Margin (ODO4945- | 0.0 | Breast Cancer A209073 | 0.0 |
| 03) | | | |
| Lung Malignant Cancer | 0.0 | Breast Margin A2090734 | 46.0 |
| (OD03126) | | | |
| Lung Margin (OD03126) | 0.0 | Breast cancer (OD06083) | 0.0 |
| Lung Cancer (OD05014A) | 0.0 | Breast cancer node | 0.0 |
| | | metastasis (OD06083) | |
| Lung Margin (OD05014B) | 54.7 | Normal Liver | 0.0 |
| Lung cancer (OD06081) | 0.0 | Liner Cancer 1026 | 27.4 |
| Lung Margin (OD06081) | 0.0 | Liver Cancer 1025 | 34.6 |
| Lung Cancer (OD04237-01) | 26.8 | Liver Cancer 6004-T | 0.0 |
| Lung Margin (OD04237-02) | 25.0 | Liver Tissue 6004-N | 0.0 |
| Ocular Mel Met to Liver | 0.0 | Liver Cancer 6005-T | 0.0 |
| (ODO4310) | | | |
| Liver Margin (ODO4310) | 0.0 | Liver Tissue 6005-N | 0.0 |
| Melanoma Metastasis | 100.0 | Liver Cancer | 0.0 |
| Lung Margin (OD04321) | 0.0 | Normal Bladder | 0.0 |
| Normal Kidney | 0.0 | Bladder Cancer | 0.0 |
| Kidney Ca, Nuclear grade 2 | 0.0 | Bladder Cancer | 31.4 |
| (OD04338) | | | |
| Kidney Margin (OD04338) | 0.0 | Normal Stomach | 0.0 |
| Kidney Ca Nuclear grade | 0.0 | Gastric Cancer 9060397 | 0.0 |
| 1/2 (OD04339) | | | |
| Kidney Margin (OD04339) | 0.0 | Stomach Margin 9060396 | 0.0 |
| Kidney Ca, Clear cell type | 0.0 | Gastric Cancer 9060395 | 0.0 |
| (OD04340) | | | |
| Kidney Margin (OD04340) | 0.0 | Stomach Margin 9060394 | 0.0 |
| Kidney Ca, Nuclear grade 3 | 0.0 | Gastric Cancer 064005 | 0.0 |
| (OD04348) | | | |

TABLE JD
Panel 4D

| | Rel. Exp.(%) | | Rel. Exp.(%) |
|---|---|---|---|
| | Ag2342, Run | | Ag2342, Run |
| Tissue Name | 163921290 | Tissue Name | 163921290 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 9.1 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC | 0.0 |
| act | | TNFalpha + IL-1beta | |
| CD8 lymphocyte act | 0.0 | Astrocytes rest | 0.0 |
| Secondary CD8 | 0.0 | Astrocytes TNFalpha + IL- | 6.7 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 21.6 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 38.7 |
| LAK cells IL-2 + IL-12 | 0.0 | Lupus kidney | 0.0 |
| LAK cells IL-2 + IFN | 8.5 | NCI-H292 none | 19.5 |
| gamma | | | |
| LAK cells IL-2 + IL-18 | 0.0 | NCI-H292 IL-4 | 5.2 |
| LAK cells | 0.0 | NCI-H292 IL-9 | 4.5 |
| PMA/ionomycin | | | |
| NK Cells IL-2 rest | 0.0 | NCI-H292 IL-13 | 0.0 |
| Two Way MLR 3 day | 14.1 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 7.8 |
| PBMC rest | 8.4 | Lung fibroblast none | 0.0 |
| PBMC PWM | 7.2 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 0.0 | Dermal fibroblast CCD1070 | 0.0 |
| IL-4 | | rest | |
| EOL-1 dbcAMP | 0.0 | Dermal fibroblast CCD1070 | 7.8 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 40.9 |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

Panel 1.3D Summary: Ag2342 This gene is expressed at low but significant levels in spleen, an important site of secondary immune responses. Therefore, antibodies or small molecule therapeutics that block the function of this GPCR may be useful as anti-inflammatory therapeutics for the treatment of allergies, autoimmune diseases, and inflammatory diseases.

Panel 2.2 Summary: Ag2342 Expression of this gene is low/undetectable (CTS >35) in all of the samples on this panel (data not shown).

Panel 4D Summary: Ag2342 Expression of this transcript is restricted to resting monocytes (CT=34.1). Therefore, expression of this gene could be used to differentiate this sample from other samples on this panel.

K. CG55958-01/GMAP002512_F: Olfactory receptor

Expression of gene CG55958-01 was assessed using the primer-probe sets Ag2331, Ag2332 and Ag1804, described in Tables KA, KB and KC. Results of the RTQ-PCR runs are shown in Table KD.

TABLE KA
Probe Name Ag2331

| | | | Start | SEQ ID |
|---|---|---|---|---|
| Primers | Sequences | Length | Position | NO |
| Forward | 5′-aaatggtggctgtgttttacac-3′ | 22 | 885 | 166 |
| Probe | TET-5′-tgttgaatcccatgatctacagtctga-3′-TAMRA | 27 | 921 | 167 |
| Reverse | 5′-tgctttgttgactgcttctttt-3′ | 22 | 961 | 168 |

TABLE KB
Probe Name Ag2332

| | | | Start | SEQ ID |
|---|---|---|---|---|
| Primers | Sequences | Length | Position | NO |
| Forward | 5′-aaatggtggctgtgttttacac-3′ | 22 | 885 | 169 |
| Probe | TET-5′-tgttgaatcccatgatctacagtctga-3′-TAMRA | 27 | 921 | 170 |
| Reverse | 5′-tgctttgttgactgcttctttt-30′ | 22 | 961 | 171 |

TABLE KC
Probe Name Ag1804

| | | | Start | SEQ ID |
|---|---|---|---|---|
| Primers | Sequences | Length | Position | NO |
| Forward | 5′-aaatggtggctgtgttttacac-3′ | 22 | 885 | 172 |
| Probe | TET-5′-tgttgaatcccatgatctacagtctga-3′-TAMRA | 27 | 921 | 173 |
| Reverse | 5′-tgctttgttgactgcttctttt-3′ | 22 | 961 | 174 |

TABLE KD
Panel 4D

| | Rel. Exp.(%) | | Rel. Exp.(%) |
|---|---|---|---|
| | Ag1804, Run | | Ag1804, Run |
| Tissue Name | 165812558 | Tissue Name | 165812558 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 100.0 |
| LAK cells IL-2 + IL-12 | 0.0 | Lupus kidney | 3.6 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti-CD40 | 0.0 | IBD Colitis 2 | 8.3 |
| Monocytes rest | 0.0 | IBD Crohn's | 0.0 |
| Monocytes LPS | 0.0 | Colon | 4.4 |
| Macrophages rest | 0.0 | Lung | 0.0 |
| Macrophages LPS | 0.0 | Thymus | 7.4 |
| HUVEC none | 0.0 | Kidney | 0.0 |
| HUVEC starved | 0.0 | | |

CNS_neurodegeneration_v1.0 Summary: Ag2331/Ag2332 Expression of this gene is low/undetectable (CTs>35) across all of the samples on this panel (data not shown).

Panel 1.3D Summary: Ag1804/Ag2331/Ag2332 Expression is low/undetectable in all samples in this panel. (Data not shown.)

Panel 2.2 Summary: Ag1804/Ag233 1/Ag2332 Expression of this gene is low/undetectable (CTs>35) across all of the samples on this panel (data not shown).

Panel 4D Summary: Ag1804 Expression of this gene is restricted to liver cirrhosis (CT=33.6). Furthermore, this transcript is not detected in normal liver in Panel 1.3D, suggesting that this gene expression is unique to liver cirrhosis. The protein encoded by this gene is a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis. Please note that two additional runs with probe and primer sets Ag2331 and Ag2332 produced results that were too low to be evaluated. (CTs>35). (Data not shown.)

L. CG137823-01/GMAC023106_A: Olfactory Receptor

Expression of gene CG137823-01 was assessed using the primer-probe set Ag2323, described in Table LA. Results of the RTQ-PCR runs are shown in Tables LB, LC and LD.

TABLE LA
Probe Name Ag2323

| | | | Start | SEQ ID |
|---|---|---|---|---|
| Primers | Sequences | Length | Position | NO |
| Forward | 5′-caataggaccgctgttgct-3′ | 19 | 65 | 175 |
| Probe | TET-5′-tcattctactgggcctagtgcaaaca-3′-TAMRA | 26 | 88 | 176 |
| Reverse | 5′-acaaagacaactggctgcat-3′ | 20 | 120 | 177 |

TABLE LB
CNS_neurodegeneration_v1.0

| | Rel. Exp.(%) Ag2323, | | Rel. Exp.(%) Ag2323, |
|---|---|---|---|
| Tissue Name | Run 207929053 | Tissue Name | Run 207929053 |
| AD 1 Hippo | 3.0 | Control (Path) 3 | 6.7 |
| | | Temporal Ctx | |
| AD 2 Hippo | 26.1 | Control (Path) 4 | 12.9 |
| | | Temporal Ctx | |
| AD 3 Hippo | 2.2 | AD 1 Occipital Ctx | 8.7 |
| AD 4 Hippo | 2.5 | AD 2 Occipital Ctx | 0.0 |
| | | (Missing) | |
| AD 5 Hippo | 97.9 | AD 3 Occipital Ctx | 3.0 |
| AD 6 Hippo | 45.7 | AD 4 Occipital Ctx | 11.0 |
| Control 2 Hippo | 11.1 | AD 5 Occipital Ctx | 13.2 |
| Control 4 Hippo | 2.9 | AD 6 Occipital Ctx | 15.9 |
| Control (Path) 3 | 4.9 | Control 1 Occipital Ctx | 13.6 |
| Hippo | | | |
| AD 1 Temporal Ctx | 14.5 | Control 2 Occipital Ctx | 21.6 |
| AD 2 Temporal Ctx | 23.0 | Control 3 Occipital Ctx | 5.7 |
| AD 3 Temporal Ctx | 5.7 | Control 4 Occipital Ctx | 4.9 |
| AD 4 Temporal Ctx | 12.9 | Control (Path) 1 | 47.0 |
| | | Occipital Ctx | |
| AD 5 Inf Temporal | 100.00 | Control (Path) 2 | 6.0 |
| Ctx | | Occipital Ctx | |
| AD 5 Sup Temporal | 23.0 | Control (Path) 3 | 2.2 |
| Ctx | | Occipital Ctx | |
| AD 6 Inf Temporal Ctx | 36.3 | Control (Path) 4 | 8.6 |
| | | Occipital Ctx | |
| AD 6 Sup Temporal | 59.5 | Control 1 Parietal Ctx | 16.7 |
| Ctx | | | |
| Control 1 Temporal | 21.9 | Control 2 Parietal Ctx | 34.9 |
| Ctx | | | |
| Control 2 Temporal | 17.0 | Control 3 Parietal Ctx | 1.6 |
| Ctx | | | |
| Control 3 Temporal | 9.0 | Control (Path) 1 | 54.7 |
| Ctx | | Parietal Ctx | |
| Control 3 Temporal | 3.8 | Control (Path) 2 | 18.0 |
| Ctx | | Parietal Ctx | |
| Control (Path) 1 | 46.0 | Control (Path) 3 | 4.0 |
| Temporal Ctx | | Parietal Ctx | |
| Control (Path) 2 | 17.8 | Control (Path) 4 | 20.0 |
| Temporal Ctx | | Parietal Ctx | |

TABLE LC
Panel 1.3D

| | Rel. Exp.(%) Ag2323, | | Rel. Exp.(%) Ag2323, |
|---|---|---|---|
| Tissue Name | Run 165974935 | Tissue Name | Run 165974935 |
| Liver adenocarcinoma | 100.0 | Kidney (fetal) | 0.0 |
| Pancreas | 3.8 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 15.1 | Renal ca. TK-10 | 0.0 |
| Brain (fetal) | 17.2 | Liver | 0.0 |
| Brain (whole) | 34.9 | Liver (fetal) | 0.0 |
| Brain (amygdala) | 43.5 | Liver ca. (hepatoblast) | 0.0 |
| | | HepG2 | |
| Brain (cerebellum) | 6.8 | Lung | 0.0 |
| Brain (hippocampus) | 9.7 | Lung (fetal) | 0.0 |
| Brain (substantia nigra) | 33.9 | Lung ca. (small cell) | 0.0 |
| | | LX-1 | |
| Brain (thalamus) | 44.1 | Lung ca. (small cell) | 0.0 |
| | | NCI-H69 | |
| Cerebral Cortex | 25.0 | Lung ca. (s. cell var.) | 32.8 |
| | | SHP-77 | |
| Spinal cord | 28.5 | Lung ca. (large | 0.0 |
| | | cell)NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 6.6 |
| glioma U251 | 0.0 | Breast ca.* (pl. ef) MCF- | 0.0 |
| | | 7 | |
| glioma SF-295 | 0.0 | Breast ca.* (pl. ef) MDA- | 0.0 |
| | | MB-231 | |
| Heart (fetal) | 0.0 | Breast ca.* (pl. ef) T47D | 0.0 |
| Heart | 7.0 | Breast ca. BT-549 | 0.0 |
| Skeletal muscle (fetal) | 8.1 | Breast ca. MDA-N | 0.0 |
| Skeletal muscle | 11.4 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 7.7 | Ovarian ca.* (ascites) | 0.0 |
| | | SK-OV-3 | |
| Small intestine | 6.3 | Uterus | 3.3 |
| Colon ca. SW480 | 0.0 | Plancenta | 3.5 |
| Colon ca.* SW620 (SW480 | 0.0 | Prostate | 0.0 |
| met) | | | |
| Colon ca. HT29 | 0.0 | Prostate ca.* (bone | 0.0 |
| | | met)PC-3 | |
| Colon ca. HCT-116 | 0.0 | Testis | 62.4 |
| Colon ca. CaCo-2 | 0.0 | Melanoma Hs688(A).T | 4.0 |
| Colon ca. | 0.0 | Melanoma* (met) | 2.7 |
| tissue(ODO3866) | | Hs688(B).T | |
| Colon ca. HCC-2998 | 3.6 | Melanoma UACC-62 | 0.0 |
| Gastric ca.* (liver met) | 0.0 | Melanoma M14 | 0.0 |
| NCI-N87 | | | |
| Bladder | 4.2 | Melanoma LOX IMVI | 4.0 |
| Trachea | 0.0 | Melanoma* (met) SK- | 0.0 |
| | | MEL-5 | |
| Kidney | 0.0 | Adipose | 0.0 |

TABLE LD
Panel 4D

| | Rel. Exp.(%) | | Rel. Exp.(%) |
|---|---|---|---|
| | Ag2323, Run | | Ag2323, Run |
| Tissue Name | 163975720 | Tissue Name | 163975720 |
| Secondary Th1 act | 11.6 | HUVEC IL-1beta | 4.2 |
| Secondary Th2 act | 9.0 | HUVEC IFN gamma | 2.4 |
| Secondary Tr1 act | 19.2 | HUVEC TNF alpha + IFN | 15.8 |
| | | gamma | |
| Secondary Th1 rest | 23.2 | HUVEC TNF alpha + IL4 | 10.3 |
| Secondary Th2 rest | 21.3 | HUVEC IL-11 | 2.0 |
| Secondary Tr1 rest | 20.7 | Lung Microvascular EC none | 0.0 |
| Primary Th1 act | 37.6 | Lung Microvascular EC | 0.0 |
| | | TNFalpha + IL-1beta | |
| Primary Th2 act | 47.0 | Microvascular Dermal EC | 0.0 |
| | | none | |
| Primary Tr1 act | 81.2 | Microsvasular Dermal EC | 0.0 |
| | | TNFalpha + IL-1beta | |
| Primary Th1 rest | 44.8 | Bronchial epithelium | 0.0 |
| | | TNFalpha + IL1beta | |
| Primary Th2 rest | 17.3 | Small airway epithelium none | 0.0 |
| Primary Tr1 rest | 14.8 | Small airway epithelium | 2.4 |
| | | TNFalpha + IL-1beta | |
| CD45RA CD4 lymphocyte | 11.1 | Coronery artery SMC rest | 1.6 |
| act | | | |
| CD45RO CD4 lymphocyte | 20.2 | Coronery artery SMC | 0.0 |
| act | | TNFalpha + IL-1beta | |
| CD8 lymphocyte act | 18.8 | Astrocytes rest | 2.4 |
| Secondary CD8 | 25.2 | Astrocytes TNFalpha + IL- | 4.3 |
| lymphocyte rest | | 1beta | |
| Secondary CD8 | 0.0 | KU-812 (Basophil) rest | 0.0 |
| lymphocyte act | | | |
| CD4 lymphocyte none | 8.2 | KU-812 (Basophil) | 0.0 |
| | | PMA/ionomycin | |
| 2ry Th1/Th2/Tr1_anti- | 13.4 | CCD1106 (Keratinocytes) | 14.3 |
| CD95 CH11 | | none | |
| LAK cells rest | 0.0 | CCD1106 (Keratinocytes) | 10.3 |
| | | TNFalpha + IL-1beta | |
| LAK cells IL-2 | 0.0 | Liver cirrhosis | 18.0 |
| LAK cells IL-2 + IL-12 | 20.4 | Lupus kidney | 0.0 |
| LAK cells IL-2 + IFN | 59.9 | NCI-H292 none | 0.0 |
| gamma | | | |
| LAK cells IL-2 + IL-18 | 45.4 | NCI-H292 IL-4 | 0.0 |
| LAK cells | 1.7 | NCI-H292 IL-9 | 6.6 |
| PMA/ionomycin | | | |
| NK Cells IL-2 rest | 20.4 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 2.2 | HPAEC TNF alpha + IL-1 beta | 0.0 |
| PBMC rest | 5.6 | Lung fibroblast none | 1.4 |
| PBMC PWM | 100.0 | Lung fibroblast TNF alpha + | 1.9 |
| | | IL-1 beta | |
| PBMC PHA-L | 21.9 | Lung fibroblast IL-4 | 11.4 |
| Ramos (B cell) none | 0.0 | Lung fibroblast IL-9 | 10.7 |
| Ramos (B cell) ionomycin | 0.0 | Lung fibroblast IL-13 | 7.5 |
| B lymphocytes PWM | 49.0 | Lung fibroblast IFN gamma | 13.9 |
| B lymphocytes CD40L and | 10.4 | Dermal fibroblast CCD1070 | 0.0 |
| IL-4 | | rest | |
| EOL-1 dbcAMP | 87.1 | Dermal fibroblast CCD1070 | 11.5 |
| | | TNF alpha | |
| EOL-1 dbcAMP | 90.8 | Dermal fibroblast CCD1070 | 1.9 |
| PMA/ionomycin | | IL-1 beta | |
| Dendritic cells none | 11.0 | Dermal fibroblast IFN gamma | 0.0 |
| Dendritic cells LPS | 0.0 | Dermal fibroblast IL-4 | 2.2 |
| Dendritic cells anti-CD40 | 3.6 | IBD Colitis 2 | 0.0 |
| Monocytes rest | 0.0 | IBD Crohn's | 0.0 |
| Monocytes LPS | 0.0 | Colon | 0.0 |
| Macrophages rest | 9.2 | Lung | 0.0 |
| Macrophages LPS | 2.1 | Thymus | 4.5 |
| HUVEC none | 0.0 | Kidney | 8.6 |
| HUVEC starved | 9.9 | | |

CNS_neurodegeneration_v1.0 Summary: Ag2323 No difference is detected in the expression of the CG137823-01 gene in the postmortem brains of Alzheimer's diseased patients when compared to controls; however this panel demonstrates the expression of this gene in the brains of an independent group of subjects. See panel 1.3d for a discussion of utility of this gene in the central nervous system.

Panel 1.3D Summary: Ag2323 The expression of the CG137823-01 gene appears to be highest in a sample derived from a liver cancer (CT=33.5). In addition, there is substantial expression associated with testis tissue, and two samples derived from specific brain regions (amygdala and thalamus). Thus, the expression of this gene could be used to distinguish the liver cancer sample form the other samples in the panel. Moreover, therapeutic modulation of this gene, through the use of small molecule drugs, antibodies or protein therapeutics might be of benefit in the treatment of liver cancer.

This gene represents a novel G-protein coupled receptor (GPCR) and also shows expression in the brain. The GPCR family of receptors contains a large number of neurotransmitter receptors, including the dopamine, serotonin, a and b-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore, this gene may be of use as a small molecule target for the treatment of any of the described diseases.

### REFERENCES

El Yacoubi M, Ledent C, Parmentier M, Bertorelli R, Ongini E, Costentin J, Vaugeois JM. Adenosine A2A receptor antagonists are potential antidepressants: evidence based on pharmacology and A2A receptor knockout mice. Br J Pharmacol September 2001; 134(1):68-77.

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(-1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15:12-7

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSR1 treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini M E, Reggiani A. Glycine-site antagonists and stroke. Expert Opin Investig Drugs November 1999; 8(11):1837-1848.

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport December 1, 1998 ;9(17):3955-9 Related Articles, Books, LinkOut

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 4D Summary: Ag2323 The CG137823-01 transcript is expressed in EOL-1 cells and in activated lymphocytes (CTs=31-32). Non-activated CD4 cells do not express the transcript, however T cells induced with specific activators (CD3/CD28 regardless of the presence of polarizing cytokines) or with mitogens such as phytohemaglutinin (PHA) express the transcript. Likewise, no expression of the transcript is seen in PBMC that contain normal B cells, but the transcript is induced when PBMC are treated with the B cell selective pokeweed mitogen. In addition, the transcript is not seen in the B cell lymphoma Ramos regardless of stimulation and conversely, EOL-1 cells express the transcript regardless of stimulation. Therefore, the putative GPCR encoded by this gene could potentially be used diagnostically to identify activated B or T cells. In addition, the gene product could also potentially be used therapeutically in the treatment of asthma, emphysema, IBD, lupus or arthritis and in other diseases in which T cells and B cells are activated.

M. CG56818-02/GMAC027367_A: Olfactory Receptor

Expression of gene CG56818-02 was assessed using the primer-probe sets Ag5288 and Ag2612, described in Tables MA and MB. Results of the RTQ-PCR runs are shown in Tables MC, and MD.

TABLE MA
Probe Name Ag5288

| | | | Start | SEQ ID |
|---|---|---|---|---|
| Primers | Sequences | Length | Position | NO |
| Forward | 5′-tctgacttggaaggcagttg-3′ | 20 | 678 | 178 |
| Probe | TET-5′-aaggagatgaacattacgtccacgcc-3′-TAMRA | 26 | 621 | 179 |
| Reverse | 5′-ttactgccattctgctggtc-3′ | 20 | 598 | 180 |

TABLE MB
Probe Name Ag2612

| | | | Start | SEQ ID |
|---|---|---|---|---|
| Primers | Sequences | Length | Postion | NO |
| Forward | 5′-tattggcctctcagtggtacac-3′ | 22 | 764 | 181 |
| Probe | TET-5′-tttggaaacagccttcatcccattgt-3′-TAMRA | 26 | 789 | 182 |
| Reverse | 5′-ggtagatgtcacccatgacaac-3′ | 22 | 819 | 183 |

TABLE MC
Panel 1.3D

| | Rel. Exp.(%) Ag2612, | | Rel. Exp.(%) Ag2612, |
|---|---|---|---|
| Tissue Name | Run 166162990 | Tissue Name | Run 166162990 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.3 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 0.0 | Lung (fetal) | 0.7 |
| Brain (substantia nigra) | 0.6 | Lung ca. (small cell) | 0.0 |
| | | LX-1 | |
| Brain (thalamus) | 0.0 | Lung ca. (small cell) | 27.5 |
| | | NCI-H69 | |
| Cerebral Cortex | 0.0 | Lung ca. (s. cell var.) | 100.0 |
| | | SHP-77 | |
| Spinal cord | 0.4 | Lung ca. (large | 1.0 |
| | | cell)NCI-H460 | |
| glio/astro U87-MG | 0.0 | Lung ca. (non-sm. cell) | 5.2 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.3 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 20.4 |
| | | H596 | |
| glioma SNB-19 | 0.0 | Mammary gland | 0.0 |
| glioma U251 | 0.0 | Breast ca.* (pl. ef) | 0.0 |
| | | MCF-7 | |
| glioma SF-295 | 1.2 | Breast ca.* (pl. ef) | 0.0 |
| | | MDA-MB-231 | |
| Heart (fetal) | 0.2 | Breast ca.* (pl. ef) T47D | 0.0 |
| Heart | 0.0 | Breast ca. BT-549 | 0.0 |
| Skeletal muscle (fetal) | 0.3 | Breast ca. MDA-N | 0.0 |
| Skeletal muscle | 0.7 | Ovary | 0.0 |
| Bone marrow | 0.3 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.7 | Ovarian ca. IGROV-1 | 0.0 |
| Stomach | 0.0 | Ovarian ca.* (ascites) | 0.0 |
| | | SK-OV-3 | |
| Small intestine | 1.4 | Uterus | 1.4 |
| Colon ca. SW480 | 0.0 | Plancenta | 0.6 |
| Colon ca.* | 0.3 | Prostate | 55.1 |
| SW620(SW480 met) | | | |
| Colon ca. HT29 | 0.3 | Prostate ca.* (bone | 0.0 |
| | | met)PC-3 | |
| Colon ca. HCT-116 | 0.0 | Testis | 0.3 |
| Colon ca. CaCo-2 | 0.0 | Melanoma Hs688(A).T | 0.0 |
| Colon ca. | 2.7 | Melanoma* (met) | 0.3 |
| tissue(ODO3866) | | Hs688(B).T | |
| Colon ca. HCC-2998 | 0.4 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.2 |

TABLE MD
Panel 2.2

| | Rel. Exp.(%) Ag2612, | | Rel. Exp.(%) Ag2612, |
|---|---|---|---|
| Tissue Name | Run 174929488 | Tissue Name | Run 174929488 |
| Normal Colon | 27.9 | Kidney Margin (OD04348) | 2.9 |
| Colon cancer (OD06064) | 0.4 | Kidney malignant cancer | 0.8 |
| | | (OD06204B) | |
| Colon Margin (OD06064) | 0.6 | Kidney normal adjacent | 0.0 |
| | | tissue (OD06204E) | |
| Colon cancer (OD06159) | 0.5 | Kidney Cancer (OD04450- | 0.0 |
| | | 01) | |
| Colon Margin (OD06159) | 4.3 | Kidney Margin (OD04450- | 0.5 |
| | | 03) | |
| Colon cancer (OD06297-04) | 0.4 | Kidney Cancer 8120613 | 0.0 |
| Colon Margin (OD06297- | 3.8 | Kidney Margin 8120614 | 0.0 |
| 015) | | | |
| CC Gr.2 ascend colon | 0.9 | Kidney Cancer 9010320 | 0.0 |
| (ODO3921) | | | |
| CC Margin (ODO3921) | 2.9 | Kidney Margin 9010321 | 0.0 |
| Colon cancer metastasis | 0.0 | Kidney Cancer 8120607 | 0.0 |
| (OD06104) | | | |
| Lung Margin (OD06104) | 0.0 | Kidney Margin 8120608 | 0.0 |
| Colon mets to lung | 0.0 | Normal Uterus | 2.3 |
| (OD04451-01) | | | |
| Lung Margin (OD04451-02) | 0.0 | Uterine Cancer 064011 | 0.0 |
| Normal Prostate | 31.0 | Normal Thyroid | 0.0 |
| Prostate Cancer (OD04410) | 100.0 | Thyroid Cancer 064010 | 0.0 |
| Prostate Margin (OD04410) | 48.3 | Thyroid Cancer A302152 | 0.0 |
| Normal Ovary | 1.3 | Thyroid Margin A302153 | 0.0 |
| Ovarian cancer (OD06283- | 0.0 | Normal Breast | 0.5 |
| 03) | | | |
| Ovarian Margin (OD06283- | 0.3 | Breast Cancer (OD04566) | 0.5 |
| 07) | | | |
| Ovarian Cancer 064008 | 1.6 | Breast Cancer 1024 | 0.4 |
| Ovarian cancer (OD06145) | 0.0 | Breast Cancer (OD04590- | 0.0 |
| | | 01) | |
| Ovarian Margin (OD06145) | 0.0 | Breast Cancer Mets | 0.0 |
| | | (OD04590-03) | |
| Ovarian cancer (OD06455- | 0.0 | Breast Cancer Metastasis | 0.0 |
| 03) | | (OD04655-05) | |
| Ovarian Margin (OD06455- | 0.0 | Breast Cancer 064006 | 0.0 |
| 07) | | | |
| Normal Lung | 0.0 | Breast Cancer 9100266 | 0.0 |
| Invasive poor diff. lung | 0.4 | Breast Margin 9100265 | 0.0 |
| adeno (ODO4945-01 | | | |
| Lung Margin (ODO4945- | 0.0 | Breast Cancer A209073 | 0.4 |
| 03) | | | |
| Lung Malignant Cancer | 1.0 | Breast Margin A2090734 | 0.0 |
| (OD03126) | | | |
| Lung Margin (OD03126) | 0.0 | Breast cancer (OD06083) | 0.0 |
| Lung Cancer (OD05014A) | 0.0 | Breast cancer node | 0.5 |
| | | metastasis (OD06083) | |
| Lung Margin (OD05014B) | 0.0 | Normal Liver | 0.0 |
| Lung cancer (OD06081) | 0.0 | Liver Cancer 1026 | 0.0 |
| Lung Margin (OD06081) | 0.0 | Liver Cancer 1025 | 0.0 |
| Lung Cancer (OD04237-01) | 0.0 | Liver Cancer 6004-T | 0.0 |
| Lung Margin (OD04237-02) | 0.0 | Liver Tissue 6004-N | 0.0 |
| Ocular Melanoma | 0.0 | Liver Cancer 6005-T | 0.0 |
| Metastasis | | | |
| Ocular Melanoma Margin | 0.0 | Liver Tissue 6005-N | 0.0 |
| (Liver) | | | |
| Melanoma Metastasis | 0.0 | Liver Cancer 064003 | 0.0 |
| Melanoma Margin (Lung) | 0.0 | Normal Bladder | 0.3 |
| Normal Kidney | 0.0 | Bladder Cancer 1023 | 0.0 |
| Kidney Ca, Nuclear grade 2 | 0.4 | Bladder Cancer A302173 | 0.0 |
| (OD04338) | | | |
| Kidney Margin (OD04338) | 0.5 | Normal Stomach | 0.4 |
| Kidney Ca Nuclear grade | 0.0 | Gastric Cancer 9060397 | 0.5 |
| 1/2 (OD04339) | | | |
| Kidney Margin (OD04339) | 0.0 | Stomach Margin 9060396 | 0.0 |
| Kidney Ca, Clear cell type | 0.8 | Gastric Cancer 9060395 | 0.5 |
| (OD04340) | | | |
| Kidney Margin (OD04340) | 0.0 | Stomach Margin 9060394 | 0.0 |
| Kidney Ca, Nuclear grade 3 | 0.7 | Gastric Cancer 064005 | 0.0 |
| (OD04348) | | | |

CNS_neurodegeneration_v1.0 Summary: Ag5288 Expression of the CG56818-02 gene is low/undetectable (CTs>34.5) across all of the samples on this panel (data not shown).

General_screening_panel_v1.5 Summary: Ag5288 One experiment using this probe and primer set and the CG56818-02 gene is not included because the amp plot indicates that there were experimental difficulties with this run.

Panel 1.3D Summary: Ag2612 The expression of the CG56818-02 gene appears to be highest in a sample derived from a lung cancer cell line (SHP-77)(CT=30.2). In addition, there is substantial expression associated with other lung cancer cell lines and a sample derived from normal prostate tissue. Thus, the expression of this gene could be used to distinguish these listed samples from other samples in the panel. Moreover, therapeutic modulation of this gene, through the use of small molecule drugs, antibodies or protein therapeutics might be of benefit in the treatment of lung cancer.

Panel 2.2 Summary: Ag2612 The expression of the CG56818-02 gene appears to be highest in a sample derived from malignant prostate tissue (CT=30.5). In addition, there is substantial expression associated with normal prostate tissue, including normal colon tissue and normal tissue adjacent to the malignant prostate tissue mentioned above. Expression in these normal tissue samples is, however, lower than that seen in the malignant tissue. Thus, the expression of this gene could be used to distinguish these samples from other samples in the panel. Moreover, therapeutic modulation of this gene, through the use of small molecule drugs, antibodies or protein therapeutics might be of benefit in the treatment of prostate cancer.

Panel 4.1D Summary: Ag5288 Expression of the CG56818-02 gene is low/undetectable (CTs>34.5) across all of the samples on this panel (data not shown).

Panel 4D Summary: Ag5288 Expression of the CG56818-02 gene is low/undetectable (CTs>34.5) across all of the samples on this panel (data not shown).

N. CG56826-01/GMAC026090_D: Olfactory Receptor

Expression of gene CG56826-01 was assessed using the primer-probe set Ag2604, described in Table NA. Results of the RTQ-PCR runs are shown in Tables NB, NC and ND.

TABLE NA
Probe Name Ag2604

| | | | Start | SEQ ID |
|---|---|---|---|---|
| Primers | Sequences | Length | Position | NO |
| Forward | 5′-agtcttgcacaagcctgtgtac-3′ | 22 | 190 | 184 |
| Probe | TET-5′-ctgtgcatgctctcaaccatcgacct-3′-TAMRA | 26 | 218 | 185 |
| Reverse | 5′-gatagccagtagcttgggaact-3′ | 22 | 262 | 186 |

TABLE NB
Panel 1.3D

| | Rel. Exp.(%) Ag2604, | | Rel. Exp.(%) Ag2604, |
|---|---|---|---|
| Tissue Name | Run 166162873 | Tissue Name | Run 166162873 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 10.2 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 7.6 | Liver | 0.0 |
| Brain (whole) | 4.2 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 23.8 |
| Brain (hippocampus) | 9.6 | Lung (fetal) | 0.0 |
| Brain (substantia nigra) | 7.5 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 15.2 | Lung ca. (large | 0.0 |
| | | cell)NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 4.6 |
| glioma U251 | 0.0 | Breast ca.* (pl. ef) MCF- | 0.0 |
| | | 7 | |
| glioma SF-295 | 0.0 | Breast ca.* (pl. ef) MDA- | 0.0 |
| | | MB-231 | |
| Heart (fetal) | 0.0 | Breast ca.* (pl. ef) T47D | 39.2 |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 10.2 |
| Bone marrow | 12.9 | Ovarian ca. OVCAR-3 | 0.0 |
| Thymus | 19.9 | Ovarian ca. OVCAR-4 | 0.0 |
| Spleen | 10.2 | Ovarian ca. OVCAR-5 | 0.0 |
| Lymph node | 19.9 | Ovarian ca. OVCAR-8 | 0.0 |
| Colorectal | 13.8 | Ovarian ca. IGROV-1 | 0.0 |
| Stomach | 0.0 | Ovarian ca.* (ascites) | 100.0 |
| | | SK-OV-3 | |
| Small intestine | 0.0 | Uterus | 0.0 |
| Colon ca. SW480 | 0.0 | Plancenta | 11.2 |
| Colon ca.* SW620(SW480 | 0.0 | Prostate | 0.0 |
| met) | | | |
| Colon ca. HT29 | 0.0 | Prostate ca.* (bone | 0.0 |
| | | met)PC-3 | |
| Colon ca. HCT-116 | 0.0 | Testis | 10.3 |
| Colon ca. CaCo-2 | 0.0 | Melanoma Hs688(A).T | 0.0 |
| Colon ca. | 27.4 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 7.3 | Melanoma LOX IMVI | 0.0 |
| Trachea | 2.6 | Melanoma* (met) SK- | 0.0 |
| | | MEL-5 | |
| Kidney | 0.0 | Adipose | 7.5 |

TABLE NC
Panel 2.2

| | Rel. Exp.(%) Ag2604, | | Rel. Exp.(%) Ag2604, |
|---|---|---|---|
| Tissue Name | Run 174929485 | Tissue Name | Run 174929485 |
| Normal Colon | 8.8 | Kidney Margin (OD04348) | 53.2 |
| Colon cancer (OD06064) | 22.8 | Kidney malignant cancer | 0.0 |
| | | (OD06204B) | |
| Colon Margin (OD06064) | 2.4 | Kidney normal adjacent | 0.0 |
| | | tissue (OD06204E) | |
| Colon cancer (OD06159) | 0.0 | Kidney Cancer (OD04450- | 11.8 |
| | | 01) | |
| Colon Margin (OD06159) | 29.1 | Kidney Margin (OD04450- | 0.0 |
| | | 03) | |
| Colon cancer (OD06297-04) | 0.0 | Kidney Cancer 8120613 | 0.0 |
| Colon Margin (OD06297- | 16.4 | Kidney Margin 8120614 | 0.0 |
| 015) | | | |
| CC Gr.2 ascend colon | 0.0 | Kidney Cancer 9010320 | 0.0 |
| (ODO3921) | | | |
| CC Margin (ODO3921) | 0.0 | Kidney Margin 9010321 | 8.4 |
| Colon cancer metastasis | 0.0 | Kidney Cancer 8120607 | 0.0 |
| (OD06104) | | | |
| Lung Margin (OD06104) | 0.0 | Kidney Margin 8120608 | 0.0 |
| Colon mets to lung | 49.7 | Normal Uterus | 8.2 |
| (OD04451-01) | | | |
| Lung Margin (OD04451-02) | 31.2 | Uterine Cancer 064011 | 55.5 |
| Normal Prostate | 0.0 | Normal Thyroid | 0.0 |
| Prostate Cancer (OD04410) | 12.0 | Thyroid Cancer 064010 | 0.0 |
| Prostate Margin (OD04410) | 12.1 | Thyroid Cancer A302152 | 6.7 |
| Normal Ovary | 0.0 | Thyroid Margin A302153 | 0.0 |
| Ovarian cancer (OD06283- | 0.0 | Normal Breast | 85.9 |
| 03) | | | |
| Ovarian Margin (OD06283- | 30.1 | Breast Cancer (OD04566) | 0.0 |
| 07) | | | |
| Ovarian Cancer 064008 | 19.1 | Breast Cancer 1024 | 0.0 |
| Ovarian cancer (OD06145) | 0.0 | Breast Cancer (OD04590- | 0.0 |
| | | 01) | |
| Ovarian Margin (OD06145) | 11.3 | Breast Cancer Mets | 13.6 |
| | | (OD04590-03) | |
| Ovarian cancer (OD06455- | 0.0 | Breast Cancer Metastasis | 10.4 |
| 03) | | (OD04655-05) | |
| Ovarian Margin (OD06455-07) | 42.9 | Breast Cancer 064006 | 2.4 |
| Normal Lung | 12.9 | Breast Cancer 9100266 | 0.0 |
| Invasive poor diff. lung | 100.0 | Breast Margin 9100265 | 0.0 |
| adeno (ODO4945-01 | | | |
| Lung Margin (ODO4945- | 62.4 | Breast Cancer A209073 | 0.0 |
| 03) | | | |
| Lung Malignant Cancer | 0.0 | Breast Margin A2090734 | 0.0 |
| (OD03126) | | | |
| Lung Margin (OD03126) | 25.7 | Breast cancer (OD06083) | 51.8 |
| Lung Cancer (OD05014A) | 0.0 | Breast cancer node | 68.3 |
| | | metastasis (OD06083) | |
| Lung Margin (OD05014B) | 63.7 | Normal Liver | 30.6 |
| Lung cancer (OD06081) | 0.0 | Liver Cancer 1026 | 0.0 |
| Lung Margin (OD06081) | 27.7 | Liver Cancer 1025 | 11.0 |
| Lung Cancer (OD04237-01) | 0.0 | Liver Cancer 6004-T | 0.0 |
| Lung Margin (OD04237-02) | 40.9 | Liver Tissue 6004-N | 0.0 |
| Ocular Melanoma | 0.0 | Liver Cancer 6005-T | 0.0 |
| Metastasis | | | |
| Ocular Melanoma Margin | 0.0 | Liver Tissue 6005-N | 0.0 |
| (Liver) | | | |
| Melanoma Metastasis | 0.0 | Liver Cancer 064003 | 11.6 |
| Melanoma Margin (Lung) | 0.0 | Normal Bladder | 3.0 |
| Normal Kidney | 9.4 | Bladder Cancer 1023 | 0.0 |
| Kidney Ca, Nuclear grade 2 | 12.5 | Bladder Cancer A302173 | 50.3 |
| (OD04338) | | | |
| Kidney Margin (OD04338) | 0.0 | Normal Stomach | 60.7 |
| Kidney Ca Nuclear grade | 0.0 | Gastric Cancer 9060397 | 0.0 |
| 1/2 (OD04339) | | | |
| Kidney Margin (OD04339) | 12.9 | Stomach Margin 9060396 | 0.0 |
| Kidney Ca, Clear cell type | 0.0 | Gastric Cancer 9060395 | 14.7 |
| (OD04340) | | | |
| Kidney Margin (OD04340) | 0.0 | Stomach Margin 9060394 | 0.0 |
| Kidney Ca, Nuclear grade 3 | 13.4 | Gastric Cancer 064005 | 16.7 |
| (OD04348) | | | |

TABLE ND
Panel 4D

| | Rel. Exp.(%) | | Rel. Exp.(%) |
|---|---|---|---|
| | Ag2323, Run | | Ag2323, Run |
| Tissue Name | 164216437 | Tissue Name | 164216437 |
| Secondary Th1 act | 1.3 | HUVEC IL-1beta | 1.1 |
| Secondary Th2 act | 5.0 | HUVEC IFN gamma | 1.7 |
| Secondary Tr1 act | 1.4 | HUVEC TNF alpha + IFN | 0.0 |
| | | gamma | |
| Secondary Th1 rest | 1.8 | HUVEC TNF alpha + IL4 | 0.0 |
| Secondary Th2 rest | 1.0 | HUVEC IL-11 | 0.0 |
| Secondary Tr1 rest | 4.1 | Lung Microvascular EC none | 0.0 |
| Primary Th1 act | 4.7 | Lung Microvascular EC | 0.0 |
| | | TNFalpha + IL-1beta | |
| Primary Th2 act | 3.6 | Microvascular Dermal EC | 0.0 |
| | | none | |
| Primary Tr1 act | 5.8 | Microsvasular Dermal EC | 1.8 |
| | | TNFalpha + IL-1beta | |
| Primary Th1 rest | 60.7 | Bronchial epithelium | 4.7 |
| | | TNFalpha + IL1beta | |
| Primary Th2 rest | 12.0 | Small airway epithelium none | 0.0 |
| Primary Tr1 rest | 14.1 | Small airway epithelium | 0.8 |
| | | TNFalpha + IL-1beta | |
| CD45RA CD4 lymphocyte | 8.9 | Coronery artery SMC rest | 5.0 |
| act | | | |
| CD45RO CD4 lymphocyte | 16.4 | Coronery artery SMC | 0.0 |
| act | | TNFalpha + IL-1beta | |
| CD8 lymphocyte act | 0.0 | Astrocytes rest | 0.0 |
| Secondary CD8 | 17.3 | Astrocytes TNFalpha + IL- | 0.0 |
| lymphocyte rest | | 1beta | |
| Secondary CD8 | 3.0 | KU-812 (Basophil) rest | 0.0 |
| lymphocyte act | | | |
| CD4 lymphocyte none | 11.1 | KU-812 (Basophil) | 6.7 |
| | | PMA/ionomycin | |
| 2ry Th1/Th2/Tr1_anti- | 18.7 | CCD1106 (Keratinocytes) | 0.0 |
| CD95 CH11 | | none | |
| LAK cells rest | 13.3 | CCD1106 (Keratinocytes) | 3.0 |
| | | TNFalpha + IL-1beta | |
| LAK cells IL-2 | 43.8 | Liver cirrhosis | 3.3 |
| LAK cells IL-2 + IL-12 | 39.5 | Lupus kidney | 1.7 |
| LAK cells IL-2 + IFN | 100.0 | NCI-H292 none | 2.5 |
| gamma | | | |
| LAK cells IL-2 + IL-18 | 56.3 | NCI-H292 IL-4 | 0.0 |
| LAK cells | 4.3 | NCI-H292 IL-9 | 0.0 |
| PMA/ionomycin | | | |
| NK Cells IL-2 rest | 13.3 | NCI-H292 IL-13 | 0.0 |
| Two Way MLR 3 day | 51.4 | NCI-H292 IFN gamma | 0.0 |
| Two Way MLR 5 day | 10.6 | HPAEC none | 0.0 |
| Two Way MLR 7 day | 4.3 | HPAEC TNF alpha + IL-1 beta | 0.0 |
| PBMC rest | 0.0 | Lung fibroblast none | 0.0 |
| PBMC PWM | 39.8 | Lung fibroblast TNF alpha + | 0.0 |
| | | IL-1 beta | |
| PBMC PHA-L | 9.2 | Lung fibroblast IL-4 | 0.0 |
| Ramos (B cell) none | 0.0 | Lung fibroblast IL-9 | 3.6 |
| Ramos (B cell) ionomycin | 0.0 | Lung fibroblast IL-13 | 1.4 |
| B lymphocytes PWM | 20.4 | Lung fibroblast IFN gamma | 2.6 |
| B lymphocytes CD40L and | 4.8 | Dermal fibroblast CCD1070 | 0.0 |
| IL-4 | | rest | |
| EOL-1 dbcAMP | 6.6 | Dermal fibroblast CCD1070 | 4.8 |
| | | TNF alpha | |
| EOL-1 dbcAMP | 1.3 | Dermal fibroblast CCD1070 | 0.0 |
| PMA/ionomycin | | IL-1 beta | |
| Dendritic cells none | 10.7 | Dermal fibroblast IFN gamma | 5.7 |
| Dendritic cells LPS | 5.7 | Dermal fibroblast IL-4 | 2.1 |
| Dendritic cells anti-CD40 | 10.7 | IBD Colitis 2 | 2.9 |
| Monocytes rest | 4.7 | IBD Crohn's | 3.8 |
| Monocytes LPS | 2.2 | Colon | 17.8 |
| Macrophages rest | 7.3 | Lung | 2.3 |
| Macrophages LPS | 0.0 | Thymus | 2.0 |
| HUVEC none | 0.0 | Kidney | 27.2 |
| HUVEC starved | 0.0 | | |

Panel 1.3D Summary: Ag2604 Expression of the CG56826-01 gene is restricted to an sample derived from an ovarian cancer cell line (CT=33.4). This cell line is unusual in that it is derived from ascites. Thus, expression of this gene could potentially be used to differentiate between this sample and other samples on this panel. Furthermore, expression of this gene could also be useful in differentiating between ascites derived samples and other samples.

Panel 2.2 Summary: Ag2604 Expression of the CG56826-01 on this panel is restricted to a lung cancer derived sample (CT=34.4). Thus, expression of this gene could be used to differentiate between this sample and other samples on this panel.

Panel 4D Summary: Ag2604 The CG56826-01 transcript is expressed in normal kidney and colon as well as in activated LAK cells (CTs=31-33). The gene is also expressed at lower but still significant levels in acutely activated primary T cells (highest in Th1 cells). The putative GPCR encoded by this transcript could be important in the function of LAK cells. LAK cells are important in immunosurveillance against bacterial and viral infected cells, as well as transformed cells. Therapeutics designed with this transcript or the protein encoded by it could be important in the treatment of viral and bacterial diseases and cancer.

O. CG149547-01/GMAP002418_D: Olfactory Receptor

Expression of gene CG149547-01 was assessed using the primer-probe set Ag1949, described in Table OA. Results of the RTQ-PCR runs are shown in Table OB.

TABLE OA
Probe Name Ag1949

| Primers | Sequences | Length | Start Position | SEQ ID NO |
|---|---|---|---|---|
| Forward | 5′-acccatgtattttgtcattgga-3′ | 22 | 170 | 187 |
| Probe | TET-5′-tcttctgtccacaccccaaagatcct-3′-TAMRA | 26 | 219 | 188 |
| Reverse | 5′-gtcttcagagatgcaggtcact-3′ | 22 | 245 | 189 |

TABLE OB
Panel 4D

| | Rel. Exp.(%) | | Rel. Exp.(%) |
|---|---|---|---|
| | Ag1949, Run | | Ag1949, Run |
| Tissue Name | 165870454 | Tissue Name | 165870454 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 7.7 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 5.8 |
| | | TNFalpha + IL1beta | |
| Primary Th2 rest | 0.0 | Small airway epithelium none | 0.0 |
| Primary Tr1 rest | 0.0 | Small airway epithelium | 10.9 |
| | | TNFalpha + IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC | 0.0 |
| act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 9.2 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 13.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 23.5 |
| B lymphocytes PWM | 0.0 | Lung fibroblast IFN gamma | 0.0 |
| B lymphocytes CD40L and | 0.0 | Dermal fibroblast CCD1070 | 0.0 |
| IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 14.8 |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

Panel 4D Summary: Ag1949 Significant expression of this gene is detected in a liver cirrhosis sample (CT=34.3). Furthermore, expression of this gene is not detected in normal liver in Panel 1.3D, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

P. CG146028-01/GMAL160314_B: Olfactory Receptor

Expression of gene CG146028-01 was assessed using the primer-probe set Ag1823, described in Table PA. Results of the RTQ-PCR runs are shown in Tables PB and PC.

TABLE PA
Probe Name Ag1823

| | | | Start | SEQ ID |
|---|---|---|---|---|
| Primers | Sequences | Length | Postion | NO |
| Forward | 5′-ttccaagttttcttgtccttca-3′ | 22 | 472 | 190 |
| Probe | TET-5′-tggccccaacatcattaaccatttct-3′-TAMRA | 26 | 506 | 191 |
| Reverse | 5′-aaatgagtttcaagaggggaaa-3′ | 22 | 543 | 192 |

TABLE PB
Panel 1.3D

| | Rel. Exp.(%) Ag1823, | | Rel. Exp.(%) Ag1823, |
|---|---|---|---|
| Tissue Name | Run 165975010 | Tissue Name | Run 165975010 |
| Liver adenocarcinoma | 0.0 | Kidney (fetal) | 7.0 |
| Pancreas | 13.7 | Renal ca. 786-0 | 0.0 |
| Pancreatic ca. CAPAN 2 | 0.0 | Renal ca. A498 | 6.5 |
| Adrenal gland | 2.6 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 8.7 | Renal ca. TK-10 | 0.0 |
| Brain (fetal) | 0.0 | Liver | 0.0 |
| Brain (whole) | 39.2 | Liver (fetal) | 8.0 |
| Brain (amygdala) | 21.8 | Liver ca. (hepatoblast) | 0.0 |
| | | HepG2 | |
| Brain (cerebellum) | 6.2 | Lung | 0.0 |
| Brain (hippocampus) | 0.0 | Lung (fetal) | 7.2 |
| Brain (substantia nigra) | 4.0 | Lung ca. (small cell) | 0.0 |
| | | LX-1 | |
| Brain (thalamus) | 30.8 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 100.0 | Lung ca. (large | 0.0 |
| | | cell)NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 18.4 | Mammary gland | 0.0 |
| glioma U251 | 0.0 | Breast ca.* (pl. ef) MCF- | 12.5 |
| | | 7 | |
| glioma SF-295 | 0.0 | Breast ca.* (pl. ef) MDA- | 0.0 |
| | | MB-231 | |
| Heart (fetal) | 0.0 | Breast ca.* (pl. ef) T47D | 0.0 |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 73.7 | Ovarian ca. OVCAR-5 | 0.0 |
| Lymph node | 4.6 | Ovarian ca. OVCAR-8 | 0.0 |
| Colorectal | 3.8 | Ovarian ca. IGROV-1 | 0.0 |
| Stomach | 10.4 | Ovarian ca.* (ascites) | 0.0 |
| | | SK-OV-3 | |
| Small intestine | 1.6 | Uterus | 12.9 |
| Colon ca. SW480 | 0.0 | Plancenta | 0.0 |
| Colon ca.* SW620(SW480 | 0.0 | Prostate | 0.0 |
| met) | | | |
| Colon ca. HT29 | 0.0 | Prostate ca.* (bone | 0.0 |
| | | met)PC-3 | |
| Colon ca. HCT-116 | 0.0 | Testis | 9.7 |
| Colon ca. CaCo-2 | 1.3 | Melanoma Hs688(A).T | 0.0 |
| Colon ca. | 0.0 | Melanoma* (met) | 0.0 |
| tissue(ODO3866) | | Hs688(B).T | |
| Colon ca. HCC-2998 | 6.7 | Melanoma UACC-62 | 0.0 |
| Gastric ca.* (liver met) | 0.0 | Melanoma M14 | 0.0 |
| NCI-N87 | | | |
| Bladder | 10.1 | Melanoma LOX IMVI | 0.0 |
| Trachea | 0.0 | Melanoma* (met) SK- | 0.0 |
| | | MEL-5 | |
| Kidney | 9.0 | Adipose | 0.0 |

TABLE PC
Panel 4D

| | Rel. Exp.(%) | | Rel. Exp.(%) |
|---|---|---|---|
| | Ag1823, Run | | Ag1823, Run |
| Tissue Name | 165823136 | Tissue Name | 165823136 |
| Secondary Th1 act | 8.8 | HUVEC IL-1beta | 8.7 |
| Secondary Th2 act | 0.0 | HUVEC IFN gamma | 19.3 |
| Secondary Tr1 act | 4.7 | HUVEC TNF alpha + IFN | 0.0 |
| | | gamma | |
| Secondary Th1 rest | 4.1 | HUVEC TNF alpha + IL4 | 13.8 |
| Secondary Th2 rest | 15.6 | HUVEC IL-11 | 11.3 |
| Secondary Tr1 rest | 13.9 | Lung Microvascular EC none | 15.4 |
| Primary Th1 act | 4.7 | Lung Microvascular EC | 3.4 |
| | | TNFalpha + IL-1beta | |
| Primary Th2 act | 8.7 | Microvascular Dermal EC | 4.5 |
| | | none | |
| Primary Tr1 act | 3.9 | Microsvasular Dermal EC | 7.8 |
| | | TNFalpha + IL-1beta | |
| Primary Th1 rest | 4.8 | Bronchial epithelium | 14.2 |
| | | TNFalpha + IL1beta | |
| Primary Th2 rest | 19.6 | Small airway epithelium none | 3.6 |
| Primary Tr1 rest | 5.3 | Small airway epithelium | 17.6 |
| | | TNFalpha + IL-1beta | |
| CD45RA CD4 lymphocyte | 0.0 | Coronery artery SMC rest | 22.5 |
| act | | | |
| CD45RO CD4 lymphocyte | 0.0 | Coronery artery SMC | 9.3 |
| act | | TNFalpha + IL-1beta | |
| CD8 lymphocyte act | 0.0 | Astrocytes rest | 6.7 |
| Secondary CD8 | 10.3 | Astrocytes TNFalpha + IL- | 15.2 |
| lymphocyte rest | | 1beta | |
| Secondary CD8 | 0.0 | KU-812 (Basophil) rest | 32.3 |
| lymphocyte act | | | |
| CD4 lymphocyte none | 16.8 | KU-812 (Basophil) | 100.0 |
| | | PMA/ionomycin | |
| 2ry Th1/Th2/Tr1_anti- | 2.5 | CCD1106 (Keratinocytes) | 22.5 |
| CD95 CH11 | | none | |
| LAK cells rest | 3.4 | CCD1106 (Keratinocytes) | 18.7 |
| | | TNFalpha + IL-1beta | |
| LAK cells IL-2 | 14.1 | Liver cirrhosis | 95.3 |
| LAK cells IL-2 + IL-12 | 14.9 | Lupus kidney | 44.1 |
| LAK cells IL-2 + IFN | 36.1 | NCI-H292 none | 12.2 |
| gamma | | | |
| LAK cells IL-2 + IL-18 | 26.6 | NCI-H292 IL-4 | 10.7 |
| LAK cells | 0.0 | NCI-H292 IL-9 | 12.1 |
| PMA/ionomycin | | | |
| NK Cells IL-2 rest | 9.0 | NCI-H292 IL-13 | 6.7 |
| Two Way MLR 3 day | 14.8 | NCI-H292 IFN gamma | 14.4 |
| Two Way MLR 5 day | 9.0 | HPAEC none | 8.0 |
| Two Way MLR 7 day | 0.0 | HPAEC TNF alpha + IL-1 beta | 18.6 |
| PBMC rest | 6.0 | Lung fibroblast none | 3.6 |
| PBMC PWM | 0.0 | Lung fibroblast TNF alpha + | 1.9 |
| | | IL-1 beta | |
| PBMC PHA-L | 0.0 | Lung fibroblast IL-4 | 0.0 |
| Ramos (B cell) none | 18.4 | Lung fibroblast IL-9 | 0.0 |
| Ramos (B cell) ionomycin | 13.1 | Lung fibroblast IL-13 | 0.0 |
| B lymphocytes PWM | 13.6 | Lung fibroblast IFN gamma | 0.0 |
| B lymphocytes CD40L and | 12.7 | Dermal fibroblast CCD1070 | 0.0 |
| IL-4 | | rest | |
| EOL-1 dbcAMP | 16.4 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 13.2 | Dermal fibroblast IFN gamma | 0.0 |
| Dendritic cells LPS | 4.1 | Dermal fibroblast IL-4 | 0.0 |
| Dendritic cells anti-CD40 | 14.0 | IBD Colitis 2 | 36.3 |
| Monocytes rest | 3.7 | IBD Crohn's | 6.2 |
| Monocytes LPS | 3.0 | Colon | 56.6 |
| Macrophages rest | 29.5 | Lung | 0.0 |
| Macrophages LPS | 6.0 | Thymus | 77.9 |
| HUVEC none | 49.3 | Kidney | 14.7 |
| HUVEC starved | 74.2 | | |

CNS_neurodegeneration_v1.0 Summary: Ag1823 Expression of the CG146028-01 gene is low/undetectable in all samples on this panel (CTs>35). (Data not shown.) Data from a second experiment with the same probe and primer is not included because the amp plot suggests there was a problem in one of the sample wells.

Panel 1.3D Summary: Ag1823 Expression of the CG146028-01 gene is highest in the spinal cord (CT=32) with low but significant expression also seen in the adult brain, thalamus and amygdala. This gene represents a novel G-protein coupled receptor (GPCR). The GPCR family of receptors contains a large number of neurotransmitter receptors, including the dopamine, serotonin, a and b-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore this gene may be of use as a small molecule target for the treatment of any of the described diseases.

This gene is also expressed at low levels in the spleen (CT=32.6), an important site of secondary immune responses. Therefore, antibodies or small molecule therapeutics that block the function of this GPCR may be useful as anti-inflammatory therapeutics for the treatment of allergies, autoimmune diseases, and inflammatory diseases.

### REFERENCES

El Yacoubi M, Ledent C, Parmentier M, Bertorelli R, Ongini E, Costentin J, Vaugeois JM. Adenosine A2A receptor antagonists are potential antidepressants: evidence based on pharmacology and A2A receptor knockout mice. Br J Pharmacol September 2001;134(1):68-77.

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical . antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15:12-7.

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSR1 treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini ME, Reggiani A. Glycine-site antagonists and stroke. Expert Opin Investig Drugs November 1999;8(11):1837-1848.

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport Dec. 1, 1998;9(17):3955-9.

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 2.2 Summary: Ag1823 Expression of the CG146028-01 gene is low/undetectable in all samples on this panel (CTs>35). (Data not shown.)

Panel 4D Summary: Ag1823 The expression of the CG146028-01 transcript is downregulated in cytokine treated HUVEC cells and is expressed in normal colon, thymus and kidney. The transcript is also induced in a basophil cell line, untreated umbilical vein endothelium. This suggests that this gene may be expressed on normal endothelium in the thymus, kidney and colon and may thus function in the normal homeostasis of these organs. Therefore, therapeutics designed with the putative GPCR may be important in the treatment of diseases such as lupus and IBD or after chemotherapy that disrupts normal thymic function.

Panel CNS_{—}1 Summary: Ag1823 Expression of the CG146028-01 gene is low/undetectable in all samples on this panel (CTs>35). (Data not shown.)

Q. CG149848-01/GMAP002509_A: Olfactory Receptor

Expression of gene CG149848-01 was assessed using the primer-probe set Ag1791, described in Table QA. Results of the RTQ-PCR runs are shown in Table QB.

TABLE QA
Probe Name Ag1791

| | | | Start | SEQ ID |
|---|---|---|---|---|
| Primers | Sequences | Length | Position | NO |
| Forward | 5′-catacccttggtctctttgttg-3′ | 22 | 571 | 193 |
| Probe | TET-5′-ctgccaacagtgggttcatctgctta-3′-TAMRA | 26 | 593 | 194 |
| Reverse | 5′-cacataggataccacccagaga-3′ | 22 | 630 | 195 |

TABLE QB
Panel 4D

| | Rel. Exp.(%) | | Rel. Exp.(%) |
|---|---|---|---|
| | Ag1791, Run | | Ag1791, Run |
| Tissue Name | 165809196 | Tissue Name | 165809196 |
| Secondary Th1 act | 8.8 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 17.0 | Small airway epithelium | 0.0 |
| | | TNFalpha + IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC | 0.0 |
| act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 2.9 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 0.0 | Dermal fibroblast CCD1070 | 0.0 |
| IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 11.2 |
| Monocytes rest | 0.0 | IBD Crohn's | 2.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

Panel 1.3D Summary: Ag1791 Expression of this gene is low/undetectable (CTs>35) across all of the samples on this panel (data not shown).

Panel 2.2 Summary: Ag1791 Expression of this gene is low/undetectable (CTs>35) across all of the samples on this panel (data not shown).

Panel 4D Summary: Ag1791 Significant expression of this gene is detected in a liver cirrhosis sample (CT=33.8). Furthermore, expression of this gene is not detected in normal liver in Panel 1.3D, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

R. CG149895-01/GMAL356019_F: Olfactory Receptor

Expression of gene CG149895-01 was assessed using the primer-probe set Ag1787, described in Table RA. Results of the RTQ-PCR runs are shown in Tables RB and RC.

TABLE RA
Probe Name Ag1787

| | | | Start | SEQ ID |
|---|---|---|---|---|
| Primers | Sequences | Length | Position | NO |
| Forward | 5′-acaagcactatgacggaatttg-3′ | 22 | 73 | 196 |
| Probe | TET-5′-ttctccttggctttcctggtgtcag-3′-TAMRA | 26 | 95 | 197 |
| Reverse | 5′-acagggagaagaggaaactttg-3′ | 22 | 127 | 198 |

TABLE RB
Panel 1.3D

| | Rel. Exp.(%) Ag1787, | | Rel. Exp.(%) Ag1787, |
|---|---|---|---|
| Tissue Name | Run 165941631 | Tissue Name | Run 165941631 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 12.3 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 8.7 |
| | | HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 0.0 | Lung ca. (large | 0.0 |
| | | cell)NCI-H460 | |
| glio/astro U87-MG | 0.0 | Lung ca. (non-sm. cell) | 8.4 |
| | | A549 | |
| glio/astro U-118-MG | 0.0 | Lung ca. (non-s. cell) | 0.0 |
| | | NCI-H23 | |
| astrocytoma SW1783 | 2.8 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl. ef) MCF- | 0.0 |
| | | 7 | |
| glioma SF-295 | 0.0 | Breast ca.* (pl. ef) MDA- | 0.0 |
| | | MB-231 | |
| Heart (fetal) | 0.0 | Breast ca.* (pl. ef) T47D | 0.0 |
| Heart | 0.0 | Breast ca. BT-549 | 0.0 |
| Skeletal muscle (fetal) | 0.0 | Breast ca. MDA-N | 0.0 |
| Skeletal muscle | 10.4 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 100.0 | Ovarian ca. OVCAR-5 | 0.0 |
| Lymph node | 0.0 | Ovarian ca. OVCAR-8 | 0.0 |
| Colorectal | 10.2 | Ovarian ca. IGROV-1 | 0.0 |
| Stomach | 0.0 | Ovarian ca.* (ascites) | 14.3 |
| | | SK-OV-3 | |
| Small intestine | 0.0 | Uterus | 0.0 |
| Colon ca. SW480 | 0.0 | Plancenta | 0.0 |
| Colon ca.* SW620(SW480 | 0.0 | Prostate | 0.0 |
| met) | | | |
| Colon ca. HT29 | 0.0 | Prostate ca.* (bone | 0.0 |
| | | met)PC-3 | |
| Colon ca. HCT-116 | 0.0 | Testis | 0.0 |
| Colon ca. CaCo-2 | 0.0 | Melanoma Hs688(A).T | 0.0 |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE RC
Panel 4D

| | Rel. Exp.(%) Ag1787, | | Rel. Exp.(%) Ag1787, |
|---|---|---|---|
| Tissue Name | Run 165809115 | Tissue Name | Run 165809115 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 11.9 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 4.9 | Microsvasular Dermal EC | 2.9 |
| | | TNFalpha + IL-1beta | |
| Primary Th1 rest | 6.2 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 4.2 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 21.9 |
| | | PMA/ionomycin | |
| 2ry Th1/Th2/Tr1_anti- | 6.2 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 85.3 |
| LAK cells IL-2 + IL-12 | 0.0 | Lupus kidney | 8.9 |
| LAK cells IL-2 + IFN | 0.0 | NCI-H292 none | 2.8 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 8.1 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 8.8 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 4.9 | Dermal fibroblast CCD1070 | 23.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 5.4 |
| Monocytes rest | 0.0 | IBD Crohn's | 3.6 |
| Monocytes LPS | 0.0 | Colon | 100.0 |
| Macrophages rest | 0.0 | Lung | 22.1 |
| Macrophages LPS | 0.0 | Thymus | 5.6 |
| HUVEC none | 0.0 | Kidney | 0.0 |
| HUVEC starved | 0.0 | | |

Panel 1.3D Summary: Ag1787 Expression of the CG149895-01 gene is highest in spleen, an important site of secondary immune responses (CT=33.7). Therefore, expression of this gene in spleen can be used to distinguish spleen from the other samples on this panel. Furthermore, antibodies or small molecule therapeutics that block the function of this GPCR may be useful as anti-inflammatory therapeutics for the treatment of allergies, autoimmune diseases, and inflammatory diseases.

Panel 2.2 Summary: Ag1787 Data from one experiment with this probe and primer set and the CG149895-01 gene is not included because the amp plot suggests that there were experimental difficulties with this run.

Panel 4D Summary: Ag1787 The CG149895-01 gene target is expressed in liver cirrhosis and in the colon. Normal liver does not express this transcript in panels 1.3 and 2.2, but this gene is expressed during liver cancer. This expression profile suggests that expression may be induced by liver damage or associated inflammation. Therefore, the transcript or the protein encoded for the transcript could be used diagnostically to identify liver cirrhosis or inflammation. Furthermore, the protein encoded by this transcript could potentially be used to design therapeutics against liver cirrhosis or inflammation.

S. CG53785-02/GMAC022998_A: Olfactory Receptor

Expression of gene CG53785-02 was assessed using the primer-probe sets Ag2686 and Ag1744, described in Tables SA and SB. Results of the RTQ-PCR runs are shown in Tables SC and SD.

TABLE SA
Probe Name Ag2686

| | | | Start | SEQ ID |
|---|---|---|---|---|
| Primers | Sequences | Length | Position | NO |
| Forward | 5′-tgtggacattgggtattccata-3′ | 22 | 204 | 199 |
| Probe | TET-5′-tcagttacgccaatcattctcatcaa-3′-TAMRA | 26 | 226 | 200 |
| Reverse | 5′-ctgtgctatacagcctgtgaca-3′ | 22 | 279 | 201 |

TABLE SB
Probe Name Ag1744

| | | | Start | SEQ ID |
|---|---|---|---|---|
| Primers | Sequences | Length | Position | NO |
| Forward | 5′-ttacaggctgtttgatgaacct-3′ | 22 | 461 | 202 |
| Probe | TET-5′-ttctgcggtccaaataaaatcaacca-3′-TAMRA | 26 | 487 | 203 |
| Reverse | 5′-aaagcttcaagagtgggaagag-3′ | 22 | 525 | 204 |

TABLE SC
Panel 2.2

| | Rel. Exp.(%) Ag1744, | | Rel. Exp.(%) Ag1744, |
|---|---|---|---|
| Tissue Name | Run 174112222 | Tissue Name | Run 174112222 |
| Normal Colon | 6.5 | Kidney Margin (OD04348) | 7.4 |
| Colon cancer (OD06064) | 0.0 | Kidney malignant cancer | 0.0 |
| | | (OD06204B) | |
| Colon Margin (OD06064) | 0.0 | Kidney normal adjacent | 0.0 |
| | | tissue (OD06204E) | |
| Colon cancer (OD06159) | 0.0 | Kidney Cancer (OD04450- | 0.0 |
| | | 01) | |
| Colon Margin (OD06159) | 0.0 | Kidney Margin (OD04450- | 0.0 |
| | | 03) | |
| Colon cancer (OD06297-04) | 0.0 | Kidney Cancer 8120613 | 0.0 |
| Colon Margin (OD06297- | 0.0 | Kidney Margin 8120614 | 0.0 |
| 015) | | | |
| CC Gr.2 ascend colon | 0.0 | Kidney Cancer 9010320 | 0.0 |
| (ODO3921) | | | |
| CC Margin (ODO3921) | 0.0 | Kidney Margin 9010321 | 0.0 |
| Colon cancer metastasis | 0.0 | Kidney Cancer 8120607 | 0.0 |
| (OD06104) | | | |
| Lung Margin (OD06104) | 0.0 | Kidney Margin 8120608 | 0.0 |
| Colon mets to lung | 0.0 | Normal Uterus | 0.0 |
| (OD04451-01) | | | |
| Lung Margin (OD04451-02) | 17.1 | Uterine Cancer 064011 | 0.0 |
| Normal Prostate | 17.2 | Normal Thyroid | 0.0 |
| Prostate Cancer (OD04410) | 0.0 | Thyroid Cancer 064010 | 0.0 |
| Prostate Margin (OD04410) | 0.0 | Thyroid Cancer A302152 | 0.0 |
| Normal Ovary | 0.0 | Thyroid Margin A302153 | 0.0 |
| Ovarian cancer (OD06283- | 0.0 | Normal Breast | 100.0 |
| 03) | | | |
| Ovarian Margin (OD06283- | 0.0 | Breast Cancer (OD04566) | 0.0 |
| 07) | | | |
| Ovarian Cancer 064008 | 0.0 | Breast Cancer 1024 | 32.1 |
| Ovarian cancer (OD06145) | 0.0 | Breast Cancer (OD04590- | 0.0 |
| | | 01) | |
| Ovarian Margin (OD06145) | 0.0 | Breast Cancer Mets | 0.0 |
| | | (OD04590-03) | |
| Ovarian cancer (OD06455- | 0.0 | Breast Cancer Metastasis | 0.0 |
| 03) | | (OD04655-05) | |
| Ovarian Margin (OD06455- | 0.0 | Breast Cancer 064006 | 2.3 |
| 07) | | | |
| Normal Lung | 22.5 | Breast Cancer 9100266 | 6.4 |
| Invasive poor diff. lung | 34.9 | Breast Margin 9100265 | 12.2 |
| adeno (ODO4945-01 | | | |
| Lung Margin (ODO4945- | 44.8 | Breast Cancer A209073 | 9.9 |
| 03) | | | |
| Lung Malignant Cancer | 0.0 | Breast Margin A2090734 | 90.8 |
| (OD03126) | | | |
| Lung Margin (OD03126) | 11.5 | Breast cancer (OD06083) | 32.8 |
| Lung Cancer (OD05014A) | 0.0 | Breast cancer node | 0.0 |
| | | metastasis (OD06083) | |
| Lung Margin (OD05014B) | 42.0 | Normal Liver | 5.9 |
| Lung cancer (OD06081) | 9.6 | Liver Cancer 1026 | 0.0 |
| Lung Margin (OD06081) | 52.1 | Liver Cancer 1025 | 0.0 |
| Lung Cancer (OD04237-01) | 0.0 | Liver Cancer 6004-T | 0.0 |
| Lung Margin (OD04237-02) | 9.7 | Liver Tissue 6004-N | 0.0 |
| Ocular Melanoma | 0.0 | Liver Cancer 6005-T | 0.0 |
| Metastasis | | | |
| Ocular Melanoma Margin | 0.0 | Liver Tissue 6005-N | 0.0 |
| (Liver) | | | |
| Melanoma Metastasis | 0.0 | Liver Cancer 064003 | 0.0 |
| Melanoma Margin (Lung) | 29.9 | Normal Bladder | 0.0 |
| Normal Kidney | 0.0 | Bladder Cancer 1023 | 0.0 |
| Kidney Ca, Nuclear grade 2 | 4.5 | Bladder Cancer A302173 | 0.0 |
| (OD04338) | | | |
| Kidney Margin (OD04338) | 0.0 | Normal Stomach | 0.0 |
| Kidney Ca Nuclear grade | 2.3 | Gastric Cancer 9060397 | 0.0 |
| 1/2 (OD04339) | | | |
| Kidney Margin (OD04339) | 3.0 | Stomach Margin 9060396 | 0.0 |
| Kidney Ca, Clear cell type | 0.0 | Gastric Cancer 9060395 | 4.1 |
| (OD04340) | | | |
| Kidney Margin (OD04340) | 0.0 | Stomach Margin 9060394 | 0.0 |
| Kidney Ca, Nuclear grade 3 | 6.1 | Gastric Cancer 064005 | 4.6 |
| (OD04348) | | | |

TABLE SD
Panel 4D

| | Rel. Exp. (%) | Rel. Exp. (%) | | Rel. Exp. (%) | Rel. Exp. (%) |
|---|---|---|---|---|---|
| | Ag1744, Run | Ag2686, Run | | Ag1744, Run | Ag2686, Run |
| Tissue Name | 165806385 | 153324740 | Tissue Name | 165806385 | 153324740 |
| Secondary Th1 act | 0.0 | 0.0 | HUVEC IL-1 beta | 0.0 | 0.0 |
| Secondary Th2 act | 0.0 | 0.0 | HUVEC IFN gamma | 0.0 | 0.0 |
| Secondary Tr1 act | 0.0 | 0.0 | HUVEC TNF alpha + | 0.0 | 0.0 |
| | | | IFN gamma | | |
| Secondary Th1 rest | 0.0 | 0.0 | HUVEC TNF alpha + | 0.0 | 0.0 |
| | | | IL4 | | |
| Secondary Th2 rest | 0.0 | 0.0 | HUVEC IL-11 | 0.0 | 0.0 |
| Secondary Tr1 rest | 0.0 | 0.0 | Lung Microvascular | 0.0 | 0.0 |
| | | | EC none | | |
| Primary Th1 act | 0.0 | 0.0 | Lung Microvascular | 0.0 | 0.0 |
| | | | EC TNF alpha + IL- | 0.0 | 0.0 |
| | | | 1 beta | | |
| Primary Th2 act | 0.0 | 0.0 | Microvascular | 0.2 | 0.0 |
| | | | Dermal EC none | | |
| Primary Tr1 act | 0.0 | 0.0 | Microsvasular | 0.0 | 0.0 |
| | | | Dermal EC TNF | | |
| | | | alpha + IL-1 beta | | |
| Primary Th1 rest | 0.0 | 0.0 | Bronchial epithelium | 1.6 | 0.0 |
| | | | TNF alpha + IL1 beta | | |
| Primary Th2 rest | 0.2 | 0.0 | Small airway | 1.6 | 2.0 |
| | | | epithelium none | | |
| Primary Tr1 rest | 0.0 | 0.0 | Small airway | 22.4 | 20.4 |
| | | | epithelium TNF | | |
| | | | alpha + IL-1 beta | | |
| CD45RA CD4 | 0.2 | 0.0 | Coronery artery SMC | 0.0 | 0.0 |
| lymphocyte act | | | rest | | |
| CD45RO CD4 | 0.0 | 0.0 | Coronery artery SMC | 0.0 | 0.0 |
| lymphocyte act | | | TNF alpha + IL-1 beta | | |
| CD8 lymphocyte act | 0.0 | 0.0 | Astrocytes rest | 3.8 | 1.4 |
| Secondary CD8 | 0.0 | 0.0 | Astrocytes TNF | 6.2 | 1.8 |
| lymphocyte rest | | | alpha + IL-1 beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD1106 | 3.1 | 3.0 |
| Th1/Th2/Tr1_anti- | | | (Keratinocytes) none | | |
| CD95 CH11 | | | | | |
| LAK cells rest | 0.0 | 0.0 | CCD1106 | 9.9 | 0.0 |
| | | | (Keratinocytes) | | |
| | | | TNF alpha + IL-1 beta | | |
| LAK cells IL-2 | 0.0 | 0.0 | Liver cirrhosis | 4.3 | 0.6 |
| LAK cells IL-2 + IL-12 | 0.0 | 0.0 | Lupus kidney | 2.4 | 0.0 |
| LAK cells IL-2 + IFN | 0.0 | 0.0 | NCI-H292 none | 51.8 | 61.1 |
| gamma | | | | | |
| LAK cells IL-2 + IL- | 0.0 | 0.0 | NCI-H292 IL-4 | 100.0 | 100.0 |
| 18 | | | | | |
| LAK cells | 0.0 | 0.0 | NCI-H292 IL-9 | 32.3 | 25.7 |
| PMA/ionomycin | | | | | |
| NK Cells IL-2 rest | 0.0 | 0.0 | NCI-H292 IL-13 | 43.8 | 42.9 |
| Two Way MLR 3 day | 0.0 | 0.0 | NCI-H292 IFN | 10.4 | 12.9 |
| | | | gamma | | |
| Two Way MLR 5 day | 0.0 | 0.0 | HPAEC none | 0.0 | 0.0 |
| Two Way MLR 7 day | 0.0 | 0.0 | HPAEC TNF alpha + | 0.0 | 0.0 |
| | | | IL-1 beta | | |
| PBMC rest | 0.0 | 0.0 | Lung fibroblast none | 0.2 | 0.0 |
| PBMC PWM | 0.0 | 0.0 | Lung fibroblast TNF | 0.1 | 0.0 |
| | | | alpha + IL-1 beta | | |
| PBMC PHA-L | 0.0 | 0.0 | Lung fibroblast IL-4 | 0.0 | 0.0 |
| Ramos (B cell) none | 0.0 | 0.0 | Lung fibroblast IL-9 | 0.0 | 0.0 |
| Ramos (B cell) | 0.0 | 0.0 | Lung fibroblast IL-13 | 0.3 | 0.0 |
| ionomycin | | | | | |
| B lymphocytes PWM | 0.0 | 0.0 | Lung fibroblast IFN | 0.2 | 0.0 |
| | | | gamma | | |
| B lymphocytes | 0.0 | 0.0 | Dermal fibroblast | 0.2 | 0.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF alpha | | |
| EOL-1 dbcAMP | 0.0 | 0.0 | Dermal fibroblast | 0.0 | 0.0 |
| PMA/ionomycin | | | CCD1070 IL-1 beta | | |
| Dendritic cells none | 0.0 | 0.0 | Dermal fibroblast | 0.0 | 0.0 |
| | | | IFN gamma | | |
| Dendritic cells LPS | 0.0 | 0.0 | Dermal fiboblast IL-4 | 0.0 | 0.0 |
| Dendritic cells anti- | 0.0 | 0.0 | IBD Colitis 2 | 1.1 | 0.0 |
| CD40 | | | | | |
| Monocytes rest | 0.0 | 0.0 | IBD Crohn's | 0.0 | 0.0 |
| Monocytes LPS | 0.0 | 0.0 | Colon | 0.2 | 0.0 |
| Macrophages rest | 0.0 | 0.0 | Lung | 1.8 | 2.1 |
| Macrophages LPS | 0.0 | 0.0 | Thymus | 1.1 | 1.2 |
| HUVEC none | 0.0 | 0.0 | Kidney | 1.7 | 1.5 |
| HUVEC starved | 0.0 | 0.0 | | | |

CNS_neurodegeneration_v1.0 Summary: Ag2686 Expression of the CG53785-02 gene is low/undetectable (CTs>35) across all of the samples on this panel (data not shown).

Panel 1.3D Summary: Ag1744/Ag2686 Expression of the CG53785-02 gene is low/undetectable (CTs>35) across all of the samples on this panel (data not shown).

Panel 2.2 Summary: Ag1744 The expression of the CG53785-02 gene appears to be highest in a sample derived from normal breast tissue (CT=32.6). In addition, there is substantial expression associated with other normal breast tissue samples as well as normal lung tissue adjacent to malignant lung tissue. Thus, the expression of this gene could be used to distinguish between these tissues and other tissues in the panel, particularly to distinguish between the normal breast or lung and malignant breast or lung. Moreover, therapeutic modulation of this gene, through the use of small molecule drugs, antibodies or protein therapeutics might be of benefit in the treatment of lung or breast cancer.

Panel 2D Summary: Ag2686 Expression of the CG53785-02 gene is low/undetectable (CTs>35) across all of the samples on this panel (data not shown).

Panel 4D Summary: Ag1744/Ag2686 The CG53785-02 transcript is most highly expressed in NCI-H292 cells stimulated by IL-4 (CTs=28-32). The gene is also expressed in a cluster of treated and untreated NCI-H292 mucoepidermoid cell line samples. The transcript is also expressed at lower but still significant levels in small airway epithelium treated with IL-1 beta and TNF-alpha. In comparison, expression in the normal lung is relatively low. The expression of the transcript in activated normal epithelium as well as a cell line that is often used as a model for airway epithelium (NCI-H292 cells) suggests that this transcript may be important in the proliferation or activation of airway epithelium. Therefore, therapeutics designed with the GPCR encoded for by the transcript could be important in the treatment of diseases which include lung airway inflammation such as asthma and COPD.

T. CG56113-01 and GMAC022882_G: Olfactory Receptor Expression of gene CG56113-01 and variant GMAC022882_G was assessed using the primer-probe sets Ag1743 and Ag1802, described in Tables TA and TB. Results of the RTQ-PCR runs are shown in Tables TC and TD.

TABLE TA
Probe Name Ag1743

| | | | Start | SEQ ID |
|---|---|---|---|---|
| Primers | Sequences | Length | Position | NO |
| Forward | 5′-tgttgactctcgacttcaaacc-3′ | 22 | 170 | 205 |
| Probe | TET-5′-ttttcctgcaacatctggctctcatt-3′-TAMRA | 26 | 202 | 206 |
| Reverse | 5′-cattttaggggcaatgacagta-3′ | 22 | 242 | 207 |

TABLE TB
Probe Name Ag1802

| | | | Start | SEQ ID |
|---|---|---|---|---|
| Primers | Sequences | Length | Position | NO |
| Forward | 5′-tgttgactctcgacttcaaacc-3′ | 22 | 170 | 208 |
| Probe | TET-5′-ttttcctgcaacatctggctctcatt-3′-TAMRA | 26 | 202 | 209 |
| Reverse | 5′-cattttaggggcaatgacagta-3′ | 22 | 242 | 210 |

TABLE TC
Panel 1.3D

| | Rel. Exp. (%) | Rel. Exp. (%) | | Rel. Exp. (%) | Rel. Exp. (%) |
|---|---|---|---|---|---|
| | Ag1743, Run | Ag1802, Run | | Ag1743, Run | Ag1802, Run |
| Tissue Name | 165940869 | 165974811 | Tissue Name | 165940869 | 165974811 |
| Liver | 2.1 | 0.0 | Kidney (fetal) | 0.0 | 0.0 |
| adenocarcinoma | | | | | |
| Pancreas | 0.0 | 0.0 | Renal ca. 786-0 | 0.0 | 0.0 |
| Pancreatic ca. | 0.0 | 0.0 | Renal ca. A498 | 6.6 | 0.0 |
| CAPAN 2 | | | | | |
| Adrenal gland | 0.0 | 0.0 | Renal ca. RXF | 14.8 | 31.9 |
| | | | 393 | | |
| Thyroid | 0.0 | 12.0 | Renal ca. ACHN | 0.0 | 0.0 |
| Salivary gland | 0.0 | 0.0 | Renal ca. UO-31 | 0.0 | 9.4 |
| Pituitary gland | 0.0 | 0.0 | Renal ca. TK-10 | 0.0 | 30.4 |
| Brain (fetal) | 0.0 | 0.0 | Liver | 0.0 | 0.0 |
| Brain (whole) | 0.0 | 0.0 | Liver (fetal) | 0.0 | 0.0 |
| Brain (amygdala) | 0.0 | 0.0 | Liver ca. | 0.0 | 85.3 |
| | | | (hepatoblast) | | |
| | | | HepG2 | | |
| Brain (cerebellum) | 0.0 | 0.0 | Lung | 0.0 | 0.0 |
| Brain (hippocampus) | 0.0 | 0.0 | Lung (fetal) | 0.0 | 0.0 |
| Brain (substantia | 0.0 | 0.0 | Lung ca. (small | 0.0 | 0.0 |
| nigra) | | | cell) LX-1 | | |
| Brain (thalamus) | 0.0 | 0.0 | Lung ca. (small | 0.0 | 0.0 |
| | | | cell) NCI-H69 | | |
| Cerebral Cortex | 0.0 | 0.0 | Lung ca. (s.cell | 0.0 | 0.0 |
| | | | var.) SHP-77 | | |
| Spinal cord | 0.0 | 10.5 | Lung ca. (large | 0.0 | 0.0 |
| | | | cell) NCI-H460 | | |
| glio/astro U87-MG | 0.0 | 0.0 | Lung ca. (non- | 0.0 | 0.0 |
| | | | sm. cell) A549 | | |
| glio/astro U-118-MG | 0.0 | 0.0 | Lung ca. (non- | 0.0 | 0.0 |
| | | | s.cell) NCI-H23 | | |
| astrocytoma SW1783 | 0.0 | 0.0 | Lung ca. (non- | 0.0 | 0.0 |
| | | | s.cell) HOP-62 | | |
| neuro*; met SK-N- | 0.0 | 0.0 | Lung ca. (non- | 0.0 | 0.0 |
| AS | | | s.cl) NCI-H522 | | |
| astrocytoma SF-539 | 0.0 | 0.0 | Lung ca. | 0.0 | 8.2 |
| | | | (squam.) SW 900 | | |
| astrocytoma SNB-75 | 0.0 | 0.0 | Lung ca. | 0.0 | 0.0 |
| | | | (squam.) NCI- | | |
| | | | H596 | | |
| glioma SNB-19 | 0.0 | 0.0 | Mammary gland | 0.0 | 0.0 |
| glioma U251 | 0.0 | 13.8 | Breast ca.* | 0.0 | 0.0 |
| | | | (pl.ef) MCF-7 | | |
| glioma SF-295 | 0.0 | 7.2 | Breast ca.* | 0.0 | 0.0 |
| | | | (pl.ef) MDA- | | |
| | | | MB-231 | | |
| Heart (fetal) | 0.0 | 0.0 | Breast ca.* | 0.0 | 0.0 |
| | | | (pl.ef) T47D | | |
| Heart | 0.0 | 0.0 | Breast ca. BT- | 0.0 | 0.0 |
| | | | 549 | | |
| Skeletal muscle | 0.0 | 0.0 | Breast ca. MDA-N | 0.0 | 0.0 |
| (fetal) | | | | | |
| Skeletal muscle | 0.0 | 0.0 | Ovary | 0.0 | 0.0 |
| Bone marrow | 0.0 | 0.0 | Ovarian ca. | 0.0 | 0.0 |
| | | | OVCAR-3 | | |
| Thymus | 0.0 | 0.0 | Ovarian ca. | 0.0 | 0.0 |
| | | | OVCAR-4 | | |
| Spleen | 100.0 | 100.0 | Ovarian ca. | 0.0 | 0.0 |
| | | | OVCAR-5 | | |
| Lymph node | 0.0 | 0.0 | Ovarian ca. | 0.0 | 9.0 |
| | | | OVCAR-8 | | |
| Colorectal | 0.0 | 12.5 | Ovarian ca. | 0.0 | 0.0 |
| | | | IGROV-1 | | |
| Stomach | 0.0 | 0.0 | Ovarian ca.* | 19.9 | 30.6 |
| | | | (ascites) SK-OV-3 | | |
| Small intestine | 0.0 | 0.0 | Uterus | 0.0 | 0.0 |
| Colon ca. SW480 | 0.0 | 0.0 | Plancenta | 0.0 | 0.0 |
| Colon ca.* | 0.0 | 0.0 | Prostate | 0.0 | 0.0 |
| SW620 (SW480 met) | | | | | |
| Colon ca. HT29 | 0.0 | 8.3 | Prostate ca.* | 0.0 | 0.0 |
| | | | (bone met) PC-3 | | |
| Colon ca. HCT-116 | 0.0 | 0.0 | Testis | 0.0 | 0.0 |
| Colon ca. CaCo-2 | 0.0 | 0.0 | Melanoma | 0.0 | 0.0 |
| | | | Hs688(A).T | | |
| Colon ca. | 0.0 | 0.0 | Melanoma* | 7.2 | 0.0 |
| tissue(ODO3866) | | | (met) | | |
| | | | Hs688(B).T | | |
| Colon ca. HCC-2998 | 0.0 | 0.0 | Melanoma | 0.0 | 0.0 |
| | | | UACC-62 | | |
| Gastric ca.* (liver | 0.0 | 0.0 | Melanoma M14 | 0.0 | 0.0 |
| met) NCI-N87 | | | | | |
| Bladder | 0.0 | 8.1 | Melanoma LOX | 0.0 | 0.0 |
| | | | IMVI | | |
| Trachea | 0.0 | 0.0 | Melanoma* | 0.0 | 0.0 |
| | | | (met) SK-MEL-5 | | |
| Kidney | 0.0 | 0.0 | Adipose | 0.0 | 0.0 |

TABLE TD
Panel 4D

| | Rel. Exp. (%) | Rel. Exp. (%) | | Rel. Exp. (%) | Rel. Exp. (%) |
|---|---|---|---|---|---|
| | Ag1743, Run | Ag1802, Run | | Ag1743, Run | AG1802, Run |
| Tissue Name | 165812605 | 165812410 | Tissue Name | 165812605 | 165812410 |
| Secondary Th1 act | 0.0 | 0.0 | HUVEC IL-1 beta | 0.0 | 0.0 |
| Secondary Th2 act | 0.0 | 0.0 | HUVEC IFN gamma | 0.0 | 0.0 |
| Secondary Tr1 act | 0.0 | 0.0 | HUVEC TNF alpha + | 0.0 | 0.0 |
| | | | IFN gamma | | |
| Secondary Th1 rest | 0.0 | 0.0 | HUVEC TNF alpha + | 0.0 | 0.0 |
| | | | IL4 | | |
| Secondary Th2 rest | 0.0 | 0.0 | HUVEC IL-11 | 0.0 | 0.0 |
| Secondary Tr1 rest | 0.0 | 0.0 | Lung Microvascular | 0.0 | 0.0 |
| | | | EC none | | |
| Primary Th1 act | 0.0 | 0.0 | Lung Microvascular | 0.0 | 0.0 |
| | | | EC TNF alpha + IL- | | |
| | | | 1 beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 0.0 |
| | | | Dermal EC TNF | | |
| | | | alpha + IL-1 beta | | |
| Primary Th1 rest | 0.0 | 0.0 | Bronchial epithelium | 0.0 | 0.0 |
| | | | TNF alpha + IL1 beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium TNF | | |
| | | | alpha + IL-1 beta | | |
| CD45RA CD4 | 0.0 | 0.0 | Coronery artery SMC | 0.0 | 0.0 |
| lymphocyte act | | | rest | | |
| CD45RO CD4 | 0.0 | 0.0 | Coronery artery SMC | 0.0 | 0.0 |
| lymphocyte act | | | TNF alpha + IL-1 beta | | |
| CD8 lymphocyte act | 0.0 | 0.0 | Astrocytes rest | 0.0 | 0.0 |
| Secondary CD8 | 0.0 | 0.0 | Astrocytes TNF | 0.0 | 0.0 |
| lymphocyte rest | | | alpha + IL-1 beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 3.5 | 0.0 | CCD1106 | 0.0 | 0.0 |
| Th1/Th2/Tr1_anti- | | | (Keratinocytes) none | | |
| CD95 CH11 | | | | | |
| LAK cells rest | 0.0 | 0.0 | CCD1106 | 0.0 | 0.0 |
| | | | (Keratinocytes) | | |
| | | | TNF alpha + IL-1 beta | | |
| LAK cells IL-2 | 0.0 | 0.0 | Liver cirrhosis | 100.0 | 100.0 |
| LAK cells IL-2 + IL-12 | 0.0 | 0.0 | Lupus kidney | 0.0 | 4.0 |
| LAK cells IL-2 + IFN | 0.0 | 0.0 | NCI-H292 none | 0.0 | 0.0 |
| gamma | | | | | |
| LAK cells IL-2 + IL- | 0.0 | 0.0 | NCI-H292 IL-4 | 0.0 | 0.0 |
| 18 | | | | | |
| LAK cells | 0.0 | 0.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 day | 0.0 | 0.0 | NCI-H292 IFN | 0.0 | 0.0 |
| | | | gamma | | |
| Two Way MLR 5 day | 0.0 | 0.0 | HPAEC none | 0.0 | 0.0 |
| Two Way MLR 7 day | 0.0 | 0.0 | HPAEC TNF alpha + | 0.0 | 0.0 |
| | | | IL-1 beta | | |
| PBMC rest | 0.0 | 0.0 | Lung fibroblast none | 0.0 | 0.0 |
| PBMC PWM | 0.0 | 0.0 | Lung fibroblast TNF | 4.3 | 0.0 |
| | | | alpha + IL-1 beta | | |
| PBMC PHA-L | 0.0 | 0.0 | Lung fibroblast IL-4 | 0.0 | 0.0 |
| Ramos (B cell) none | 0.0 | 0.0 | Lung fibroblast IL-9 | 0.0 | 0.0 |
| Ramos (B cell) | 0.0 | 0.0 | Lung fibroblast IL-13 | 0.0 | 0.0 |
| ionomycin | | | | | |
| B lymphocytes PWM | 0.0 | 0.0 | Lung fibroblast IFN | 0.0 | 0.0 |
| | | | gamma | | |
| B lymphocytes | 0.0 | 0.0 | Dermal fibroblast | 2.7 | 0.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF alpha | | |
| EOL-1 dbcAMP | 0.0 | 0.0 | Dermal fibroblast | 0.0 | 0.0 |
| PMA/ionomycin | | | CCD1070 IL-1 beta | | |
| Dendritic cells none | 0.0 | 0.0 | Dermal fibroblast | 0.0 | 0.0 |
| | | | IFN gamma | | |
| Dendritic cells LPS | 0.0 | 0.0 | Dermal fibroblast IL-4 | 0.0 | 0.0 |
| Dendritic cells anti- | 0.0 | 0.0 | IBD Colitis 2 | 37.6 | 45.7 |
| CD40 | | | | | |
| Monocytes rest | 0.0 | 0.0 | IBD Crohn's | 2.5 | 2.1 |
| Monocytes LPS | 0.0 | 0.0 | Colon | 0.0 | 0.0 |
| Macrophages rest | 0.0 | 0.0 | Lung | 0.0 | 0.0 |
| Macrophages LPS | 0.0 | 0.0 | Thymus | 0.0 | 0.0 |
| HUVEC none | 0.0 | 0.0 | Kidney | 0.0 | 0.0 |
| HUVEC starved | 0.0 | 0.0 | | | |

Panel 1.3D Summary: Ag1743/Ag1802 Results from two experiments using identical probe/primer sets are in good agreement. Expression of the CG56113-01 gene is restricted to the spleen (CTs=33-34), an important site of secondary immune responses. Therefore, expression of this gene in spleen can be used to distinguish spleen from the other samples on this panel. Furthermore, antibodies or small molecule therapeutics that block the function of this GPCR may be useful as anti-inflammatory therapeutics for the treatment of allergies, autoimmune diseases, and inflammatory diseases.

Panel 2.2 Summary: Ag1743/Ag1802 Expression of the CG561 13-01 gene is low/undetectable (CTs>35) across all of the samples on this panel (data not shown).

Panel 4D Summary: Ag1802/1743 The CG561 13-01 gene is expressed in liver cirrhosis and colitis. Normal liver and colon do not express this transcript (see panel 1.3 and 2.2 for liver) suggesting that expression may be induced by cirrhosis. The transcript or the protein encoded by the transcript could be used diagnostically to identify liver cirrhosis or colitis. Therapeutically, the protein encoded by this transcript could be used to design therapeutics against liver cirrhosis or colitis.

U. CG50245-03/SC134912167_A: Olfactory Receptor-like Expression of gene CG50245-03 was assessed using the primer-probe set Ag1726, described in Table UA. Results of the RTQ-PCR runs are shown in Tables UB, and UC.

TABLE UA
Probe Name Ag1726

| | | | Start | SEQ ID |
|---|---|---|---|---|
| Primers | Sequences | Length | Position | NO |
| Forward | 5′-acctcccaacaaccttctgtag-3′ | 22 | 903 | 211 |
| Probe | TET-5′-ccgtgacatccttgttcctaaggctg-3′-TAMRA | 26 | 872 | 212 |
| Reverse | 5′-ccatgctcaatccactcattta-3′ | 22 | 850 | 213 |

TABLE UB
Panel 2.2

| | Rel. Exp.(%) | | Rel. Exp.(%) |
|---|---|---|---|
| | Ag1726, | | Ag1726, |
| Tissue Name | Run 173761836 | Tissue Name | Run 173761836 |
| Normal Colon | 0.0 | Kidney Margin (OD04348) | 20.3 |
| Colon cancer (OD06064) | 0.0 | Kidney malignant cancer | 3.1 |
| | | (OD06204B) | |
| Colon Margin (OD06064) | 0.0 | Kidney normal adjacent | 0.0 |
| | | tissue (OD06204E) | |
| Colon cancer (OD06159) | 0.0 | Kidney Cancer (OD04450- | 4.0 |
| | | 01) | |
| Colon Margin (OD06159) | 3.3 | Kidney Margin (OD04450- | 0.0 |
| | | 03) | |
| Colon cancer (OD06297-04) | 0.0 | Kidney Cancer 8120613 | 0.0 |
| Colon Margin (OD06297- | 0.0 | Kidney Margin 8120614 | 2.6 |
| 015) | | | |
| CC Gr.2 ascend colon | 0.0 | Kidney Cancer 9010320 | 0.0 |
| (ODO3921) | | | |
| CC Margin (ODO3921) | 0.0 | Kidney Margin 9010321 | 0.0 |
| Colon cancer metastasis | 0.0 | Kidney Cancer 8120607 | 0.0 |
| (OD06104) | | | |
| Lung Margin (OD06104) | 0.0 | Kidney Margin 8120608 | 0.0 |
| Colon mets to lung | 0.0 | Normal Uterus | 0.0 |
| (OD04451-01) | | | |
| Lung Margin (OD04451-02) | 0.0 | Uterine Cancer 064011 | 0.0 |
| Normal Prostate | 0.0 | Normal Thyroid | 0.0 |
| Prostate Cancer (OD04410) | 0.0 | Thyroid Cancer | 0.0 |
| Prostate Margin (OD04410) | 0.0 | Thyroid Cancer A302152 | 0.0 |
| Normal Ovary | 0.0 | Thyroid Margin A302153 | 0.0 |
| Ovarian cancer (OD06283- | 0.0 | Normal Breast | 0.0 |
| 03) | | | |
| Ovarian Margin (OD06283- | 0.0 | Breast Cancer | 3.3 |
| 07) | | | |
| Ovarian Cancer | 100.0 | Breast Cancer | 0.0 |
| Ovarian cancer (OD06145) | 0.0 | Breast Cancer (OD04590- | 0.0 |
| | | 01) | |
| Ovarian Margin (OD06145) | 0.0 | Breast Cancer Mets | 0.0 |
| | | (OD04590-03) | |
| Ovarian cancer (OD06455- | 0.0 | Breast Cancer Metastasis | 0.0 |
| 03) | | | |
| Ovarian Margin (OD06455- | 0.0 | Breast Cancer | 0.0 |
| 07) | | | |
| Normal Lung | 0.0 | Breast Cancer 9100266 | 0.0 |
| Invasive poor diff. lung | 0.0 | Breast Margin 9100265 | 0.0 |
| adeno (ODO4945-01 | | | |
| Lung Margin (ODO4945- | 0.0 | Breast Cancer A209073 | 0.0 |
| 03) | | | |
| Lung Malignant Cancer | 0.0 | Breast Margin A2090734 | 0.0 |
| (OD03126) | | | |
| Lung Margin (OD03126) | 0.0 | Breast cancer (OD06083) | 0.0 |
| Lung Cancer (OD05014A) | 0.0 | Breast cancer node | 0.0 |
| | | metastasis (OD06083) | |
| Lung Margin (OD05014B) | 0.0 | Normal Liver | 0.0 |
| Lung cancer (OD06081) | 0.0 | Liver Cancer 1026 | 0.0 |
| Lung Margin (OD06081) | 0.0 | Liver Cancer 1025 | 11.3 |
| Lung Cancer (OD04237-01) | 0.0 | Liver Cancer 6004-T | 0.0 |
| Lung Margin (OD04237-02) | 0.0 | Liver Tissue 6004-N | 0.0 |
| Ocular Mel Met to Liver | 0.0 | Liver Cancer 6005-T | 0.0 |
| (ODO4310) | | | |
| Liver Margin (ODO4310) | 0.0 | Liver Tissue 6005-N | 0.0 |
| Melanoma Metastasis | 0.0 | Liver Cancer 064003 | 0.0 |
| Lung Margin (OD04321) | 0.0 | Normal Bladder | 0.0 |
| Normal Kidney | 0.0 | Bladder Cancer | 0.0 |
| Kidney Ca, Nuclear grade 2 | 0.0 | Bladder Cancer | 0.0 |
| (OD04338) | | | |
| Kidney Margin (OD04338) | 0.7 | Normal Stomach | 0.0 |
| Kidney Ca Nuclear grade | 0.0 | Gastric Cancer 9060397 | 0.0 |
| 1/2 (OD04339) | | | |
| Kidney Margin (OD04339) | 4.3 | Stomach Margin 9060396 | 12.9 |
| Kidney Ca, Clear cell type | 0.0 | Gastric Cancer 9060395 | 13.3 |
| (OD04340) | | | |
| Kidney Margin (OD04340) | 0.0 | Stomach Margin 9060394 | 0.0 |
| Kidney Ca, Nuclear grade 3 | 0.0 | Gastric Cancer 064005 | 0.0 |
| (OD04348) | | | |

TABLE UC
Panel 4D

| | Rel. Exp.(%) | | Rel. Exp.(%) |
|---|---|---|---|
| | Ag1726, | | Ag1726, |
| Tissue Name | Run 165364124 | Tissue Name | Run 165364124 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 7.2 | Small airway epithelium none | 0.0 |
| Primary Tr1 rest | 0.0 | Small airway epithelium | 0.0 |
| | | TNFalpha + IL-1beta | |
| CD45RA CD4 lymphocyte | 0.0 | Coronery artery SMC rest | 0.0 |
| act | | | |
| CD45RO CD4 lymphocyte | 6.0 | Coronery artery SMC | 0.0 |
| act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 6.7 | CCD1106 (Keratinocytes) | 7.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 4.7 | Liver cirrhosis | 100.0 |
| LAK cells IL-2 + IL-12 | 0.0 | Lupus kidney | 5.4 |
| LAK cells IL-2 + IFN | 7.1 | NCI-H292 none | 0.0 |
| gamma | | | |
| LAK cells IL-2 + IL-18 | 12.7 | NCI-H292 IL-4 | 0.0 |
| LAK cells | 0.0 | NCI-H292 IL-9 | 0.0 |
| PMA/ionomycin | | | |
| NK Cells IL-2 rest | 3.8 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 2.4 | HPAEC TNF alpha + IL-1 beta | 0.0 |
| PBMC rest | 0.0 | Lung fibroblast none | 0.0 |
| PBMC PWM | 10.3 | Lung fibroblast TNF alpha + | 8.8 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 6.4 | Lung fibroblast IFN gamma | 0.0 |
| B lymphocytes CD40L and | 31.2 | Dermal fibroblast CCD1070 | 0.0 |
| IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 3.3 |
| Dendritic cells anti-CD40 | 6.1 | IBD Colitis 2 | 5.3 |
| Monocytes rest | 0.0 | IBD Crohn's | 7.8 |
| Monocytes LPS | 0.0 | Colon | 6.7 |
| Macrophages rest | 0.0 | Lung | 0.0 |
| Macrophages LPS | 0.0 | Thymus | 19.6 |
| HUVEC none | 0.0 | Kidney | 0.0 |
| HUVEC starved | 0.0 | | |

Panel 1.3D Summary: Ag1726 Expression of this gene is low/undetectable (CT values<35) across all of the samples on this panel (data not shown).

Panel 2.2 Summary: Ag1726 This gene is expressed at moderate levels in a sample derived from ovarian cancer (CT=3 1.4). Thus, expression of this gene could be used to distinguish ovarian cancer from other tissues. In addition, low level of gene expression is observed in a tissue sample from a normal kidney.

Panel 4D Summary: Ag1726 Expression of this gene is detected at low levels (CT=33.3) in liver cirrhosis, but not in normal liver (no expression in normal liver is detected on Panel 1.3D). The putative GPCR encoded for by this gene could potentially allow cells within the liver to respond to specific microenvironmental signals. Therefore, therapies designed with the protein encoded for by this gene may potentially modulate liver function and play a role in the identification and treatment of inflammatory or autoimmune diseases which effect the liver including liver cirrhosis and fibrosis.

### REFERENCES

1. Mark M D, Wittemann S, Herlitze S (2000) G protein modulation of recombinant P/Q-type calcium channels by regulators of G protein signalling proteins. J. Physiol. 528 Pt 1:65-77.

Fast synaptic transmission is triggered by the activation of presynaptic Ca2+ channels which can be inhibited by Gbetagamma subunits via G protein-coupled receptors (GPCR). Regulators of G protein signalling (RGS) proteins are GTPase-accelerating proteins (GAPs), which are responsible for >100-fold increases in the GTPase activity of G proteins and might be involved in the regulation of presynaptic Ca2+ channels. In this study we investigated the effects of RGS2 on G protein modulation of recombinant P/Q-type channels expressed in a human embryonic kidney (HEK293) cell line using whole-cell recordings. 2. RGS2 markedly accelerates transmitter-mediated inhibition and recovery from inhibition of Ba2+ currents (IBa) through P/Q-type channels heterologously expressed with the muscarinic acetylcholine receptor M2 (mAChR M2). 3. Both RGS2 and RGS4 modulate the prepulse facilitation properties of P/Q-type Ca2+ channels. G protein reinhibition is accelerated, while release from inhibition is slowed. These kinetics depend on the availability of G protein alpha and betagamma subunits which is altered by RGS proteins. 4. RGS proteins unmask the Ca2+ channel beta subunit modulation of Ca2+ channel G protein inhibition. In the presence of RGS2, P/Q-type channels containing the beta2a and beta3 subunits reveal significantly altered kinetics of G protein modulation and increased facilitation compared to Ca2+ channels coexpressed with the beta1b or beta4 subunit.

PMID: 11018106

V. CG150218-01/SC135011098_A: Olfactory Receptor

Expression of gene CG150218-01 was assessed using the primer-probe sets Gpcr12 and Ag1724, described in Tables VA and VB. Results of the RTQ-PCR runs are shown in Tables VC, and VD.

TABLE VA
Probe Name Gper12

| | | | Start | SEQ ID |
|---|---|---|---|---|
| Primers | Sequences | Length | Position | NO |
| Forward | 5′-gcccaagatgctcctgga-3′ | 18 | 256 | 214 |
| Probe | TET-5′-caggtcatgggtgtgaataagatctcagcc-3′-TAMRA | 30 | 275 | 215 |
| Reverse | 5′-ggaacatctgcatcccacact-3′ | 21 | 309 | 216 |

TABLE VB
Probe Name Ag1724

| | | | Start | SEQ ID |
|---|---|---|---|---|
| Primers | Sequences | Length | Position | NO |
| Forward | 5′-gatttcatcctcatgggactct-3′ | 22 | 53 | 217 |
| Probe | TET-5′-tcagacgatccaaacatccagctcta-3′-TAMRA | 26 | 75 | 218 |
| Reverse | 5′-tcaggaaaaccacaaagatgac-3′ | 22 | 110 | 219 |

TABLE VC
Panel 1

| | Rel. Exp. (%) | Rel. Exp. (%) | | Rel. Exp. (%) | Rel. Exp. (%) |
|---|---|---|---|---|---|
| | Gpcr12, Run | Gpcr12, Run | | Gpcr12, Run | Gpcr12, Run |
| Tissue Name | 87588213 | 94275890 | Tissue Name | 87588213 | 94275890 |
| Endothelial cells | 0.0 | 0.0 | Renal ca. 786-0 | 0.0 | 0.0 |
| Endothelial cells | 0.0 | 0.0 | Renal ca. A498 | 8.5 | 0.0 |
| (treated) | | | | | |
| Pancreas | 0.9 | 0.1 | Renal ca. RXF | 0.0 | 0.0 |
| | | | 393 | | |
| Pancreatic ca. | 11.8 | 0.0 | Renal ca. ACHN | 0.0 | 0.0 |
| CAPAN 2 | | | | | |
| Adrenal gland | 0.7 | 0.0 | Renal ca. UO-31 | 4.9 | 0.0 |
| Thyroid | 0.0 | 0.0 | Renal ca. TK-10 | 1.7 | 0.0 |
| Salivary gland | 3.8 | 0.0 | Liver | 0.2 | 0.1 |
| Pituitary gland | 0.0 | 0.0 | Liver (fetal) | 0.0 | 0.0 |
| Brain (fetal) | 1.7 | 0.0 | Liver ca. | 0.0 | 0.0 |
| | | | (hepatoblast) | | |
| | | | HepG2 | | |
| Brain (whole) | 19.8 | 0.0 | Lung | 0.0 | 100.0 |
| Brain (amygdala) | 2.8 | 0.0 | Lung (fetal) | 0.0 | 0.0 |
| Brain (cerebellum) | 2.3 | 0.0 | Lung ca. (small | 0.0 | 0.0 |
| | | | cell) LX-1 | | |
| Brain | 6.3 | 0.0 | Lung ca. (small | 31.0 | 0.0 |
| (hippocampus) | | | cell) NCI-H69 | | |
| Brain (substantia | 6.4 | 0.0 | Lung ca. (s.cell | 0.0 | 0.0 |
| nigra) | | | var.) SHP-77 | | |
| Brain (thalamus) | 3.7 | 49.3 | Lung ca. (large | 0.0 | 0.0 |
| | | | cell) NCI-H460 | | |
| Brain | 0.0 | 0.0 | Lung ca. (non- | 7.9 | 0.0 |
| (hypothalamus) | | | sm. cell) A549 | | |
| Spinal cord | 1.3 | 0.0 | Lung ca. (non- | 0.0 | 0.0 |
| | | | s.cell) NCI-H23 | | |
| glio/astro U87-MG | 0.0 | 0.0 | Lung ca. (non- | 0.4 | 0.0 |
| | | | s.cell) HOP-62 | | |
| glio/astro U-118- | 1.4 | 0.0 | Lung ca. (non- | 0.0 | 0.0 |
| MG | | | s.cl) NCI-H522 | | |
| astrocytoma | 0.8 | 0.0 | Lung ca. | 0.6 | 0.0 |
| SW1783 | | | (squam.) SW | | |
| | | | 900 | | |
| neuro*; met SK-N- | 3.2 | 0.0 | Lung ca. | 12.7 | 0.0 |
| AS | | | (squam.) NCI- | | |
| | | | H596 | | |
| astrocytoma SF- | 0.8 | 0.0 | Mammary gland | 6.9 | 0.0 |
| 539 | | | | | |
| astrocytoma SNB- | 1.1 | 0.0 | Breast ca.* | 0.0 | 0.0 |
| 75 | | | (pl.ef) MCF-7 | | |
| glioma SNB-19 | 8.5 | 0.0 | Breast ca.* | 0.0 | 0.0 |
| | | | pl.ef) MDA- | | |
| | | | MB-231 | | |
| glioma U251 | 6.9 | 0.0 | Breast ca.* (pl. | 30.1 | 0.0 |
| | | | ef) T47D | | |
| glioma SF-295 | 0.2 | 0.0 | Breast ca. BT- | 0.0 | 0.0 |
| | | | 549 | | |
| Heart | 0.0 | 0.0 | Breast ca. MDA-N | 3.6 | 0.0 |
| Skeletal muscle | 0.0 | 0.0 | Ovary | 1.1 | 0.0 |
| Bone marrow | 0.0 | 0.0 | Ovarian ca. | 0.0 | 0.0 |
| | | | OVCAR-3 | | |
| Thymus | 15.0 | 0.0 | Ovarian ca. | 2.4 | 0.0 |
| | | | OVCAR-4 | | |
| Spleen | 9.2 | 0.0 | Ovarian ca. | 23.0 | 0.0 |
| | | | OVCAR-5 | | |
| Lymph node | 9.6 | 1.0 | Ovarian ca. | 8.2 | 0.0 |
| | | | OVCAR-8 | | |
| Colon (ascending) | 100.0 | 71.7 | Ovarian ca. | 0.0 | 0.0 |
| | | | IGROV-1 | | |
| Stomach | 5.0 | 0.0 | Ovarian ca. | 1.6 | 0.0 |
| | | | (ascites) SK-OV-3 | | |
| Small intestine | 0.0 | 0.0 | Uterus | 50.3 | 0.0 |
| Colon ca. SW480 | 0.0 | 0.0 | Placenta | 14.7 | 0.0 |
| Colon ca.* SW620 | 0.0 | 0.0 | Prostate | 9.7 | 0.0 |
| (SW480 met) | | | | | |
| Colon ca. HT29 | 3.3 | 0.0 | Prostate ca.* | 0.0 | 0.0 |
| | | | (bone met) PC-3 | | |
| Colon ca. HCT- | 0.0 | 0.0 | Testis | 33.0 | 0.0 |
| 116 | | | | | |
| Colon ca. CaCo-2 | 0.0 | 0.0 | Melanoma | 0.0 | 0.0 |
| | | | Hs688(A).T | | |
| Colon ca. HCT-15 | 12.0 | 0.0 | Melanoma* | 11.6 | 0.0 |
| | | | (met) | | |
| | | | Hs688(B).T | | |
| Colon ca. HCC- | 0.8 | 0.0 | Melanoma | 0.0 | 0.0 |
| 2998 | | | UACC-62 | | |
| Gastric ca. (liver | 1.3 | 0.0 | Melanoma M14 | 18.3 | 0.0 |
| met) NCI-N87 | | | | | |
| Bladder | 2.3 | 0.0 | Melanoma LOX | 0.9 | 0.0 |
| | | | IMVI | | |
| Trachea | 0.6 | 0.0 | Melanoma* | 0.0 | 0.0 |
| | | | (met) SK-MEL-5 | | |
| Kidney | 17.7 | 92.0 | Melanoma SK- | 0.0 | 0.0 |
| | | | MEL-28 | | |
| Kidney (fetal) | 1.0 | 0.0 | | | |

TABLE VD
Panel 4D

| | Rel. Exp.(%) | | Rel. Exp.(%) |
|---|---|---|---|
| | Ag1724, | | Ag1724, |
| Tissue Name | Run 165364093 | Tissue Name | Run 165364093 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 lymphocyte | 1.4 | Coronery artery SMC rest | 0.0 |
| act | | | |
| CD45RO CD4 lymphocyte | 0.0 | Coronery artery SMC | 0.0 |
| act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 1.8 | Liver cirrhosis | 8.4 |
| LAK cells IL-2 + IL-12 | 3.7 | Lupus kidney | 0.0 |
| LAK cells IL-2 + IFN | 6.3 | NCI-H292 none | 0.0 |
| gamma | | | |
| LAK cells IL-2 + IL-18 | 9.7 | NCI-H292 IL-4 | 0.0 |
| LAK cells | 0.0 | NCI-H292 IL-9 | 0.0 |
| PMA/ionomycin | | | |
| NK Cells IL-2 rest | 1.6 | NCI-H292 IL-13 | 0.0 |
| Two Way MLR 3 day | 3.0 | NCI-H292 IFN gamma | 0.0 |
| Two Way MLR 5 day | 2.3 | HPAEC none | 0.0 |
| Two Way MLR 7 day | 1.2 | HPAEC TNF alpha + IL-1 beta | 0.0 |
| PBMC rest | 0.0 | Lung fibroblast none | 0.0 |
| PBMC PWM | 4.9 | Lung fibroblast TNF alpha + | 0.0 |
| | | IL-1 beta | |
| PBMC PHA-L | 1.5 | Lung fibroblast IL-4 | 0.0 |
| Ramos (B cell) none | 0.0 | Lung fibroblast IL-9 | 0.0 |
| Ramos (B cell) ionomycin | 4.9 | Lung fibroblast IL-13 | 0.0 |
| B lymphocytes PWM | 58.2 | Lung fibroblast IFN gamma | 0.0 |
| B lymphocytes CD40L and | 100.0 | Dermal fibroblast CCD1070 | 0.0 |
| IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.9 | Dermal fibroblast IFN gamma | 0.0 |
| Dendritic cells LPS | 0.0 | Dermal fibroblast IL-4 | 0.0 |
| Dendritic cells | 0.0 | IBD Colitis 2 | 0.0 |
| anti-CD40 | | | |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 54.7 |
| HUVEC none | 0.0 | Kidney | 0.0 |
| HUVEC starved | 0.0 | | |

Panel 1 Summary: Gpcr12 Two experiments using the same probe and primer set both show similar levels of significant expression in the thalamus (CTs=30-34), colon (CTs=29) and kidney (CTs=30-32). These results suggest that the protein encoded by this gene could be used to differentiate these tissues from other tissue types.

In addition, expression of this gene in the brain appears to be restricted to the thalamus.

This specific pattern of expression in the thalamus suggests that agents that modulate the putative protein product of this gene could be useful in the targeted treatment of schizophrenia, since the thalamus has been identified by numerous studies to play an important role in schizophrenia. Furthermore, all current treatments for schizophrenia target a combination of GPCRs, from dopamine to serotonin receptors, that are expressed in the thalamus and other brain regions involved in schizophrenia.

### REFERENCES

Xiberas X, Martinot J L, Mallet L, Artiges E, Canal M, Loc'h C, Maziere B, Paillere-Martinot M L. (2001) In vivo extrastriatal and striatal D2 dopamine receptor blockade by amisulpride in schizophrenia. J Clin Psychopharmacol. 21:207-214.

Amisulpride, a substituted benzamide with high affinity for dopamine D2 and D3 receptors only, has been reported to have therapeutic effects on both negative and positive schizophrenic symptoms, although at distinct dose ranges (50-300 mg/day vs. 400-1,200 mg/day). The purpose of this study was to investigate the binding of amisulpride to extrastriatal (i.e., thalamus and temporal cortex) and striatal D2 dopamine receptors with respect to plasma amisulpride determinations. Ten patients with schizophrenia treated with amisulpride over a wide range of doses (25-1,200 mg/day) were studied. Positron emission tomography images were acquired by using 76Br—FLB-457, a highly specific antagonist of the D2 and D3 dopamine receptors. Binding indexes (BI) in the regions studied were estimated with reference to values from six healthy subjects. A curvilinear relationship was demonstrated between plasma concentration of amisulpride and the BI in extrastriatal regions. The BI also varied as a function of plasma concentration in striatum. Furthermore, the data provide evidence for different binding profiles: low plasma concentrations (28-92 ng/mL) induced marked extrastriatal binding and low striatal binding, whereas higher plasma concentrations (>153 ng/mL) induced marked binding both in extrastriatal and striatal regions. Dose-dependent differential binding profiles of amisulpride to D2 receptors in extrastriatal and striatal regions were demonstrated, and two therapeutic ranges of plasma concentrations for negative and positive schizophrenic symptoms, respectively, are suggested.

PMID: 11270918

Panel 1.3D Summary: Ag2106/Ag1724 Expression of this gene is low/undetectable (CT values >35) across all of the samples on this panel (data not shown).

Panel 2.2 Summary: Ag1724 Expression of this gene is low/undetectable (CT values>35) across all of the samples on this panel (data not shown).

Panel 4D Summary: Ag2106/Ag1724 Results from two experiments using two different probe and primer sets that respond to this gene are in very good agreement. Moderate to low expression is detected in activated B cells (CTs=30-33) and the thymus (CTs=32-34). Expression of this gene in the thymus may reflect the expression of this antigen on rapidly dividing or differentiating cells. Antibody or small molecule therapeutics designed with the protein encoded for by this gene may potentially regulate T cell development, LAK cell and B cell activation and play a role in treating autoimmune diseases such as asthma, lupus, and arthritis.

W. CG53306-01/GMAC006271_A: GPCR

Expression of gene CG53306-01 was assessed using the primer-probe set Ag1718, described in Table WA. Results of the RTQ-PCR runs are shown in Table WB.

TABLE WA
Probe Name Ag1718

| | | | Start | SEQ ID |
|---|---|---|---|---|
| Primers | Sequences | Length | Position | NO |
| Forward | 5′-caagatgctagtgagcattcca-3′ | 21 | 241 | 220 |
| Probe | TET-5′-acatctcctacatggggtgcctcact-3′-TAMRA | 26 | 276 | 221 |
| Reverse | 5′-tatccattccagcaaacatcat-3′ | 22 | 317 | 277 |

TABLE WB
Panel 2.2

| | Rel. Exp.(%) Ag1718, | | Rel. Exp.(%) Ag1718, |
|---|---|---|---|
| Tissue Name | Run 173761462 | Tissue Name | Run 173761462 |
| Normal Colon | 0.0 | Kidney Margin (OD04348) | 0.0 |
| Colon cancer (OD06064) | 0.0 | Kidney malignant cancer | 5.8 |
| | | (OD06204B) | |
| Colon Margin (OD06064) | 0.0 | Kidney normal adjacent | 0.0 |
| | | tissue (OD06204E) | |
| Colon cancer (OD06159) | 0.0 | Kidney Cancer (OD04450- | 0.0 |
| | | 01) | |
| Colon Margin (OD06159) | 0.0 | Kidney Margin (OD04450- | 0.0 |
| | | 03) | |
| Colon cancer (OD06297-04) | 0.0 | Kidney Cancer 8120613 | 0.0 |
| Colon Margin (OD06297- | 0.0 | Kidney Margin 8120614 | 0.0 |
| 015) | | | |
| CC Gr.2 ascend colon | 0.0 | Kidney Cancer 9010320 | 0.0 |
| (ODO3921) | | | |
| CC Margin (ODO3921) | 0.0 | Kidney Margin 9010321 | 0.0 |
| Colon cancer metastasis | 0.0 | Kidney Cancer 8120607 | 3.1 |
| (OD06104) | | | |
| Lung Margin (OD06104) | 0.0 | Kidney Margin 8120608 | 0.0 |
| Colon mets to lung | 0.0 | Normal Uterus | 0.0 |
| (OD04451-01) | | | |
| Lung Margin (OD04451-02) | 0.0 | Uterine Cancer 064011 | 0.0 |
| Normal Prostate | 0.0 | Normal Thyroid | 0.0 |
| Prostate Cancer (OD04410) | 0.0 | Thyroid Cancer | 0.0 |
| Prostate Margin (OD04410) | 0.0 | Thyroid Cancer A302152 | 0.0 |
| Normal Ovary | 9.3 | Thyroid Margin A302153 | 0.0 |
| Ovarian cancer (OD06283- | 0.0 | Normal Breast | 0.0 |
| 03) | | | |
| Ovarian Margin (OD06283- | 0.0 | Breast Cancer | 11.7 |
| 07) | | | |
| Ovarian Cancer | 100.0 | Breast Cancer | 0.0 |
| Ovarian cancer (OD06145) | 0.0 | Breast Cancer (OD04590- | 0.0 |
| | | 01) | |
| Ovarian Margin (OD06145) | 0.0 | Breast Cancer Mets | 0.0 |
| | | (OD04590-03) | |
| Ovarian cancer (OD06455- | 0.0 | Breast Cancer Metastasis | 0.0 |
| 03) | | | |
| Ovarian Margin (OD06455- | 0.0 | Breast Cancer | 0.0 |
| 07) | | | |
| Normal Lung | 0.0 | Breast Cancer 9100266 | 0.0 |
| Invasive poor diff. lung adeno | 0.0 | Breast Margin 9100265 | 0.0 |
| (ODO4945-01 | | | |
| Lung Margin (ODO4945- | 0.0 | Breast Cancer A209073 | 0.0 |
| 03) | | | |
| Lung Malignant Cancer | 0.0 | Breast Margin A2090734 | 0.0 |
| (OD03126) | | | |
| Lung Margin (OD03126) | 0.0 | Breast cancer (OD06083) | 0.0 |
| Lung Cancer (OD05014A) | 0.0 | Breast cancer node | 0.0 |
| | | metastasis (OD06083) | |
| Lung Margin (OD05014B) | 0.0 | Normal Liver | 0.0 |
| Lung cancer (OD06081) | 0.0 | Liver Cancer 1026 | 0.0 |
| Lung Margin (OD06081) | 0.0 | Liver Cancer 1025 | 11.9 |
| Lung Cancer (OD04237-01) | 6.4 | Liver Cancer 6004-T | 0.0 |
| Lung Margin (OD04237-02) | 0.0 | Liver Tissue 6004-N | 0.0 |
| Ocular Mel Met to Liver | 0.0 | Liver Cancer 6005-T | 0.0 |
| (ODO4310) | | | |
| Liver Margin (ODO4310) | 0.0 | Liver Tissue 6005-N | 0.0 |
| Melanoma Metastasis | 0.0 | Liver Cancer | 0.0 |
| Lung Margin (OD04321) | 0.0 | Normal Bladder | 0.0 |
| Normal Kidney | 0.0 | Bladder Cancer | 8.3 |
| Kidney Ca, Nuclear grade 2 | 0.0 | Bladder Cancer | 0.0 |
| (OD04338) | | | |
| Kidney Margin (OD04338) | 0.0 | Normal Stomach | 0.0 |
| Kidney Ca Nuclear grade | 0.0 | Gastric Cancer 9060397 | 0.0 |
| 1/2 (OD04339) | | | |
| Kidney Margin (OD04339) | 0.0 | Stomach Margin 9060396 | 5.2 |
| Kidney Ca, Clear cell type | 1.6 | Gastric Cancer 9060395 | 10.6 |
| (OD04340) | | | |
| Kidney Margin (OD04340) | 0.0 | Stomach Margin 9060394 | 0.0 |
| Kidney Ca, Nuclear grade 3 | 0.0 | Gastric Cancer 064005 | 0.0 |
| (OD04348) | | | |

Panel 1.3D Summary: Ag1718 Expression of this gene is low/undetectable (CTs>35) across all of the samples on this panel (data not shown).

Panel 2.2 Summary: Ag1718 Significant expression of this gene is seen exclusively in an ovarian cancer sample (CT=33.1). Therefore, expression of this gene may be used to distinguish ovarian cancers from the other samples on this panel. Furthermore, therapeutic modulation of the activity of the GPCR encoded by this gene may be beneficial in the treatment of ovarian cancer.

Panel 4D Summary: Ag1718 Expression of this gene is low/undetectable (CTs>35) across all of the samples on this panel (data not shown).

X. CG100307-01/GMAL359218_C and GMAL163152_A: GPCR1

Expression of gene CG 100307-01 and variant GMAL163152_A was assessed using the primer-probe sets Ag1574 and Ag1576, described in Tables XA and XB. Results of the RTQ-PCR runs are shown in Table XC.

TABLE XA
Probe Name Ag1574

| | | | Start | SEQ ID |
|---|---|---|---|---|
| Primers | Sequences | Length | Position | NO |
| Forward | 5′-cctcctcttgcttgtctcctat-3′ | 22 | 641 | 223 |
| Probe | TET-5′-ccgtgctgctagtcgatcctctaagg-3′-TAMRA | 26 | 689 | 224 |
| Reverse | 5′-tgagctgagagagtggagaaag-3′ | 22 | 715 | 225 |

TABLE XB
Probe Name Ag1576

| | | | Start | SEQ ID |
|---|---|---|---|---|
| Primers | Sequences | Length | Position | NO |
| Forward | 5′-cctcctcttgcttgtctcctat-3′ | 22 | 641 | 226 |
| Probe | TET-5′-ccgtgctgctagtcgatcctctaagg-3′-TAMRA | 26 | 689 | 227 |
| Reverse | 5′-tgagctgagagagtggagaaag-3′ | 22 | 715 | 228 |

TABLE XC
Panel 4D

| | Rel. Exp. (%) | Rel. Exp. (%) | | Rel. Exp. (%) | Rel. Exp. (%) |
|---|---|---|---|---|---|
| | Ag1574, Run | Ag1576, Run | | Ag1574, Run | Ag1576, Run |
| Tissue Name | 165321440 | 165322438 | Tissue Name | 165321440 | 165322438 |
| Secondary Th1 act | 0.0 | 0.0 | HUVEC IL-1 beta | 0.0 | 0.0 |
| Secondary Th2 act | 0.0 | 0.0 | HUVEC IFN gamma | 0.0 | 0.0 |
| Secondary Tr1 act | 0.0 | 0.0 | HUVEC TNF alpha + | 0.0 | 0.0 |
| | | | IFN gamma | | |
| Secondary Th1 rest | 0.0 | 0.0 | HUVEC TNF alpha + | 0.0 | 0.0 |
| | | | IL4 | | |
| Secondary Th2 rest | 0.0 | 0.0 | HUVEC IL-11 | 0.0 | 0.0 |
| Secondary Tr1 rest | 0.0 | 0.0 | Lung Microvascular | 0.0 | 0.0 |
| | | | EC none | | |
| Primary Th1 act | 0.0 | 0.0 | Lung Microvascular | 0.0 | 0.0 |
| | | | EC TNF alpha + IL- | | |
| | | | 1 beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 0.0 |
| | | | Dermal EC TNF | | |
| | | | alpha + IL-1 beta | | |
| Primary Th1 rest | 0.0 | 0.0 | Bronchial epithelium | 0.0 | 0.0 |
| | | | TNF alpha + IL1 beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium TNF | | |
| | | | alpha + IL-1 beta | | |
| CD45RA CD4 | 0.0 | 0.0 | Coronery artery SMC | 0.0 | 0.0 |
| lymphocyte act | | | rest | | |
| CD45RO CD4 | 0.0 | 0.0 | Coronery artery SMC | 0.0 | 0.0 |
| lymphocyte act | | | TNF alpha + IL-1 beta | | |
| CD8 lymphocyte act | 0.0 | 0.0 | Astrocytes rest | 0.0 | 0.0 |
| Secondary CD8 | 0.0 | 0.0 | Astrocytes TNF | 0.0 | 0.0 |
| lymphocyte rest | | | alpha + IL-1 beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD1106 | 0.0 | 0.0 |
| Th1/Th2/Tr1_anti- | | | (Keratinocytes) none | | |
| CD95 CH11 | | | | | |
| LAK cells rest | 0.0 | 0.0 | CCD1106 | 0.0 | 0.0 |
| | | | (Keratinocytes) | | |
| | | | TNF alpha + IL-1 beta | | |
| LAK cells IL-2 | 0.0 | 0.0 | Liver cirrhosis | 43.8 | 57.8 |
| LAK cells IL-2 + IL-12 | 0.0 | 0.0 | Lupus Kidney | 0.0 | 0.0 |
| LAK cells IL-2 + IFN | 0.0 | 0.0 | NCI-H292 none | 0.0 | 0.0 |
| gamma | | | | | |
| LAK cells IL-2 + IL- | 0.0 | 0.0 | NCI-H292 IL-4 | 0.0 | 0.0 |
| 18 | | | | | |
| LAK cells | 0.0 | 100.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 day | 0.0 | 0.0 | NCI-H292 IFN | 0.0 | 0.0 |
| | | | gamma | | |
| Two Way MLR 5 day | 0.0 | 0.0 | HPAEC none | 59.5 | 0.0 |
| Two Way MLR 7 day | 0.0 | 0.0 | HPAEC TNF alpha + | 0.0 | 0.0 |
| | | | IL-1 beta | | |
| PBMC rest | 0.0 | 0.0 | Lung fibroblast none | 0.0 | 0.0 |
| PBMC PWM | 0.0 | 0.0 | Lung fibroblast TNF | 0.0 | 0.0 |
| | | | alpha + IL-1 beta | | |
| PBMC PHA-L | 0.0 | 0.0 | Lung fibroblast IL-4 | 0.0 | 0.0 |
| Ramos (B cell) none | 0.0 | 0.0 | Lung fibroblast IL-9 | 0.0 | 0.0 |
| Ramos (B cell) | 0.0 | 0.0 | Lung fibroblast IL-13 | 0.0 | 0.0 |
| ionomycin | | | | | |
| B lymphocytes PWM | 0.0 | 0.0 | Lung fibroblast IFN | 0.0 | 0.0 |
| | | | gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF alpha | | |
| EOL-1 dbcAMP | 0.0 | 0.0 | Dermal fibroblast | 0.0 | 0.0 |
| PMA/ionomycin | | | CCD1070 IL-1 beta | | |
| Dendritic cells none | 0.0 | 0.0 | Dermal fibroblast | 0.0 | 0.0 |
| | | | IFN gamma | | |
| Dendritic cells LPS | 0.0 | 0.0 | Dermal fibroblast IL-4 | 50.7 | 0.0 |
| Dendritic cells anti- | 0.0 | 0.0 | IBD Colitis 2 | 52.5 | 77.9 |
| CD40 | | | | | |
| Monocytes rest | 0.0 | 0.0 | IBD Crohn's | 100.0 | 0.0 |
| Monocytes LPS | 0.0 | 0.0 | Colon | 0.0 | 0.0 |
| Macrophages rest | 0.0 | 0.0 | Lung | 0.0 | 8.2 |
| Macrophages LPS | 0.0 | 0.0 | Thymus | 0.0 | 0.0 |
| HUVEC none | 0.0 | 0.0 | Kidney | 0.0 | 0.0 |
| HUVEC starved | 0.0 | 0.0 | | | |

Panel 1.3D Summary: Ag1574/Ag1 576 Expression of the CG100307-01 gene is low/undetectable (CTs>35) across all of the samples on this panel (data not shown).

Panel 2.2 Summary: Ag1574/Ag1576 Expression of the CG100307-01 gene is low/undetectable (CTs>35) across all of the samples on this panel (data not shown).

Panel 4D Summary: Ag1576 The CG100307-01 gene is expressed in liver cirrohsis and colitis. Normal liver and colon do not express this transcript (see panel 1.3 and 2.2 for liver) suggesting that expression may be induced by cirrhosis. The transcript is also expressed in LAK cells. Thus, the transcript or the protein encoded by the transcript could be used diagnostically to identify liver cirrhosis, colitis or LAK cells.

The putative GPCR encoded for by this transcript could also be important in the function of LAK cells. LAK cells are important for immunosurveillance against bacterial and viral infected cells as well as transformed cells. Thus, the protein encoded by this transcript could be used to design therapeutics against liver cirrhosis or colitis. In addition, therapeutics that enhance LAK activity and serve as treatments for viral and bacterial diseases and cancer could potentially be designed with this gene product.

Ag1574 Expression of the CG100307-01 gene is low/undetectable (CTs>35) across all of the samples on this panel (data not shown).

Y. CG151693-01/GMAP001465_A: Olfactory Receptor

Expression of gene CG151693-01 was assessed using the primer-probe set Ag1571, described in Table YA. Results of the RTQ-PCR runs are shown in Table YB.

TABLE YA
Probe Name Ag1571

| | | | Start | SEQ ID |
|---|---|---|---|---|
| Primers | Sequences | Length | Position | NO |
| Forward | 5′-aaatggtgctcctagtttccat-3′ | 22 | 348 | 229 |
| Probe | TET-5′-tgttgctatatgcaaacctccccact-3′-TAMRA | 26 | 385 | 230 |
| Reverse | 5′-tacacagcagctcatgattgtc-3′ | 22 | 415 | 231 |

TABLE YB
Panel 1.3D

| | Rel. Exp.(%) Ag1571, | | Rel. Exp.(%) Ag1571, |
|---|---|---|---|
| Tissue Name | Run 165529571 | Tissue Name | Run 165529571 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 28.7 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 0.0 | Lung ca. (large | 0.0 |
| | | cell) NCI-H460 | |
| glio/astro U87-MG | 0.0 | Lung ca. (non-sm. cell) | 0.0 |
| | | A549 | |
| glio/astro U-118-MG | 92.7 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl. ef) MCF- | 0.0 |
| | | 7 | |
| glioma SF-295 | 100.0 | Breast ca.* (pl. ef) MDA- | 0.0 |
| | | MB-231 | |
| Heart (Fetal) | 0.0 | Breast ca.* (pl. ef) T47D | 0.0 |
| Heart | 0.0 | Breast ca. BT-549 | 17.6 |
| Skeletal muscle (Fetal) | 0.0 | Breast ca. MDA-N | 13.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 6.3 |
| Lymph node | 0.0 | Ovarian ca. OVCAR-8 | 0.0 |
| Colorectal | 12.5 | Ovarian ca. IGROV-1 | 29.5 |
| Stomach | 0.0 | Ovarian ca. (ascites) SK- | 22.8 |
| | | OV-3 | |
| Small intestine | 0.0 | Uterus | 0.0 |
| Colon ca. SW480 | 0.0 | Placenta | 15.8 |
| Colon ca.* SW620 | 0.0 | Prostate | 0.0 |
| (SW480 met) | | | |
| Colon ca. HT29 | 0.0 | Prostate ca.* (bone met) | 0.0 |
| | | PC-3 | |
| Colon ca. HCT-116 | 0.0 | Testis | 51.4 |
| Colon ca. CaCo-2 | 0.0 | Melanoma Hs688(A).T | 0.0 |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

Panel 1.3D Summary: Ag1571 Expression of this gene is highest in two astrocytoma cell lines (CTs=34). Therefore, expression of this gene may be used to distinguish astrocytoma cell lines from the other samples on this panel. Furthermore, therapeutic modulation of the activity of the GPCR encoded by this gene may be beneficial in the treatment of astrocytoma.

Panel 2.2 Summary: Ag1571 Expression of this gene is low/undetectable (CTs>35) across all of the samples on this panel (data not shown).

Panel 4D Summary: Ag1571 Expression of this gene is low/undetectable (CTs>35) across all of the samples on this panel (data not shown).

Z. CG151832-01/GMAC004908_A: Olfactory Receptor

Expression of gene CG151832-01 was assessed using the primer-probe sets Ag1566, Ag1570 and Ag1733, described in Tables ZA, ZB and ZC. Results of the RTQ-PCR runs are shown in Tables ZD, ZE, ZF, and ZG.

TABLE ZA
Probe Name Ag1566

| | | | Start | SEQ ID |
|---|---|---|---|---|
| Primers | Sequences | Length | Position | NO |
| Forward | 5′-cagctcctcctcctagtgtttt-3′ | 22 | 82 | 232 |
| Probe | TET-5′-cctctgtgctctatgtggcaagcatt-3′-TAMRA | 26 | 104 | 233 |
| Reverse | 5′-tggtcacagaaaacacaatgag-3′ | 22 | 142 | 234 |

TABLE ZB
Probe Name Ag1570

| | | | Start | SEQ ID |
|---|---|---|---|---|
| Primers | Sequences | Length | Position | NO |
| Forward | 5′-tttgatgcagttctcactcctt-3′ | 22 | 832 | 235 |
| Probe | TET-5′-tctgaatccagttgtctatacattcagga-3′-TAMRA | 29 | 855 | 236 |
| Reverse | 5′-tattgctgccttcatctcctta-3′ | 22 | 885 | 237 |

TABLE ZC
Probe Name Ag1733

| | | | Start | SEQ ID |
|---|---|---|---|---|
| Primers | Sequences | Length | Position | NO |
| Forward | 5′-tttgatgcagttctcactcctt-3′ | 22 | 832 | 238 |
| Probe | TET-5′-tctgaatccagttgtctatacattcagga-3′-TAMRA | 29 | 855 | 239 |
| Reverse | 5′-tattgctgccttcatctcctta-3′ | 22 | 885 | 240 |

TABLE ZD
General_screening_panel_v1.5

| | Rel. Exp.(%) Ag1566, | | Rel. Exp.(%) Ag1566, |
|---|---|---|---|
| Tissue Name | Run 228632352 | Tissue Name | Run 228632352 |
| Adipose | 5.1 | Renal ca. TK-10 | 24.8 |
| Melanoma* | 6.4 | Bladder | 24.8 |
| Hs688(A).T | | | |
| Melanoma* | 12.5 | Gastric ca. (liver met.) | 20.2 |
| Hs688(B).T | | NCI-N87 | |
| Melanoma* M14 | 6.1 | Gastric ca. KATO III | 14.7 |
| Melanoma* LOXIMVI | 9.0 | Colon ca. SW-948 | 2.3 |
| Melanoma* SK-MEL-5 | 8.2 | Colon ca. SW480 | 5.1 |
| Squamous cell | 1.0 | Colon ca.* | 1.3 |
| carcinoma SCC-4 | | (SW480 met) SW620 | |
| Testis Pool | 36.3 | Colon ca. HT29 | 3.0 |
| Prostate ca.* (bone | 1.9 | Colon ca.HCT-116 | 25.3 |
| met) PC-3 | | | |
| Prostate Pool | 10.7 | Colon ca. CaCo-2 | 7.5 |
| Placenta | 12.6 | Colon cancer tissue | 9.7 |
| Uterus Pool | 11.7 | Colon ca. SW1116 | 3.9 |
| Ovarian ca. OVCAR-3 | 11.6 | Colon ca. Colo-205 | 0.8 |
| Ovarian ca. SK-OV-3 | 69.3 | Colon ca. SW-48 | 0.7 |
| Ovarian ca. OVCAR-4 | 1.3 | Colon Pool | 25.2 |
| Ovarian ca. OVCAR-5 | 18.4 | Small Intestine Pool | 17.0 |
| Ovarian ca. IGROV-1 | 6.4 | Stomach Pool | 12.4 |
| Ovarian ca. OVCAR-8 | 11.8 | Bone Marrow Pool | 10.8 |
| Ovary | 6.0 | Fetal Heart | 5.1 |
| Breast ca. MCF-7 | 5.0 | Heart Pool | 7.4 |
| Breast ca. MDA-MB-231 | 14.9 | Lymph Node Pool | 12.3 |
| Breast ca. BT 549 | 2.5 | Fetal Skeletal Muscle | 11.2 |
| Breast ca. T47D | 0.5 | Skeletal Muscle Pool | 22.5 |
| Breast ca. MDA-N | 8.2 | Spleen Pool | 9.0 |
| Breast Pool | 23.0 | Thymus Pool | 11.4 |
| Trachea | 7.8 | CNS cancer (glio/astro) | 23.2 |
| | | U87-MG | |
| Lung | 11.5 | CNS cancer (glio/astro) U- | 37.4 |
| | | 118-MG | |
| Fetal Lung | 33.7 | CNS cancer (neuro; met) | 12.2 |
| | | SK-N-AS | |
| Lung ca. NCI-N417 | 0.6 | CNS cancer (astro) SF-539 | 6.0 |
| Lung ca. LX-1 | 15.3 | CNS cancer (astro) SNB- | 17.7 |
| | | 75 | |
| Lung ca. NCI-H146 | 1.8 | CNS cancer (glio) SNB-19 | 8.7 |
| Lung ca. SHP-77 | 10.3 | CNS cancer (glio) SF-295 | 45.7 |
| Lung ca. A549 | 0.4 | Brain (Amygdala) Pool | 6.8 |
| Lung ca. NCI-H526 | 1.5 | Brain (cerebellum) | 17.6 |
| Lung ca. NCI-H23 | 20.7 | Brain (fetal) | 12.2 |
| Lung ca. NCI-H460 | 55.5 | Brain (Hippocampus) Pool | 10.7 |
| Lung ca. HOP-62 | 5.0 | Cerebral Cortex Pool | 13.0 |
| Lung ca. NCI-H522 | 6.6 | Brain (Substantia nigra) | 10.2 |
| | | Pool | |
| Liver | 0.7 | Brain (Thalamus) Pool | 15.9 |
| Fetal Liver | 10.2 | Brain (whole) | 11.8 |
| Liver ca. HepG2 | 7.5 | Spinal Cord Pool | 9.5 |
| Kidney Pool | 23.3 | Adrenal Gland | 8.7 |
| Fetal Kidney | 34.9 | Pituitary gland Pool | 2.7 |
| Renal ca. 786-0 | 16.2 | Salivary Gland | 4.6 |
| Renal ca. A498 | 18.8 | Thyroid (female) | 0.4 |
| Renal ca. ACHN | 100.0 | Pancreatic ca. CAPAN2 | 8.5 |
| Renal ca. UO-31 | 13.5 | Pancreas Pool | 23.7 |

TABLE ZE
Panel 1.3D

| | Rel. Exp. (%) | Rel. Exp. (%) | | Rel. Exp. (%) | Rel. Exp. (%) |
|---|---|---|---|---|---|
| | Ag1570, Run | Ag1733, Run | | Ag1570, Run | Ag1733, Run |
| Tissue Name | 165534728 | 165933478 | Tissue Name | 165534728 | 165933478 |
| Liver | 4.9 | 3.5 | Kidney (fetal) | 4.6 | 5.9 |
| adenocarcinoma | | | | | |
| Pancreas | 11.7 | 1.6 | Renal ca. 786-0 | 22.2 | 10.9 |
| Pancreatic ca. | 2.8 | 4.6 | Renal ca. A498 | 26.2 | 2.7 |
| CAPAN 2 | | | | | |
| Adrenal gland | 0.0 | 3.9 | Renal ca. RXF | 9.6 | 13.6 |
| | | | 393 | | |
| Thyroid | 4.9 | 0.0 | Renal ca. ACHN | 57.0 | 36.3 |
| Salivary gland | 6.7 | 2.6 | Renal ca. UO-31 | 10.1 | 5.0 |
| Pituitary gland | 1.8 | 1.0 | Renal ca. TK-10 | 15.7 | 3.7 |
| Brain (fetal) | 5.1 | 1.2 | Liver | 1.8 | 2.0 |
| Brain (whole) | 32.3 | 1.5 | Liver (fetal) | 10.0 | 3.6 |
| Brain (amygdala) | 12.2 | 0.0 | Liver ca. | 21.6 | 4.7 |
| | | | (hepatoblast) | | |
| | | | HepG2 | | |
| Brain (cerebellum) | 8.7 | 9.3 | Lung | 0.0 | 0.0 |
| Brain | 0.0 | 0.0 | Lung (fetal) | 4.2 | 3.9 |
| (hippocampus) | | | | | |
| Brain (substantia | 0.0 | 8.6 | Lung ca. (small | 14.2 | 6.8 |
| nigra) | | | cell) LX-1 | | |
| Brain (thalamus) | 2.4 | 0.0 | Lung ca. (small | 12.0 | 3.9 |
| | | | cell) NCI-H69 | | |
| Cerebral Cortex | 1.7 | 2.5 | Lung ca. (s.cell | 6.9 | 2.9 |
| | | | var.) SHP-77 | | |
| Spinal cord | 6.7 | 0.0 | Lung ca. (large | 27.2 | 1.8 |
| | | | cell) NCI-H460 | | |
| glio/astro U87-MG | 11.0 | 5.0 | Lung ca. (non- | 3.0 | 0.0 |
| | | | sm. cell) A549 | | |
| glio/astro U-118- | 55.5 | 4.9 | Lung ca. (non- | 16.3 | 5.8 |
| MG | | | s.cell) NCI-H23 | | |
| astrocytoma | 37.1 | 22.2 | Lung ca. (non- | 2.3 | 0.0 |
| SW1783 | | | s.cell) HOP-62 | | |
| neuro*; met SK-N- | 3.3 | 2.4 | Lung ca. (non- | 0.0 | 0.0 |
| AS | | | s.cl) NCI-H522 | | |
| astrocytoma SF-539 | 12.7 | 7.0 | Lung ca. | 5.5 | 0.0 |
| | | | (squam.) SW | | |
| | | | 900 | | |
| astrocytoma SNB- | 9.6 | 0.6 | Lung ca. | 13.6 | 3.5 |
| 75 | | | (squam.) NCI- | | |
| | | | H596 | | |
| glioma SNB-19 | 37.4 | 14.2 | Mammary gland | 0.0 | 0.0 |
| glioma U251 | 100.0 | 2.9 | Breast ca.* | 11.8 | 4.5 |
| | | | (pl.ef) MCF-7 | | |
| glioma SF-295 | 30.8 | 2.2 | Breast ca.* | 5.3 | 1.3 |
| | | | (pl.ef) MDA- | | |
| | | | MB-231 | | |
| Heart (Fetal) | 0.0 | 0.0 | Breast ca.* (pl. | 0.0 | 0.0 |
| | | | ef) T47D | | |
| Heart | 7.7 | 0.0 | Breast ca. BT- | 5.1 | 0.0 |
| | | | 549 | | |
| Skeletal muscle | 2.5 | 4.2 | Breast ca. MDA-N | 10.9 | 0.5 |
| (Fetal) | | | | | |
| Skeletal muscle | 0.0 | 2.9 | Ovary | 0.0 | 0.0 |
| Bone marrow | 2.4 | 1.9 | Ovarian ca. | 11.6 | 5.1 |
| | | | OVCAR-3 | | |
| Thymus | 0.0 | 1.3 | Ovarian ca. | 2.4 | 0.0 |
| | | | OVCAR-4 | | |
| Spleen | 0.0 | 100.0 | Ovarian ca. | 11.5 | 2.2 |
| | | | OVCAR-5 | | |
| Lymph node | 9.1 | 0.0 | Ovarian ca. | 13.2 | 4.6 |
| | | | OVCAR-8 | | |
| Colorectal | 6.3 | 9.5 | Ovarian ca. | 0.0 | 2.4 |
| | | | IGROV-1 | | |
| Stomach | 5.8 | 3.0 | Ovarian ca. | 25.0 | 47.6 |
| | | | (ascites) SK- | | |
| | | | OV-3 | | |
| Small intestine | 9.9 | 0.0 | Uterus | 2.1 | 0.0 |
| Colon ca. SW480 | 0.0 | 0.0 | Placenta | 0.0 | 2.4 |
| Colon ca.* SW620 | 0.0 | 0.0 | Prostate | 2.2 | 0.0 |
| (SW480 met) | | | | | |
| Colon ca. HT29 | 0.0 | 1.5 | Prostate ca.* | 2.2 | 0.0 |
| | | | (bone met) PC-3 | | |
| Colon ca. HCT-116 | 12.2 | 3.4 | Testis | 23.7 | 10.4 |
| Colon ca. CaCo-2 | 0.0 | 1.1 | Melanoma | 2.1 | 0.0 |
| | | | Hs688(A).T | | |
| CC Well to Mod | 7.7 | 1.4 | Melanoma* | 2.5 | 1.0 |
| Diff (ODO3866) | | | (met) | | |
| | | | Hs688(B).T | | |
| Colon ca. HCC- | 20.3 | 3.3 | Melanoma | 8.7 | 2.4 |
| 2998 | | | UACC-62 | | |
| Gastric ca. (liver | 8.2 | 0.7 | Melanoma M14 | 21.2 | 15.0 |
| met) NCI-N87 | | | | | |
| Bladder | 15.8 | 8.8 | Melanoma LOX | 3.5 | 0.0 |
| | | | IMVI | | |
| Trachea | 0.0 | 0.0 | Melanoma* | 0.0 | 0.0 |
| | | | (met) SK-MEL-5 | | |
| Kidney | 0.0 | 3.0 | Adipose | 8.7 | 1.3 |

TABLE ZF
Panel 2.2

| | Rel. Exp.(%) | Rel. Exp.(%) | | Rel. Exp.(%) | Rel. Exp.(%) |
|---|---|---|---|---|---|
| | Ag1570, Run | Ag1733, Run | | Ag1570, Run | Ag1733, Run |
| Tissue Name | 173968823 | 174111806 | Tissue Name | 173968823 | 174111806 |
| Normal Colon | 8.2 | 4.2 | Kidney Margin | 80.7 | 88.9 |
| | | | (OD04348) | | |
| Colon cancer | 2.7 | 3.1 | Kidney malignant | 40.1 | 38.4 |
| (OD06064) | | | cancer | | |
| | | | (OD06204B) | | |
| Colon Margin | 0.0 | 0.0 | Kidney normal | 0.0 | 2.8 |
| (OD06064) | | | adjacent tissue | | |
| | | | (OD06204E) | | |
| Colon cancer | 0.0 | 0.0 | Kidney Cancer | 100.0 | 74.2 |
| (OD06159) | | | (OD04450-01) | | |
| Colon Margin | 10.3 | 0.0 | Kidney Margin | 10.4 | 15.6 |
| (OD06159) | | | (OD04450-03) | | |
| Colon cancer | 0.0 | 9.0 | Kidney Cancer | 0.0 | 0.0 |
| (OD06297-04) | | | 8120613 | | |
| Colon Margin | 12.2 | 4.3 | Kidney Margin | 8.4 | 0.0 |
| (OD06297-015) | | | 8120614 | | |
| CC Gr.2 ascend | 2.9 | 8.0 | Kidney Cancer | 6.8 | 4.3 |
| colon (OD03921) | | | 9010320 | | |
| CC Margin | 3.7 | 14.3 | Kidney Margin | 2.1 | 7.9 |
| (ODO3921) | | | 9010321 | | |
| Colon cancer | 0.0 | 0.0 | Kidney Cancer | 4.8 | 0.0 |
| metastasis | | | 8120607 | | |
| (OD06104) | | | | | |
| Lung Margin | 2.6 | 0.0 | Kidney Margin | 0.0 | 0.0 |
| (OD06104) | | | 8120608 | | |
| Colon mets to | 34.4 | 24.5 | Normal Uterus | 7.6 | 1.6 |
| lung (OD04451-01) | | | | | |
| Lung Margin | 22.2 | 22.5 | Uterine Cancer | 0.0 | 4.0 |
| (OD04451-02) | | | 064011 | | |
| Normal Prostate | 0.0 | 9.5 | Normal Thyroid | 0.0 | 0.0 |
| Prostate Cancer | 14.9 | 7.5 | Thyroid Cancer | 11.2 | 12.9 |
| (OD04410) | | | | | |
| Prostate Margin | 9.9 | 6.0 | Thyroid Cancer | 16.4 | 40.3 |
| (OD04410) | | | A302152 | | |
| Normal Ovary | 0.0 | 0.0 | Thyroid Margin | 0.0 | 0.0 |
| | | | A302153 | | |
| Ovarian cancer | 0.0 | 5.6 | Normal Breast | 57.8 | 23.3 |
| (OD06283-03) | | | | | |
| Ovarian Margin | 6.2 | 19.2 | Breast Cancer | 21.8 | 16.4 |
| (OD06283-07) | | | | | |
| Ovarian Cancer | 10.2 | 35.4 | Breast Cancer | 6.1 | 18.2 |
| Ovarian cancer | 0.0 | 0.0 | Breast Cancer | 6.3 | 0.0 |
| (OD06145) | | | (OD04590-01) | | |
| Ovarian Margin | 7.9 | 17.9 | Breast Cancer | 7.0 | 28.7 |
| (OD06145) | | | Mets (OD04590-03) | | |
| Ovarian cancer | 24.7 | 24.5 | Breast Cancer | 24.1 | 48.0 |
| (OD06455-03) | | | Metastasis | | |
| Ovarian Margin | 2.4 | 4.1 | Breast Cancer | 11.6 | 0.0 |
| (OD06455-07) | | | | | |
| Normal Lung | 8.7 | 18.6 | Breast Cancer | 15.8 | 4.8 |
| | | | 9100266 | | |
| Invasive poor diff. | 10.3 | 0.0 | Breast Margin | 0.0 | 0.0 |
| lung adeno | | | 9100265 | | |
| (OD04945-01 | | | | | |
| Lung Margin | 28.3 | 14.8 | Breast Cancer | 3.6 | 0.0 |
| (OD04945-03) | | | A209073 | | |
| Lung Malignant | 2.5 | 0.0 | Breast Margin | 14.1 | 17.6 |
| Cancer | | | A2090734 | | |
| (OD03126) | | | | | |
| Lung Margin | 3.6 | 0.0 | Breast cancer | 11.6 | 30.8 |
| (OD03126) | | | (OD06083) | | |
| Lung Cancer | 11.6 | 8.6 | Breast cancer | 7.8 | 18.0 |
| (OD05014A) | | | node metastasis | | |
| | | | (OD06083) | | |
| Lung Margin | 23.8 | 8.8 | Normal Liver | 46.0 | 94.6 |
| (OD05014B) | | | | | |
| Lung cancer | 0.0 | 11.3 | Liver Cancer | 0.0 | 0.0 |
| (OD06081) | | | 1026 | | |
| Lung Margin | 4.1 | 9.3 | Liver Cancer | 18.0 | 14.5 |
| (OD06081) | | | 1025 | | |
| Lung Cancer | 0.0 | 0.0 | Liver Cancer | 9.7 | 0.0 |
| (OD04237-01) | | | 6004-T | | |
| Lung Margin | 30.6 | 32.5 | Liver Tissue | 0.0 | 9.6 |
| (OD04237-02) | | | 6004-N | | |
| Ocular Mel Met to | 3.5 | 0.0 | Liver Cancer | 0.0 | 0.0 |
| Liver (ODO4310) | | | 6005-T | | |
| Liver Margin | 3.2 | 7.7 | Liver Tissue | 0.0 | 0.0 |
| (ODO4310) | | | 6005-N | | |
| Melanoma | 4.3 | 6.0 | Liver Cancer | 21.0 | 36.1 |
| Metastasis | | | | | |
| Lung Margin | 1.8 | 0.0 | Normal Bladder | 3.6 | 19.3 |
| (OD04321) | | | | | |
| Normal Kidney | 9.4 | 18.7 | Bladder Cancer | 10.3 | 0.0 |
| Kidney Ca, | 40.3 | 33.2 | Bladder Cancer | 13.5 | 26.8 |
| Nuclear grade 2 | | | | | |
| (OD04338) | | | | | |
| Kidney Margin | 34.4 | 15.1 | Normal Stomach | 16.7 | 30.4 |
| (OD04338) | | | | | |
| Kidney Ca | 46.0 | 100.0 | Gastric Cancer | 0.0 | 0.0 |
| Nuclear grade 1/2 | | | 9060397 | | |
| (OD04339) | | | | | |
| Kidney Margin | 0.0 | 9.4 | Stomach Margin | 0.0 | 8.4 |
| (OD04339) | | | 9060396 | | |
| Kidney Ca, Clear | 25.5 | 15.1 | Gastric Cancer | 11.8 | 7.6 |
| cell type | | | 9060395 | | |
| (OD04340) | | | | | |
| Kidney Margin | 14.3 | 29.7 | Stomach Margin | 11.0 | 7.4 |
| (OD04340) | | | 9060394 | | |
| Kidney Ca, | 0.0 | 0.0 | Gastric Cancer | 3.4 | 18.8 |
| Nuclear grade 3 | 0.0 | 0.0 | 064005 | | |
| (OD04348) | | | | | |

TABLE ZG
Panel 4D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1566, | Ag1570, | Ag1733, | | Ag1566, | Ag1570, | Ag1733, |
| | Run | Run | Run | | Run | Run | Run |
| Tissue Name | 163479216 | 163480432 | 165813007 | Tissue Name | 163479216 | 163480432 | 165813007 |
| Secondary | 22.5 | 29.1 | 12.7 | HUVEC IL- | 10.6 | 9.7 | 5.8 |
| Th1 act | | | | 1 beta | | | |
| Secondary | 5.6 | 6.1 | 2.1 | HUVEC IFN | 27.0 | 14.6 | 7.2 |
| Th2 act | | | | gamma | | | |
| Secondary Tr1 | 10.4 | 6.0 | 2.9 | HUVEC TNF | 17.7 | 12.4 | 4.3 |
| act | | | | alpha + IFN | | | |
| | | | | gamma | | | |
| Secondary | 0.0 | 14.3 | 7.6 | HUVEC TNF | 19.8 | 11.9 | 10.2 |
| Th1 rest | | | | alpha + IL4 | | | |
| Secondary | 7.0 | 13.7 | 1.4 | HUVEC IL-11 | 25.9 | 6.9 | 2.7 |
| Th2 rest | | | | | | | |
| Secondary Tr1 | 7.9 | 9.7 | 2.3 | Lung | 34.4 | 35.4 | 13.4 |
| rest | | | | Microvascular | | | |
| | | | | EC none | | | |
| Primary Th1 | 33.4 | 39.8 | 7.3 | Lung | 31.2 | 23.2 | 13.7 |
| act | | | | Microvascular | | | |
| | | | | EC TNF | | | |
| | | | | alpha + | | | |
| | | | | IL-1 beta | | | |
| Primary Th2 | 33.7 | 50.3 | 21.8 | Microvascular | 37.9 | 71.2 | 11.7 |
| act | | | | Dermal EC | | | |
| | | | | none | | | |
| Primary Tr1 | 47.6 | 55.9 | 24.5 | Microsvasular | 38.2 | 18.4 | 7.7 |
| act | | | | Dermal EC | | | |
| | | | | TNF alpha + | | | |
| | | | | IL-1 beta | | | |
| Primary Th1 | 39.0 | 34.9 | 18.8 | Bronchial | 59.9 | 28.1 | 3.6 |
| rest | | | | epithelium | | | |
| | | | | TNF alpha + | | | |
| | | | | IL1 beta | | | |
| Primary Th2 | 20.2 | 10.1 | 5.5 | Small airway | 6.0 | 3.7 | 1.5 |
| rest | | | | epithelium | | | |
| | | | | none | | | |
| Primary Tr1 | 15.3 | 16.3 | 5.1 | Small airway | 49.3 | 25.7 | 12.3 |
| rest | | | | epithelium | | | |
| | | | | TNF alpha + | | | |
| | | | | IL-1 beta | | | |
| CD45RA CD4 | 19.2 | 9.5 | 14.3 | Coronery | 11.8 | 4.7 | 4.0 |
| lymphocyte | | | | artery SMC | | | |
| act | | | | rest | | | |
| CD45RO CD4 | 25.7 | 16.7 | 13.4 | Coronery | 5.2 | 4.5 | 0.8 |
| lymphocyte | | | | artery SMC | | | |
| act | | | | TNF alpha + | | | |
| | | | | IL-1 beta | | | |
| CD8 | 28.9 | 27.2 | 21.6 | Astrocytes | 72.2 | 38.2 | 44.1 |
| lymphocyte | | | | rest | | | |
| act | | | | | | | |
| Secondary | 25.9 | 28.9 | 14.6 | Astrocytes | 29.3 | 50.0 | 61.6 |
| CD8 | | | | TNF alpha + | | | |
| lymphocyte | | | | IL-1 beta | | | |
| rest | | | | | | | |
| Secondary | 6.3 | 13.5 | 15.2 | KU-812 | 71.7 | 24.5 | 23.8 |
| CD8 | | | | (Basophil) | | | |
| lymphocyte | | | | rest | | | |
| act | | | | | | | |
| CD4 | 10.4 | 2.5 | 12.9 | KU-812 | 100.0 | 100.0 | 48.3 |
| lymphocyte | | | | (Basophil) | | | |
| none | | | | PMA/ | | | |
| | | | | ionomycin | | | |
| 2ry | 9.5 | 12.2 | 6.7 | CCD1106 | 20.9 | 28.9 | 5.9 |
| Th1/Th2/Tr1_{—} | | | | (Keratinocytes) | | | |
| anti-CD95 | | | | none | | | |
| CH11 | | | | | | | |
| LAK cells rest | 14.9 | 21.5 | 5.3 | CCD1106 | 0.7 | 9.0 | 29.3 |
| | | | | (Keratinocytes) | | | |
| | | | | TNF alpha + | | | |
| | | | | IL-1 beta | | | |
| LAK cells IL-2 | 30.8 | 27.7 | 17.3 | Liver cirrhosis | 50.3 | 92.7 | 100.0 |
| LAK cells | 18.8 | 9.9 | 17.7 | Lupus kidney | 20.9 | 6.0 | 15.2 |
| IL-2 + | | | | | | | |
| IL-12 | | | | | | | |
| LAK cells | 43.2 | 27.0 | 38.2 | NCI-H292 | 25.0 | 16.3 | 12.5 |
| IL-2 + | | | | none | | | |
| IFN gamma | | | | | | | |
| LAK cells IL- | 27.5 | 27.5 | 25.0 | NCI-H292 IL-4 | 23.5 | 23.5 | 13.4 |
| 2 + IL-18 | | | | | | | |
| LAK cells | 20.3 | 21.6 | 11.5 | NCI-H292 IL-9 | 19.1 | 25.0 | 10.3 |
| PMA/ionomyc | | | | | | | |
| in | | | | | | | |
| NK Cells IL-2 | 17.0 | 27.4 | 7.2 | NCI-H292 IL- | 10.7 | 12.4 | 3.3 |
| rest | | | | 13 | | | |
| Two Way | 22.2 | 31.0 | 18.3 | NCI-H292 | 24.5 | 14.4 | 6.3 |
| MLR 3 day | | | | IFN gamma | | | |
| Two Way | 17.2 | 9.0 | 17.1 | HPAEC none | 23.7 | 9.1 | 7.2 |
| MLR 5 day | | | | | | | |
| Two Way | 19.1 | 19.3 | 9.3 | HPAEC TNF | 38.7 | 12.9 | 11.7 |
| MLR 7 day | | | | alpha + IL-1 | | | |
| | | | | beta | | | |
| PBMC rest | 2.3 | 0.0 | 5.3 | Lung | 22.5 | 42.9 | 18.2 |
| | | | | fibroblast | | | |
| | | | | none | | | |
| PBMC PWM | 81.8 | 51.4 | 14.2 | Lung | 15.6 | 17.9 | 18.6 |
| | | | | fibroblast | | | |
| | | | | TNF alpha + | | | |
| | | | | IL-1 beta | | | |
| PBMC PHA-L | 23.8 | 22.4 | 6.4 | Lung | 76.3 | 46.3 | 20.0 |
| | | | | fibroblast IL-4 | | | |
| Ramos (B | 12.7 | 6.7 | 7.6 | Lung | 42.0 | 23.0 | 17.0 |
| cell) none | | | | fibroblast IL-9 | | | |
| Ramos (B | 37.9 | 12.0 | 6.7 | Lung | 44.8 | 40.1 | 16.6 |
| cell) | | | | fibroblast IL- | | | |
| ionomycin | | | | 13 | | | |
| B | 58.2 | 35.4 | 7.9 | Lung | 54.7 | 42.3 | 13.2 |
| lymphocytes | | | | fibroblast IFN | | | |
| PWM | | | | gamma | | | |
| B | 37.6 | 20.6 | 9.6 | Dermal | 23.8 | 48.0 | 27.7 |
| lymphocytes | | | | fibroblast | | | |
| CD40L and | | | | CCD1070 rest | | | |
| IL-4 | | | | | | | |
| EOL-1 | 30.6 | 27.5 | 12.8 | Dermal | 43.2 | 34.6 | 24.1 |
| dbcAMP | | | | fibroblast | | | |
| | | | | CCD1070 | | | |
| | | | | TNF alpha | | | |
| EOL-1 | 36.1 | 24.3 | 15.2 | Dermal | 37.9 | 23.7 | 12.6 |
| dbcAMP | | | | fibroblast | | | |
| PMA/ionomyc | | | | CCD1070 IL- | | | |
| in | | | | 1 beta | | | |
| Dendritic cells | 25.2 | 32.3 | 7.9 | Dermal | 17.3 | 16.7 | 5.9 |
| none | | | | fibroblast IFN | | | |
| | | | | gamma | | | |
| Dendritic cells | 9.5 | 11.2 | 2.7 | Dermal | 47.0 | 59.9 | 12.9 |
| LPS | | | | fibroblast IL-4 | | | |
| Dendritic cells | 28.5 | 9.6 | 8.7 | IBD Colitis 2 | 12.9 | 6.9 | 4.0 |
| anti-CD40 | | | | | | | |
| Monocytes | 9.4 | 9.3 | 3.5 | IBD Crohn's | 7.5 | 0.0 | 0.5 |
| rest | | | | | | | |
| Monocytes | 19.1 | 23.2 | 14.6 | Colon | 15.1 | 28.3 | 28.3 |
| LPS | | | | | | | |
| Macrophages | 21.3 | 17.3 | 13.3 | Lung | 11.2 | 7.1 | 4.0 |
| rest | | | | | | | |
| Macrophages | 8.8 | 5.8 | 1.9 | Thymus | 66.9 | 78.5 | 23.8 |
| LPS | | | | | | | |
| HUVEC none | 20.4 | 10.4 | 21.5 | Kidney | 68.3 | 86.5 | 21.3 |
| HUVEC | 31.6 | 15.7 | 24.7 | | | | |
| starved | | | | | | | |

CNS_neurodegeneration_v1.0 Summary: Ag1566 No difference was detected in the expression of this gene in the postmortem brains of Alzheimer's diseased patients when compared to controls; however this panel demonstrates the expression of this gene in the brains of an independent group of subjects. See General_screening_panel_v1.5 for a discussion of utility in the central nervous system.

General_screening panel_v1.5 Summary: Ag1566 The expression of this gene appears to be highest in a sample derived from a renal cell cancer cell line (ACHN). In addition there is substantial expression in samples derived from a lung cancer and an ovarian cancer. There is lower level expression in numerous samples across the panel. Thus, the expression of this gene could be used to distinguish the renal cell cancer cell line, ACHN, form the other samples in the panel. Moreover, therapeutic modulation of this gene, through the use of antibodies, small molecule drugs or protein therapeutics might be of benefit in the treatment of renal cell cancer, lung cancer or ovarian cancer.

This gene represents a novel G-protein coupled receptor (GPCR) that also shows expression in the brain. The GPCR family of receptors contains a large number of neurotransmitter receptors, including the dopamine, serotonin, a and b-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore, this gene may be of use as a small molecule target for the treatment of any of the described diseases.

### REFERENCES

El Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol September 2001;134(1):68-77.

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermnates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15:12-7.

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSR1 treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alphal -adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini ME, Reggiani A. Glycine-site antagonists and stroke. Expert Opin Investig Drugs November 1999;8(11):1837-1848.

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport Dec. 1, 1998;9(17):3955-9 Related Articles, Books, LinkOut Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 1.3D Summary: Ag1570/Ag1733 Two experiments with two different probe and primer sets show highest expression in the spleen and a brain cancer cell line. Thus, expression of this gene could be used to differentiate between these samples and other samples on this panel. There is also low but significant expression in cell lines derived from renal cancer. This is in concordance with the results from panels 2.2 and screening_panel_v1.5. Please see Panel 2.2 for further discussion of potential utility of this gene.

Panel 2.2 Summary: Ag1570/1733 The expression of this gene was assessed in two independent runs on panel 2.2 using two different probe/primer sets. There appears to be good concordance between the runs. The highest expression in both panels appears to be in kidney cancer samples, although they are different samples in the two panels. There is also substantial expression in another sample derived from a kidney cancer. Thus, the expression of this gene could be used to distinguish these kidney cancer samples from other samples in the panel. Moreover, therapeutic modulation of this gene, through the use of antibodies, small molecule drugs or protein therapeutics might be of benefit in the treatment of kidney cancer.

Panel 4D Summary: Ag1566/1570/Ag1733 This transcript is highly expressed in the PMA and ionomycin treated basophil cell line KU-812 (CT=29.2) and to a lesser extent in untreated KU-812 cells. It is also expressed predominantly in activated B cells and lung fibroblasts treated with IL-4. Basophils play an important role in many allergic diseases and other diseases including asthma and inflammatory bowel disease. This gene encodes a putative GPCR. GPCR-type receptors are important in multiple physiological responses mediated by basophils (ref. 1). Therefore, antibody or small molecule therapies designed with the protein encoded by this gene could block or inhibit inflammation or tissue damage due to basophil activation in response to asthma, allergies, hypersensitivity reactions, psoriasis, and viral infections. In addition, the expression of this GPCR receptor homolog on activated B cells suggests that antibody or small molecule therapies designed with the protein encoded for by this gene could be beneficial for the treatment of hyperglobulinemia and B cell mediated diseases such as systemic lupus erythematosus, rheumatoid arthritis and Crohn's diseases

This transcript is also expressed in TNF-a and IL-1 treated astrocytes. This suggest that antibody or small molecule therapies designed with the protein encoded for by this gene could also be beneficial for the treatment of inflammatory CNS diseases such as multiple sclerosis or stroke.

### REFERENCES

Heinemann A., Hartnell A., Stubbs V. E., Murakami K., Soler D., LaRosa G., Askenase P. W., Williams T. J., Sabroe I. (2000) Basophil responses to chemokines are regulated by both sequential and cooperative receptor signaling. J. Immunol. 165: 7224-7233.

To investigate human basophil responses to chemokines, we have developed a sensitive assay that uses flow cytometry to measure leukocyte shape change as a marker of cell responsiveness. PBMC were isolated from the blood of volunteers. Basophilsswere identified as a single population of cells that stained positive for IL-3Ralpha (CDw123) and negative for HLA-DR, and their increase in forward scatter (as a result of cell shape change) in response to chemokines was measured. Shape change responses of basophils to chemokines were highly reproducible, with a rank order of potency: monocyte chemoattractant protein (MCP) 4 (peak at /=eotaxin-2=eotaxin-3>/=eotaxin>MCP-1=MCP-3> macrophage-inflammatory protein-1alpha>RANTES=MCP-2 IL-8. The CCR4-selective ligand macrophage-derived chemokine did not elicit a response at concentrations up to 10 nM. Blocking mAbs to CCR2 and CCR3 demonstrated that responses to higher concentrations (>10 nM) of MCP-1 were mediated by CCR3 rather than CCR2, whereas MCP-4 exhibited a biphasic response consistent with sequential activation of CCR3 at lower concentrations and CCR2 at 10 nM MCP-4 and above. In contrast, responses to MCP-3 were blocked only in the presence of both mAbs, but not after pretreatment with either anti-CCR2 or anti-CCR3 mAb alone. These patterns of receptor usage were different from those seen for eosinophils and monocytes. We suggest that cooperation between CCRs might be a mechanism for preferential recruitment of basophils, as occurs in tissue hypersensitivity responses in vivo.

AA. CG56081-02/GMAC005962_A: Olfactory Receptor

Expression of gene CG56081-02 was assessed using the primer-probe set Ag1561, described in Table AAA. Results of the RTQ-PCR runs are shown in Table AAB.

TABLE AAA
Probe Name Ag1561

| | | | Start | SEQ ID |
|---|---|---|---|---|
| Primers | Sequences | Length | Position | NO |
| Forward | 5′-tgtccaactggctctgatactt-3′ | 22 | 476 | 241 |
| Probe | TET-5′-cccttctgtggcccaatatcctaga-3′-TAMRA | 26 | 504 | 242 |
| Reverse | 5′-cttggggaacatcacagtagaa-3′ | 22 | 534 | 243 |

TABLE AAB
General_screening_panel_v1.5

| | Rel Exp.(%) Ag1561, | | Rel Exp.(%) Ag1561, |
|---|---|---|---|
| Tissue Name | Run 228720110 | Tissue Name | Run 228720110 |
| Adipose | 0.0 | Renal ca. TK-10 | 0.0 |
| Melanoma* | 0.0 | Bladder | 1.2 |
| Hs688(A).T | | | |
| Melanoma* | 6.0 | Gastric ca. (liver met.) | 0.0 |
| Hs688(B).T | | NCI-N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell | 0.0 | Colon ca.* (SW480 met) | 0.0 |
| carcinoma SCC-4 | | SW620 | |
| Testis Pool | 2.7 | Colon ca. HT29 | 0.0 |
| Prostate ca.* (bone | 0.0 | Colon ca.HCT-116 | 0.0 |
| met) PC-3 | | | |
| Prostate Pool | 0.0 | Colon ca. CaCo-2 | 2.5 |
| Placenta | 0.0 | Colon cancer tissue | 0.0 |
| Uterus Pool | 17.3 | Colon ca. SW1116 | 1.2 |
| Ovarian ca. OVCAR-3 | 0.0 | Colon ca. Colo-205 | 0.0 |
| Ovarian ca. SK-OV-3 | 12.0 | Colon ca. SW-48 | 0.0 |
| Ovarian ca. OVCAR-4 | 0.0 | Colon Pool | 10.1 |
| Ovarian ca. OVCAR-5 | 0.0 | Small Intestine Pool | 6.8 |
| Ovarian ca. IGROV-1 | 0.0 | Stomach Pool | 0.0 |
| Ovarian ca. OVCAR-8 | 0.0 | Bone Marrow Pool | 1.2 |
| Ovary | 0.0 | Fetal Heart | 0.0 |
| Breast ca. MCF-7 | 0.0 | Heart Pool | 0.0 |
| Breast ca. MDA-MB- | 8.2 | Lymph Node Pool | 1.2 |
| 231 | | | |
| Breast ca. BT 549 | 3.1 | Fetal Skeletal Muscle | 0.01 |
| Breast ca. T47D | 0.0 | Skeletal Muscle Pool | 0.0 |
| Breast ca. MDA-N | 0.0 | Spleen Pool | 12.1 |
| Breast Pool | 0.0 | Thymus Pool | 0.0 |
| Trachea | 0.0 | CNS cancer (glio/astro) | 0.0 |
| | | U87-MG | |
| Lung | 0.0 | CNS cancer (glio/astro) | 12.3 |
| | | U-118-MG | |
| Fetal Lung | 0.0 | CNS cancer (neuro; met) | 0.0 |
| | | SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) SNB- | 0.0 |
| | | 75 | |
| Lung ca. NCI-H146 | 0.0 | CNS cancer (glio) SNB- | 0.0 |
| | | 19 | |
| Lung ca. SHP-77 | 4.5 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 1.4 |
| Lung ca. NCI-H23 | 5.9 | Brain (fetal) | 2.7 |
| Lung ca. NCI-H460 | 100.0 | Brain (Hippocampus) | 0.0 |
| | | Pool | |
| Lung ca. HOP-62 | 0.0 | Cerebral Cortex Pool | 0.0 |
| Lung ca. NCI-H522 | 3.2 | Brain (Substantia nigra) | 1.6 |
| | | Pool | |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 4.3 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

General_screening_panel_v1.5 Summary: Ag1561 Significant expression of this gene is seen exclusively in lung cancer cell line (CT=3. 1). Therefore, expression of this gene may be used to distinguish specific lung cancer cell lines from the other samples on this panel. Furthermore, therapeutic modulation of the activity of the GPCR encoded by this gene may be beneficial in the treatment of lung cancer.

Panel 1.3D Summary: Ag1561 Expression of this gene is low/undetectable (CTs>35) across all of the samples on this panel (data not shown).

Panel 2D Summary: Ag1561 Expression of this gene is low/undetectable (CTs>35) across all of the samples on this panel (data not shown).

AB. CG50275-01: Olfactory Receptor

Expression of gene CG50275-01 was assessed using the primer-probe sets Ag2511 and Ag1704, described in Tables ABA and ABB. Results of the RTQ-PCR runs are shown in Table ABC.

TABLE ABA
Probe Name Ag2511

| | | | Start | SEQ ID |
|---|---|---|---|---|
| Primers | Sequences | Length | Position | NO |
| Forward | 5′-gcctccctcatcttctctctac-3′ | 22 | 475 | 244 |
| Probe | TET-5′-acccgatcatcccgcactttctct-3′-TAMRA | 24 | 509 | 245 |
| Reverse | 5′-ctcagtactggcaggatgtca-3′ | 21 | 534 | 246 |

TABLE ABB
Probe Name Ag1704

| | | | Start | SEQ ID |
|---|---|---|---|---|
| Primers | Sequences | Length | Position | NO |
| Forward | 5′-gcctccctcatcttctctctac-3′ | 22 | 465 | 247 |
| Probe | TET-5′-acccgatcatcccgcactttctct-3′-TAMRA | 24 | 509 | 248 |
| Reverse | 5′-ctcagtactggcaggatgtca-3′ | 21 | 534 | 249 |

TABLE ABC
Panel 4D

| | Rel. Exp. (%) | Rel. Exp. (%) | | Rel. Exp. (%) | Rel. Exp. (%) |
|---|---|---|---|---|---|
| | Ag1704, Run | Ag2511, Run | | Ag1704, Run | Ag2511, Run |
| Tissue Name | 164729522 | 164320651 | Tissue Name | 164729522 | 164320651 |
| Secondary Th1 act | 32.1 | 20.3 | HUVEC IL-1 beta | 0.0 | 0.0 |
| Secondary Th2 act | 21.2 | 0.0 | HUVEC IFN | 0.0 | 0.0 |
| | | | gamma | | |
| Secondary Tr1 act | 0.0 | 0.0 | HUVEC TNF alpha + | 0.0 | 0.0 |
| | | | IFN gamma | | |
| Secondary Th1 rest | 15.7 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 39.0 | 16.3 | Lung Microvascular | 0.0 | 0.0 |
| | | | EC none | | |
| Primary Th1 act | 0.0 | 0.0 | Lung Microvascular | 0.0 | 0.0 |
| | | | EC TNF alpha + IL- | | |
| | | | 1 beta | | |
| Primary Th2 act | 0.0 | 0.0 | Microvascular | 0.0 | 17.0 |
| | | | Dermal EC none | | |
| Primary Tr1 act | 17.0 | 0.0 | Microsvasular | 0.0 | 0.0 |
| | | | Dermal EC | | |
| | | | TNF alpha + IL- | | |
| | | | 1 beta | | |
| Primary Th1 rest | 0.0 | 0.0 | Bronchial | 0.0 | 0.0 |
| | | | epithelium | | |
| | | | TNF alpha + IL1 beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNF alpha + IL- | | |
| | | | 1 beta | | |
| CD45RA CD4 | 0.0 | 0.0 | Coronery artery | 0.0 | 0.0 |
| lymphocyte act | | | SMC rest | | |
| CD45RO CD4 | 0.0 | 15.3 | Coronery artery | 0.0 | 0.0 |
| lymphocyte act | | | SMC TNF alpha + | | |
| | | | IL-1 beta | | |
| CD8 lymphocyte act | 0.0 | 0.0 | Astrocytes rest | 0.0 | 0.0 |
| Secondary CD8 | 0.0 | 0.0 | Astrocytes | 0.0 | 0.0 |
| lymphocyte rest | | | TNF alpha + IL- | | |
| | | | 1 beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 11.6 | 0.0 |
| none | | | PMA/ionomycin | | |
| 2ry | 23.5 | 38.2 | CCD1106 | 0.0 | 0.0 |
| Th1/Th2/Tr1_anti- | | | (Keratinocytes) | | |
| CD95 CH11 | | | none | | |
| LAK cells rest | 42.0 | 32.3 | CCD1106 | 0.0 | 0.0 |
| | | | (Keratinocytes) | | |
| | | | TNF alpha + IL- | | |
| | | | 1 beta | | |
| LAK cells IL-2 | 0.0 | 0.0 | Liver cirrhosis | 85.9 | 76.3 |
| LAK cells IL-2 + IL- | 0.0 | 0.0 | Lupus kidney | 0.0 | 0.0 |
| 12 | | | | | |
| LAK cells IL-2 + IFN | 16.6 | 0.0 | NCI-H292 none | 0.0 | 0.0 |
| gamma | | | | | |
| LAK cells IL-2 + IL- | 15.0 | 0.0 | NCI-H292 IL-4 | 0.0 | 0.0 |
| 18 | | | | | |
| LAK cells | 100.0 | 100.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 | 27.7 | 0.0 | NCI-H292 IFN | 0.0 | 0.0 |
| day | | | gamma | | |
| Two Way MLR 5 | 0.0 | 0.0 | HPAEC none | 0.0 | 0.0 |
| day | | | | | |
| Two Way MLR 7 | 0.0 | 13.2 | HPAEC TNF | 0.0 | 0.0 |
| day | | | alpha + IL-1 beta | | |
| PBMC rest | 0.0 | 0.0 | Lung fibroblast none | 0.0 | 0.0 |
| PBMC PWM | 0.0 | 44.4 | Lung fibroblast TNF | 0.0 | 0.0 |
| | | | alpha + IL-1 beta | | |
| PBMC PHA-L | 15.2 | 13.4 | Lung fibroblast IL-4 | 0.0 | 0.0 |
| Ramos (B cell) none | 0.0 | 0.0 | Lung fibroblast IL-9 | 0.0 | 0.0 |
| Ramos (B cell) | 0.0 | 0.0 | Lung fibroblast IL- | 0.0 | 0.0 |
| ionomycin | | | 13 | | |
| B lymphocytes | 0.0 | 0.0 | Lung fibroblast IFN | 0.0 | 0.0 |
| PWM | | | gamma | | |
| B lymphocytes | 0.0 | 15.9 | Dermal fibroblast | 23.8 | 0.0 |
| CD40L and IL-4 | | | CCD1070 rest | | |
| EOL-1 dbcAMP | 0.0 | 0.0 | Dermal fibroblast | 17.2 | 0.0 |
| | | | CCD1070 TNF | | |
| | | | alpha | | |
| EOL-1 dbcAMP | 0.0 | 0.0 | Dermal fibroblast | 0.0 | 0.0 |
| PMA/ionomycin | | | CCD1070 IL-1 beta | | |
| Dendritic cells none | 15.1 | 0.0 | Dermal fibroblast | 0.0 | 0.0 |
| | | | IFN gamma | | |
| Dendritic cells LPS | 0.0 | 0.0 | Dermal fibroblast | 0.0 | 0.0 |
| | | | IL-4 | | |
| Dendritic cells anti- | 0.0 | 0.0 | IBD Colitis 2 | 26.8 | 28.1 |
| CD40 | | | | | |
| Monocytes rest | 0.0 | 0.0 | IBD Crohn's | 0.0 | 14.6 |
| Monocytes LPS | 26.4 | 17.4 | Colon | 38.4 | 0.0 |
| Macrophages rest | 0.0 | 0.0 | Lung | 11.5 | 55.5 |
| Macrophages LPS | 0.0 | 0.0 | Thymus | 0.0 | 28.7 |
| HUVEC none | 0.0 | 0.0 | Kidney | 0.0 | 0.0 |
| HUVEC starved | 0.0 | 0.0 | | | |

CNS_neurodegeneration_v1.0 Summary: Ag2511 Expression of the CG50275-01 gene is low/undetectable (CTs>35) across all of the samples on this panel (data not shown).

Panel 1.3D Summary: Ag1704/Ag2511 Expression of the CG50275-01 gene is low/undetectable (CTs>35) across all of the samples on this panel (data not shown).

Panel 2.2 Summary: Ag1704 Expression of the CG50275-01 gene is low/undetectable (CTs>35) across all of the samples on this panel (data not shown).

Panel 4D Summary: ag1704/2511 Two experiments both show highest expression of the CG50275-01 gene in LAK cells treated with PMA/ionomycin (CT=32-34). Significant expression of this gene is also seen in liver cirrhosis. Normal liver does not express this transcript (see panel 1.3 and 2.2) suggesting that expression may be specific to cirrhosis. Thus, the transcript or the protein encoded by the transcript could be used diagnostically to identify liver cirrhosis or LAK cells. Furthermore, the protein encoded for by this transcript could be used to design therapeutics against liver cirrhosis. The high expression in LAK cells suggests that the putative GPCR encoded by this transcript could also be important in the function of LAK cells. LAK cells are important for immunosurveillance against bacterial and viral infected cells as well as transformed cells. Thus, therapeutics that enhance LAK activity and serve as treatments for viral and bacterial diseases and cancer could potentially be designed with this gene product.

AC. CG55970-02: GPCR

Expression of gene CG55970-02 was assessed using the primer-probe set Ag5095, described in Table ACA. Results of the RTQ-PCR runs are shown in Tables ACB, and ACC.

TABLE ACA
Probe Name Ag5095

| | | | Start | SEQ ID |
|---|---|---|---|---|
| Primers | Sequences | Length | Position | NO |
| Forward | 5′-ttctgctgctgctttatgct-3′ | 20 | 89 | 250 |
| Probe | TET-5′-cctgggcaacatcctcatcctcttta-3′-TAMRA | 26 | 117 | 251 |
| Reverse | 5′-gcaagctctgctcttccttt-3′ | 20 | 147 | 252 |

TABLE ACB
General_screening_panel_v1.5

| | Rel. Exp.(%) | Rel. Exp.(%) | | Rel. Exp.(%) | Rel. Exp.(%) |
|---|---|---|---|---|---|
| | Ag5095, Run | Ag5095, Run | | Ag5095, Run | Ag5095, Run |
| Tissue Name | 228727262 | 229384819 | Tissue Name | 228727262 | 229384819 |
| Adipose | 0.1 | 0.1 | Renal ca. TK-10 | 0.1 | 0.0 |
| Melanoma* | 0.0 | 0.0 | Bladder | 0.4 | 0.4 |
| Hs688(A).T | | | | | |
| Melanoma* | 0.0 | 0.0 | Gastric ca. (liver | 0.2 | 0.2 |
| Hs688(B).T | | | met.) NCI-N87 | | |
| Melanoma* | 0.2 | 0.1 | Gastric ca. KATO | 0.2 | 0.3 |
| M14 | | | III | | |
| Melanoma* | 2.0 | 1.5 | Colon ca. SW-948 | 0.0 | 0.0 |
| LOXIMVI | | | | | |
| Melanoma* | 23.3 | 24.3 | Colon ca. SW480 | 0.2 | 0.2 |
| SK-MEL-5 | | | | | |
| Squamous cell | 0.0 | 0.0 | Colon ca.* | 1.6 | 1.1 |
| carcinoma | | | (SW480 met) | | |
| SCC-4 | | | SW620 | | |
| Testis Pool | 0.2 | 0.2 | Colon ca. HT29 | 0.0 | 0.0 |
| Prostate ca.* | 0.0 | 0.0 | Colon ca. HCT- | 100.0 | 100.0 |
| (bone met) PC-3 | | | 116 | | |
| Prostate Pool | 0.1 | 0.2 | Colon ca. CaCo-2 | 0.1 | 0.0 |
| Placenta | 0.0 | 0.1 | Colon cancer | 0.0 | 0.1 |
| | | | tissue | | |
| Uterus Pool | 0.2 | 0.1 | Colon ca. SW1116 | 0.0 | 0.0 |
| Ovarian ca. | 0.1 | 0.1 | Colon ca. Colo-205 | 0.0 | 0.0 |
| OVCAR-3 | | | | | |
| Ovarian ca. | 0.5 | 0.7 | Colon ca. SW-48 | 0.0 | 0.0 |
| SK-OV-3 | | | | | |
| Ovarian ca. | 0.0 | 0.0 | Colon Pool | 0.7 | 0.5 |
| OVCAR-4 | | | | | |
| Ovarian ca. | 0.0 | 0.0 | Small Intestine | 0.6 | 1.1 |
| OVCAR-5 | | | Pool | | |
| Ovarian ca. | 0.0 | 0.0 | Stomach Pool | 0.3 | 0.3 |
| IGROV-1 | | | | | |
| Ovarian ca. | 0.0 | 0.0 | Bone Marrow Pool | 0.3 | 0.2 |
| OVCAR-8 | | | | | |
| Ovary | 0.2 | 0.3 | Fetal Heart | 0.3 | 0.3 |
| Breast ca. | 0.0 | 0.0 | Heart Pool | 0.2 | 0.1 |
| MCF-7 | | | | | |
| Breast ca. | 0.0 | 0.0 | Lymph Node Pool | 0.5 | 0.6 |
| MDA-MB-231 | | | | | |
| Breast ca. BT | 0.0 | 0.0 | Fetal Skeletal | 0.1 | 0.1 |
| 549 | | | Muscle | | |
| Breast ca. | 0.0 | 0.0 | Skeletal Muscle | 0.1 | 0.0 |
| T47D | | | Pool | | |
| Breast ca. | 2.2 | 3.0 | Spleen Pool | 0.1 | 0.1 |
| MDA-N | | | | | |
| Breast Pool | 0.6 | 0.4 | Thymus Pool | 0.3 | 0.4 |
| Trachea | 0.1 | 0.2 | CNS cancer | 0.0 | 0.0 |
| | | | (glio/astro) U87- | | |
| | | | MG | | |
| Lung | 0.3 | 0.2 | CNS cancer | 0.2 | 0.2 |
| | | | (glio/astro) U-118- | | |
| | | | MG | | |
| Fetal Lung | 0.4 | 0.7 | CNS cancer | 0.0 | 0.0 |
| | | | (neuro;met) SK-N- | | |
| | | | AS | | |
| Lung ca. NCI-N417 | 0.0 | 0.0 | CNS cancer (astro) | 0.0 | 0.0 |
| | | | SF-539 | | |
| Lung ca. LX-1 | 16.7 | 19.5 | CNS cancer (astro) | 0.0 | 0.0 |
| | | | SNB-75 | | |
| Lung ca. NCI- | 0.1 | 0.0 | CNS cancer (glio) | 0.0 | 0.0 |
| H146 | | | SNB-19 | | |
| Lung ca. SHP- | 0.3 | 0.1 | CNS cancer (glio) | 0.3 | 0.2 |
| 77 | | | SF-295 | | |
| Lung ca. A549 | 0.1 | 0.0 | Brain (Amygdala) | 0.0 | 0.0 |
| | | | Pool | | |
| Lung ca. NCI- | 0.0 | 0.0 | Brain (cerebellum) | 0.0 | 0.0 |
| H526 | | | | | |
| Lung ca. NCI- | 0.0 | 0.0 | Brain (fetal) | 0.1 | 0.1 |
| H23 | | | | | |
| Lung ca. NCI- | 0.9 | 0.0 | Brain | 0.0 | 0.0 |
| H460 | | | (Hippocampus) | | |
| | | | Pool | | |
| Lung ca. HOP- | 0.0 | 0.0 | Cerebral Cortex | 0.0 | 0.0 |
| 62 | | | Pool | | |
| Lung ca. NCI- | 0.0 | 0.0 | Brain (Substantia | 0.0 | 0.0 |
| H522 | | | nigra) Pool | | |
| Liver | 0.0 | 0.0 | Brain (Thalamus) | 0.1 | 0.1 |
| | | | Pool | | |
| Fetal Liver | 0.0 | 0.1 | Brain (whole) | 0.1 | 0.1 |
| Liver ca. | 0.0 | 0.0 | Spinal Cord Pool | 0.0 | 0.1 |
| HepG2 | | | | | |
| Kidney Pool | 0.4 | 0.9 | Adrenal Gland | 0.1 | 0.1 |
| Fetal Kidney | 0.7 | 0.5 | Pituitary gland | 0.0 | 0.1 |
| | | | Pool | | |
| Renal ca. 786-0 | 0.0 | 0.0 | Salivary Gland | 0.0 | 0.0 |
| Renal ca. A498 | 0.0 | 0.0 | Thyroid (female) | 0.0 | 0.0 |
| Renal ca. | 0.0 | 0.1 | Pancreatic ca. | 0.0 | 0.0 |
| ACHN | | | CAPAN2 | | |
| Renal ca. UO-31 | 0.0 | 0.0 | Pancreas Pool | 0.5 | 0.6 |

TABLE ACC
Panel 4.1D

| | Rel. Exp.(%) Ag5095, | | Rel. Exp.(%) Ag5095, |
|---|---|---|---|
| Tissue Name | Run 225001774 | Tissue Name | Run 225001774 |
| Secondary Th1 act | 0.0 | HUVEC IL-1beta | 0.0 |
| Secondary Th2 act | 0.0 | HUVEC IFN gamma | 0.9 |
| Secondary Tr1 act | 0.2 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.6 |
| Primary Th1 act | 0.0 | Lung Microvascular EC | 0.0 |
| | | TNFalpha + IL-1beta | |
| Primary Th2 act | 0.3 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.7 | Bronchial epithelium | 0.0 |
| | | TNFalpha + IL1beta | |
| Primary Th2 rest | 0.5 | Small airway epithelium none | 0.0 |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.8 | Coronery artery SMC | 0.4 |
| lymphocyte act | | TNFalpha + IL-1beta | |
| CD8 lymphocyte act | 0.0 | Astrocytes rest | 0.8 |
| Secondary CD8 | 0.2 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.8 |
| | | PMA/ionomycin | |
| 2ry Th1/Th2/Tr1_anti- | 1.0 | CCD1106 (Keratinocytes) | 0.0 |
| CD95 CH11 | | none | |
| LAK cells rest | 0.3 | CCD1106 (Keratinocytes) | 0.0 |
| | | TNFalpha + IL-1beta | |
| LAK cells IL-2 | 1.2 | Liver cirrhosis | 0.0 |
| LAK cells IL-2 + IL-12 | 0.6 | NCI-H292 none | 0.4 |
| LAK cells IL-2 + IFN | 0.0 | NCI-H292 IL-4 | 0.0 |
| gamma | | | |
| LAK cells IL-2 + IL-18 | 0.2 | NCI-H292 IL-9 | 0.0 |
| LAK cells | 0.0 | NCI-H292 IL-13 | 0.0 |
| PMA/ionomycin | | | |
| NK Cells IL-2 rest | 0.6 | NCI-H292 IFN gamma | 0.0 |
| Two Way MLR 3 day | 1.8 | HPAEC none | 0.0 |
| Two Way MLR 5 day | 1.1 | HPAEC TNF alpha + IL-1 | 0.0 |
| | | beta | |
| Two Way MLR 7 day | 0.0 | Lung fibroblast none | 0.0 |
| PBMC rest | 0.8 | Lung fibroblast TNF alpha + | 0.8 |
| | | IL-1 beta | |
| PBMC PWM | 0.0 | Lung fibroblast IL-4 | 0.4 |
| PBMC PHA-L | 0.0 | Lung fibroblast IL-9 | 0.0 |
| Ramos (B cell) none | 17.0 | Lung fibroblast IL-13 | 1.2 |
| Ramos (B cell) | 13.2 | Lung fibroblast IFN gamma | 0.4 |
| ionomycin | | | |
| B lymphocytes PWM | 0.3 | Dermal fibroblast CCD1070 | 0.0 |
| | | rest | |
| B lymphocytes CD40L | 0.7 | Dermal fibroblast CCD1070 | 0.0 |
| and IL-4 | | TNF alpha | |
| EOL-1 dbcAMP | 0.0 | Dermal fibroblast CCD1070 | 0.0 |
| | | IL-1beta | |
| EOL-1 dbcAMP | 0.0 | Dermal fibroblast IFN gamma | 1.4 |
| PMA/ionomycin | | | |
| Dendritic cells none | 0.0 | Dermal fibroblast IL-4 | 0.0 |
| Dendritic cells LPS | 0.3 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 2.0 |
| Monocytes LPS | 0.8 | Colon | 1.6 |
| Macrophages rest | 1.4 | Lung | 4.6 |
| Macrophages LPS | 0.0 | Thymus | 9.4 |
| HUVEC none | 0.2 | Kidney | 100.0 |
| HUVEC starved | 0.4 | | |

CNS_neurodegeneration_v1.0 Summary: Ag5095 Expression low/undetectable in all samples on this panel. (Data not shown.)

General_screening panel_v1.5 Summary: Ag5095 The expression of this gene appears to be highest in a samples derived from a colon cancer cell line (HCT 11 6). In addition there appears to be expression in a lung cancer cell line (LX-1) and a melanoma cell line (SK-Mel-5). Thus, the expression of this gene could be used to distinguish samples derived from these cell lines when compared to the other samples in the panel. Moreover, therapeutic modulation of this gene, through the use of antibodies, small molecule drugs or protein therapeutics might be of benefit in the treatment of colon cancer, lung cancer or melanoma.

Among tissues with metabolic activity, this gene is expressed at low levels in pancreas and fetal heart. Therefore, the GPCR encoded by this gene may play a role in cardiovascular diseases and/or metabolic diseases, such as diabetes and obesity.

Low expression is also seen in a number of other normal tissues including thymus, lymph node, bone marrow, small intestine, stomach, colon, bladder, lung, breast, and ovary (CTs=31-35).

Panel 4.1D Summary: Ag5095 Expression of this gene is highest in kidney (CT=30). Therefore, the putative GPCR encoded for by this gene could allow cells within the kidney to respond to specific microenvironmental signals. Thus, antibody or small molecule therapies designed with the protein encoded for by this gene could modulate kidney function and be important in the treatment of inflammatory or autoimmune diseases that affect the kidney, including lupus and glomerulonephritis.

In addition, this gene is expressed at low levels in Ramos B cells (CT=33), consistent with what is observed in Panel 4D. Expression of this transcript in B cells suggests that this gene may be involved in rheumatic disease including rheumatoid arthritis, lupus, osteoarthritis, and hyperproliferative B cell disorders.

AD. CG56119-01: Olfactory Receptor

Expression of gene CG56119-01 was assessed using the primer-probe set Ag2200, described in Table ADA. Results of the RTQ-PCR runs are shown in Table ADB.

TABLE ADA
Probe Name Ag2200

| | | | Start | SEQ ID |
|---|---|---|---|---|
| Primers | Sequences | Length | Position | NO |
| Forward | 5′-tatttcatcctgctgggattct-3′ | 22 | 37 | 253 |
| Probe | TET-5′-tcccaggatcataaaagtgctcttca-3′-TAMRA | 26 | 66 | 254 |
| Reverse | 5′-tccaggccagagatgtaatgta-3′ | 22 | 109 | 255 |

TABLE ABD
Panel 2D

| | Rel. Exp.(%) Ag2200, | | Rel. Exp.(%) Ag2200, |
|---|---|---|---|
| Tissue Name | Run 164025299 | Tissue Name | Run 164025299 |
| Normal Colon | 0.1 | Kidney Margin 8120608 | 0.0 |
| CC Well to Mod Diff | 1.3 | Kidney Cancer 8120613 | 0.0 |
| (ODO3866) | | | |
| CC Margin (ODO3866) | 0.0 | Kidney Margin 8120614 | 0.0 |
| CC Gr.2 rectosigmoid | 0.0 | Kidney Cancer 9010320 | 0.2 |
| (ODO3868) | | | |
| CC Margin (ODO3868) | 0.0 | Kidney Margin 9010321 | 0.0 |
| CC Mod Diff (ODO3920) | 0.0 | Normal Uterus | 0.0 |
| CC Margin (ODO3920) | 0.0 | Uterine Cancer 064011 | 0.0 |
| CC Gr.2 ascend colon | 0.0 | Normal Thyroid | 0.0 |
| (ODO3921) | | | |
| CC Margin (ODO3921) | 0.2 | Thyroid Cancer | 2.8 |
| CC from Partial | 0.2 | Thyroid Cancer | 100.0 |
| Hepatectomy (ODO4309) | | A302152 | |
| Mets | | | |
| Liver Margin (ODO4309) | 0.8 | Thyroid Margin | 0.0 |
| | | A302153 | |
| Colon mets to lung | 0.4 | Normal Breast | 0.0 |
| (OD04451-01) | | | |
| Lung Margin (OD04451-02) | 0.0 | Breast Cancer | 0.4 |
| Normal Prostate 6546-1 | 0.0 | Breast Cancer | 0.4 |
| | | (OD04590-01) | |
| Prostate Cancer (OD04410) | 0.0 | Breast Cancer Mets | 0.0 |
| | | (OD04590-03) | |
| Prostate Margin (OD04410) | 0.0 | Breast Cancer | 0.0 |
| | | Metastasis | |
| Prostate Cancer (OD04720- | 0.0 | Breast Cancer | 0.0 |
| 01) | | | |
| Prostate Margin (OD04720- | 0.0 | Breast Cancer | 0.0 |
| 02) | | | |
| Normal Lung | 0.6 | Breast Cancer 9100266 | 0.0 |
| Lung Met to Muscle | 0.3 | Breast Margin 9100265 | 0.0 |
| (ODO4286) | | | |
| Muscle Margin (ODO4286) | 0.0 | Breast Cancer A209073 | 0.0 |
| Lung Malignant Cancer | 0.0 | Breast Margin A2090734 | 0.2 |
| (OD03126) | | | |
| Lung Margin (OD03126) | 0.0 | Normal Liver | 0.8 |
| Lung Cancer (OD04404) | 0.3 | Liver Cancer | 0.4 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 0.1 | Liver Cancer 6004-T | 0.6 |
| Lung Cancer (OD04237-01) | 0.0 | Liver Tissue 6004-N | 0.2 |
| Lung Margin (OD04237-02) | 0.0 | Liver Cancer 6005-T | 0.0 |
| Ocular Mel Met to Liver | 0.0 | Liver Tissue 6005-N | 0.0 |
| (ODO4310) | | | |
| Liver Margin (ODO4310) | 0.0 | Normal Bladder | 0.0 |
| Melanoma Metastasis | 0.2 | Bladder Cancer | 0.2 |
| Lung Margin (OD04321) | 0.0 | Bladder Cancer | 1.6 |
| Normal Kidney | 0.0 | Bladder Cancer | 0.0 |
| | | (OD04718-01) | |
| Kidney Ca, Nuclear grade 2 | 10.5 | Bladder Normal | 0.0 |
| (OD04338) | | Adjacent (OD04718-03) | |
| Kidney Margin (OD04338) | 0.0 | Normal Ovary | 0.0 |
| Kidney Ca Nuclear grade 1/2 | 0.4 | Ovarian Cancer | 0.0 |
| (OD04339) | | | |
| Kidney Margin (OD04339) | 0.0 | Ovarian Cancer | 0.0 |
| | | (OD04768-07) | |
| Kidney Ca, Clear cell type | 0.0 | Ovary Margin | 0.0 |
| (OD04340) | | (OD04768-08) | |
| Kidney Margin (OD04340) | 0.2 | Normal Stomach | 0.3 |
| Kidney Ca, Nuclear grade 3 | 0.0 | Gastric Cancer 9060358 | 0.0 |
| (OD04348) | | | |
| Kidney Margin (OD04348) | 0.3 | Stomach Margin | 0.3 |
| | | 9060359 | |
| Kidney Cancer (OD04622- | 0.8 | Gastric Cancer 9060395 | 0.0 |
| 01) | | | |
| Kidney Margin (OD04622- | 0.0 | Stomach Margin | 0.0 |
| 03) | | 9060394 | |
| Kidney Cancer (OD04450- | 0.0 | Gastric Cancer 9060397 | 0.0 |
| 01) | | | |
| Kidney Margin (OD04450- | 0.0 | Stomach Margin | 0.0 |
| 03) | | 9060396 | |
| Kidney Cancer 8120607 | 0.0 | Gastric Cancer 064005 | 0.5 |

CNS_neurodegeneration_v1.0 Summary: Ag2200 Expression of this gene is low/undetectable (CTs>35) across all of the samples on this panel (data not shown).

Panel 1.3D Summary: Ag2200 Expression of this gene is low/undetectable (CTs>35) across all of the samples on this panel (data not shown).

Panel 2D Summary: Ag2200 This gene is most highly expressed in a thyroid cancer sample (CT=29). Interestingly, expression of this gene is not detectable in the matched adjacent normal thyroid tissue. This gene is also expressed at low but significant levels in an additional thyroid tumor. Therefore, expression of this gene may be used to distinguish thyroid cancer from normal thyroid tissue. Furthermore, therapeutic modulation of the activity of the GPCR encoded by this gene may be beneficial in the treatment of thyroid cancer.

Panel 3D Summary: Ag2200 Expression of this gene is low/undetectable (CTs>35) across all of the samples on this panel (data not shown).

Panel 4D Summary: Ag2200 Expression of this gene is low/undetectable (CTs>35) across all of the samples on this panel (data not shown).

AE. CG50155-01/GMAL359218_D_dal: Olfactory Receptor

Expression of gene CG50155-01 was assessed using the primer-probe sets Ag1575, Ag2464 and Ag2465, described in Tables AEA, AEB and AEC. Results of the RTQ-PCR runs are shown in Table AED.

TABLE AEA
Probe Name Ag1575

| Primers | Sequences | Length | Start Position | SEQ ID NO |
|---|---|---|---|---|
| Forward | 5′-aacctagctttcctggacatgt-3′ | 22 | 208 | 256 |
| Probe | TET-5′-tcatttgccactcccaagatgatcag-3′-TAMRA | 26 | 238 | 257 |
| Reverse | 5′-acatcctccaaaggagatgagt-3′ | 22 | 285 | 258 |

TABLE AEB
Probe Name Ag2464

| Primers | Sequences | Length | Start Position | SEQ ID NO |
|---|---|---|---|---|
| Forward | 5′-cagaatttgtgttgcatgga-3′ | 20 | 41 | 259 |
| Probe | TET-5′-ctctgcacttcacgacatcttcaaaa-3′-TAMRA | 26 | 61 | 260 |
| Reverse | 5′-ccagcataatggccacatag-3′ | 20 | 114 | 261 |

TABLE AEC
Probe Name Ag2465

| Primers | Sequences | Length | Start Position | SEQ ID NO |
|---|---|---|---|---|
| Forward | 5′-cagaatttgtgttgcatgga-3′ | 20 | 41 | 262 |
| Probe | TET-5′-ctctgcacttcacgacatcttcaaaa-3′-TAMRA | 26 | 61 | 263 |
| Reverse | 5′-ccagcataatggccacatag-3′ | 20 | 114 | 264 |

TABLE AED
Panel 4D

| | Rel. Exp.(%) Ag1575, | | Rel. Exp.(%) Ag1575, |
|---|---|---|---|
| Tissue Name | Run 165725924 | Tissue Name | Run 165725924 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFaplha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.9 | Liver cirrhosis | 25.2 |
| LAK cells IL-2 + IL-12 | 100.0 | Lupus kidney | 0.0 |
| LAK cells IL-2 + IFN | 0.0 | NCI-H292 none | 2.2 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.6 |
| | | beta | |
| PBMC rest | 0.5 | Lung fibroblast none | 1.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 1.6 |
| Monocytes rest | 1.1 | IBD Crohn's | 0.0 |
| Monocytes LPS | 0.0 | Colon | 0.8 |
| Macrophages rest | 0.0 | Lung | 0.0 |
| Macrophages LPS | 2.9 | Thymus | 0.0 |
| HUVEC none | 0.0 | Kidney | 0.0 |
| HUVEC starved | 0.0 | | |

CNS_neurodegeneration_v1.0 Summary: Ag1575/Ag2464/Ag2465 Expression of the CG50155-01 gene is low/undetectable (CTs>35) across all of the samples on this panel (data not shown).

Panel 1.3D Summary: Ag1575/Ag2464/Ag2465 Expression of the CG50155-01 gene is low/undetectable (CTs>35) across all of the samples on this panel (data not shown).

Panel 2.2 Summary: Ag1575 Expression of the CG50155-01 gene is low/undetectable (CTs>35) across all of the samples on this panel (data not shown).

Panel 4D Summary: Ag1575 Expression of this gene is highest in lymphokine-activated killer (LAK cells) treated with IL-2 and IL-12 (CT=31.8). Since these cells are involved in tumor immunology and tumor cell clearance, as well as virally and bacterial infected cells. Therefore, modulation of the activity of this gene or its protein product with a small molecule drug or antibody may alter the functions of these cells and lead to improvement of symptoms associated with these conditions.

In addition, low expression is also detected in a liver cirrhosis sample. Furthermore, no expression in normal liver is seen in Panel 1.3D, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

Ag2464/Ag2465 Expression of the CG50155-01 gene is low/undetectable (CTs>35) across all of the samples on this panel (data not shown).

AF. CG56880-01: Olfactory Receptor

Expression of gene CG56880-01 was assessed using the primer-probe set Ag1509, described in Table AFA. Results of the RTQ-PCR runs are shown in Table AFB.

TABLE AFA
Probe Name Ag1509

| | | | Start | |
|---|---|---|---|---|
| Primers | Sequences | Length | Position | SEQ ID NO |
| Forward | 5′-aattgctcaaactatcctgcaa-3′ | 22 | 554 | 265 |
| Probe | TET-5′-tcacggagtttatcctcttcttaatggctg-3′-TAMRA | 30 | 587 | 266 |
| Reverse | 5′-agggatcaaagaaccaaagaga-3′ | 22 | 624 | 267 |

TABLE AFB
Panel 1.2

| | Rel. Exp.(%) Ag1509, | | Rel. Exp.(%) Ag1509, |
|---|---|---|---|
| Tissue Name | Run 141961835 | Tissue Name | Run 141961835 |
| Endothelial cells | 0.0 | Renal ca. 786-0 | 1.5 |
| Heart (Fetal) | 1.3 | Renal ca. A498 | 5.1 |
| Pancreas | 0.5 | Renal ca. RXF 393 | 0.0 |
| Pancreatic ca. CAPAN 2 | 0.2 | Renal ca. ACHN | 2.5 |
| Adrenal Gland | 4.9 | Renal ca. UO-31 | 3.8 |
| Thyroid | 0.5 | Renal ca. TK-10 | 58.6 |
| Salivary gland | 13.7 | Liver | 5.9 |
| Pituitary gland | 0.5 | Liver (fetal) | 3.3 |
| Brain (fetal) | 2.5 | Liver ca. (hepatoblast) | 16.8 |
| | | HepG2 | |
| Brain (whole) | 2.2 | Lung | 0.0 |
| Brain (amygdala) | 5.9 | Lung (fetal) | 1.0 |
| Brain (cerebellum) | 3.2 | Lung ca. (small cell) | 11.0 |
| | | LX-1 | |
| Brain (hippocampus) | 20.4 | Lung ca. (small cell) | 35.4 |
| | | NCI-H69 | |
| Brain (thalamus) | 5.1 | Lung ca. (s. cell var.) | 2.5 |
| | | SHP-77 | |
| Cerebral Cortex | 17.7 | Lung ca. (large | 7.5 |
| | | cell) NCI-H460 | |
| Spinal cord | 0.7 | Lung ca. (non-sm. cell) | 5.7 |
| | | A549 | |
| glio/astro U87-MG | 0.6 | Lung ca. (non-s. cell) | 61.1 |
| | | NCI-H23 | |
| glio/astro U-118-MG | 2.3 | Lung ca. (non-s. cell) | 18.2 |
| | | HOP-62 | |
| astrocytoma SW1783 | 1.3 | Lung ca. (non-s. cl) | 0.0 |
| | | NCI-H522 | |
| neuro*; met SK-N-AS | 0.0 | Lung ca. (squam.) SW | 6.9 |
| | | 900 | |
| astrocytoma SF-539 | 0.7 | Lung ca. (squam.) NCI- | 11.4 |
| | | H596 | |
| astrocytoma SNB-75 | 2.3 | Mammary gland | 4.8 |
| glioma SNB-19 | 37.1 | Breast ca.* (pl. ef) | 18.3 |
| | | MCF-7 | |
| glioma U251 | 1.1 | Breast ca.* (pl. ef) | 0.7 |
| | | MDA-MB-231 | |
| glioma SF-295 | 0.2 | Breast ca.* (pl. ef) | 4.0 |
| | | T47D | |
| Heart | 11.8 | Breast ca. BT-549 | 4.1 |
| Skeletal Muscle | 4.9 | Breast ca MDA-N | 11.5 |
| Bone marrow | 6.5 | Ovary | 0.0 |
| Thymus | 0.5 | Ovarian ca. OVCAR-3 | 0.7 |
| Spleen | 0.5 | Ovarian ca. OVCAR-4 | 43.8 |
| Lymph node | 0.0 | Ovarian ca. OVCAR-5 | 27.0 |
| Colorectal | 2.0 | Ovarian ca. OVCAR-8 | 34.6 |
| Stomach | 0.5 | Ovarian ca. IGROV-1 | 20.3 |
| Small intestine | 7.3 | Ovarian ca. (ascites) | 24.8 |
| | | SK-OV-3 | |
| Colon ca. SW480 | 0.6 | Uterus | 3.1 |
| Colon ca.* SW620 | 0.3 | Placenta | 1.2 |
| (SW480 met) | | | |
| Colon ca. HT29 | 4.1 | Prostate | 18.6 |
| Colon ca. HCT-116 | 13.6 | Prostate ca.* (bone | 0.5 |
| | | met) PC-3 | |
| Colon ca. CaCo-2 | 0.5 | Testis | 6.8 |
| CC Well to Mod Diff | 8.0 | Melanoma Hs688(A).T | 0.0 |
| (ODO3866) | | | |
| Colon ca. HCC-2998 | 10.2 | Melanoma* (met) | 6.2 |
| | | Hs688(B).T | |
| Gastric ca. (liver met) | 9.1 | Melanoma UACC-62 | 0.0 |
| NCI-N87 | | | |
| Bladder | 23.8 | Melanoma M14 | 33.7 |
| Trachea | 0.4 | Melanoma LOX IMVI | 1.2 |
| Kidney | 100.0 | Melanoma* (met) SK- | 1.8 |
| | | MEL-5 | |
| Kidney (fetal) | 16.5 | | |

Panel 1.2 Summary: Ag1509 Highest expression of this gene is seen in the normal kidney (CT=30.1). Overall, however, this gene appears to show a higher association in cell lines derived from cancers than in normal tissues. There is significant expression in a cluster of cell lines derived form ovarian, lung and colon cancers. Thus, expression of this gene could be used to differentiate between these samples and other samples on this panel. Furthermore, expression of this gene could potentially be used as a marker for ovarian, colon or lung cancers.

Among tissues with metabolic function, this GPCR homolog is expressed at low but significant levels in the heart (CT=33.2). Furthermore, this gene is expressed at higher levels in the adult heart when compared to expression in the fetal heart (CT=36.4). Thus, expression of this gene could also be used to differentiate between adult and fetal source of this tissue.

This gene represents a novel G-protein coupled receptor (GPCR) that also shows expression in the brain, including the amygdala, hippocampus, thalamus and cerebral cortex. The GPCR family of receptors contains a large number of neurotransmitter receptors, including the dopamine, serotonin, a and b-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT IA and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore, this gene may be of use as a small molecule target for the treatment of any of the described diseases.

### REFERENCES

El Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol September 2001;134(1):68-77.

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCCH 58261 at 10 mg kg(−1) reduced the immobility time by 61%, while KW 6002 decreased the total immobility time at the doses of 1 and 10 mg kg(−1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15:12-7.

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSR1 treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alphal-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini M E, Reggiani A. Glycine-site antagonists and stroke. Expert Opin Investig Drugs November 1999;8(11):1837-1848.

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport Dec. 1, 1998;9(17):3955-9 Related Articles, Books, LinkOut.

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

AG. CG57423-01: Olfactory Receptor

Expression of gene CG57423-01 was assessed using the primer-probe set Ag1741, described in Table AGA. Results of the RTQ-PCR runs are shown in Tables AGB, and AGC.

TABLE AGA
Probe Name Ag1741

| | | | Start | |
|---|---|---|---|---|
| Pimers | Sequences | Length | Position | SEQ ID NO |
| Forward | 5′-gctcttctccctctcaattgtt-3′ | 22 | 639 | 268 |
| Probe | TET-5′-catgtttattctagtggccattctcage-3′-TAMRA | 28 | 672 | 269 |
| Reverse | 5′-tgtacctccctttccttgagtt-3′ | 22 | 703 | 270 |

TABLE AGB
Panel 1.3D

| | Rel. Exp.(%) Ag1741, | | Rel. Exp.(%) Ag1741, |
|---|---|---|---|
| Tissue Name | Run 165974806 | Tissue Name | Run 165974806 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 13.6 | Renal ca. A498 | 0.0 |
| Adrenal gland | 0.0 | Renal ca. RXF 393 | 13.2 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 0.0 | Lung ca. (large | 0.0 |
| | | cell) NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl. ef) | 0.0 |
| | | MCF-7 | |
| glioma SF-295 | 0.0 | Breast ca.* (pl. ef) | 0.0 |
| | | MDA-MB-231 | |
| Heart (Fetal) | 0.0 | Breast ca.* (pl. ef) | 0.0 |
| | | T47D | |
| Heart | 0.0 | Breast ca. BT-549 | 0.0 |
| Skeletal muscle (Fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 100.0 | Ovarian ca. OVCAR-5 | 0.0 |
| Lymph node | 0.0 | Ovarian ca. OVCAR-8 | 0.0 |
| Colorectal | 5.9 | Ovarian ca. IGROV-1 | 0.0 |
| Stomach | 0.0 | Ovarian ca. (ascites) | 25.5 |
| | | SK-OV-3 | |
| Small intestine | 0.0 | Uterus | 0.0 |
| Colon ca. SW480 | 0.0 | Placenta | 0.0 |
| Colon ca.* SW620 | 0.0 | Prostate | 0.0 |
| (SW480 met) | | | |
| Colon ca. HT29 | 0.0 | Prostate ca.* (bone | 0.0 |
| | | met) PC-3 | |
| Colon ca. HCT-116 | 0.0 | Testis | 0.0 |
| Colon ca. CaCo-2 | 0.0 | Melanoma Hs688(A).T | 0.0 |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 0.0 |
| Gastric ca. (liver met) | 8.4 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE AGC
Panel 4D

| | Rel. Exp.(%) Ag1741, | | Rel. Exp.(%) Ag1741, |
|---|---|---|---|
| Tissue Name | Run 165807997 | Tissue Name | Run 165807997 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.6 | IBD Colitis 2 | 24.8 |
| Monocytes rest | 0.0 | IBD Crohn's | 10.8 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

Panel 1.3D Summary: Ag1741 The CG57423-01 gene is only expressed in the spleen (CT=34.2), an important site of secondary immune responses. Therefore, antibodies or small molecule therapeutics that block the function of this GPCR may be useful as anti-inflammatory therapeutics for the treatment of allergies, autoimmune diseases, and inflammatory diseases.

Panel 2.2 Summary: Ag1741 Expression of this gene is low/undetectable (CTs>35) across all of the samples on this panel (data not shown).

Panel 4D Summary: Ag1741 The CG57423-01 transcript is only detected in liver cirrhosis (CT=33.1). Furthermore, this transcript is not detected in normal liver in Panel 1.3D, suggesting that the CG57423-01 gene expression is unique to liver cirrhosis. The CG57423-01 gene encodes a putative GPCR. Therefore, antibodies or small molecule therapeutics could reduce or inhibit fibrosis that occurs in liver cirrhosis. In addition, antibodies to this putative GPCR could also be used for the diagnosis of liver cirrhosis.

AH. CG57564-01: Olfactory Receptor

Expression of gene CG57564-01 was assessed using the primer-probe set Ag2616, described in Table AHA. Results of the RTQ-PCR runs are shown in Tables AHB and AHC.

TABLE AHA
Probe Name Ag2616

| | | | Start | |
|---|---|---|---|---|
| Primers | Sequences | Length | Position | SEQ ID NO |
| Forward | 5′-tcatcttcttatggccaatgtc-3′ | 22 | 830 | 271 |
| Probe | TET-5′-tgcctcccatgcttaacccaatcata-3′-TAMRA | 26 | 894 | 272 |
| Reverse | 5′-ggtggatctccttggtcttatt-30′ | 22 | 894 | 273 |

TABLE AHB
Panel 1.3D

| | Rel. Exp.(%) Ag2616, | | Rel. Exp.(%) Ag2616, |
|---|---|---|---|
| Tissue Name | Run 167644789 | Tissue Name | Run 167644789 |
| Liver adenocarcinoma | 11.0 | Kidney (fetal) | 2.3 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca.UO-31 | 0.0 |
| Pituitary gland | 0.6 | Renal ca.TK-10 | 0.0 |
| Brain (fetal) | 0.0 | Liver | 0.4 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.7 | Lung | 0.0 |
| Brain (hippocampus) | 0.3 | Lung (fetal) | 1.1 |
| Brain | 0.0 | Lung ca. | 32.1 |
| (substantia nigra) | | (small cell) LX-1 | |
| Brain (thalamus) | 0.0 | Lung ca. (small cell) NCI- | 0.0 |
| | | H69 | |
| Cerebral Cortex | 0.3 | Lung ca. (s. cell var.) SHP- | 12.9 |
| | | 77 | |
| Spinal cord | 0.4 | Lung ca. (large cell) NCI- | 0.0 |
| | | H460 | |
| glio/astro U87-MG | 0.0 | Lung ca. (non-sm. cell) | 0.6 |
| | | A549 | |
| glio/astro U-118-MG | 0.8 | Lung ca. (non-s. cell) NCI- | 0.0 |
| | | H23 | |
| astrocytoma SW1783 | 0.4 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 1.2 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.2 |
| glioma U251 | 1.3 | Breast ca.* (pl. ef) MCF-7 | 0.0 |
| glioma SF-295 | 1.5 | Breast ca.* (pl. ef) MDA- | 0.0 |
| | | MB-231 | |
| Heart (fetal) | 0.0 | Breast ca.* (pl. ef) T47D | 0.0 |
| Heart | 0.0 | Breast ca. BT-549 | 0.0 |
| Skeletal muscle (fetal) | 0.5 | Breast ca. MDA-N | 11.9 |
| Skeletal muscle | 0.0 | Ovary | 0.0 |
| Bone marrow | 0.0 | Ovarian ca. OVCAR-3 | 1.8 |
| Thymus | 0.0 | Ovarian ca. OVCAR-4 | 0.5 |
| Spleen | 0.6 | Ovarian ca. OVCAR-5 | 0.8 |
| Lymph node | 0.0 | Ovarian ca. OVCAR-8 | 0.0 |
| Colorectal | 0.9 | Ovarian ca. IGROV-1 | 0.5 |
| Stomach | 0.6 | Ovarian ca.* (ascites) SK- | 8.4 |
| | | OV-3 | |
| Small intestine | 0.0 | Uterus | 0.3 |
| Colon ca. SW480 | 0.0 | Plancenta | 0.5 |
| Colon ca.* | 13.1 | Prostate | 0.0 |
| SW620 (SW480 met) | | | |
| Colon ca. HT29 | 0.0 | Prostate ca.* (bone | 0.0 |
| | | met)PC-3 | |
| Colon ca. HCT-116 | 100.0 | Testis | 0.0 |
| Colon ca. CaCo-2 | 0.0 | Melanoma Hs688(A).T | 0.0 |
| Colon ca. | 0.0 | Melanoma* (met) | 0.0 |
| tissue (ODO3866) | | Hs688(B).T | |
| Colon ca. HCC-2998 | 0.5 | Melanoma UACC-62 | 0.8 |
| Gastric ca.* (liver met) | 0.0 | Melanoma M14 | 0.8 |
| NCI-N87 | | | |
| Bladder | 0.8 | Melanoma LOX IMVI | 14.2 |
| Trachea | 0.0 | Melanoma* (met) SK- | 51.4 |
| | | MEL-5 | |
| Kidney | 1.1 | Adipose | 1.1 |

TABLE AHC
Panel 2.2

| | Rel. Exp.(%) Ag2616, | | Rel. Exp.(%) Ag2616, |
|---|---|---|---|
| Tissue Name | Run 175063646 | Tissue Name | Run 175063646 |
| Normal Colon | 5.4 | Kidney Margin | 100.0 |
| | | (OD04348) | |
| Colon cancer (OD06064) | 0.0 | Kidney malignant cancer | 0.0 |
| | | (OD06204B) | |
| Colon Margin (OD06064) | 0.0 | Kidney normal adjacent | 0.0 |
| | | tissue (OD06204E) | |
| Colon cancer (OD06159) | 0.0 | Kidney Cancer | 0.0 |
| | | (OD04450-01) | |
| Colon Margin (OD06159) | 4.9 | Kidney Margin | 2.5 |
| | | (OD04450-03) | |
| Colon cancer (OD06297- | 0.0 | Kidney Cancer 8120613 | 0.0 |
| 04) | | | |
| Colon Margin (OD06297- | 2.9 | Kidney Margin 8120614 | 0.0 |
| 015) | | | |
| CC Gr.2 ascend colon | 0.0 | Kidney Cancer 9010320 | 5.7 |
| (ODO3921) | | | |
| CC Margin (ODO3921) | 0.0 | Kidney Margin 9010321 | 0.0 |
| Colon cancer metastasis | 0.0 | Kidney Cancer 8120607 | 0.0 |
| (OD06104) | | | |
| Lung Margin (OD06104) | 0.0 | Kidney Margin 8120608 | 0.0 |
| Colon mets to lung | 5.1 | Normal Uterus | 11.8 |
| (OD04451-01) | | | |
| Lung Margin (OD04451- | 0.0 | Uterine Cancer 064011 | 14.0 |
| 02) | | | |
| Normal Prostate | 0.0 | Normal Thyroid | 0.0 |
| Prostate Cancer | 0.0 | Thyroid Cancer 064010 | 0.0 |
| (OD04410) | | | |
| Prostate Margin | 13.9 | Thyroid Cancer A302152 | 6.6 |
| (OD04410) | | | |
| Normal Ovary | 14.1 | Thyroid Margin A302153 | 4.4 |
| Ovarian cancer (OD06283- | 0.0 | Normal Breast | 0.0 |
| 03) | | | |
| Ovarian Margin | 22.1 | Breast Cancer (OD04566) | 8.4 |
| (OD06283-07) | | | |
| Ovarian Cancer 064008 | 20.0 | Breast Cancer 1024 | 0.0 |
| Ovarian cancer (OD06145) | 0.0 | Breast Cancer (OD04590- | 5.6 |
| | | 01) | |
| Ovarian Margin | 5.6 | Breast Cancer Mets | 0.0 |
| (OD06145) | | (OD04590-03) | |
| Ovarian cancer (OD06455- | 0.0 | Breast Cancer Metastasis | 0.0 |
| 03) | | (OD04655-05) | |
| Ovarian Margin | 0.0 | Breast Cancer 064006 | 0.0 |
| (OD06455-07) | | | |
| Normal Lung | 7.7 | Breast Cancer 9100266 | 0.0 |
| Invasive poor diff. lung | 59.9 | Breast Margin 9100265 | 0.0 |
| adeno (ODO4945-01) | | | |
| Lung Margin (ODO4945- | 0.0 | Breast Cancer A209073 | 0.0 |
| 03) | | | |
| Lung Malignant Cancer | 0.0 | Breast Margin A2090734 | 0.0 |
| (OD03126) | | | |
| Lung Margin (OD03126) | 3.1 | Breast cancer (OD06083) | 22.4 |
| Lung Cancer (OD05014A) | 0.0 | Breast cancer node | 7.5 |
| | | metastasis (OD06083) | |
| Lung Margin (OD05014B) | 2.5 | Normal Liver | 3.6 |
| Lung cancer (OD06081) | 0.0 | Liver Cancer 1026 | 0.0 |
| Lung Margin (OD06081) | 0.0 | Liver Cancer 1025 | 0.0 |
| Lung Cancer (OD04237- | 0.0 | Liver Cancer 6004-T | 0.0 |
| 01) | | | |
| Lung Margin (OD04237- | 6.2 | Liver Tissue 6004-N | 5.0 |
| 02) | | | |
| Ocular Melanoma | 0.0 | Liver Cancer 6005-T | 0.0 |
| Metastasis | | | |
| Ocular Melanoma Margin | 7.9 | Liver Tissue 6005-N | 0.0 |
| (Liver) | | | |
| Melanoma Metastasis | 0.0 | Liver Cancer 064003 | 7.2 |
| Melanoma Margin (Lung) | 0.0 | Normal Bladder | 5.3 |
| Normal Kidney | 5.5 | Bladder Cancer 1023 | 0.0 |
| Kidney Ca, Nuclear grade | 12.8 | Bladder Cancer A302173 | 6.9 |
| 2 (OD04338) | | | |
| Kidney Margin (OD04338) | 0.8 | Normal Stomach | 11.6 |
| Kidney Ca Nuclear grade | 42.9 | Gastric Cancer 9060397 | 0.0 |
| 1/2 (OD04339) | | | |
| Kidney Margin (OD04339) | 7.3 | Stomach Margin 9060396 | 0.0 |
| Kidney Ca, Clear cell type | 0.0 | Gastric Cancer 9060395 | 0.0 |
| (OD04340) | | | |
| Kidney Margin (OD04340) | 26.1 | Stomach Margin 9060394 | 0.0 |
| Kidney Ca, Nuclear grade | 0.0 | Gastric Cancer 064005 | 0.0 |
| 3 (OD04348) | | | |

Panel 1.3D Summary: Ag2616 The expression of the CG57564-01 gene appears to be highest in a sample derived from a colon cancer cell lint (HCT1 16) (CT=29.3). In addition, there is expression evident in another colon cancer cell line (SW620), two melanoma cell lines (SK-MEL-5, LOX IMVI), two lung cancer cell lines (LX-1, SHP-77), and ovarian cancer cell line, a breast cancer cell line and a liver cancer. Thus, the expression of this gene could be used to distinguish HCT-116 cells for the other cells in the panel. Moreover, therapeutic modulation of this gene, through the use of antibodies, small molecule drugs or protein therapeutics might be of benefit in the treatment of colon cancer, melanoma, lung cancer, ovarian cancer, breast cancer or liver cancer.

Panel 2.2 Summary: Ag2616 The expression of the CG57564-01 gene is highest in a sample derived from normal kidney tissue adjacent to a malignancy (CT=32.9). In addition, there appears to be expression in another normal kidney tissue sample, a sample of malignant kidney and a sample from a lung cancer. Thus, the expression of this gene could be used to distinguish these samples from other samples in the panel. Moreover, therapeutic modulation of this gene, through the use of antibodies, small molecule drugs or protein therapeutics might be of benefit in the treatment of lung cancer or kidney cancer

Panel 4D Summary: Ag2616 Data from one experiment with this probe and primer is not included because the amp plot indicates that there were experimental difficulties. (Data not shown.)

AI. CG57691-01: Olfactory Receptor

Expression of gene CG57691-01 was assessed using the primer-probe sets Ag1788 and Ag1717, described in Tables AIA, AIB, and AIC. Results of the RTQ-PCR runs are shown in Tables AID and AIE.

TABLE AIA
Probe Name Ag1788

| Primers | Sequences | Lenght | Start Position | SEQ ID NO |
|---|---|---|---|---|
| Forward | 5′-atcttcctcgagtcaccaaact-3′ | 22 | 520 | 275 |
| Probe | TET-5′-tgcctgcctggactcttacatcattg-3′-TAMRA | 26 | 542 | 276 |
| Reverse | 5′-agtgcttagggaaagaattcca-3′ | 22 | 590 | 277 |

TABLE AIB
Probe Name Ag1717

| Primers | Sequences | Length | Start Position | SEQ ID NO |
|---|---|---|---|---|
| Forward | 5′-atcttcctcgagtcaccaaact-3′ | 22 | 520 | 278 |
| Probe | TET-5′-tgcctgcctggactcttacatcattg-3′-TAMRA | 26 | 542 | 279 |
| Reverse | 5′-agtgcttagggaaagaattcca-3′ | 22 | 590 | 280 |

TABLE AIC
Probe Name Ag1715

| Primers | Sequences | Length | Start Position | SEQ ID NO |
|---|---|---|---|---|
| Forward | 5′-atcttcctcgagtcaccaaact-3′ | 22 | 520 | 281 |
| Probe | TET-5′-tgcctgcctggactcctacatcattg-3′-TAMRA | 26 | 542 | 282 |
| Reverse | 5′-agtgcttagggaaagaattcca-3′ | 22 | 590 | 283 |

TABLE AID
Panel 1.3D

| | Rel. Exp.(%) Ag1788, | | Rel. Exp.(%) Ag1788, |
|---|---|---|---|
| Tissue Name | Run 165974808 | Tissue Name | Run 165974808 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 0.0 | Lung ca. (large | 0.0 |
| | | cell) NCI-H460 | |
| glio/astro U87-MG | 0.0 | Lung ca. | 0.0 |
| | | (non-sm. cell) A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl. ef) | 0.0 |
| | | MCF-7 | |
| glioma SF-295 | 0.0 | Breast ca.* (pl. ef) | 0.0 |
| | | MDA-MB-231 | |
| Heart (Fetal) | 0.0 | Breast ca.* (pl. ef) | 0.0 |
| | | T47D | |
| Heart | 0.0 | Breast ca. BT-549 | 6.9 |
| Skeletal muscle (Fetal) | 0.0 | Breast ca. MDA-N | 0.0 |
| Skeletal muscle | 0.0 | Ovary | 0.0 |
| Bone marrow | 1.6 | Ovarian ca. OVCAR-3 | 0.0 |
| Thymus | 0.0 | Ovarian ca. OVCAR-4 | 0.0 |
| Spleen | 100.0 | Ovarian ca. OVCAR-5 | 0.0 |
| Lymph node | 0.0 | Ovarian ca. OVCAR-8 | 0.0 |
| Colorectal | 2.4 | Ovarian ca. IGROV-1 | 0.0 |
| Stomach | 0.0 | Ovarian ca. (ascites) | 27.0 |
| | | SK-OV-3 | |
| Small intestine | 0.0 | Uterus | 0.0 |
| Colon ca. SW480 | 0.0 | Placenta | 0.0 |
| Colon ca.* SW620 | 0.0 | Prostate | 0.0 |
| (SW480 met) | | | |
| Colon ca. HT29 | 0.0 | Prostate ca.* (bone | 0.0 |
| | | met) PC-3 | |
| Colon ca. HCT-116 | 0.0 | Testis | 0.0 |
| Colon ca. CaCo-2 | 0.0 | Melanoma Hs688(A).T | 0.0 |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE AIE
Panel 4D

| | Rel. Exp. (%) | Rel. Exp. (%) | | Rel. Exp. (%) | Rel. Exp. (%) |
|---|---|---|---|---|---|
| | Ag1717, Run | Ag1788, Run | | Ag1717, Run | Ag1788, Run |
| Tissue Name | 165767645 | 165801807 | Tissue Name | 165767645 | 165801807 |
| Secondary Th1 act | 2.5 | 0.0 | HUVEC IL-1 beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 3.9 | HUVEC TNF | 0.0 | 0.0 |
| | | | alpha + IFN gamma | | |
| Secondary Th1 rest | 0.0 | 0.0 | HUVEC TNF | 0.0 | 0.0 |
| | | | alpha + IL4 | | |
| Secondary Th2 rest | 0.0 | 0 0 | HUVEC IL-11 | 0.0 | 0.0 |
| Secondary Tr1 rest | 0.0 | 0.0 | Lung Microvascular | 0.0 | 0.0 |
| | | | EC none | | |
| Primary Th1 act | 0.0 | 0.0 | Lung Microvascular | 0.0 | 0.0 |
| | | | EC TNF alpha + IL- | | |
| | | | 1 beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 0.0 |
| | | | Dermal EC | | |
| | | | TNF alpha + IL- | | |
| | | | 1 beta | | |
| Primary Th1 rest | 0.0 | 0.0 | Bronchial | 0.0 | 0.0 |
| | | | epithelium | | |
| | | | TNF alpha + IL1 beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium none | | |
| | | | TNF alpha + IL- | | |
| | | | 1 beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 0.0 |
| lymphocyte act | | | SMC TNF alpha + | | |
| | | | IL-1 beta | | |
| CD8 lymphocyte act | 0.0 | 0.0 | Astrocytes rest | 0.0 | 0.0 |
| Secondary CD8 | 0.0 | 0.0 | Astrocytes | 0.0 | 0.0 |
| lymphocyte rest | | | TNF alpha + IL- | | |
| | | | 1 beta | | |
| Secondary CD8 | 0.0 | 2.2 | KU-812 (Basophil) | 0.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNF alpha + IL- | | |
| | | | 1 beta | | |
| LAK cells IL-2 | 0.0 | 0.0 | Liver cirrhosis | 100.0 | 100.0 |
| LAK cells IL-2 + IL- | 0.0 | 0.0 | Lupus kidney | 0.0 | 0.0 |
| 12 | | | | | |
| LAK cells IL-2 + IFN | 0.0 | 0.0 | NCI-H292 none | 0.0 | 0.0 |
| gamma | | | | | |
| LAK cells IL-2 + IL- | 0.0 | 0.0 | NCI-H292 IL-4 | 0.0 | 0.0 |
| 18 | | | | | |
| LAK cells | 0.0 | 0.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 | 0.0 | 22.5 | NCI-H292 IFN | 0.0 | 0.0 |
| day | | | gamma | | |
| Two Way MLR 5 | 0.0 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1 beta | | |
| PBMC rest | 0.0 | 0.0 | Lung fibroblast none | 0.0 | 0.0 |
| PBMC PWM | 0.0 | 0.0 | Lung fibroblast TNF | 0.0 | 0.0 |
| | | | alpha + IL-1 beta | | |
| PBMC PHA-L | 0.0 | 0.0 | Lung fibroblast IL-4 | 0.0 | 4.0 |
| Ramos (B cell) none | 0.0 | 0.0 | Lung fibroblast IL-9 | 0.0 | 0.0 |
| Ramos (B cell) | 0.0 | 0.0 | Lung fibroblast IL- | 0.0 | 0.0 |
| ionomycin | | | 13 | | |
| B lymphocytes | 0.0 | 0.0 | Lung fibroblast IFN | 0.0 | 0.0 |
| PWM | | | gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 2.6 |
| | | | CCD1070 TNF | | |
| | | | alpha | | |
| EOL-1 dbcAMP | 0.0 | 0.0 | Dermal fibroblast | 0.0 | 0.0 |
| PMA/ionomycin | | | CCD1070 IL-1 beta | | |
| Dendritic cells none | 0.0 | 0.0 | Dermal fibroblast | 0.0 | 0.0 |
| | | | IFN gamma | | |
| Dendritic cells LPS | 0.0 | 0.0 | Dermal fibroblast | 0.0 | 0.0 |
| | | | IL-4 | | |
| Dendritic cells anti- | 0.0 | 0.0 | IBD Colitis 2 | 49.3 | 54.0 |
| CD40 | | | | | |
| Monocytes rest | 0.0 | 0.0 | IBD Crohn's | 8.8 | 2.0 |
| Monocytes LPS | 0.0 | 0.0 | Colon | 0.0 | 3.5 |
| Macrophages rest | 0.0 | 0.0 | Lung | 0.0 | 9.2 |
| Macrophages LPS | 0.0 | 0.0 | Thymus | 0.0 | 0.0 |
| HUVEC none | 0.0 | 0.0 | Kidney | 0.0 | 0.0 |
| HUVEC starved | 0.0 | 0.0 | | | |

Panel 1.3D Summary: Ag1788 The CG57691-01 gene is only expressed in the spleen, an important site of secondary immune responses. Therefore, antibodies or small molecule therapeutics that block the function of this GPCR may be useful as anti-inflammatory therapeutics for the treatment of allergies, autoimmune diseases, and inflammatory diseases.

Panel 2.2 Summary: Ag1788 Expression is low/undetectable in all samples on this panel (CTs>35). (Data not shown.)

Panel 4D Summary: Ag1717/Ag1788 Two experiments using the same probe and primer set show expression in liver cirrhosis and IBD colitis (CTs=32.2-33.3). Thus, the function of the putative GPCR encoded by the CG57691-01 gene may be important in the disease processes in both inflammatory bowel disease and in liver cirrhosis. Therefore, blocking antibodies or small molecule antagonists targeted to this GPCR may be useful as therapeutics in colitis and in cirrhosis. A third run with probe/primer set Ag 1715 had low/undetectable levels of expression in all samples in this panel (CTs>35). (Data not shown).

AJ. CG59408-01: Olfactory Receptor

Expression of gene CG59408-01 was assessed using the primer-probe set Ag1582, described in Table AJA. Results of the RTQ-PCR runs are shown in Table AJB.

TABLE AJA
Probe Name Ag1582

| Primers | Sequences | Length | Start Position | SEQ ID NO |
|---|---|---|---|---|
| Forward | 5′-atgatcttcgttccaagcattt-3′ | 22 | 753 | 284 |
| Probe | TET-5′-acctctatgcccggcccttcact-3′-TAMRA | 23 | 775 | 285 |
| Reverse | 5′-gatggacacaagcttgtccat-3′ | 21 | 807 | 286 |

TABLE AJB
Panel 4D

| | Rel. Exp.(%) | | Rel. Exp.(%) |
|---|---|---|---|
| | Ag1582, Run | | Ag1582, Run |
| Tissue Name | 165820889 | Tissue Name | 165820889 |
| Secondary Th1 act | 0.0 | HUVEC IL-1 beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 3.7 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1 beta | |
| Primary Th2 act | 0.0 | Microvascular Dermal | 0.0 |
| | | EC none | |
| Primary Tr1 act | 0.0 | Microsvasular Dermal | 0.0 |
| | | EC TNFalpha + IL-1 beta | |
| Primary Th1 rest | 8.2 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + Il-1 beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1 beta | |
| Secondary CD8 | 0.0 | KU-812 (Basophil) rest | 0.0 |
| lymphocyte act | | | |
| CD4 lymphocyte none | 4.1 | KU-812 (Basophil) | 0.0 |
| | | PMA/ionomycin | |
| 2ry Th1/Th2/Tr1_anti- | 7.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1 beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 3.0 | NCI-H292 IL-13 | 0.0 |
| Two Way MLR 3 day | 4.3 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 2.4 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 2.6 | Dermal fibroblast IFN gamma | 0.0 |
| Dendritic cells LPS | 0.0 | Dermal fibroblast IL-4 | 0.0 |
| Dendritic ceils anti-CD40 | 0.0 | IBD Colitis 2 | 0.0 |
| Monocytes rest | 0.0 | IBD Crohn's | 0.0 |
| Monocytes LPS | 0.0 | Colon | 0.0 |
| Macrophages rest | 0.0 | Lung | 12.5 |
| Macrophages LPS | 0.0 | Thymus | 4.2 |
| HUVEC none | 0.0 | Kidney | 0.0 |
| HUVEC starved | 0.0 | | |

Panel 1.3D Summary: Ag1582 Expression of this gene is low/undetected in all samples in this panel (CT>35). (Data not shown.)

Panel 2.2 Summary: Ag1582 Expression of this gene is low/undetectable (CTs>35) across all of the samples on this panel (data not shown).

Panel 4D Summary: Ag1582 The CG59408-01 transcript is only detected in liver cirrhosis. Furthermore, this transcript is not detected in normal liver in Panel 1.3D, suggesting that CG59408-01 gene expression is unique to liver cirrhosis. The CG59408-01 gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

AK. CG90352-01: Olfactory Receptor

Expression of gene CG90352-01 was assessed using the primer-probe set Ag1705, described in Table AKA. Results of the RTQ-PCR runs are shown in Table AKB.

TABLE AKA
Probe Name Ag1705

| | | | Start | |
|---|---|---|---|---|
| Primers | Sequences | Length | Position | SEQ ID NO |
| Forward | 5′-ccgtctattctactgcatttgc-3′ | 22 | 154 | 287 |
| Probe | TET-5′-cccaaaatgattgttgacttgctctctg-3′-TAMRA | 28 | 177 | 288 |
| Reverse | 5′-atacaaccctgaaaggaaatgg-3′ | 22 | 214 | 289 |

TABLE AKB
Panel 4D

| | Rel. Exp.(%) | | Rel. Exp.(%) |
|---|---|---|---|
| | Ag1705, Run | | Ag1705, Run |
| Tissue Name | 165763028 | Tissue Name | 165763028 |
| Secondary Th1 act | 0.0 | HUVEC IL-1 beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1 beta | |
| Primary Th2 act | 0.0 | Microvascular Dermal EC none | 0.0 |
| Primary Tr1 act | 0.0 | Microsvasular Dermal EC | 0.0 |
| | | TNFalpha + IL-1 beta | |
| Primary Th1 rest | 0.0 | Bronchial epithelium TNFalpha + | 0.0 |
| | | IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1 beta | |
| CD45RA CD4 | 0.0 | Coronery artery SMC rest | 0.0 |
| lymphocyte act | | | |
| CD45RO CD4 | 0.0 | Coronery artery SMC TNFalpha | 0.0 |
| lymphocyte act | | + IL-1 beta | |
| CD8 lymphocyte act | 0.0 | Astrocytes rest | 0.0 |
| Secondary CD8 | 0.0 | Astrocytes TNFalpha + IL-1 beta | 0.0 |
| lymphocyte rest | | | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) none | 0.0 |
| CD95 CH11 | | | |
| LAK cells rest | 0.0 | CCD1106 (Keratinocytes) | 0.0 |
| | | TNFalpha + IL-1 beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 | 0.0 |
| | | beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 0.0 | Dermal fibroblast CCD1070 rest | 0.0 |
| and IL-4 | | | |
| EOL-1 dbcAMP | 0.0 | Dermal fibroblast CCD1070 TNF alpha | 0.0 |
| EOL-1 dbcAMP | 0.0 | Dermal fibroblast CCD1070 IL-1 | 0.0 |
| PMA/ionomycin | | beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 52.5 |
| Monocytes rest | 0.0 | IBD Crohn's | 0.0 |
| Monocytes LPS | 0.0 | Colon | 0.0 |
| Macrophages rest | 0.0 | Lung | 4.9 |
| Macrophages LPS | 0.0 | Thymus | 0.0 |
| HUVEC none | 0.0 | Kidney | 0.0 |
| HUVEC starved | 0.0 | | |

Panel 1.3D Summary: Ag1705 Expression of this gene is low/undetectable (CTs>35) across all of the samples on this panel (data not shown).

Panel 2.2 Summary: Ag1705 Expression of this gene is low/undetectable (CTs>35) across all of the samples on this panel (data not shown).

Panel 4D Summary: Ag1705 The CG90352-01 transcript is only detected in liver cirrhosis. Furthermore, this transcript is not detected in normal liver in Panel 1.3D, suggesting that CG90352-01 gene expression is unique to liver cirrhosis. The CG90352-01 gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

AL. CG92727-01: Olfactory Receptor

Expression of gene CG92727-01 was assessed using the primer-probe set Ag1806, described in Table ALA. Results of the RTQ-PCR runs are shown in Tables ALB, ALC, and ALD.

TABLE ALA
Probe Name Ag1806

| Primers | Sequences | Length | Start Position | SEQ ID NO |
|---|---|---|---|---|
| Forward | 5′-tcttggtctttgtgctgatctt-3′ | 22 | 73 | 290 |
| Probe | TET-5′-tcatcctccctggaaattttctcatt-3′-TAMRA | 26 | 109 | 291 |
| Reverse | 5′-agggtctgaccttatggtgaaa-3′ | 22 | 137 | 292 |

TABLE ALB
General_screening_panel_v1.5

| | Rel. Exp.(%) Ag1806, | | Rel. Exp.(%) Ag1806, |
|---|---|---|---|
| Tissue Name | Run 228714747 | Tissue Name | Run 228714747 |
| Adipose | 0.0 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL- | 0.0 | Colon ca. SW480 | 0.4 |
| 5 | | | |
| Squamous cell | 0.0 | Colon ca.* (SW480 met) | 0.0 |
| carcinoma SCC-4 | | SW620 | |
| Testis Pool | 100.0 | Colon ca. HT29 | 0.0 |
| Prostate ca.* (bone | 0.4 | Colon ca. HCT-116 | 0.0 |
| met) PC-3 | | | |
| Prostate Pool | 0.4 | Colon ca. CaCo-2 | 0.0 |
| Placenta | 0.0 | Colon cancer tissue | 0.4 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.9 | Colon Pool | 0.0 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.4 | Heart Pool | 0.4 |
| Breast ca. MDA-MB- | 0.0 | Lymph Node Pool | 0.0 |
| 231 | | | |
| Breast ca. BT 549 | 0.0 | Fetal Skeletal Muscle | 0.0 |
| Breast ca. T47D | 0.0 | Skeletal Muscle Pool | 0.4 |
| Breast ca. MDA-N | 0.0 | Spleen Pool | 0.0 |
| Breast Pool | 0.0 | Thymus Pool | 0.7 |
| Trachea | 0.0 | CNS cancer (glio/astro) | 0.0 |
| | | U87-MG | |
| Lung | 0.0 | CNS cancer (glio/astro) | 0.0 |
| | | U-118-MG | |
| Fetal Lung | 0.0 | CNS cancer (neuro; met) | 0.0 |
| | | SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) SNB- | 1.4 |
| | | 75 | |
| Lung ca. NCI-H146 | 0.0 | CNS cancer (glio) SNB- | 0.0 |
| | | 19 | |
| Lung ca. SHP-77 | 0.0 | CNS cancer (glio) SF-295 | 0.0 |
| Lung ca. A549 | 0.0 | Brain (Amygdala) Pool | 0.7 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 26.4 | Brain (Hippocampus) | 1.5 |
| | | Pool | |
| Lung ca. HOP-62 | 0.0 | Cerebral Cortex Pool | 1.0 |
| Lung ca. NCI-H522 | 0.0 | Brain (Substantia nigra) | 1.9 |
| | | Pool | |
| Liver | 0.0 | Brain (Thalamus) Pool | 0.9 |
| Fetal Liver | 0.0 | Brain (whole) | 0.0 |
| Liver ca. HepG2 | 0.0 | Spinal Cord Pool | 0.7 |
| Kidney Pool | 0.0 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN 2 | 0.0 |
| Renal ca. UO-31 | 0.0 | Pancreas Pool | 0.0 |

TABLE ALC
Panel 1.3D

| | Rel. Exp.(%) Ag1806, | | Rel. Exp.(%) Ag1806, |
|---|---|---|---|
| Tissue Name | Run 165975009 | Tissue Name | Run 165975009 |
| Liver | 0.0 | Kidney (fetal) | 0.0 |
| adenocarcinoma | | | |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 3.7 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 0.7 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Luna ca. (s. cell var.) | 0.0 |
| | | SHP-77 | |
| Spinal cord | 2.9 | Lung ca. (large | 0.0 |
| | | cell) NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*;met SK-N-AS | 0.0 | Lung ca. (non-s. cl) | 0.0 |
| | | NCI-H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U25l | 0.0 | Breast ca.* (pl. ef) | 0.0 |
| | | MCF-7 | |
| glioma SF-295 | 0.0 | Breast ca.* (pl. ef) | 0.0 |
| | | MDA-MB-231 | |
| Heart (fetal) | 0.0 | Breast ca.* (pl. ef) | 0.0 |
| | | T47D | |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 1.3 | Ovarian ca. OVCAR-4 | 0.0 |
| Spleen | 6.4 | Ovarian ca. OVCAR-5 | 0.0 |
| Lymph node | 1.1 | Ovarian ca. OVCAR-8 | 0.0 |
| Colorectal | 1.1 | Ovarian ca. IGROV-1 | 0.0 |
| Stomach | 0.0 | Ovarian ca.* (ascites) | 0.0 |
| | | SK-OV-3 | |
| Small intestine | 0.0 | Uterus | 0.0 |
| Colon ca. SW480 | 0.0 | Plancenta | 0.0 |
| Colon ca.* | 0.0 | Prostate | 1.3 |
| SW620 (SW480 met) | | | |
| Colon ca. HT29 | 0.0 | Prostate ca.* (bone | 0.0 |
| | | met) PC-3 | |
| Colon ca. HCT-116 | 0.0 | Testis | 100.0 |
| Colon ca. CaCo-2 | 0.0 | Melanoma Hs688(A).T | 0.0 |
| Colon ca. | 0.0 | Melanoma* (met) | 0.0 |
| tissue (ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE ALP
Panel 4D

| | Rel. Exp.(%) Ag1806, | | Rel. Exp.(%) Ag1806, |
|---|---|---|---|
| Tissue Name | Run 165812559 | Tissue Name | Run 165812559 |
| Secondary Th1 act | 0.0 | HUVEC IL-1 beta | 0.0 |
| Secondary Th2 act | 1.6 | HUVEC IFN gamma | 0.0 |
| Secondary Tr1 act | 0.4 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNFalpha + IL-1 beta | |
| Primary Th2 act | 2.9 | Microvascular Dermal | 0.0 |
| | | EC none | |
| Primary Tr1 act | 0.0 | Microsvasular Dermal EC | 0.0 |
| | | TNFalpha + IL-1 beta | |
| Primary Th1 rest | 3.2 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1 beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1 beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1 beta | |
| LAK cells IL-2 | 0.0 | Liver cirrhosis | 87.7 |
| LAK cells IL-2 + IL-12 | 0.0 | Lupus kidney | 0.0 |
| LAK cells IL-2 + IFN | 0.0 | NCI-H292 none | 68.3 |
| gamma | | | |
| LAK cells IL-2 + IL-18 | 0.0 | NCI-H292 IL-4 | 100.0 |
| LAK cells | 0.0 | NCI-H292 IL-9 | 77.9 |
| PMA/ionomycin | | | |
| NK Cells IL-2 rest | 0.0 | NCI-H292 IL-13 | 28.1 |
| Two Way MLR 3 day | 0.0 | NCI-H292 IFN gamma | 15.3 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 1.4 | Dermal fibroblast CCD1070 | 0.0 |
| and IL-4 | | rest | |
| EOL-1 dbcAMP | 0.0 | Dermal fibroblast CCD1070 | 13.6 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 42.3 |
| Monocytes rest | 0.0 | IBD Crohn's | 15.7 |
| Monocytes LPS | 0.0 | Colon | 2.8 |
| Macrophages rest | 0.0 | Lung | 4.6 |
| Macrophages LPS | 0.0 | Thymus | 0.0 |
| HUVEC none | 0.0 | Kidney | 6.8 |
| HUVEC starved | 0.0 | | |

CNS_neurodegeneration_v1.0 Summary: Ag1806 Expression of the CG92727-01 gene is low/undetected (CT>35) in all samples in this panel. (Data not shown.)

General_screening panel_v1.5 Summary: Ag1806 Expression of the CG92727-01 gene is restricted to the testis and a lung cancer cell line in this panel (CTs=31-34). This expression profile suggests that the protein encoded by the CG92727-01 gene may be involved in fertility. Therefore, therapeutic modulation of the function or expression of this gene product may be effective in treating fertility related disorders. Furthermore, the presence of the transcript in a lung cancer cell line indicates that the expression of this gene could be used to differentiate lung cancer cell lines from other samples in this panel. Therapeutic modulation of the function or expression of the CG92727-01 protein product may also be effective in the treatment of lung cancer.

Panel 1.3D Summary: Ag1 806 Expression of the CG92727-01 gene in this panel is restricted to the testis (CT=3 1). This is the same expression profile seen in General_screening panel_vl 0.5. Please see that panel for discussion of potential utility of this gene.

Panel 2.2 Summary: Ag1806 Expression of the CG92727-01 gene is low/undetected (CT>35) in all samples in this panel. (Data not shown.)

Panel 4D Summary: Ag1806 The CG92727-01 gene is constitutively expressed in the NCI-H292 mucoepidermoid cell line (CTs=32-33). In comparison, expression in the normal lung is low. The expression of the transcript in the NCI-H292 cell line, often used as a model for airway epithelium, suggests that this transcript may be important in the proliferation or activation of airway epithelium. Therefore, therapeutics designed with the GPCR encoded by the transcript could be important in the treatment of diseases that exhibit lung airway inflammation such as asthma and COPD.

This transcript is also expressed in liver cirrhosis and colitis. Normal liver and colon do not express this transcript (see panel 1.3 and 2.2 for liver) suggesting that expression may be specific to cirrhosis. The transcript or the protein encoded for by the transcript could be used diagnostically to identify liver cirrhosis or colitis. Furthermore, the protein encoded by this transcript could be used to design therapeutics against liver cirrhosis or colitis.

AM. CG146422-01/GMAC076959_B: Olfactory Receptor

Expression of gene CG146422-01 was assessed using the primer-probe set Ag1518, described in Table AMA. Results of the RTQ-PCR runs are shown in Tables AMB, AMC, and AMD.

TABLE AMA
Probe Name Ag1518

| Primers | Sequences | Length | Start Position | SEQ ID NO |
|---|---|---|---|---|
| Forward | 5′-caaccagacatggatcacaga-3′ | 21 | 10 | 293 |
| Probe | TET-5′-atcaccctgctgggattccaggtt-3′-TAMRA | 24 | 32 | 294 |
| Reverse | 5′-aagagtccacagaggagaatcg-3′ | 22 | 69 | 295 |

TABLE AMB
General_screening_panel_1.5

| | Rel. Exp.(%) Ag1518, | | Rel. Exp.(%) Ag1518, |
|---|---|---|---|
| Tissue Name | Run 228632348 | Tissue Name | Run 228632348 |
| Adipose | 14.5 | Renal ca.TK-10 | 9.0 |
| Melanoma* | 18.9 | Bladder | 100.0 |
| Hs688(A).T | | | |
| Melanoma* | 0.0 | Gastric ca. | 12.4 |
| Hs688(B).T | | (liver met.) NCI-N87 | |
| Melanoma* M14 | 0.0 | Gastric ca. KATO III | 20.4 |
| Melanoma* LOXIMVI | 0.0 | Colon ca. SW-948 | 0.0 |
| Melanoma* SK-MEL- | 0.0 | Colon ca. SW480 | 0.0 |
| 5 | | | |
| Squamous cell | 7.3 | Colon ca.* | 5.8 |
| carcinoma SCC-4 | | (SW480 met) SW620 | |
| Testis Pool | 2.0 | Colon ca. HT29 | 6.3 |
| Prostate ca.* (bone | 3.6 | Colon ca. HCT-116 | 12.8 |
| met) PC-3 | | | |
| Prostate Pool | 3.6 | Colon ca. CaCo-2 | 13.5 |
| Placenta | 3.4 | Colon cancer tissue | 25.3 |
| Uterus Pool | 5.0 | Colon ca. SW1116 | 10.0 |
| Ovarian ca. OVCAR-3 | 10.5 | Colon ca. Colo-205 | 0.0 |
| Ovarian ca. SK-OV-3 | 14.7 | Colon ca. SW-48 | 0.0 |
| Ovarian ca. OVCAR-4 | 0.0 | Colon Pool | 3.8 |
| Ovarian ca. OVCAR-5 | 13.1 | Small Intestine Pool | 6.7 |
| Ovarian ca. IGROV-1 | 0.0 | Stomach Pool | 16.3 |
| Ovarian ca. OVCAR-8 | 9.0 | Bone Marrow Pool | 21.3 |
| Ovary | 35.1 | Fetal Heart | 0.0 |
| Breast ca. MCF-7 | 3.7 | Heart Pool | 14.2 |
| Breast ca. MDA-MB- | 0.0 | Lymph Node Pool | 18.8 |
| 231 | | | |
| Breast ca. BT 549 | 0.0 | Fetal Skeletal Muscle | 0.0 |
| Breast ca. T47D | 0.0 | Skeletal Muscle Pool | 4.0 |
| Breast ca. MDA-N | 6.6 | Spleen Pool | 5.4 |
| Breast Pool | 7.4 | Thymus Pool | 0.0 |
| Trachea | 6.5 | CNS cancer (glio/astro) | 0.0 |
| | | U87-MG | |
| Lung | 0.0 | CNS cancer (glio/astro) | 0.0 |
| | | U-118-MG | |
| Fetal Lung | 14.1 | CNS cancer (neuro; met) | 0.0 |
| | | SK-N-AS | |
| Lung ca. NCI-N417 | 0.0 | CNS cancer (astro) SF- | 0.0 |
| | | 539 | |
| Lung ca. LX-1 | 10.8 | CNS cancer (astro) SNB- | 0.0 |
| | | 75 | |
| Lung ca. NCI-H146 | 0.0 | CNS cancer (glio) SNB- | 3.7 |
| | | 19 | |
| Lung ca. SHP-77 | 0.0 | CNS cancer (glio) SF-295 | 4.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 3.8 | Brain (fetal) | 9.5 |
| Lung ca. NCI-H460 | 93.3 | Brain (Hippocampus) | 1.5 |
| | | Pool | |
| Lung ca. HOP-62 | 0.0 | Cerebral Cortex Pool | 0.0 |
| Lung ca. NCI-H522 | 0.0 | Brain (Substantia nigra) | 3.6 |
| | | Pool | |
| Liver | 0.0 | Brain (Thalamus) Pool | 0.0 |
| Fetal Liver | 30.6 | Brain (whole) | 0.0 |
| Liver ca. HepG2 | 0.0 | Spinal Cord Pool | 0.0 |
| Kidney Pool | 10.5 | Adrenal Gland | 0.0 |
| Fetal Kidney | 58.6 | Pituitary gland Pool | 3.9 |
| Renal ca. 786-0 | 46.3 | Salivary Gland | 3.3 |
| Renal ca. A498 | 13.4 | Thyroid (female) | 0.0 |
| Renal ca. ACHN | 7.4 | Pancreatic ca. CAPAN2 | 3.3 |
| Renal ca. UO-31 | 7.1 | Pancreas Pool | 10.1 |

TABLE AMC
Panel 1.2

| | Rel. Exp.(%) Ag1518, | | Rel. Exp.(%) Ag1518, |
|---|---|---|---|
| Tissue Name | Run 141990541 | Tissue Name | Run 141990541 |
| Endothelial cells | 0.0 | Renal ca. 786-0 | 8.1 |
| Heart (Fetal) | 4.1 | Renal ca. A498 | 16.5 |
| Pancreas | 1.9 | Renal ca. RXF 393 | 1.8 |
| Pancreatic ca. CAPAN 2 | 1.1 | Renal ca. ACHN | 5.4 |
| Adrenal Gland | 1.0 | Renal ca. UO-31 | 24.1 |
| Thyroid | 0.9 | Renal ca. TK-10 | 11.6 |
| Salivary gland | 14.7 | Liver | 8.0 |
| Pituitary gland | 0.0 | Liver (fetal) | 3.2 |
| Brain (fetal) | 0.0 | Liver ca. | 12.9 |
| | | (hepatoblast) HepG2 | |
| Brain (whole) | 0.0 | Lung | 0.0 |
| Brain (amygdala) | 1.5 | Lung (fetal) | 0.0 |
| Brain (cerebellum) | 0.0 | Lung ca. | 14.2 |
| | | (small cell) LX-1 | |
| Brain (hippocampus) | 0.5 | Lung ca. | 50.7 |
| | | (small cell) NCI-H69 | |
| Brain (thalamus) | 0.6 | Lung ca. | 0.0 |
| | | (s. cell var.) SHP-77 | |
| Cerebral Cortex | 0.0 | Lung ca. | 10.0 |
| | | (large cell) NCI-H460 | |
| Spinal cord | 1.7 | Lung ca. | 9.3 |
| | | (non-sm. cell) A549 | |
| glio/astro U87-MG | 1.4 | Lung ca. | 6.6 |
| | | (non-s. cell) NCI-H23 | |
| glio/astro U-118-MG | 1.6 | Lung ca. | 6.7 |
| | | (non-s. cell) HOP-62 | |
| astrocytoma SW1783 | 3.1 | Lung ca. | 3.5 |
| | | (non-s. cl) NCI-H522 | |
| neuro*; met SK-N-AS | 2.4 | Lung ca. (squam.) SW | 44.8 |
| | | 900 | |
| astrocytoma SF-539 | 0.0 | Lung ca. | 32.5 |
| | | (squam.) NCI-H596 | |
| astrocytoma SNB-75 | 0.0 | Mammary gland | 3.3 |
| glioma SNB-19 | 9.5 | Breast ca.* (pl. ef) | 3.1 |
| | | MCF-7 | |
| glioma U251 | 0.0 | Breast ca.* | 1.9 |
| | | (pl. ef) MDA-MB-231 | |
| glioma SF-295 | 1.2 | Breast ca.* | 20.7 |
| | | (pl. ef) T47D | |
| Heart | 15.5 | Breast ca. BT-549 | 3.8 |
| Skeletal Muscle | 1.4 | Breast ca. MDA-N | 12.8 |
| Bone marrow | 0.0 | Ovary | 1.5 |
| Thymus | 0.0 | Ovarian ca. OVCAR-3 | 15.9 |
| Spleen | 0.0 | Ovarian ca. OVCAR-4 | 16.4 |
| Lymph node | 0.0 | Ovarian ca. OVCAR-5 | 93.3 |
| Colorectal Tissue | 27.9 | Ovarian ca. OVCAR-8 | 28.5 |
| Stomach | 2.1 | Ovarian ca. IGROV-1 | 8.0 |
| Small intestine | 3.3 | Ovarian ca. | 8.1 |
| | | (ascites) SK-OV-3 | |
| Colon ca. SW480 | 4.1 | Uterus | 1.1 |
| Colon ca.* SW620 | 4.5 | Placenta | 1.0 |
| (SW480 met) | | | |
| Colon ca. HT29 | 10.7 | Prostate | 6.9 |
| Colon ca. HCT-116 | 14.1 | Prostate ca.* | 13.8 |
| | | (bone met) PC-3 | |
| Colon ca. CaCo-2 | 6.2 | Testis | 2.4 |
| Colon ca. Tissue | 21.8 | Melanoma Hs688(A).T | 2.0 |
| (ODO3866) | | | |
| Colon ca. HCC-2998 | 32.8 | Melanoma* (met) Hs688 | 11.3 |
| | | (B).T | |
| Gastric ca.* (liver met) | 2.2 | Melanoma UACC-62 | 0.0 |
| NCI-N87 | | | |
| Bladder | 100.0 | Melanoma M14 | 18.0 |
| Trachea | 0.0 | Melanoma LOX IMVI | 0.0 |
| Kidney | 54.7 | Melanoma* (met) | 0.0 |
| | | SK-MEL-5 | |
| Kidney (fetal) | 7.7 | | |

TABLE AMD
Panel 4.1D

| | Rel. Exp.(%) Ag1518, | | Rel. Exp.(%) Ag1518, |
|---|---|---|---|
| Tissue Name | Run 223788518 | Tissue Name | Run 223788518 |
| Secondary Th1 act | 0.0 | HUVEC IL-1 beta | 0.0 |
| Secondary Th2 act | 0.0 | HUVEC IFN gamma | 0.2 |
| Secondary Tr1 act | 0.0 | HUVEC TNF alpha + IFN | 0.0 |
| | | gamma | |
| Secondary Th1 rest | 0.4 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1 beta | |
| Primary Th2 act | 0.2 | Microvascular Dermal EC | 0.0 |
| | | none | |
| Primary Tr1 act | 0.0 | Microsvasular Dermal EC | 0.0 |
| | | TNFalpha + IL-1 beta | |
| Primary Th1 rest | 0.4 | Bronchial epithelium | 0.0 |
| | | TNFalpha + IL1beta | |
| Primary Th2 rest | 0.1 | Small airway epithelium none | 0.0 |
| Primary Tr1 rest | 0.0 | Small airway epithelium | 0.7 |
| | | TNFalpha + IL-1 beta | |
| CD45RA CD4 | 0.4 | Coronery artery SMC rest | 0.0 |
| lymphocyte act | | | |
| CD45RO CD4 | 0.0 | Coronery artery SMC | 0.0 |
| lymphocyte act | | TNFalpha + IL-1 beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1 beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 1.8 | CCD1106 (Keratinocytes) | 0.8 |
| CD95 CH11 | | none | |
| LAK cells rest | 0.0 | CCD1106 (Keratinocytes) | 0.0 |
| | | TNFalpha + IL-1 beta | |
| LAK cells IL-2 | 0.0 | Liver cirrhosis | 0.4 |
| LAK cells IL-2 + IL-12 | 0.0 | NCI-H292 none | 0.8 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.2 |
| LAK cells | 0.0 | NCI-H292 IL-13 | 0.1 |
| PMA/ionomycin | | | |
| NK Cells IL-2 rest | 0.0 | NCI-H292 IFN gamma | 0.2 |
| Two Way MLR 3 day | 0.6 | HPAEC none | 0.0 |
| Two Way MLR 5 day | 0.0 | HPAEC TNF alpha + IL-1 | 0.0 |
| | | beta | |
| Two Way MLR 7 day | 0.0 | Lung fibroblast none | 0.3 |
| PBMC rest | 0.0 | Lung fibroblast TNF alpha + | 0.0 |
| | | IL-1 beta | |
| PBMC PWM | 0.0 | Lung fibroblast IL-4 | 0.1 |
| PBMC PHA-L | 0.0 | Lung fibroblast IL-9 | 0.3 |
| Ramos (B cell) none | 0.0 | Lung fibroblast IL-13 | 0.0 |
| Ramos (B cell) | 0.0 | Lung fibroblast IFN gamma | 0.5 |
| ionomycin | | | |
| B lymphocytes PWM | 0.0 | Dermal fibroblast CCD1070 | 0.0 |
| | | rest | |
| B lymphocytes CD40L | 0.0 | Dermal fibroblast CCD1070 | 0.1 |
| and IL-4 | | TNF alpha | |
| EOL-1 dbcAMP | 0.0 | Dermal fibroblast CCD1070 | 0.2 |
| | | IL-1 beta | |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.9 |
| Dendritic cells LPS | 0.0 | Dermal Fibroblasts rest | 0.4 |
| Dendritic cells anti-CD40 | 0.0 | Neutrophils TNFa + LPS | 0.0 |
| Monocytes rest | 0.0 | Neutrophils rest | 1.0 |
| Monocytes LPS | 0.0 | Colon | 4.5 |
| Macrophages rest | 0.0 | Lung | 4.5 |
| Macrophages LPS | 0.0 | Thymus | 14.2 |
| HUVEC none | 0.0 | Kidney | 100.0 |
| HUVEC starved | 0.0 | | |

CNS_neurodegeneration_v1.0 Summary: Ag1518 Expression of the CG146422-01 gene is low/undetectable in all samples on this panel. (CTs>35). (Data not shown.)

General_screening panel_v1.5 Summary: Ag1518 Expression of the CG146422-01 gene is limited to the bladder and a lung cancer cell line (CTs=33). This result is consistent with the results seen in Panel 1.2. Thus, expression of this gene could be used to differentiate between these samples and other samples on this panel and as a marker for bladder or malignant lung tissue.

Panel 1.2 Summary: Ag1518 The expression of CG146422-01 gene is highest in a sample derived from normal bladder tissue. There appears to be substantial expression in other samples derived from normal tissue, including normal colon and normal kidney tissue. Further, there is substantial expression observed in cancer cell lines derived from colon cancer, renal cancer, lung cancer, ovarian cancer and breast cancer. Thus, the expression of this gene could be used to distinguish the above listed tissues from other tissues in the panel. Moreover, therapeutic modulation of this gene, through the use of antibodies, small molecule drugs or protein therapeutics might be of use in the treatment of colon cancer, renal cancer, lung cancer, ovarian cancer or breast cancer.

Panel 4.1D Summary: Ag1518 The CG146422-01 transcript is expressed in the thymus, kidney, lung and colon. Thus, the protein encoded for by this transcript could be important in the normal homeostatic mechanisms of these tissues. Furthermore, the transcript or the protein encoded by the transcript could be used diagnostically to identify these tissues.

AN. CG92738-01/GMAC076959_E: Olfactory Receptor

Expression of gene CG92738-01 was assessed using the primer-probe set Ag1519, described in Table ANA. Results of the RTQ-PCR runs are shown in Tables ANB, ANC and AND.

TABLE ANA
Probe Name Ag1519

| Primers | Sequences | Length | Start Position | SEQ ID NO |
|---|---|---|---|---|
| Forward | 5′-cctggccctcataaatctaatt-3′ | 22 | 461 | 296 |
| Probe | TET-5′-ctccttctaaggctgcccttctgtgg-3′-TAMRA | 26 | 483 | 297 |
| Reverse | 5′-acagacagaatttcaccgaaga-3′ | 22 | 529 | 298 |

TABLE ANB
Panel 1.2

| | Rel. Exp.(%) Ag1519, | | Rel. Exp.(%) Ag1519, |
|---|---|---|---|
| Tissue Name | Run 142098791 | Tissue Name | Run 142098791 |
| Endothelial cells | 0.0 | Renal ca. 786-0 | 32.1 |
| Heart (Fetal) | 1.3 | Renal ca. A498 | 10.7 |
| Pancreas | 1.5 | Renal ca. RXF 393 | 7.5 |
| Pancreatic ca. CAPAN 2 | 3.6 | Renal ca. ACHN | 10.2 |
| Adrenal Gland | 4.7 | Renal ca. UO-31 | 26.8 |
| Thyroid | 0.4 | Renal ca. TK-10 | 14.0 |
| Salivary gland | 27.7 | Liver | 7.2 |
| Pituitary gland | 0.0 | Liver (fetal) | 3.5 |
| Brain (fetal) | 0.0 | Liver ca. (hepatoblast) | 0.9 |
| | | HepG2 | |
| Brain (whole) | 0.0 | Lung | 0.0 |
| Brain (amygdala) | 0.0 | Lung (fetal) | 1.3 |
| Brain (cerebellum) | 0.0 | Lung ca. (small cell) | 22.8 |
| | | LX-1 | |
| Brain (hippocampus) | 0.3 | Lung ca. (small cell) | 11.5 |
| | | NCI-H69 | |
| Brain (thalamus) | 0.2 | Lung ca. (s. cell var.) | 0.0 |
| | | SHP-77 | |
| Cerebral Cortex | 0.3 | Lung ca. (large | 2.3 |
| | | cell) NCI-H460 | |
| Spinal cord | 0.0 | Lung ca. (non-sm. cell) | 5.1 |
| | | A549 | |
| glio/astro U87-MG | 0.4 | Lung ca. (non-s. cell) | 7.7 |
| | | NCI-H23 | |
| glio/astro U-118-MG | 0.9 | Lung ca. (non-s. cell) | 6.8 |
| | | HOP-62 | |
| Astrocytoma SW1783 | 0.0 | Lung ca. (non-s. cl) | 0.9 |
| | | NCI-H522 | |
| neuro*; met SK-N-AS | 0.0 | Lung ca. (squam.) SW | 52.1 |
| | | 900 | |
| astrocytoma SF-539 | 0.0 | Lung ca. (squam.) NCI- | 4.1 |
| | | H596 | |
| astrocytoma SNB-75 | 0.0 | Mammary Gland | 11.8 |
| glioma SNB-19 | 4.0 | Breast ca.* (pl. ef) | 11.3 |
| | | MCF-7 | |
| glioma U251 | 0.0 | Breast ca.* (pl. ef) | 1.4 |
| | | MDA-MB-231 | |
| glioma SF-295 | 2.8 | Breast ca.* (pl. ef) | 6.3 |
| | | T47D | |
| Heart | 13.9 | Breast ca. BT-549 | 0.0 |
| Skeletal Muscle | 0.2 | Breast ca. MDA-N | 12.5 |
| Bone marrow | 0.7 | Ovary | 1.5 |
| Thymus | 0.0 | Ovarian ca. OVCAR-3 | 12.4 |
| Spleen | 0.7 | Ovarian ca. OVCAR-4 | 23.8 |
| Lymph node | 0.0 | Ovarian ca. OVCAR-5 | 38.2 |
| Colorectal | 4.5 | Ovarian ca. OVCAR-8 | 35.6 |
| Stomach | 2.6 | Ovarian ca. IGROV-1 | 2.4 |
| Small intestine | 2.6 | Ovarian ca. (ascites) | 11.2 |
| | | SK-OV-3 | |
| Colon ca. SW480 | 3.1 | Uterus | 1.7 |
| Colon ca.* SW620 | 12.3 | Placenta | 0.8 |
| (SW480 met) | | | |
| Colon ca. HT29 | 12.3 | Prostate | 14.2 |
| Colon ca. HCT-116 | 14.3 | Prostate ca.* (bone | 12.6 |
| | | met) PC-3 | |
| Colon ca. CaCo-2 | 11.5 | Testis | 0.4 |
| CC Well to Mod Diff | 5.7 | Melanoma Hs688(A).T | 6.5 |
| (ODO3866) | | | |
| Colon ca. HCC-2998 | 100.0 | Melanoma* (met) | 12.2 |
| | | Hs688(B).T | |
| Gastric ca. (liver met) | 15.7 | Melanoma UACC-62 | 0.0 |
| NCI-N87 | | | |
| Bladder | 95.3 | Melanoma M14 | 10.3 |
| Trachea | 1.0 | Melanoma LOX IMVI | 0.0 |
| Kidney | 55.9 | Melanoma* (met) SK- | 0.0 |
| | | MEL-5 | |
| Kidney (fetal) | 7.7 | | |

TABLE ANC
Panel 1.3D

| | Rel. Exp.(%) Ag1519, | | Rel. Exp.(%) Ag1519, |
|---|---|---|---|
| Tissue Name | Run 165529518 | Tissue Name | Run 165529518 |
| Liver adenocarcinoma | 0.0 | Kidney (fetal) | 0.0 |
| Pancreas | 38.7 | Renal ca. 786-0 | 8.1 |
| Pancreatic ca. CAPAN 2 | 7.9 | Renal ca. A498 | 0.0 |
| Adrenal gland | 29.9 | Renal ca. RXF 393 | 29.7 |
| Thyroid | 26.6 | Renal ca. ACHN | 13.4 |
| Salivary gland | 0.0 | Renal ca. UO-31 | 0.0 |
| Pituitary gland | 17.2 | Renal ca. TK-10 | 16.8 |
| Brain (fetal) | 0.0 | Liver | 0.0 |
| Brain (whole) | 0.0 | Liver (fetal) | 27.4 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 0.0 | Lung (fetal) | 15.6 |
| Brain (substantia nigra) | 0.0 | Lung ca. (small cell) | 50.7 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 25.0 |
| | | SHP-77 | |
| Spinal cord | 0.0 | Lung ca. (large | 26.1 |
| | | cell) NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 27.4 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) SW | 18.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 27.0 |
| glioma U251 | 18.8 | Breast ca.* (pl. ef) | 27.0 |
| | | MCF-7 | |
| glioma SF-295 | 0.0 | Breast ca.* (pl. ef) | 45.7 |
| | | MDA-MB-231 | |
| Heart (Fetal) | 16.4 | Breast ca.* (pl. ef) | 13.7 |
| | | T47D | |
| Heart | 0.0 | Breast ca. BT-549 | 0.0 |
| Skeletal muscle (Fetal) | 0.0 | Breast ca. MDA-N | 13.8 |
| Skeletal muscle | 18.8 | Ovary | 0.0 |
| Bone marrow | 0.0 | Ovarian ca. OVCAR-3 | 0.0 |
| Thymus | 0.0 | Ovarian ca. OVCAR-4 | 11.4 |
| Spleen | 0.0 | Ovarian ca. OVCAR-5 | 2.6 |
| Lymph node | 34.4 | Ovarian ca. OVCAR-8 | 12.3 |
| Colorectal | 100.0 | Ovarian ca. IGROV-1 | 0.0 |
| Stomach | 0.0 | Ovarian ca. (ascites) | 33.7 |
| | | SK-OV-3 | |
| Small intestine | 0.0 | Uterus | 0.0 |
| Colon ca. SW480 | 22.8 | Placenta | 17.6 |
| Colon ca.* SW620 | 10.0 | Prostate | 0.0 |
| (SW480 met) | | | |
| Colon ca. HT29 | 16.7 | Prostate ca.* (bone | 0.0 |
| | | met) PC-3 | |
| Colon ca. HCT-116 | 16.8 | Testis | 0.0 |
| Colon ca. CaCo-2 | 15.6 | Melanoma Hs688(A).T | 15.1 |
| CC Well to Mod Diff | 28.9 | Melanoma* (met) | 9.0 |
| (ODO3866) | | Hs688(B).T | |
| Colon ca. HCC-2998 | 31.0 | Melanoma UACC-62 | 0.0 |
| Gastric ca. (liver met) | 36.6 | Melanoma M14 | 26.2 |
| NCI-N87 | | | |
| Bladder | 51.4 | Melanoma LOX IMVI | 0.0 |
| Trachea | 0.0 | Melanoma* (met) SK- | 14.1 |
| | | MEL-5 | |
| Kidney | 56.3 | Adipose | 0.0 |

TABLE AND
Panel 2D

| | Rel. Exp.(%) Ag1519, | | Rel. Exp.(%) Ag1519, |
|---|---|---|---|
| Tissue Name | Run 145158010 | Tissue Name | Run 145158010 |
| Normal Colon | 81.8 | Kidney Margin 8120608 | 5.0 |
| CC Well to Mod Diff | 6.0 | Kidney Cancer 8120613 | 0.0 |
| (ODO3866) | | | |
| CC Margin (ODO3866) | 7.3 | Kidney Margin 8120614 | 1.9 |
| CC Gr.2 rectosigmoid | 5.8 | Kidney Cancer 9010320 | 7.6 |
| (ODO3868) | | | |
| CC Margin (ODO3868) | 0.0 | Kidney Margin 9010321 | 5.8 |
| CC Mod Diff (ODO3920) | 18.6 | Normal Uterus | 4.2 |
| CC Margin (ODO3920) | 10.6 | Uterine Cancer 064011 | 47.3 |
| CC Gr.2 ascend colon | 8.2 | Normal Thyroid | 21.6 |
| (ODO3921) | | | |
| CC Margin (ODO3921) | 4.8 | Thyroid Cancer | 42.3 |
| CC from Partial | 47.6 | Thyroid Cancer | 20.9 |
| Hepatectomy (ODO4309) | | A302152 | |
| Mets | | | |
| Liver Margin (ODO4309) | 10.4 | Thyroid Margin | 59.5 |
| | | A302153 | |
| Colon mets to lung | 12.2 | Normal Breast | 71.2 |
| (OD04451-01) | | | |
| Lung Margin (OD04451-02) | 6.5 | Breast Cancer | 15.7 |
| Normal Prostate 6546-1 | 11.6 | Breast Cancer | 19.9 |
| | | (OD04590-01) | |
| Prostate Cancer (OD04410) | 31.6 | Breast Cancer Mets | 41.5 |
| | | (OD04590-03) | |
| Prostate Margin (OD04410) | 25.5 | Breast Cancer | 33.7 |
| | | Metastasis | |
| Prostate Cancer (OD04720- | 27.2 | Breast Cancer | 27.0 |
| 01) | | | |
| Prostate Margin (OD04720- | 31.4 | Breast Cancer | 48.0 |
| 02) | | | |
| Normal Lung | 25.2 | Breast Cancer 9100266 | 3.3 |
| Lung Met to Muscle | 6.2 | Breast Margin 9100265 | 7.8 |
| (ODO4286) | | | |
| Muscle Margin (ODO4286) | 0.0 | Breast Cancer A209073 | 24.8 |
| Lung Malignant Cancer | 39.0 | Breast Margin | 32.3 |
| (OD03126) | | A2090734 | |
| Lung Margin (OD03126) | 12.0 | Normal Liver | 3.5 |
| Lung Cancer (OD04404) | 4.9 | Liver Cancer | 56.6 |
| Lung Margin (OD04404) | 27.9 | Liver Cancer 1025 | 7.2 |
| Lung Cancer (OD04565) | 11.9 | Liver Cancer 1026 | 1.8 |
| Lung Margin (OD04565) | 1.4 | Liver Cancer 6004-T | 6.0 |
| Lung Cancer (OD04237-01) | 52.5 | Liver Tissue 6004-N | 0.0 |
| Lung Margin (OD04237-02) | 20.7 | Liver Cancer 6005-T | 5.6 |
| Ocular Mel Met to Liver | 5.6 | Liver Tissue 6005-N | 0.0 |
| (ODO4310) | | | |
| Liver Margin (ODO4310) | 2.2 | Normal Bladder | 24.0 |
| Melanoma Metastasis | 0.0 | Bladder Cancer | 3.3 |
| Lung Margin (OD04321) | 24.7 | Bladder Cancer | 5.7 |
| Normal Kidney | 100.0 | Bladder Cancer | 2.9 |
| | | (OD04718-01) | |
| Kidney Ca, Nuclear grade 2 | 34.4 | Bladder Normal | 0.0 |
| (OD04338) | | Adjacent (OD04718-03) | |
| Kidney Margin (OD04338) | 54.7 | Normal Ovary | 3.9 |
| Kidney Ca Nuclear grade 1/2 | 81.8 | Ovarian Cancer | 7.2 |
| (OD04339) | | | |
| Kidney Margin (OD04339) | 48.3 | Ovarian Cancer | 38.4 |
| | | (OD04768-07) | |
| Kidney Ca, Clear cell type | 11.0 | Ovary Margin | 5.1 |
| (OD04340) | | (OD04768-08) | |
| Kidney Margin (OD04340) | 56.6 | Normal Stomach | 11.4 |
| Kidney Ca, Nuclear grade 3 | 3.4 | Gastric Cancer 9060358 | 6.5 |
| (OD04348) | | | |
| Kidney Margin (OD04348) | 43.2 | Stomach Margin | 0.0 |
| | | 9060359 | |
| Kidney Cancer (OD04622- | 11.5 | Gastric Cancer 9060395 | 6.7 |
| 01) | | | |
| Kidney Margin (OD04622- | 3.5 | Stomach Margin | 4.5 |
| 03) | | 9060394 | |
| Kidney Cancer (OD04450- | 17.8 | Gastric Cancer 9060397 | 0.0 |
| 01) | | | |
| Kidney Margin (OD04450- | 42.0 | Stomach Margin | 6.7 |
| 03) | | 9060396 | |
| Kidney Cancer 8120607 | 0.0 | Gastric Cancer 064005 | 16.6 |

Panel 1.2 Summary: Ag1519 The expression of this gene appears to be highest in a sample derived from a colon cancer cell line (HCC-2998)(CT=28.2). In addition, there is substantial expression associated with normal kidney and bladder. Thus, the expression of this gene could be used to distinguish these tissues from other tissues in the panel. In addition there was noted expression clustered in ovarian, renal and colon cancer cell lines. Therefore, therapeutic modulation of this gene, through the use of small molecule drugs, antibodies or protein therapeutics might be of use in the treatment of colon cancer, renal cancer or ovarian cancer.

Among tissues with metabolic function, there is moderate expression in fetal and adult heart, adrenal, and pancreas. This expression suggests that therapeutic modulation of the expression or function of the protein encoded by this gene may be useful in the treatment of diseases that involve these tissues, including obesity and diabetes.

In addition, there appears to be higher levels of expression in adult heart (CT=3 1) when compared to expression in fetal heart (CT=34.4). Thus, expression of this gene could be used to differentiate between adult and fetal heart tissue. Conversely, expression of this gene is higher in fetal lung (CT=34.5) than in adult lung (CT=40). Thus, expression of this gene could also be used to differentiate between adult and fetal lung.

Panel 1.3D Summary: Ag1519 Significant expression is limited to a sample derived from colorectal tissue (CT=34.3). Thus, expression of this gene could be used to differentiate between this sample and other samples on this panel, and between colorectal tissue and other normal or malignant tissues.

Panel 2D Summary: Ag1519 The expression of this gene in panel 2 appears to be highest in a samples derived from normal kidney tissue (CT=32). In addition there appears to be substantial difference in expression between normal kidney adjacent to cancer tissue and the cancer tissue itself. Thus, the expression of this gene could be used to distinguish normal kidney tissue from other samples in the panel. In addition, the expression of this gene could be used to distinguish normal kidney from malignant tissue. Moreover, therapeutic modulation of this gene, through the use of small molecule drugs, antibodies or protein therapeutics might be of use in the treatment of kidney cancer.

AO. CG95462-03/GMbA144L1_B and SC122737711_A_{—}2: Olfactory Receptor

Expression of gene CG95462-03 and variant SC12273771 1_A_{—}2 was assessed using the primer-probe sets Ag2435, Ag1362, Ag1393, Ag1397, Ag1400, Ag1529, Ag1624 and Ag1630, described in Tables AOA, AOB, AOC, AOD, AOE, AOF, AOG and AOH. Results of the RTQ-PCR runs are shown in Tables AOI, AOJ, AOK, and AOL. Please note that SC122737711_A2 was previously designated as SC122737711_A.

TABLE AOA
Probe Name Ag2435

| Primers | Sequences | Length | Start Position | SEQ ID NO |
|---|---|---|---|---|
| Forward | 5′-actgggtaggtgcaaagctt-3′ | 20 | 729 | 299 |
| Probe | TET-5′-tctcacctcattattgtcactgtcca-3′-TAMRA | 26 | 763 | 300 |
| Reverse | 5′-ataaaggaggcacagccatag-3′ | 21 | 789 | 301 |

TABLE AOB
Probe Name Ag1362

| Primers | Sequences | Length | Start Position | SEQ ID NO |
|---|---|---|---|---|
| Forward | 5′-cagcagctgctctttgttatct-3′ | 22 | 106 | 302 |
| Probe | TET-5′-ctacctgttcactctgggcaccaatg-3′-TAMRA | 26 | 138 | 303 |
| Reverse | 5′-gacaatggtggaaatgatgatt-3′ | 22 | 165 | 304 |

TABLE AOC
Probe Name Ag1393

| Primers | Sequences | Length | Start Position | SEQ ID NO |
|---|---|---|---|---|
| Forward | 5′-cagcagctgctctttgttatct-3′ | 22 | 106 | 305 |
| Probe | TET-5′-ctacctgttcactctgggcaccaatg-3′-TAMRA | 26 | 138 | 306 |
| Reverse | 5′-gacaatggtggaaatgatgatt-3′ | 22 | 165 | 307 |

TABLE AOD
Probe Name Ag1397

| Primers | Sequences | Length | Start Position | SEQ ID NO |
|---|---|---|---|---|
| Forward | 5′-cagcagctgctctttgttatct-3′ | 22 | 106 | 308 |
| Probe | TET-5′-ctacctgttcactctgggcaccaatg-3′-TAMRA | 26 | 138 | 309 |
| Reserve | 5′-gacaatggtggaaatgatgatt-3′ | 22 | 165 | 310 |

TABLE AOE
Probe Name Ag1400

| Primers | Sequences | Length | Start Position | SEQ ID NO |
|---|---|---|---|---|
| Forward | 5′-cagcagctgctctttgttatct-3′ | 22 | 106 | 311 |
| Probe | TET-5═-ctacctgttcactctgggcaccaatg-3′-TAMRA | 26 | 138 | 312 |
| Reverse | 5′-gacaatggtggaaatgatgatt-3′ | 22 | 165 | 313 |

TABLE AOF
Probe Name Ag1529

| Primers | Sequences | Length | Start Position | SEQ ID NO |
|---|---|---|---|---|
| Forward | 5′-cagcagctgctctttgttatct-3′ | 22 | 106 | 314 |
| Probe | TET-5′-ctacctgttcactctgggcaccaatg-3′-TAMRA | 26 | 138 | 315 |
| Reverse | 5′-gacaatggtggaaatgatgatt-3′ | 22 | 165 | 316 |

TABLE AOG
Probe Name Ag1624

| Primers | Sequences | Length | Start Position | SEQ ID NO |
|---|---|---|---|---|
| Forward | 5′-cagcagctgctctttgttatct-3′ | 22 | 106 | 317 |
| Probe | TET-5′-ctacctgttcactctgggcaccaatg-3′-TAMRA | 26 | 138 | 318 |
| Reverse | 5′-gacaatggtggaaatgatgatt-3′ | 22 | 165 | 319 |

TABLE AOH
Probe Name Ag1630

| Primers | Sequences | Length | Start Position | SEQ ID NO |
|---|---|---|---|---|
| Forward | 5′cagcagctgctctttgttatct-3′ | 22 | 106 | 320 |
| Probe | TET-5′-ctacctgttcactctgggcaccaatg-3′-TAMRA | 26 | 138 | 321 |
| Reverse | 5′-gacaatggtggaaatgatgatt-3′ | 22 | 165 | 322 |

TABLE AOI
Panel 1.2

| | Rel. | Rel. | Rel. | Rel. | Rel. | Rel. | Rel. | Rel. |
|---|---|---|---|---|---|---|---|---|
| | Exp. (%) | Exp. (%) | Exp. (%) | Exp. (%) | Exp. (%) | Exp. (%) | Exp. (%) | Exp. (%) |
| | Ag1362, | Ag1393, | Ag1393, | Ag1397, | Ag1397, | Ag1400, | Ag1400, | Ag1529, |
| | Run | Run | Run | Run | Run | Run | Run | Run |
| Tissue Name | 133491072 | 135067950 | 138253093 | 138382684 | 139508451 | 138383072 | 139483136 | 142147887 |
| Endothelial | 0.0 | 0.0 | 0.0 | 0.4 | 0.0 | 0.0 | 0.0 | 0.0 |
| cells | | | | | | | | |
| Heart (Fetal) | 0.0 | 0.0 | 2.8 | 0.0 | 1.0 | 0.4 | 0.0 | 1.1 |
| Pancreas | 0.0 | 0.2 | 0.0 | 0.0 | 0.0 | 0.0 | 0.0 | 0.6 |
| Pancreatic ca. | 0.0 | 0.0 | 2.0 | 0.0 | 0.0 | 0.0 | 0.0 | 1.6 |
| CAPAN 2 | | | | | | | | |
| Adrenal gland | 0.0 | 0.0 | 0.0 | 0.0 | 0.0 | 0.0 | 0.0 | 0.0 |
| Thyroid | 0.0 | 0.0 | 0.0 | 0.0 | 0.0 | 0.2 | 0.0 | 0.0 |
| Salivary gland | 0.0 | 0.5 | 1.7 | 0.0 | 0.0 | 3.0 | 0.0 | 1.4 |
| Pituitary gland | 0.0 | 0.0 | 0.0 | 0.0 | 0.0 | 0.0 | 0.0 | 0.9 |
| Brain (fetal) | 0.0 | 0.0 | 1.9 | 0.0 | 0.0 | 0.0 | 0.0 | 2.1 |
| Brain (whole) | 0.0 | 0.0 | 0.0 | 0.0 | 0.0 | 0.0 | 0.0 | 0.7 |
| Brain | 0.0 | 0.2 | 0.0 | 0.2 | 0.0 | 0.0 | 0.0 | 0.0 |
| (amygdala) | | | | | | | | |
| Brain | 0.0 | 0.3 | 2.0 | 0.0 | 0.0 | 2.0 | 0.0 | 1.1 |
| (cerebellum) | | | | | | | | |
| Brain | 0.0 | 0.0 | 0.0 | 0.0 | 0.0 | 0.0 | 0.0 | 1.4 |
| (hippocampus) | | | | | | | | |
| Brain | 0.0 | 0.0 | 0.0 | 0.5 | 0.0 | 0.0 | 0.0 | 0.0 |
| (thalamus) | | | | | | | | |
| Cerebral | 0.0 | 0.0 | 0.0 | 0.0 | 0.0 | 0.0 | 0.0 | 2.8 |
| Cortex | | | | | | | | |
| Spinal cord | 0.0 | 0.0 | 0.0 | 0.0 | 0.0 | 0.0 | 0.0 | 0.0 |
| glio/astro | 0.0 | 0.0 | 1.9 | 0.3 | 0.0 | 1.1 | 1.5 | 0.0 |
| U87-MG | | | | | | | | |
| glio/astro U- | 0.0 | 1.3 | 2.8 | 2.2 | 0.9 | 1.9 | 3.4 | 0.0 |
| 118-MG | | | | | | | | |
| astrocytoma | 0.3 | 0.2 | 3.4 | 0.3 | 0.9 | 1.0 | 2.6 | 0.8 |
| SW1783 | | | | | | | | |
| neuro*; met | 0.0 | 1.1 | 0.3 | 1.2 | 0.0 | 3.1 | 3.1 | 2.5 |
| SK-N-AS | | | | | | | | |
| astrocytoma | 0.0 | 1.2 | 3.7 | 2.7 | 1.5 | 1.8 | 1.2 | 1.3 |
| SF-539 | | | | | | | | |
| astrocytoma | 0.1 | 0.6 | 3.2 | 0.4 | 2.7 | 0.6 | 3.5 | 0.0 |
| SNB-75 | | | | | | | | |
| glioma SNB-19 | 2.1 | 8.6 | 23.3 | 19.6 | 8.1 | 11.3 | 6.3 | 14.1 |
| glioma U251 | 0.3 | 0.3 | 3.3 | 0.0 | 0.8 | 0.0 | 1.0 | 3.7 |
| glioma SF-295 | 0.0 | 0.6 | 0.0 | 0.2 | 0.0 | 0.0 | 2.4 | 0.9 |
| Heart | 0.0 | 0.4 | 3.5 | 0.0 | 0.0 | 0.0 | 4.1 | 1.6 |
| Skeletal muscle | 0.8 | 0.2 | 0.0 | 0.0 | 0.0 | 0.0 | 0.0 | 0.0 |
| Bone marrow | 0.0 | 0.3 | 0.0 | 0.0 | 0.9 | 0.0 | 1.1 | 0.0 |
| Thymus | 100.0 | 100.0 | 87.1 | 84.7 | 0.0 | 59.5 | 93.3 | 82.9 |
| Spleen | 0.0 | 0.0 | 0.0 | 0.5 | 0.0 | 0.0 | 0.0 | 0.0 |
| Lymph node | 0.0 | 0.5 | 0.0 | 0.1 | 0.0 | 0.0 | 0.0 | 0.0 |
| Colorectal | 1.8 | 1.4 | 11.0 | 2.8 | 21.8 | 3.0 | 17.4 | 13.7 |
| Stomach | 0.0 | 0.2 | 0.0 | 0.0 | 0.0 | 0.0 | 0.0 | 0.0 |
| Small intestine | 0.0 | 0.0 | 2.1 | 0.0 | 0.0 | 0.0 | 0.0 | 0.0 |
| Colon ca. | 0.1 | 0.2 | 0.0 | 0.0 | 0.0 | 0.0 | 0.8 | 0.4 |
| SW480 | | | | | | | | |
| Colon ca.* | 0.0 | 0.0 | 0.0 | 0.0 | 0.0 | 0.0 | 0.0 | 0.6 |
| SW620 | | | | | | | | |
| (SW480 met) | | | | | | | | |
| Colon ca. | 0.6 | 11.2 | 10.5 | 3.4 | 6.3 | 3.6 | 4.4 | 3.9 |
| HT29 | | | | | | | | |
| Colon ca. | 0.0 | 0.0 | 1.7 | 1.0 | 0.0 | 0.4 | 0.0 | 0.9 |
| HCT-116 | | | | | | | | |
| Colon ca. | 0.0 | 0.7 | 1.8 | 0.6 | 1.8 | 0.0 | 0.0 | 1.7 |
| CaCo-2 | | | | | | | | |
| CC Well to | 11.7 | 9.7 | 29.9 | 16.0 | 27.0 | 18.6 | 31.2 | 6.5 |
| Mod Diff | | | | | | | | |
| (ODO3866) | | | | | | | | |
| Colon ca. | 0.1 | 0.6 | 3.3 | 1.0 | 1.9 | 1.5 | 6.5 | 3.6 |
| HCC-2998 | | | | | | | | |
| Gastric ca. | 0.5 | 0.8 | 1.6 | 0.7 | 1.8 | 2.6 | 3.3 | 1.5 |
| (liver met) | | | | | | | | |
| NCI-N87 | | | | | | | | |
| Bladder | 1.1 | 2.4 | 22.2 | 4.9 | 8.0 | 2.5 | 9.8 | 6.4 |
| Trachea | 0.2 | 0.0 | 2.4 | 0.4 | 0.0 | 0.7 | 0.0 | 1.1 |
| Kidney | 0.0 | 1.8 | 1.6 | 4.5 | 3.7 | 1.2 | 1.8 | 11.1 |
| Kidney (fetal) | 0.3 | 1.2 | 0.0 | 1.7 | 0.8 | 0.0 | 0.0 | 0.0 |
| Renal ca. 786-0 | 0.0 | 0.4 | 0.0 | 0.0 | 0.0 | 0.0 | 0.0 | 0.0 |
| Renal ca. | 0.6 | 1.1 | 5.0 | 3.7 | 3.8 | 1.4 | 3.3 | 7.5 |
| A498 | | | | | | | | |
| Renal ca. RXF | 0.0 | 0.0 | 0.0 | 0.0 | 0.0 | 0.4 | 0.0 | 0.0 |
| 393 | | | | | | | | |
| Renal ca. | 0.3 | 0.2 | 1.7 | 0.9 | 3.2 | 0.7 | 0.0 | 2.7 |
| ACHN | | | | | | | | |
| Renal ca. UO-31 | 0.3 | 0.8 | 10.0 | 2.4 | 5.9 | 2.8 | 4.0 | 4.9 |
| Renal ca. TK-10 | 0.3 | 2.2 | 10.6 | 2.3 | 7.5 | 2.8 | 8.4 | 6.3 |
| Liver | 0.0 | 0.0 | 0.0 | 0.0 | 0.0 | 0.0 | 0.0 | 1.2 |
| Liver (fetal) | 0.0 | 0.0 | 0.0 | 0.0 | 0.0 | 0.0 | 1.2 | 0.0 |
| Liver ca. | 0.3 | 0.0 | 0.0 | 0.4 | 0.0 | 0.0 | 0.0 | 2.1 |
| (hepatoblast) | | | | | | | | |
| HepG2 | | | | | | | | |
| Lung | 0.1 | 0.0 | 1.3 | 0.0 | 0.0 | 0.4 | 0.0 | 0.0 |
| Lung (fetal) | 0.0 | 0.0 | 0.0 | 0.0 | 0.0 | 0.0 | 1.2 | 0.8 |
| Lung ca. | 0.0 | 0.5 | 1.4 | 0.5 | 0.0 | 0.7 | 2.1 | 2.6 |
| (small cell) | | | | | | | | |
| LX-1 | | | | | | | | |
| Lung ca. | 5.0 | 51.8 | 100.0 | 100.0 | 75.8 | 34.4 | 100.0 | 100.0 |
| (small cell) | | | | | | | | |
| NCI-H69 | | | | | | | | |
| Lung ca. | 0.0 | 0.6 | 3.0 | 1.6 | 2.0 | 1.1 | 4.3 | 0.6 |
| (s.cell var.) | | | | | | | | |
| SHP-77 | | | | | | | | |
| Lung ca. | 4.3 | 2.6 | 20.3 | 12.2 | 13.5 | 5.7 | 17.1 | 11.7 |
| (large | | | | | | | | |
| cell) NCI- | | | | | | | | |
| H460 | | | | | | | | |
| Lung ca. (non- | 1.9 | 5.4 | 34.6 | 32.8 | 29.3 | 3.4 | 17.3 | 28.5 |
| sm. cell) A549 | | | | | | | | |
| Lung ca. (non- | 0.0 | 0.0 | 0.9 | 1.8 | 1.7 | 0.0 | 1.8 | 1.1 |
| s.cell) NCI- | | | | | | | | |
| H23 | | | | | | | | |
| Lung ca. (non- | 2.0 | 1.0 | 33.2 | 2.2 | 11.6 | 0.0 | 10.3 | 14.6 |
| s.cell) HOP-62 | | | | | | | | |
| Lung ca. (non- | 0.0 | 0.2 | 6.7 | 1.1 | 0.0 | 1.1 | 2.9 | 4.0 |
| s.cl) NCI- | | | | | | | | |
| H522 | | | | | | | | |
| Lung ca. | 0.5 | 0.9 | 11.3 | 6.6 | 11.2 | 2.9 | 7.7 | 10.7 |
| (squam.) SW | | | | | | | | |
| 900 | | | | | | | | |
| Lung ca. | 0.6 | 6.5 | 40.1 | 13.3 | 29.1 | 14.1 | 45.7 | 27.7 |
| (squam.) NCI- | | | | | | | | |
| H596 | | | | | | | | |
| Mammary | 0.0 | 0.3 | 0.0 | 0.0 | 0.0 | 0.0 | 0.0 | 0.9 |
| gland | | | | | | | | |
| Breast ca.* | 8.0 | 9.5 | 73.2 | 8.1 | 0.0 | 100.0 | 47.0 | 30.4 |
| (pl.ef) MCF-7 | | | | | | | | |
| Breast ca.* | 0.2 | 0.2 | 0.0 | 0.3 | 0.0 | 0.0 | 1.0 | 0.9 |
| (pl.ef) MDA- | | | | | | | | |
| MB-231 | | | | | | | | |
| Breast ca.* | 2.5 | 1.9 | 44.8 | 34.2 | 46.7 | 20.9 | 23.5 | 39.8 |
| (pl. ef) T47D | | | | | | | | |
| Breast ca. BT- | 0.9 | 4.7 | 9.0 | 1.1 | 3.7 | 8.5 | 3.6 | 9.2 |
| 549 | | | | | | | | |
| Breast ca. | 1.4 | 1.1 | 10.9 | 2.7 | 13.4 | 5.9 | 19.1 | 16.3 |
| MDA-N | | | | | | | | |
| Ovary | 0.0 | 0.2 | 0.0 | 0.0 | 0.0 | 0.0 | 0.0 | 0.0 |
| Ovarian ca. | 0.5 | 1.3 | 3.2 | 7.4 | 1.7 | 1.6 | 2.6 | 2.4 |
| OVCAR-3 | | | | | | | | |
| Ovarian ca. | 0.0 | 0.3 | 0.0 | 0.5 | 2.0 | 0.0 | 1.9 | 0.6 |
| OVCAR-4 | | | | | | | | |
| Ovarian ca. | 4.5 | 33.4 | 97.9 | 71.2 | 100.0 | 37.9 | 88.3 | 95.9 |
| OVCAR-5 | | | | | | | | |
| Ovarian ca. | 0.9 | 4.8 | 1.6 | 8.2 | 1.9 | 5.9 | 1.0 | 0.6 |
| OVCAR-8 | | | | | | | | |
| Ovarian ca. | 0.1 | 4.0 | 12.9 | 14.6 | 7.2 | 7.2 | 9.0 | 10.0 |
| IGROV-1 | | | | | | | | |
| Ovarian ca. | 0.7 | 2.5 | 4.0 | 3.1 | 7.7 | 2.9 | 8.4 | 3.6 |
| (ascites) SK- | | | | | | | | |
| OV-3 | | | | | | | | |
| Uterus | 0.0 | 0.0 | 0.0 | 0.0 | 0.0 | 0.0 | 0.0 | 0.3 |
| Placenta | 0.0 | 0.0 | 0.0 | 0.0 | 0.0 | 0.0 | 0.0 | 0.0 |
| Prostate | 0.1 | 0.0 | 4.9 | 0.3 | 0.8 | 0.2 | 0.0 | 3.7 |
| Prostate ca.* | 0.4 | 0.7 | 5.0 | 1.8 | 5.1 | 0.5 | 6.3 | 2.1 |
| (bone met) | | | | | | | | |
| PC-3 | | | | | | | | |
| Testis | 0.9 | 0.0 | 1.7 | 0.9 | 0.0 | 1.0 | 0.3 | 1.4 |
| Melanoma | 0.0 | 0.0 | 0.0 | 0.0 | 0.0 | 0.0 | 0.0 | 0.0 |
| Hs688(A).T | | | | | | | | |
| Melanoma* | 0.3 | 2.0 | 12.4 | 10.4 | 11.0 | 2.2 | 8.3 | 5.0 |
| (met) | | | | | | | | |
| Hs688(B).T | | | | | | | | |
| Melanoma | 0.0 | 0.0 | 0.0 | 0.0 | 0.0 | 0.0 | 1.5 | 0.6 |
| UACC-62 | | | | | | | | |
| Melanoma | 6.0 | 9.2 | 70.2 | 10.9 | 44.4 | 12.3 | 47.0 | 62.4 |
| M14 | | | | | | | | |
| Melanoma | 0.4 | 0.0 | 2.8 | 0.8 | 1.7 | 0.0 | 1.1 | 1.5 |
| LOX IMVI | | | | | | | | |
| Melanoma* | 0.0 | 0.0 | 0.0 | 0.0 | 0.8 | 0.0 | 0.8 | 0.0 |
| (met) SK- | | | | | | | | |
| MEL-5 | | | | | | | | |

TABLE AOJ
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1400, | Ag1624, | Ag1630, | | Ag1400, | Ag1624, | Ag1630, |
| | Run | Run | Run | Tissue | Run | Run | Run |
| Tissue Name | 147633649 | 165924144 | 155604421 | Name | 147633649 | 165924144 | 155604421 |
| Liver | 0.0 | 1.5 | 0.0 | Kidney | 0.0 | 0.0 | 0.0 |
| adenocarcinoma | | | | (fetal) | | | |
| Pancreas | 0.0 | 0.0 | 2.9 | Renal ca. | 0.0 | 0.0 | 0.0 |
| | | | | 786-0 | | | |
| Pancreatic ca. | 0.0 | 0.0 | 0.0 | Renal ca. | 0.0 | 0.0 | 0.0 |
| CAPAN 2 | | | | A498 | | | |
| Adrenal gland | 0.0 | 0.0 | 0.0 | Renal ca. | 0.0 | 2.5 | 0.0 |
| | | | | RXF 393 | | | |
| Thyroid | 0.0 | 0.0 | 0.0 | Renal ca. | 3.1 | 0.0 | 0.0 |
| | | | | ACHN | | | |
| Salivary gland | 3.2 | 0.0 | 0.0 | Renal ca. | 0.0 | 0.0 | 0.0 |
| | | | | UO-31 | | | |
| Pituitary gland | 0.0 | 0.0 | 0.0 | Renal ca. | 0.0 | 0.0 | 0.0 |
| | | | | TK-10 | | | |
| Brain (fetal) | 0.0 | 0.0 | 0.0 | Liver | 0.0 | 0.0 | 0.0 |
| Brain (whole) | 0.0 | 0.0 | 0.0 | Liver (fetal) | 0.0 | 0.0 | 0.0 |
| Brain | 0.0 | 0.0 | 0.0 | Liver ca. | 0.0 | 0.0 | 0.0 |
| (amygdala) | | | | (hepatoblast) | | | |
| | | | | HepG2 | | | |
| Brain | 0.0 | 0.0 | 0.0 | Lung | 0.0 | 0.0 | 2.3 |
| (cerebellum) | | | | | | | |
| Brain | 0.0 | 0.0 | 0.0 | Lung (fetal) | 0.0 | 0.0 | 0.0 |
| (hippocampus) | | | | | | | |
| Brain | 3.6 | 0.0 | 0.0 | Lung ca. | 0.0 | 0.0 | 0.0 |
| (substantia | | | | (small cell) | | | |
| nigra) | | | | LX-1 | | | |
| Brain | 0.0 | 0.0 | 0.0 | Lung ca. | 0.0 | 0.0 | 0.0 |
| (thalamus) | | | | (small cell) | | | |
| | | | | NCI-H69 | | | |
| Cerebral Cortex | 0.0 | 0.0 | 0.0 | Lung ca. | 0.0 | 0.0 | 0.0 |
| | | | | (s.cell var.) | | | |
| | | | | SHP-77 | | | |
| Spinal cord | 0.0 | 0.0 | 0.0 | Lung ca. | 0.0 | 0.0 | 2.6 |
| | | | | (large | | | |
| | | | | cell) NCI- | | | |
| | | | | H460 | | | |
| glio/astro U87-MG | 0.0 | 0.0 | 2.0 | Lung ca. | 0.0 | 0.0 | 0.0 |
| | | | | (non-sm. | | | |
| | | | | cell) A549 | | | |
| glio/astro U- | 2.3 | 0.0 | 0.0 | Lung ca. | 0.0 | 0.0 | 0.0 |
| 118-MG | | | | (non-s.cell) | | | |
| | | | | NCI-H23 | | | |
| astrocytoma | 3.7 | 0.0 | 0.0 | Lung ca. | 0.0 | 0.0 | 0.0 |
| SW1783 | | | | (non-s.cell) | | | |
| | | | | HOP-62 | | | |
| neuro*; met SK- | 0.0 | 0.0 | 0.0 | Lung ca. | 7.6 | 0.0 | 0.0 |
| N-AS | | | | (non-s.cl) | | | |
| | | | | NCI-H522 | | | |
| astrocytoma SF- | 0.0 | 0.0 | 4.4 | Lung ca. | 0.0 | 0.0 | 0.0 |
| 539 | | | | (squam.) SW | | | |
| | | | | 900 | | | |
| astrocytoma | 4.0 | 0.0 | 0.0 | Lung ca. | 0.0 | 0.0 | 0.0 |
| SNB-75 | | | | (squam.) | | | |
| | | | | NCI-H596 | | | |
| glioma SNB-19 | 0.0 | 0.0 | 3.5 | Mammary | 0.0 | 0.0 | 0.0 |
| | | | | gland | | | |
| glioma U251 | 0.0 | 0.0 | 0.0 | Breast ca.* | 15.8 | 0.0 | 19.6 |
| | | | | (pl.ef) | | | |
| | | | | MCF-7 | | | |
| glioma SF-295 | 0.0 | 0.0 | 0.0 | Breast ca.* | 0.0 | 0.0 | 0.0 |
| | | | | (pl.ef) | | | |
| | | | | MDA-MB- | | | |
| | | | | 231 | | | |
| Heart (Fetal) | 0.0 | 0.0 | 0.0 | Breast ca.* | 0.0 | 0.0 | 0.0 |
| | | | | (pl. ef) T47D | | | |
| Heart | 0.0 | 0.0 | 0.0 | Breast ca.* | 3.7 | 0.0 | 2.0 |
| | | | | BT-549 | | | |
| Skeletal muscle | 0.0 | 27.2 | 1.8 | Breast ca. | 0.0 | 0.0 | 0.0 |
| (Fetal) | | | | MDA-N | | | |
| Skeletal muscle | 0.0 | 0.0 | 0.0 | Ovary | 0.0 | 0.0 | 0.0 |
| Bone marrow | 0.0 | 0.0 | 0.0 | Ovarian ca. | 0.0 | 0.0 | 0.0 |
| | | | | OVCAR-3 | | | |
| Thymus | 100.0 | 56.6 | 100.0 | Ovarian ca. | 2.6 | 0.0 | 0.0 |
| | | | | OVCAR-4 | | | |
| Spleen | 0.0 | 100.0 | 0.0 | Ovarian ca. | 0.0 | 0.0 | 0.0 |
| | | | | OVCAR-5 | | | |
| Lymph node | 0.0 | 0.0 | 0.0 | Ovarian ca. | 0.0 | 0.0 | 0.0 |
| | | | | OVCAR-8 | | | |
| Colorectal | 17.2 | 5.7 | 7.4 | Ovarian ca. | 0.0 | 4.9 | 0.0 |
| | | | | IGROV-1 | | | |
| Stomach | 0.0 | 0.0 | 0.0 | Ovarian ca. | 0.0 | 5.4 | 0.0 |
| | | | | (ascites) SK- | | | |
| | | | | OV-3 | | | |
| Small intestine | 0.0 | 0.0 | 0.0 | Uterus | 0.0 | 0.0 | 0.0 |
| Colon ca. | 0.0 | 0.0 | 0.0 | Placenta | 0.0 | 0.0 | 0.0 |
| SW480 | | | | | | | |
| Colon ca.* | 0.0 | 0.0 | 0.0 | Prostate | 0.0 | 0.0 | 0.0 |
| SW620 (SW480 | | | | | | | |
| met) | | | | | | | |
| Colon ca. HT29 | 3.4 | 0.0 | 4.6 | Prostate ca.* | 0.0 | 0.0 | 0.0 |
| | | | | (bone met) | | | |
| | | | | PC-3 | | | |
| Colon ca. HCT- | 0.0 | 0.0 | 0.0 | Testis | 0.0 | 0.0 | 1.9 |
| 116 | | | | | | | |
| Colon ca. CaCo-2 | 0.0 | 0.0 | 0.0 | Melanoma | 0.0 | 0.0 | 0.0 |
| | | | | Hs688(A).T | | | |
| CC Well to Mod | 0.0 | 0.0 | 0.0 | Melanoma* | 0.0 | 0.0 | 0.0 |
| Diff | | | | (met) | | | |
| (ODO3866) | | | | Hs688(B).T | | | |
| Colon ca. HCC- | 0.0 | 0.0 | 0.0 | Melanoma | 0.0 | 0.0 | 0.0 |
| 2998 | | | | UACC-62 | | | |
| Gastric ca. (liver | 0.0 | 0.0 | 1.2 | Melanoma | 0.0 | 0.0 | 0.0 |
| met) NCI-N87 | | | | M14 | | | |
| Bladder | 2.6 | 0.0 | 2.3 | Melanoma | 0.0 | 0.0 | 0.0 |
| | | | | LOX IMVI | | | |
| Trachea | 0.0 | 0.0 | 3.2 | Melanoma* | 0.0 | 0.0 | 0.0 |
| | | | | (met) SK- | | | |
| | | | | MEL-5 | | | |
| Kidney | 0.0 | 0.0 | 0.0 | Adipose | 0.0 | 0.0 | 0.0 |

TABLE AOK
Panel 2D

| | Rel. Exp.(%) Ag1630, | | Rel. Exp.(%) Ag1630, |
|---|---|---|---|
| Tissue Name | Run 155604902 | Tissue Name | Run 155604902 |
| Normal Colon | 9.7 | Kidney Margin 8120608 | 0.0 |
| CC Well to Mod Diff | 71.7 | Kidney Cancer 8120613 | 0.0 |
| (ODO3866) | | | |
| CC Margin (ODO3866) | 0.0 | Kidney Margin 8120614 | 0.0 |
| CC Gr.2 rectosigmoid | 0.0 | Kidney Cancer 9010320 | 0.0 |
| (ODO3868) | | | |
| CC Margin (ODO3868) | 10.2 | Kidney Margin 9010321 | 0.0 |
| CC Mod Diff (ODO3920) | 0.0 | Normal Uterus | 0.0 |
| CC Margin (ODO3920) | 0.0 | Uterine Cancer 064011 | 0.0 |
| CC Gr.2 ascend colon | 0.0 | Normal Thyroid | 0.0 |
| (ODO3921) | | | |
| CC Margin (ODO3921) | 100.0 | Thyroid Cancer | 0.0 |
| CC from Partial | 0.0 | Thyroid Cancer | 0.0 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 0.0 | Normal Breast | 0.0 |
| (OD04451-01) | | | |
| Lung Margin (OD04451-02) | 0.0 | Breast Cancer | 0.0 |
| Normal Prostate 6546-1 | 0.0 | Breast Cancer | 0.0 |
| | | (OD04590-01) | |
| Prostate Cancer (OD04410) | 0.0 | Breast Cancer Mets | 0.0 |
| | | (OD04590-03) | |
| Prostate Margin (OD04410) | 0.0 | Breast Cancer | 0.0 |
| | | Metastasis | |
| Prostate Cancer (OD04720- | 0.0 | Breast Cancer | 0.0 |
| 01) | | | |
| Prostate Margin (OD04720- | 0.0 | Breast Cancer | 0.0 |
| 02) | | | |
| Normal Lung | 0.0 | Breast Cancer 9100266 | 0.0 |
| Lung Met to Muscle | 10.4 | Breast Margin 9100265 | 0.0 |
| (ODO4286) | | | |
| Muscle Margin (ODO4286) | 0.0 | Breast Cancer A209073 | 17.7 |
| Lung Malignant Cancer | 0.0 | Breast Margin | 0.0 |
| (OD03126) | | A2090734 | |
| Lung Margin (OD03126) | 0.0 | Normal Liver | 0.0 |
| Lung Cancer (OD04404) | 0.0 | Liver Cancer | 0.0 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 3.4 | Liver Cancer 6004-T | 0.0 |
| Lung Cancer (OD04237-01) | 0.0 | Liver Tissue 6004-N | 21.6 |
| Lung Margin (OD04237-02) | 0.0 | Liver Cancer 6005-T | 0.0 |
| Ocular Mel Met to Liver | 0.0 | Liver Tissue 6005-N | 0.0 |
| (ODO4310) | | | |
| Liver Margin (ODO4310) | 0.0 | Normal Bladder | 0.0 |
| Melanoma Metastasis | 0.0 | Bladder Cancer | 23.8 |
| Lung Margin (OD04321) | 0.0 | Bladder Cancer | 83.5 |
| Normal Kidney | 0.0 | Bladder Cancer | 0.0 |
| | | (OD04718-01) | |
| Kidney Ca, Nuclear grade 2 | 0.0 | Bladder Normal | 0.0 |
| (OD04338) | | Adjacent (OD04718-03) | |
| Kidney Margin (OD04338) | 0.0 | Normal Ovary | 0.0 |
| Kidney Ca Nuclear grade 1/2 | 4.7 | Ovarian Cancer | 0.0 |
| (OD04339) | | | |
| Kidney Margin (OD04339) | 7.2 | Ovarian Cancer | 0.0 |
| | | (OD04768-07) | |
| Kidney Ca, Clear cell type | 0.0 | Ovary Margin | 0.0 |
| (OD04340) | | (OD04768-08) | |
| Kidney Margin (OD04340) | 0.0 | Normal Stomach | 0.0 |
| Kidney Ca, Nuclear grade 3 | 0.0 | Gastric Cancer 9060358 | 0.0 |
| (OD04348) | | | |
| Kidney Margin (OD04348) | 0.0 | Stomach Margin | 0.0 |
| | | 9060359 | |
| Kidney Cancer (OD04622-01) | 0.0 | Gastric Cancer 9060395 | 0.0 |
| Kidney Margin (OD04622-03) | 0.0 | Stomach Margin | 14.9 |
| | | 9060394 | |
| Kidney Cancer (OD04450-01) | 4.5 | Gastric Cancer 9060397 | 0.0 |
| Kidney Margin (OD04450-03) | 0.0 | Stomach Margin | 0.0 |
| | | 9060396 | |
| Kidney Cancer 8120607 | 0.0 | Gastric Cancer 064005 | 0.0 |

TABLE AOL
Panel 4D

| | Rel. | Rel. | Rel. | Rel. | Rel. | Rel. |
|---|---|---|---|---|---|---|
| | Exp. (%) | Exp. (%) | Exp. (%) | Exp. (%) | Exp. (%) | Exp. (%) |
| | Ag1397, | Ag1400, | Ag1400, | Ag1624, | Ag1630, | Ag2435, |
| | Run | Run | Run | Run | Run | Run |
| Tissue Name | 138273346 | 138273367 | 138986251 | 165762855 | 155605294 | 164184028 |
| Secondary Th1 act | 0.0 | 0.0 | 0.0 | 0.3 | 0.0 | 0.0 |
| Secondary Th2 act | 0.0 | 0.0 | 0.0 | 0.2 | 0.6 | 0.0 |
| Secondary Tr1 act | 0.2 | 0.0 | 0.5 | 0.1 | 0.0 | 0.0 |
| Secondary Th1 rest | 0.0 | 0.0 | 0.6 | 0.2 | 0.0 | 0.0 |
| Secondary Th2 rest | 0.0 | 0.0 | 0.0 | 0.0 | 0.0 | 0.0 |
| Secondary Tr1 rest | 0.0 | 0.0 | 0.0 | 0.0 | 0.0 | 0.0 |
| Primary Th1 act | 0.0 | 0.0 | 0.0 | 0.0 | 0.6 | 2.0 |
| Primary Th2 act | 0.0 | 0.0 | 0.0 | 0.0 | 0.0 | 0.0 |
| Primary Tr1 act | 0.0 | 0.0 | 0.0 | 0.0 | 0.0 | 0.0 |
| Primary Th1 rest | 0.0 | 0.0 | 0.0 | 0.0 | 0.0 | 0.0 |
| Primary Th2 rest | 0.0 | 0.0 | 0.0 | 0.0 | 0.0 | 0.0 |
| Primary Tr1 rest | 0.0 | 0.0 | 0.0 | 0.0 | 0.0 | 0.0 |
| CD45RA CD4 | 0.0 | 0.0 | 0.0 | 0.2 | 0.0 | 0.0 |
| lymphocyte act | | | | | | |
| CD45RO CD4 | 0.0 | 0.0 | 0.0 | 0.0 | 0.0 | 0.0 |
| lymphocyte act | | | | | | |
| CD8 lymphocyte act | 0.0 | 0.0 | 0.0 | 0.0 | 0.0 | 0.0 |
| Secondary CD8 | 0.0 | 0.0 | 0.0 | 0.0 | 0.0 | 0.0 |
| lymphocyte rest | | | | | | |
| Secondary CD8 | 0.0 | 0.0 | 0.0 | 0.2 | 0.0 | 0.0 |
| lymphocyte act | | | | | | |
| CD4 lymphocyte none | 0.0 | 0.0 | 0.5 | 0.0 | 0.0 | 0.0 |
| 2ry Th1/Th2/Tr1_anti- | 0.2 | 0.0 | 0.0 | 0.0 | 0.3 | 0.0 |
| CD95 CH11 | | | | | | |
| LAK cells rest | 0.0 | 0.0 | 0.0 | 0.0 | 0.0 | 0.0 |
| LAK cells IL-2 | 0.0 | 0.0 | 0.0 | 0.0 | 0.0 | 1.5 |
| LAK cells IL-2 + IL-12 | 0.0 | 0.0 | 0.0 | 0.0 | 0.4 | 0.0 |
| LAK cells IL-2 + IFN | 0.0 | 0.0 | 0.0 | 0.0 | 0.0 | 0.0 |
| gamma | | | | | | |
| LAK cells IL-2 + IL-18 | 0.0 | 0.0 | 0.0 | 0.0 | 0.0 | 0.0 |
| LAK cells | 0.0 | 0.0 | 0.0 | 0.0 | 0.0 | 0.0 |
| PMA/ionomycin | | | | | | |
| NK Cells IL-2 rest | 0.0 | 0.0 | 0.0 | 0.2 | 0.0 | 3.5 |
| Two Way MLR 3 day | 0.0 | 0.0 | 0.0 | 0.0 | 0.0 | 0.0 |
| Two Way MLR 5 day | 0.0 | 0.0 | 0.0 | 0.0 | 0.0 | 0.0 |
| Two Way MLR 7 day | 0.0 | 0.0 | 0.0 | 0.0 | 0.0 | 0.0 |
| PBMC rest | 0.0 | 0.0 | 0.0 | 0.0 | 0.0 | 0.0 |
| PBMC PWM | 0.0 | 0.0 | 0.0 | 0.0 | 0.0 | 0.0 |
| PBMC PHA-L | 0.0 | 0.0 | 0.0 | 0.0 | 0.0 | 0.0 |
| Ramos (B cell) none | 0.0 | 0.0 | 0.0 | 0.0 | 0.0 | 0.0 |
| Ramos (B cell) | 0.0 | 0.0 | 0.0 | 0.0 | 0.0 | 0.0 |
| ionomycin | | | | | | |
| B lymphocytes PWM | 0.0 | 0.0 | 0.0 | 0.0 | 0.3 | 0.0 |
| B lymphocytes CD40L | 0.0 | 0.0 | 0.0 | 0.0 | 0.0 | 0.0 |
| and IL-4 | | | | | | |
| EOL-1 dbcAMP | 0.0 | 0.0 | 0.0 | 0.0 | 0.0 | 0.0 |
| EOL-1 dbcAMP | 0.9 | 0.0 | 0.0 | 0.1 | 0.0 | 0.0 |
| PMA/ionomycin | | | | | | |
| Dendritic cells none | 0.0 | 0.0 | 0.0 | 0.0 | 0.0 | 0.0 |
| Dendritic cells LPS | 0.0 | 0.0 | 0.0 | 0.1 | 0.0 | 0.0 |
| Dendritic cells anti- | 0.0 | 0.0 | 0.0 | 0.0 | 0.0 | 0.0 |
| CD40 | | | | | | |
| Monocytes rest | 0.0 | 0.0 | 0.0 | 0.0 | 0.0 | 0.0 |
| Monocytes LPS | 0.0 | 0.0 | 0.0 | 0.0 | 0.0 | 0.0 |
| Macrophages rest | 0.1 | 0.0 | 0.0 | 0.0 | 0.0 | 0.0 |
| Macrophages LPS | 0.0 | 0.4 | 0.0 | 0.0 | 0.0 | 0.0 |
| HUVEC none | 0.0 | 0.0 | 0.0 | 0.0 | 0.0 | 0.0 |
| HUVEC starved | 0.0 | 0.0 | 0.0 | 0.0 | 0.0 | 0.0 |
| HUVEC IL-1 beta | 0.0 | 0.0 | 0.0 | 0.0 | 0.0 | 0.0 |
| HUVEC IFN gamma | 0.0 | 0.0 | 0.0 | 0.0 | 0.0 | 0.0 |
| HUVEC TNF alpha + | 0.0 | 0.0 | 0.0 | 0.8 | 0.0 | 0.0 |
| IFN gamma | | | | | | |
| HUVEC TNF alpha + | 0.0 | 0.0 | 0.0 | 0.0 | 0.0 | 0.0 |
| IL4 | | | | | | |
| HUVEC IL-11 | 0.0 | 0.0 | 0.0 | 0.0 | 0.0 | 0.0 |
| Lung Microvascular EC | 0.0 | 0.0 | 0.0 | 0.0 | 0.6 | 0.0 |
| none | | | | | | |
| Lung Microvascular EC | 0.0 | 0.0 | 0.0 | 0.0 | 0.0 | 0.0 |
| TNF alpha + IL-1 beta | | | | | | |
| Microvascular Dermal | 0.0 | 0.0 | 0.0 | 0.0 | 0.0 | 0.0 |
| EC none | | | | | | |
| Microvascular Dermal | 0.0 | 0.0 | 0.0 | 0.0 | 0.0 | 0.0 |
| EC TNF alpha + IL- | | | | | | |
| 1 beta | | | | | | |
| Bronchial epithelium | 0.0 | 0.0 | 0.0 | 0.0 | 0.0 | 0.0 |
| TNF alpha + IL1 beta | | | | | | |
| Small airway | 0.0 | 0.0 | 0.0 | 0.6 | 0.0 | 0.0 |
| epithelium none | | | | | | |
| Small airway | 0.0 | 0.0 | 0.0 | 0.0 | 0.0 | 0.0 |
| epithelium TNF alpha + | | | | | | |
| IL-1 beta | | | | | | |
| Coronery artery SMC | 0.0 | 0.0 | 0.0 | 0.0 | 0.0 | 0.0 |
| rest | | | | | | |
| Coronery artery SMC | 0.0 | 0.0 | 0.0 | 0.0 | 0.0 | 0.0 |
| TNF alpha + IL-1 beta | | | | | | |
| Astrocytes rest | 0.0 | 0.0 | 0.0 | 0.0 | 0.0 | 0.0 |
| Astrocytes TNF alpha + | 0.0 | 0.0 | 0.0 | 0.0 | 0.0 | 0.0 |
| IL-1 beta | | | | | | |
| KU-812 (Basophil) rest | 0.0 | 0.0 | 0.0 | 0.0 | 0.0 | 0.0 |
| KU-812 (Basophil) | 0.0 | 0.0 | 0.0 | 0.0 | 0.0 | 0.0 |
| PMA/ionomycin | | | | | | |
| CCD1106 | 0.0 | 0.0 | 0.0 | 0.0 | 0.0 | 0.0 |
| (Keratinocytes) none | | | | | | |
| 93580_CCD1106 | 0.0 | 0.0 | 0.0 | 0.0 | 0.0 | 0.0 |
| (Keratinocytes)_TNFa | | | | | | |
| and IFNg | | | | | | |
| Liver cirrhosis | 3.5 | 6.9 | 4.5 | 14.8 | 3.4 | 13.8 |
| Lupus kidney | 0.0 | 0.0 | 0.0 | 0.2 | 0.0 | 0.0 |
| NCI-H292 none | 0.0 | 0.0 | 0.0 | 0.0 | 0.0 | 0.0 |
| NCI-H292 IL-4 | 0.0 | 0.0 | 0.0 | 0.0 | 0.0 | 0.0 |
| NCI-H292 IL-9 | 0.0 | 0.0 | 0.0 | 0.0 | 0.0 | 0.0 |
| NCI-H292 IL-13 | 0.0 | 0.0 | 0.0 | 0.0 | 0.0 | 0.0 |
| NCI-H292 IFN gamma | 0.5 | 0.0 | 0.0 | 0.0 | 0.0 | 0.0 |
| HPAEC none | 0.0 | 0.0 | 0.0 | 0.0 | 0.0 | 0.0 |
| HPAEC TNF alpha + | 0.0 | 0.0 | 0.0 | 0.0 | 0.0 | 0.0 |
| IL-1 beta | | | | | | |
| Lung fibroblast none | 0.0 | 0.0 | 0.0 | 0.5 | 0.0 | 0.0 |
| Lung fibroblast TNF | 0.0 | 0.0 | 0.0 | 0.0 | 0.0 | 0.0 |
| alpha + IL-1 beta | | | | | | |
| Lung fibroblast IL-4 | 0.0 | 0.0 | 0.0 | 0.0 | 0.0 | 0.0 |
| Lung fibroblast IL-9 | 0.0 | 0.0 | 0.0 | 0.0 | 0.0 | 0.0 |
| Lung fibroblast IL-13 | 0.0 | 0.0 | 0.0 | 0.0 | 0.0 | 0.0 |
| Lung fibroblast IFN | 0.0 | 0.0 | 0.0 | 0.0 | 0.0 | 0.0 |
| gamma | | | | | | |
| Dermal fibroblast | 0.0 | 0.0 | 0.0 | 0.0 | 0.0 | 0.0 |
| CCD1070 rest | | | | | | |
| Dermal fibroblast | 0.0 | 0.0 | 0.3 | 0.0 | 0.0 | 0.0 |
| CCD1070 TNF alpha | | | | | | |
| Dermal fibroblast | 0.0 | 0.0 | 0.0 | 0.0 | 0.0 | 0.0 |
| CCD1070 IL-1 beta | | | | | | |
| Dermal fibroblast IFN | 0.0 | 0.0 | 0.0 | 0.0 | 0.0 | 0.0 |
| gamma | | | | | | |
| Dermal fibroblast IL-4 | 0.0 | 0.0 | 0.0 | 0.0 | 0.3 | 0.0 |
| IBD Colitis 2 | 0.2 | 1.4 | 1.4 | 3.1 | 0.4 | 2.0 |
| IBD Crohn's | 0.0 | 0.0 | 0.0 | 0.4 | 0.3 | 2.0 |
| Colon | 0.0 | 1.0 | 0.0 | 0.2 | 0.3 | 0.0 |
| Lung | 0.0 | 0.0 | 0.0 | 0.0 | 0.3 | 0.0 |
| Thymus | 0.4 | 0.9 | 0.0 | 0.0 | 0.0 | 0.0 |
| Kidney | 100.0 | 100.0 | 100.0 | 100.0 | 100.0 | 100.0 |

CNS_neurodegeneration_v1.0 Summary: Ag2435 Expression of the CG95462-03 gene is low/undet. in all samples in this panel (CT>35). (Data not shown.)

Panel 1.2 Summary: Ag1362/Ag1393/Ag1400/Ag1527 Multiple experiments show high expression of the CG95462-03 gene in the thymus. Thus, this gene might be useful as a marker of thymic tissue. In addition, the putative GPCR encoded for by this gene could play an important role in T cell development. Small molecule therapeutics, or antibody therapeutics designed against the GPCR encoded for by this gene could be utilized to modulate immune function (T cell development) and be important for organ transplant, AIDS treatment or post chemotherapy immune reconstitution.

High expression of this gene is also associated with cell lines derived from lung cancer, breast cancer and ovarian cancer. Thus, therapeutic modulation of this gene product, through down-regulation of function by small molecule drugs or antibodies, may be of utility in the treatment of lung, breast or ovarian cancer.

Panel 1.3D Summary: Ag1400/Ag1630 Significant expression of the CG95462-03 gene is limited to thymus (CTs=32-33). Thus, this gene might be useful as a marker of thymic tissue. Please see Panel 1.2 for further discussion of potential utility of this gene. An additional experiment with the probe and primer set Ag2435 shows low/undetectable (CT values>34.5) across the samples on this panel. (Data not shown.)

Panel 2.2 Summary: Ag1624/Ag2435 Expression of the CG95462-03 gene is low/undetectable (CT values>35) across the samples on this panel.(Data not shown.)

Panel 2D Summary: Ag1630 The CG95462-03 gene is expressed in one normal colon margin sample. Thus, expression of this gene could be used to differentiate between this sample and other samples on this panel and between normal colon tissue and other normal or malignant tissue samples. Please note that two experiments with the probe/primer set Ag1400 shows low/undetectable expression in all samples on this panel. (Data not shown.)

Panel 4D Summary: Ag1397/Ag1400/Ag1624/Ag1630/Ag243 5 The CG95462-03 transcript is detected in liver cirrhosis (CT=34) and kidney (CT=29). This transcript is not detected in normal liver in Panel 1.3D suggesting that its expression is unique to liver cirrhosis. This gene encodes a putative GPCR and therefore antibodies or small molecule therapeutics could reduce on inhibit liver fibrosis. Antibodies to this putative GPCR could also be used for the diagnosis of liver cirrhosis. The putative GPCR encoded for by the transcript could also allow cells within the kidney to respond to specific microenvironmental signals. Antibody or small molecule therapies designed with the protein encoded for by this gene could modulate kidney function and be important in the treatment of inflammatory or autoimmune diseases that affect the kidney, including glomerulonephritis.

### REFERENCES

1. Mark M D, Wittemann S, Herlitze S (2000) G protein modulation of recombinant P/Q-type calcium channels by regulators of G protein signalling proteins. J. Physiol. 528 Pt 1:65-77.

Fast synaptic transmission is triggered by the activation of presynaptic Ca2+ channels which can be inhibited by Gbetagamma subunits via G protein-coupled receptors (GPCR). Regulators of G protein signalling (RGS) proteins are GTPase-accelerating proteins (GAPs), which are responsible for >100-fold increases in the GTPase activity of G proteins and might be involved in the regulation of presynaptic Ca2+ channels. In this study we investigated the effects of RGS2 on G protein modulation of recombinant P/Q-type channels expressed in a human embryonic kidney (HEK293) cell line using whole-cell recordings. 2. RGS2 markedly accelerates transmitter-mediated inhibition and recovery from inhibition of Ba2+ currents (IBa) through P/Q-type channels heterologously expressed with the muscarinic acetylcholine receptor M2 (mAChR M2). 3. Both RGS2 and RGS4 modulate the prepulse facilitation properties of P/Q-type Ca2+ channels. G protein reinhibition is accelerated, while release from inhibition is slowed. These kinetics depend on the availability of G protein alpha and betagamma subunits which is altered by RGS proteins. 4. RGS proteins unmask the Ca2+ channel beta subunit modulation of Ca2+ channel G protein inhibition. In the presence of RGS2, P/Q-type channels containing the beta2a and beta3 subunits reveal significantly altered kinetics of G protein modulation and increased facilitation compared to Ca2+ channels coexpressed with the betalb or beta4 subunit.

PMID: 11018106

AP. SC128993196_A: Olfactory Receptor

Expression of gene SC128993196_A was assessed using the primer-probe sets Ag1392 and Ag1623, described in Tables APA and APB. Results of the RTQ-PCR runs are shown in Tables APC, APD, and APE.

TABLE APA
Probe Name Ag1392

| | | | Start | |
|---|---|---|---|---|
| Primers | Sequences | Length | Position | SEQ ID NO |
| Forward | 5′-cctccacacaccaatgtacttc-3′ | 22 | 222 | 323 |
| Probe | TET-5′-tccttggcattctctcaacatctgaga-3′-TAMRA | 27 | 245 | 324 |
| Reverse | 5′-gcatcttggggtagaatgacaaa-3′ | 22 | 283 | 325 |

TABLE APR
Probe Name Ag1623

| Primers | Sequences | Length | Start Position | SEQ ID NO |
|---|---|---|---|---|
| Forward | 5′-ggacaatctccttcaactgttg-3′ | 22 | 329 | 326 |
| Probe | TET-5′-cttggttttgccattaccaactgcct-3′-TAMRA | 26 | 373 | 327 |
| Reverse | 5′-cataacccatcacacccaatag-3′ | 22 | 400 | 328 |

TABLE APC
Panel 1.2

| | Rel. Exp. (%) | Rel. Exp. (%) | | Rel. Exp. (%) | Rel. Exp. (%) |
|---|---|---|---|---|---|
| | Ag1392, Run | Ag1392, Run | | Ag1392, Run | Ag1392, Run |
| Tissue Name | 135067789 | 138343677 | Tissue Name | 135067789 | 138343677 |
| Endothelial cells | 0.0 | 0.0 | Renal ca. 786-0 | 0.0 | 0.0 |
| Heart (Fetal) | 0.0 | 0.0 | Renal ca. A498 | 0.1 | 0.3 |
| Pancreas | 0.0 | 0.1 | Renal ca. RXF | 0.0 | 0.1 |
| | | | 393 | | |
| Pancreatic ca. | 0.0 | 0.0 | Renal ca. | 0.2. | 0.3 |
| CAPAN 2 | | | ACHN | | |
| Adrenal gland | 0.1 | | Renal ca. UO-31 | 0.3 | 0.6 |
| Thyroid | 0.0 | 0.0 | Renal ca. TK-10 | 0.2 | 0.7 |
| Salivary gland | 0.0 | 0.0 | Liver | 0.0 | 0.0 |
| Pituitary gland | 0.0 | 0.0 | Liver (fetal) | 0.0 | 0.0 |
| Brain (fetal) | 0.0 | 0.0 | Liver ca. | 0.0 | 0.0 |
| | | | (hepatoblast) | | |
| | | | HepG2 | | |
| Brain (whole) | 0.0 | 0.0 | Lung | 0.0 | 0.0 |
| Brain (amygdala) | 0.0 | 0.0 | Lung (fetal) | 0.0 | 0.0 |
| Brain | 0.0 | 0.0 | Lung ca. (small | 0.0 | 0.0 |
| (cerebellum) | | | cell) LX-1 | | |
| Brain | 0.0 | 0.0 | Lung ca. (small | 2.3 | 3.3 |
| (hippocampus) | | | cell) NCI-H69 | | |
| Brain (thalamus) | 0.0 | 0.0 | Lung ca. (s.cell | 0.0 | 0.1 |
| | | | var.) SHP-77 | | |
| Cerebral Cortex | 0.0 | 0.0 | Lung ca. (large | 0.2 | 0.8 |
| | | | cell)NCI-H460 | | |
| Spinal cord | 0.0 | 0.1 | Lung ca. (non- | 0.8 | 1.3 |
| | | | sm. cell) A549 | | |
| glio/astro U87- | 0.0 | 0.0 | Lung ca. (non- | 0.0 | 0.0 |
| MG | | | s.cell) NCI-H23 | | |
| glio/astro U-118- | 0.1 | 0.2 | Lung ca. (non- | 0.5 | 0.5 |
| MG | | | s.cell) HOP-62 | | |
| astrocytoma | 0.0 | 0.0 | Lung ca. (non- | 0.0 | 0.0 |
| SW1783 | | | s.cl) NCI-H522 | | |
| neuro*; met SK- | 0.1 | 0.2 | Lung ca. | 0.0 | 0.1 |
| N-AS | | | (squam.) SW | | |
| | | | 900 | | |
| astrocytoma SF- | 0.0 | 0.2 | Lung ca. | 0.4 | 1.0 |
| 539 | | | (squam.) NCI- | | |
| | | | H596 | | |
| astrocytoma SNB- | 0.0 | 0.1 | Mammary gland | 0.0 | 0.0 |
| 75 | | | | | |
| glioma SNB-19 | 0.2 | 0.3 | Breast ca.* | 0.0 | 0.0 |
| | | | (pl.ef) MCF-7 | | |
| glioma U251 | 0.0 | 0.1 | Breast ca.* | 0.0 | 0.0 |
| | | | (pl.ef) MDA- | | |
| | | | MB-231 | | |
| glioma SF-295 | 0.1 | 0.1 | Breast ca.* (pl. | 0.4 | 2.1 |
| | | | ef) T47D | | |
| Heart | 0.0 | 0.5 | Breast ca. BT- | 0.0 | 0.2 |
| | | | 549 | | |
| Skeletal muscle | 0.0 | | Breast ca. | 0.0 | 0.5 |
| | | | MDA-N | | |
| Bone marrow | 0.0 | 0.0 | Ovary | 0.0 | 0.0 |
| Thymus | 100.0 | 100.0 | Ovarian ca. | 0.0 | 0.2 |
| | | | OVCAR-3 | | |
| Spleen | 0.0 | 0.0 | Ovarian ca. | 0.0 | 0.1 |
| | | | OVCAR-4 | | |
| Lymph node | 0.0 | 0.0 | Ovarian ca. | 4.1 | 6.0 |
| | | | OVCAR-5 | | |
| Colorectal | 0.2 | 0.2 | Ovarian ca. | 0.0 | 0.3 |
| | | | OVCAR-8 | | |
| Stomach | 0.0 | 0.0 | Ovarian ca. | 0.2 | 0.3 |
| | | | IGROV-1 | | |
| Small intestine | 0.0 | 0.0 | Ovarian ca. | 0.1 | 0.6 |
| | | | (ascites) SK- | | |
| | | | OV-3 | | |
| Colon ca. SW480 | 0.0 | 0.1 | Uterus | 0.0 | 0.0 |
| Colon ca.* | 0.0 | 0.0 | Placenta | 0.0 | 0.0 |
| SW620 (SW480 | | | | | |
| met) | | | | | |
| Colon ca. HT29 | 0.0 | 0.2 | Prostate | 0.2 | 0.7 |
| Colon ca. HCT- | 0.0 | 0.0 | Prostate ca.* | 0.1 | 0.2 |
| 116 | | | (bone met) PC-3 | | |
| Colon ca. CaCo-2 | 0.0 | 0.2 | Testis | 0.2 | 0.0 |
| CC Well to Mod | 0.3 | 0.8 | Melanoma | 0.1 | 0.1 |
| Diff (ODO3866) | | | Hs688(A).T | | |
| Colon ca. HCC- | 0.0 | 0.6 | Melanoma* | 0.1 | 0.4 |
| 2998 | | | (met) | | |
| | | | Hs688(B).T | | |
| Gastric ca. (liver | 0.1 | 0.0 | Melanoma | 0.0 | 0.0 |
| met) NCI-N87 | | | UACC-62 | | |
| Bladder | 0.2 | 0.5 | Melanoma M14 | 1.0 | 1.6 |
| Trachea | 0.1 | 0.2 | Melanoma LOX | 0.1 | 0.0 |
| | | | IMVI | | |
| Kidney | 0.0 | 0.2 | Melanoma* | 0.1 | 0.0 |
| | | | (met) SK-MEL-5 | | |
| Kidney (fetal) | 0.0 | 0.1 | | | |

TABLE APD
Panel 1.3D

| | Rel. Exp.(%) Ag1623, | | Rel. Exp.(%) Ag1623, |
|---|---|---|---|
| Tissue Name | Run 165531482 | Tissue Name | Run 165531482 |
| Liver adenocarcinoma | 0.0 | Kidney (fetal) | 0.0 |
| Pancreas | 0.5 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 0.0 | Lung ca. (large | 0.0 |
| | | cell) NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.7 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.0 |

TABLE APE
Panel 4D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1392, | Ag1392, | Ag1623, | | Ag1392, | Ag1392, | Ag1623, |
| | Run | Run | Run | | Run | Run | Run |
| Tissue Name | 138175387 | 138289262 | 164739993 | Tissue Name | 138175387 | 138289262 | 164739993 |
| Secondary Th1 | 0.0 | 0.0 | 0.0 | HUVEC IL- | 0.0 | 0.0 | 0.0 |
| act | | | | 1 beta | | | |
| Secondary Th2 | 0.0 | 0.0 | 0.0 | HUVEC IFN | 0.0 | 0.0 | 0.0 |
| act | | | | gamma | | | |
| Secondary Tr1 | 0.0 | 0.0 | 0.0 | HUVEC TNF | 0.0 | 0.0 | 0.0 |
| act | | | | alpha + IFN | | | |
| | | | | gamma | | | |
| Secondary Th1 | 0.0 | 0.0 | 0.0 | HUVEC TNF | 0.0 | 0.0 | 0.0 |
| rest | | | | alpha + IL4 | | | |
| Secondary Th2 | 0.0 | 0.0 | 0.0 | HUVEC IL-11 | 0.0 | 0.0 | 0.0 |
| rest | | | | | | | |
| Secondary Tr1 | 0.0 | 0.0 | 0.0 | Lung | 0.0 | 0.0 | 0.0 |
| rest | | | | Microvascular | | | |
| | | | | EC none | | | |
| Primary Th1 | 0.0 | 0.0 | 0.0 | Lung | 0.0 | 0.0 | 0.0 |
| act | | | | Microvascular | | | |
| | | | | EC TNF alpha + | | | |
| | | | | IL-1 beta | | | |
| Primary Th2 | 0.0 | 0.0 | 0.0 | Microvascular | 0.0 | 0.0 | 0.0 |
| act | | | | Dermal EC none | | | |
| Primary Tr1 | 0.0 | 0.0 | 0.0 | Microsvascular | 0.0 | 0.1 | 0.0 |
| act | | | | Dermal EC | | | |
| | | | | TNF alpha + IL- | | | |
| | | | | 1 beta | | | |
| Primary Th1 | 0.0 | 0.0 | 0.0 | Bronchial | 0.0 | 0.0 | 0.0 |
| rest | | | | epithelium | | | |
| | | | | TNF alpha + | | | |
| | | | | IL1 beta | | | |
| Primary Th2 | 0.0 | 0.0 | 0.0 | Small airway | 0.0 | 0.0 | 0.0 |
| rest | | | | epithelium none | | | |
| Primary Tr1 | 0.0 | 0.0 | 0.0 | Small airway | 0.0 | 0.0 | 0.0 |
| rest | | | | epithelium | | | |
| | | | | TNF alpha + IL- | | | |
| | | | | 1 beta | | | |
| CD45RA CD4 | 0.0 | 0.0 | 0.0 | Coronery artery | 0.0 | 0.0 | 0.0 |
| lymphocyte act | | | | SMC rest | | | |
| CD45RO CD4 | 0.0 | 0.0 | 0.0 | Coronery artery | 0.0 | 0.0 | 0.0 |
| lymphocyte act | | | | SMC TNF | | | |
| | | | | alpha + IL-1 beta | | | |
| CD8 | 0.0 | 0.0 | 0.0 | Astrocytes rest | 0.0 | 0.0 | 0.0 |
| lymphocyte act | | | | | | | |
| Secondary | 0.0 | 0.0 | 0.0 | Astrocytes | 0.0 | 0.0 | 0.0 |
| CD8 | | | | TNF alpha + IL- | | | |
| lymphocyte | | | | 1 beta | | | |
| rest | | | | | | | |
| Secondary | 0.0 | 0.0 | 0.0 | KU-812 | 0.0 | 0.0 | 0.0 |
| CD8 | | | | (Basophil) rest | | | |
| lymphocyte act | | | | | | | |
| CD4 | 0.0 | 0.0 | 0.0 | KU-812 | 0.0 | 0.0 | 0.0 |
| lymphocyte | | | | (Basophil) | | | |
| none | | | | PMA/ionomycin | | | |
| 2ry | 0.0 | 0.0 | 0.0 | CCD1106 | 0.0 | 0.0 | 0.0 |
| Th1/Th2/Tr1_{—} | | | | (Keratinocytes) | | | |
| anti-CD95 | | | | none | | | |
| CH11 | | | | | | | |
| LAK cells rest | 0.0 | 0.0 | 0.0 | 93580_CCD1106 | 0.0 | 0.0 | |
| | | | | (Keratinocytes)_{—} | | | |
| | | | | TNFa and IFNg | | | |
| LAK cells IL-2 | 0.0 | 0.0 | 0.0 | Liver cirrhosis | 0.2 | 0.4 | 0.0 |
| LAK cells IL- | 0.0 | 0.1 | 0.0 | Lupus kidney | 0.0 | 0.0 | 0.0 |
| 2 + IL-12 | | | | | | | |
| LAK cells | 0.0 | 0.0 | 0.0 | NCI-H292 none | 0.0 | 0.0 | 0.0 |
| IL-2 + | | | | | | | |
| IFN gamma | | | | | | | |
| LAK cells IL- | 0.0 | 0.0 | 0.0 | NCI-H292 IL-4 | 0.0 | 0.0 | 0.0 |
| 2 + IL-18 | | | | | | | |
| LAK cells | 0.0 | 0.0 | 0.0 | NCI-H292 IL-9 | 0.0 | 0.0 | 0.0 |
| PMA/ | | | | | | | |
| ionomycin | | | | | | | |
| NK Cells IL-2 | 0.0 | 0.0 | 0.0 | NCI-H292 IL-13 | 0.0 | 0.0 | 0.0 |
| rest | | | | | | | |
| Two Way | 0.0 | 0.0 | 0.0 | NCI-H292 IFN | 0.0 | 0.0 | 0.0 |
| MLR 3 day | | | | gamma | | | |
| Two Way | 0.0 | 0.0 | 0.0 | HPAEC none | 0.0 | 0.0 | 0.0 |
| MLR 5 day | | | | | | | |
| Two Way | 0.0 | 0.0 | 0.0 | HPAEC TNF | 0.0 | 0.0 | 0.0 |
| MLR 7 day | | | | alpha + IL-1 | | | |
| | | | | beta | | | |
| PBMC rest | 0.0 | 0.0 | 0.0 | Lung fibroblast | 0.0 | 0.0 | 0.0 |
| | | | | none | | | |
| PBMC PWM | 0.0 | 0.0 | 0.0 | Lung fibroblast | 0.0 | 0.0 | 0.0 |
| | | | | TNF alpha + IL- | | | |
| | | | | 1 beta | | | |
| PBMC PHA-L | 0.0 | 0.0 | 0.0 | Lung fibroblast | 0.0 | 0.0 | 0.0 |
| | | | | IL-4 | | | |
| Ramos (B cell) | 0.0 | 0.0 | 0.0 | Lung fibroblast | 0.0 | 0.0 | 0.0 |
| none | | | | IL-9 | | | |
| Ramos (B cell) | 0.0 | 0.0 | 0.0 | Lung fibroblast | 0.0 | 0.0 | 0.0 |
| ionomycin | | | | IL-13 | | | |
| B lymphocytes | 0.0 | 0.0 | 0.0 | Lung fibroblast | 0.0 | 0.0 | 0.0 |
| PWM | | | | IFN gamma | | | |
| B lymphocytes | 0.1 | 0.0 | 0.0 | Dermal | 0.0 | 0.0 | 0.0 |
| CD40L and | | | | fibroblast | | | |
| IL-4 | | | | CCD1070 rest | | | |
| EOL-1 | 0.0 | 0.0 | 0.0 | Dermal | 0.0 | 0.0 | 0.0 |
| dbcAMP | | | | fibroblast | | | |
| | | | | CCD1070 TNF | | | |
| | | | | alpha | | | |
| EOL-1 | 0.0 | 0.0 | 0.0 | Dermal | 0.0 | 0.0 | 0.0 |
| dbcAMP | | | | fibroblast | | | |
| PMA/ | | | | CCD1070 IL-1 | | | |
| ionomycin | | | | beta | | | |
| Dendritic cells | 0.0 | 0.0 | 0.0 | Dermal | 0.0 | 0.1 | 0.0 |
| none | | | | fibroblast IFN | | | |
| | | | | gamma | | | |
| Dendritic cells | 0.0 | 0.0 | 0.0 | Dermal | 0.0 | 0.0 | 0.0 |
| LPS | | | | fibroblast IL-4 | | | |
| Dendritic cells | 0.0 | 0.0 | 0.0 | IBD Colitis 2 | 0.0 | 0.0 | 0.0 |
| anti-CD40 | | | | | | | |
| Monocytes rest | 0.0 | 0.0 | 0.1 | IBD Crohn's | 0.0 | 0.0 | 0.0 |
| Monocytes | 0.0 | 0.0 | 0.0 | Colon | 0.0 | 0.0 | 0.0 |
| LPS | | | | | | | |
| Macrophages | 0.0 | 0.0 | 0.0 | Lung | 0.0 | 0.0 | 0.0 |
| rest | | | | | | | |
| Macrophages | 0.0 | 0.0 | 0.0 | Thymus | 0.0 | 0.0 | 0.0 |
| LPS | | | | | | | |
| HUVEC none | 0.0 | 0.0 | 0.1 | Kidney | 100.0 | 100.0 | 100.0 |
| HUVEC | 0.0 | 0.0 | 0.0 | | | | |
| starved | | | | | | | |

Panel 1.2 Summary: Ag1392 Results from two experiments using the same probe/primer set are in reasonable agreement. Highest expression of the SC 128993196_A is seen in thymus (CT=27); see Panel 1.3D summary for utility discussion. In addition, low but significant gene expression is also seen in a single ovarian and a single lung cancer cell line. Therefore, the therapeutic inhibition of this gene activity, through the use of small molecule drugs or antibodies, could provide treatment of the ovarian and lung cancers.

Panel 1.3D Summary: Ag1623 Significant expression of the SC128993196_A gene on this panel is found only in thymus (CT=29.3). This is in concordance with the results from Panel 1.2. The putative GPCR encoded for by this gene could therefore play an important role in T cell development. Small molecule therapeutics, or antibody therapeutics designed against the GPCR encoded for by this gene could be utilized to modulate immune function (T cell development) and be important for organ transplant, AIDS treatment or post chemotherapy immune reconstitiution.

Panel 2.2 Summary: Ag1623 Expression of the SC128993196_A gene is low/undetectable (CT values

Panel 4D Summary: Ag1392/1623: The SC128993196A gene is only expressed at detectable levels in the kidney. The putative GPCR encoded for by this gene could allow cells within the kidney to respond to specific microenvironmental signals (For example, ref. 1). Therefore, antibody or small molecule therapies designed with the protein encoded for by this gene could modulate kidney function and be important in the treatment of inflammatory or autoimmune diseases that affect the kidney, including lupus and glomerulonephritis.

### REFERENCES

1. Mark M. D., Wittemann S., Herlitze S. (2000) G protein modulation of recombinant P/Q-type calcium channels by regulators of G protein signalling proteins. J. Physiol. 528 Pt 1: 65-77.

1. Fast synaptic transmission is triggered by the activation of presynaptic Ca2+ channels which can be inhibited by Gbetagamma subunits via G protein-coupled receptors (GPCR). Regulators of G protein signalling (RGS) proteins are GTPase-accelerating proteins (GAPs), which are responsible for >100-fold increases in the GTPase activity of G proteins and might be involved in the regulation of presynaptic Ca2+ channels. In this study we investigated the effects of RGS2 on G protein modulation of recombinant P/Q-type channels expressed in a human embryonic kidney (HEK293) cell line using whole-cell recordings. 2. RGS2 markedly accelerates transmitter-mediated inhibition and recovery from inhibition of Ba2+ currents (IBa) through P/Q-type channels heterologously expressed with the muscarinic acetylcholine receptor M2 (mAChR M2). 3. Both RGS2 and RGS4 modulate the prepulse facilitation properties of P/Q-type Ca2+ channels. G protein reinhibition is accelerated, while release from inhibition is slowed. These kinetics depend on the availability of G protein alpha and betagamma subunits which is altered by RGS proteins. 4. RGS proteins unmask the Ca2+ channel beta subunit modulation of Ca2+ channel G protein inhibition. In the presence of RGS2, P/Q-type channels containing the beta2a and beta3 subunits reveal significantly altered kinetics of G protein modulation and increased facilitation compared to Ca2+ channels coexpressed with the betalb or beta4 subunit.

PMID: 11018106

AQ. CG148698-01/SC35113271_A: Olfactory Receptor

Expression of gene CG148698-01 was assessed using the primer-probe sets Ag1533, Ag2617 and Ag2862, described in Tables AQA, AQB and AQC. Results of the RTQ-PCR runs are shown in Tables AQD, AQE, AQF, AQG, AQH, AQI and AQJ.

TABLE AQA
Probe Name Ag1533

| Primers | Sequences | Length | Start Position | SEQ ID NO |
|---|---|---|---|---|
| Forward | 5′-accatcatcaagagtgctatgg-3′ | 22 | 446 | 329 |
| Probe | TET-5′-tcctttcgaagcttctgcatcatcct-3′-TAMRA | 26 | 473 | 330 |
| Reverse | 5′-aggcatgtcagcaagaatacat-3′ | 22 | 504 | 331 |

TABLE AQB
Probe Name Ag2617

| | | | | SEQ ID |
|---|---|---|---|---|
| Primers | Sequences | Length | Start Positon | NO |
| Forward | 5′-ccatggcatttgatcactatgt-3′ | 22 | 378 | 332 |
| Probe | TET-5′-tgagatataccaccatcttgactccca-3′-TAMRA | 27 | 417 | 333 |
| Reverse | 5′-ccatagcactcttgatgatggt-3′ | 22 | 446 | 334 |

TABLE AQC
Probe Name Ag2862

| | | | | SEQ ID |
|---|---|---|---|---|
| Primers | Sequences | Length | Start Position | NO |
| Forward | 5′-ccatggcatttgatcactatgt-3′ | 22 | 378 | 335 |
| Probe | TET-5′-tgagatataccaccatcctgactccca-3′-TAMRA | 27 | 417 | 336 |
| Reverse | 5′-ccatagcactcttgatgatggt-3′ | 22 | 446 | 337 |

TABLE AQD
CNS_neurodegeneration_v1.0

| | Rel. Exp.(%) Ag1533, Run | | Rel. Exp.(%) Ag1533, |
|---|---|---|---|
| Tissue Name | 225432469 | Tissue Name | Run 225432469 |
| AD 1 Hippo | 36.6 | Control (Path) 3 | 16.5 |
| | | Temporal Ctx | |
| AD 2 Hippo | 11.6 | Control (Path) 4 | 42.9 |
| | | Temporal Ctx | |
| AD 3 Hippo | 50.7 | AD 1 Occipital Ctx | 39.2 |
| AD 4 Hippo | 75.3 | AD 2 Occipital Ctx | 0.0 |
| | | (Missing) | |
| AD 5 Hippo | 20.4 | AD 3 Occipital Ctx | 25.9 |
| AD 6 Hippo | 69.3 | AD 4 Occipital Ctx | 82.9 |
| Control 2 Hippo | 22.5 | AD 5 Occipital Ctx | 11.0 |
| Control 4 Hippo | 53.2 | AD 5 Occipital Ctx | 7.1 |
| Control (Path) 3 | 29.7 | Control 1 Occipital | 11.9 |
| Hippo | | Ctx | |
| AD 1 Temporal Ctx | 96.6 | Control 2 Occipital | 29.1 |
| | | Ctx | |
| AD 2 Temporal Ctx | 34.6 | Control 3 Occipital | 41.2 |
| | | Ctx | |
| AD 3 Temporal Ctx | 54.3 | Control 4 Occipital | 11.9 |
| | | Ctx | |
| AD 4 Temporal Ctx | 68.3 | Control (Path) 1 | 23.3 |
| | | Occipital Ctx | |
| AD 5 Inf Temporal | 90.8 | Control (Path) 2 | 14.3 |
| Ctx | | Occipital Ctx | |
| AD 5 Sup Temporal | 53.2 | Control (Path) 3 | 35.1 |
| Ctx | | Occipital Ctx | |
| AD 6 Inf Temporal | 62.0 | Control (Path) 4 | 30.8 |
| Ctx | | Occipital Ctx | |
| AD 6 Sup Temporal | 55.5 | Control 1 Parietal Ctx | 18.2 |
| Ctx | | | |
| Control 1 Temporal | 5.3 | Control 2 Parietal Ctx | 69.3 |
| Ctx | | | |
| Control 2 Temporal | 19.6 | Control 3 Parietal Ctx | 6.3 |
| Ctx | | | |
| Control 3 Temporal | 38.7 | Control (Path) 1 | 33.9 |
| Ctx | | Parietal Ctx | |
| Control 3 Temporal | 17.0 | Control (Path) 2 | 18.4 |
| Ctx | | Parietal Ctx | |
| Control (Path) 1 | 29.1 | Control (Path) 3 | 26.8 |
| Temporal Ctx | | Parietal Ctx | |
| Control (Path) 2 | 23.7 | Control (Path) 4 | 100.0 |
| Temporal Ctx | | Parietal Ctx | |

TABLE AQE
General_screening_panel_v1.5

| | | | Rel. Exp.(%) |
|---|---|---|---|
| | Rel. Exp.(%) Ag1533, | | Ag1533, Run |
| Tissue Name | Run 228632846 | Tissue Name | 228632846 |
| Adipose | 12.9 | Renal ca. TK-10 | 2.4 |
| Melanoma* | 1.5 | Bladder | 43.8 |
| Hs688(A).T | | | |
| Melanoma* | 0.4 | Gastric ca. (liver met.) | 54.7 |
| Hs688(B).T | | NCI-N87 | |
| Melanoma* M14 | 0.0 | Gastric ca. KATO III | 0.4 |
| Melanoma* LOXIMVI | 0.5 | Colon ca. SW-948 | 0.0 |
| Melanoma* SK-MEL-5 | 0.3 | Colon ca. SW480 | 0.0 |
| Squamous cell | 0.0 | Colon ca.* (SW480 met) | 0.0 |
| carcinoma SCC-4 | | SW620 | |
| Testis Pool | 15.1 | Colon ca. HT29 | 0.0 |
| (Prostate ca.* (bone | 1.9 | Colon ca. HCT-116 | 2.8 |
| met) PC-3 | | | |
| Prostate Pool | 23.7 | Colon ca. CaCo-2 | 0.7 |
| Placenta | 2.9 | Colon cancer tissue | 1.5 |
| Uterus Pool | 11.5 | Colon ca. SW1116 | 0.3 |
| Ovarian ca. OVCAR-3 | 25.5 | Colon ca. Colo-205 | 0.0 |
| Ovarian ca. SK-OV-3 | 87.7 | Colon ca. SW-48 | 0.0 |
| Ovarian ca. OVCAR-4 | 0.0 | Colon Pool | 37.4 |
| Ovarian ca. OVCAR-5 | 10.1 | Small Intestine Pool | 43.5 |
| Ovarian ca. IGROV-1 | 0.0 | Stomach Pool | 27.4 |
| Ovarian ca. OVCAR-8 | 2.4 | Bone Marrow Pool | 22.1 |
| Ovary | 22.2 | Fetal Heart | 57.0 |
| Breast ca. MCF-7 | 0.0 | Heart Pool | 12.2 |
| Breast ca. MDA-MB- | 1.3 | Lymph Node Pool | 66.9 |
| 231 | | | |
| Breast ca. BT 549 | 1.4 | Fetal Skeletal Muscle | 11.4 |
| Breast ca. T47D | 0.8 | Skeletal Muscle Pool | 7.5 |
| Breast ca. MDA-N | 0.7 | Spleen Pool | 21.3 |
| Breast Pool | 59.5 | Thymus Pool | 38.7 |
| Trachea | 19.1 | CNS cancer (glio/astro) | 1.6 |
| | | U87-MG | |
| Lung | 27.2 | CNS cancer (glio/astro) | 1.5 |
| | | U-118-MG | |
| Fetal Lung | 100.0 | CNS cancer (neuro; met) | 0.0 |
| | | SK-N-AS | |
| Lung ca. NCI-N417 | 0.0 | CNS cancer (astro) SF- | 1.1 |
| | | 539 | |
| Lung ca. LX-1 | 0.4 | CNS cancer (astro) SNB- | 4.5 |
| | | 75 | |
| Lung ca. NCI-H146 | 4.7 | CNS cancer (glio) SNB- | 1.1 |
| | | 19 | |
| Lung ca. SHP-77 | 0.0 | CNS cancer (glio) SF-295 | 20.6 |
| Lung ca. A549 | 2.9 | Brain (Amygdala) Pool | 2.1 |
| Lung ca. NCI-H526 | 0.0 | Brain (cerebellum) | 0.9 |
| Lung ca. NCI-H23 | 6.7 | Brain (fetal) | 7.9 |
| Lung ca. NCI-H460 | 9.2 | Brain (Hippocampus) | 1.6 |
| | | Pool | |
| Lung ca. HOP-62 | 8.4 | Cerebral Cortex Pool | 2.6 |
| Lung ca. NCI-H522 | 0.0 | Brain (Substantia nigra) | 2.0 |
| | | Pool | |
| Liver | 0.4 | Brain (Thalamus) Pool | 2.5 |
| Fetal Liver | 8.7 | Brain (whole) | 3.8 |
| Liver ca. HepG2 | 0.7 | Spinal Cord Pool | 5.0 |
| Kidney Pool | 67.8 | Adrenal Gland | 11.0 |
| Fetal Kidney | 94.6 | Pituitary gland Pool | 5.3 |
| Renal ca. 786-0 | 1.2 | Salivary Gland | 1.7 |
| Renal ca. A498 | 1.8 | Thyroid (female) | 2.5 |
| Renal ca. ACHN | 3.6 | Pancreatic ca. CAPAN2 | 4.7 |
| Renal ca. UO-31 | 5.8 | Pancreas Pool | 39.8 |

TABLE AQF
Panel 1.2

| | | | Rel. Exp.(%) |
|---|---|---|---|
| | Rel. Exp.(%) Ag1533, | | Ag1533, Run |
| Tissue Name | Run 142223910 | Tissue Name | 142223910 |
| Endothelial cells | 4.4 | Renal ca. 786-0 | 1.8 |
| Heart (Fetal) | 3.9 | Renal ca. A498 | 7.5 |
| Pancreas | 4.8 | Renal ca. RXF 393 | 1.8 |
| Pancreatic ca. CAPAN 2 | 1.1 | Renal ca. ACHN | 5.0 |
| Adrenal Gland | 20.9 | Renal ca. UO-31 | 9.0 |
| Thyroid | 1.1 | Renal ca. TK-10 | 2.3 |
| Salivary gland | 52.1 | Liver | 9.8 |
| Pituitary gland | 0.7 | Liver (fetal) | 6.0 |
| Brain (fetal) | 0.7 | Liver ca. (hepatoblast) | 1.0 |
| | | HepG2 | |
| Brain (whole) | 0.0 | Lung | 1.5 |
| Brain (amygdala) | 1.0 | Lung (fetal) | 2.9 |
| Brain (cerebellum) | 0.3 | Lung ca. (small cell) | 0.2 |
| | | LX-1 | |
| Brain (hippocampus) | 6.3 | Lung ca. (small cell) | 5.2 |
| | | NCI-H69 | |
| Brain (thalamus) | 1.9 | Lung ca. (s. cell var.) | 0.0 |
| | | SHP-77 | |
| Cerebral Cortex | 9.2 | Lung ca. (large | 2.8 |
| | | cell) NCI-H460 | |
| Spinal cord | 2.9 | Lung ca. (non-sm. cell) | 5.1 |
| | | A549 | |
| glio/astro U87-MG | 1.6 | Lung ca. (non-s. cell) | 10.2 |
| | | NCI-H23 | |
| glio/astro U-118-MG | 1.1 | Lung ca. (non-s. cell) | 28.3 |
| | | HOP-62 | |
| Astrocytoma SW1783 | 1.2 | Lung ca. (non-s. cl) | 2.0 |
| | | NCI-H522 | |
| neuro*; met SK-N-AS | 0.2 | Lung ca. (squam.) SW | 3.6 |
| | | 900 | |
| astrocytoma SF-539 | 7.4 | Lung ca. (squam.) NCI- | 0.7 |
| | | H596 | |
| astrocytoma SNB-75 | 1.6 | Mammary gland | 3.4 |
| glioma SNB-19 | 8.1 | Breast ca.* (pl. ef) | 1.3 |
| | | MCF-7 | |
| glioma U251 | 5.9 | Breast ca.* (pl. ef) | 0.3 |
| | | MDA-MB-231 | |
| glioma SF-295 | 12.2 | Breast ca.* (pl. ef) | 12.8 |
| | | T47D | |
| Heart | 15.3 | Breast ca. BT-549 | 2.9 |
| Skeletal Muscle | 4.9 | Breast ca. MDA-N | 0.8 |
| Bone marrow | 4.4 | Ovary | 9.3 |
| Thymus | 0.8 | Ovarian ca. OVCAR-3 | 42.0 |
| Spleen | 5.6 | Ovarian ca. OVCAR-4 | 1.4 |
| Lymph node | 1.6 | Ovarian ca. OVCAR-5 | 18.2 |
| Colorectal | 10.9 | Ovarian ca. OVCAR-8 | 0.0 |
| Stomach | 3.2 | Ovarian ca. IGROV-1 | 0.0 |
| Small intestine | 7.8 | Ovarian ca. (ascites) | 100.0 |
| | | SK-OV-3 | |
| Colon ca. SW480 | 0.0 | Uterus | 6.5 |
| Colon ca.* SW620 | 0.0 | Placenta | 1.8 |
| (SW480 met) | | | |
| Colon ca. HT29 | 0.9 | Prostate | 23.7 |
| Colon ca. HCT-116 | 1.8 | Prostate ca.* bone | 4.5 |
| | | met) PC-3 | |
| Colon ca. CaCo-2 | 1.5 | Testis | 2.7 |
| CC Well to Mod Diff | 2.9 | Melanoma Hs688(A).T | 0.2 |
| (ODO3866) | | | |
| Colon ca. HCC-2998 | 11.6 | Melanoma* (met) | 0.9 |
| | | Hs688(B).T | |
| Gastric ca. (liver met) | 44.4 | Melanoma UACC-62 | 4.5 |
| NCI-N87 | | | |
| Bladder | 98.6 | Melanoma M14 | 4.2 |
| Trachea | 1.5 | Melanoma LOX IMVI | 0.4 |
| Kidney | 40.1 | Melanoma* (met) SK- | 0.0 |
| | | MEL-5 | |
| Kidney (fetal) | 25.5 | | |

TABLE AQG
Panel 1.3D

| | Rel. Exp.(%) Ag2617, | | Rel. Exp.(%) Ag2617, |
|---|---|---|---|
| Tissue Name | Run 167644078 | Tissue Name | Run 167644078 |
| Liver adenocarcinoma | 2.3 | Kidney (fetal) | 15.0 |
| Pancreas | 2.2 | Renal ca. 786-0 | 1.5 |
| Pancreatic ca. CAPAN 2 | 0.0 | Renal ca. A498 | 3.1 |
| Adrenal gland | 0.0 | Renal ca. RXF 393 | 0.0 |
| Thyroid | 1.5 | Renal ca. ACHN | 2.2 |
| Salivary gland | 3.2 | Renal ca. UO-31 | 0.0 |
| Pituitary gland | 6.1 | Renal ca. TK-10 | 2.4 |
| Brain (fetal) | 2.9 | Liver | 2.5 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 2.6 |
| Brain (hippocampus) | 3.2 | Lung (fetal) | 4.0 |
| Brain (substantia nigra) | 2.3 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 1.3 | Lung ca. (s. cell var.) | 0.0 |
| | | SHP-77 | |
| Spinal cord | 3.4 | Lung ca. (large | 0.0 |
| | | cell) NCI-H460 | |
| glio/astro U87-MG | 0.0 | Lung ca. (non-sm. cell) | 0.0 |
| | | A549 | |
| glio/astro U-118-MG | 1.5 | Lung ca. (non-s. cell) | 4.7 |
| | | NCI-H23 | |
| astrocytoma SW1783 | 2.0 | Lung ca. (non-s. cell) | 2.1 |
| | | HOP-62 | |
| neuro*; met SK-N-AS | 0.0 | Lung ca. (non-s. cl) | 0.0 |
| | | NCI-H522 | |
| astrocytoma SF-539 | 6.3 | Lung ca. (squam.) SW | 3.0 |
| | | 900 | |
| astrocytoma SNB-75 | 1.4 | Lung ca. (squam.) NCI- | 0.0 |
| | | H596 | |
| glioma SNB-19 | 2.5 | Mammary gland | 3.3 |
| glioma U251 | 16.6 | Breast ca.* (pl. ef) | 0.0 |
| | | MCF-7 | |
| glioma SF-295 | 12.5 | Breast ca.* (pl. ef) | 0.0 |
| | | MDA-MB-231 | |
| Heart (Fetal) | 0.0 | Breast ca.* (pl. ef) | 1.4 |
| | | T47D | |
| Heart | 2.1 | Breast ca. BT-549 | 1.3 |
| Skeletal muscle (Fetal) | 3.1 | Breast ca. MDA-N | 1.7 |
| Skeletal muscle | 0.0 | Ovary | 0.0 |
| Bone marrow | 1.9 | Ovarian ca. OVCAR-3 | 9.2 |
| Thymus | 4.1 | Ovarian ca. OVCAR-4 | 0.0 |
| Spleen | 1.6 | Ovarian ca. OVCAR-5 | 9.2 |
| Lymph node | 7.1 | Ovarian ca. OVCAR-8 | 5.1 |
| Colorectal | 3.8 | Ovarian ca. IGROV-1 | 0.0 |
| Stomach | 0.0 | Ovarian ca. (ascites) | 100.0 |
| | | SK-OV-3 | |
| Small intestine | 1.4 | Uterus | 5.1 |
| Colon ca. SW480 | 1.5 | Placenta | 0.0 |
| Colon ca.* SW620 | 0.0 | Prostate | 4.5 |
| (SW480 met) | | | |
| Colon ca. HT29 | 1.0 | Prostate ca.* (bone | 1.0 |
| | | met) PC-3 | |
| Colon ca. HCT-116 | 0.0 | Testis | 5.4 |
| Colon ca. CaCo-2 | 0.0 | Melanoma Hs688(A).T | 0.0 |
| CC Well to Mod Diff | 0.0 | Melanoma* (met) | 0.9 |
| (ODO3866) | | Hs688(B).T | |
| Colon ca. HCC-2998 | 0.0 | Melanoma UACC-62 | 0.0 |
| Gastric ca. (liver met) | 12.7 | Melanoma M14 | 0.0 |
| NCI-N87 | | | |
| Bladder | 16.4 | Melanoma LOX IMVI | 0.0 |
| Trachea | 4.1 | Melanoma* (met) SK- | 0.0 |
| | | MEL-5 | |
| Kidney | 0.0 | Adipose | 12.3 |

TABLE AQH
Panel 2D

| | Rel. Exp.(%) Ag1533, | | Rel. Exp.(%) Ag1533, |
|---|---|---|---|
| Tissue Name | Run 145165498 | Tissue Name | Run 145165498 |
| Normal Colon | 48.0 | Kidney Margin 8120608 | 6.1 |
| CC Well to Mod Diff | 5.6 | Kidney Cancer 8120613 | 0.0 |
| (ODO3866) | | | |
| CC Margin (ODO3866) | 4.6 | Kidney Margin 8120614 | 6.9 |
| CC Gr.2 rectosigmoid | 3.5 | Kidney Cancer 9010320 | 10.2 |
| (ODO3868) | | | |
| CC Margin (ODO3868) | 1.0 | Kidney Margin 9010321 | 15.7 |
| CC Mod Diff (ODO3920) | 4.4 | Normal Uterus | 36.3 |
| CC Margin (ODO3920) | 6.6 | Uterine Cancer 064011 | 45.7 |
| CC Gr.2 ascend colon | 1.6 | Normal Thyroid | 8.1 |
| (ODO3921) | | | |
| CC Margin (ODO3921) | 6.1 | Thyroid Cancer | 7.7 |
| CC from Partial | 0.0 | Thyroid Cancer | 27.0 |
| Hepatectomy (ODO4309) | | A302152 | |
| Mets | | | |
| Liver Margin (ODO4309) | 4.8 | Thyroid Margin | 29.5 |
| | | A302153 | |
| Colon mets to lung | 4.3 | Normal Breast | 40.9 |
| (OD04451-01) | | | |
| Lung Margin (OD04451-02) | 1.2 | Breast Cancer | 3.5 |
| Normal Prostate 6546-1 | 26.1 | Breast Cancer | 18.9 |
| | | (OD04590-01) | |
| Prostate Cancer (OD04410) | 64.2 | Breast Cancer Mets | 34.6 |
| | | (OD04590-03) | |
| Prostate Margin (OD04410) | 43.8 | Breast Cancer | 19.3 |
| | | Metastasis | |
| Prostate Cancer (OD04720- | 42.0 | Breast Cancer | 21.2 |
| 01) | | | |
| Prostate Margin (OD04720- | 34.2 | Breast Cancer | 35.1 |
| 02) | | | |
| Normal Lung | 31.9 | Breast Cancer 9100266 | 7.7 |
| Lung Met to Muscle | 0.0 | Breast Margin 9100265 | 2.9 |
| (ODO4286) | | | |
| Muscle Margin (ODO4286) | 1.9 | Breast Cancer A209073 | 14.8 |
| Lung Malignant Cancer | 13.1 | Breast Margin | 23.3 |
| (OD03126) | | A2090734 | |
| Lung Margin (OD03126) | 26.8 | Normal Liver | 21.9 |
| Lung Cancer (OD04404) | 5.4 | Liver Cancer | 22.4 |
| Lung Margin (OD04404) | 37.9 | Liver Cancer 1025 | 0.0 |
| Lung Cancer (OD04565) | 2.9 | Liver Cancer 1026 | 2.1 |
| Lung Margin (OD04565) | 9.3 | Liver Cancer 6004-T | 13.5 |
| Lung Cancer (OD04237-01) | 24.8 | Liver Tissue 6004-N | 5.9 |
| Lung Margin (OD04237-02) | 10.5 | Liver Cancer 6005-T | 0.0 |
| Ocular Mel Met to Liver | 10.1 | Liver Tissue 6005-N | 0.0 |
| (ODO4310) | | | |
| Liver Margin (ODO4310) | 8.7 | Normal Bladder | 42.0 |
| Melanoma Metastasis | 0.0 | Bladder Cancer | 5.8 |
| Lung Margin (OD04321) | 25.0 | Bladder Cancer | 65.1 |
| Normal Kidney | 100.0 | Bladder Cancer | 5.6 |
| | | (OD04718-01) | |
| Kidney Ca, Nuclear grade 2 | 52.1 | Bladder Normal | 32.8 |
| (OD04338) | | Adjacent (OD04718-03) | |
| Kidney Margin (OD04338) | 22.1 | Normal Ovary | 5.7 |
| Kidney Ca Nuclear grade 1/2 | 41.5 | Ovarian Cancer | 9.4 |
| (OD04339) | | | |
| Kidney Margin (OD04339) | 46.0 | Ovarian Cancer | 8.8 |
| | | (OD04768-07) | |
| Kidney Ca, Clear cell type | 27.0 | Ovary Margin | 3.3 |
| (OD04340) | | (OD04768-08) | |
| Kidney Margin (OD04340) | 24.7 | Normal Stomach | 30.6 |
| Kidney Ca, Nuclear grade 3 | 9.1 | Gastric Cancer 9060358 | 0.0 |
| (OD04348) | | | |
| Kidney Margin (OD04348) | 92.7 | Stomach Margin | 0.0 |
| | | 9060359 | |
| Kidney Cancer (OD04622- | 5.0 | Gastric Cancer 9060395 | 5.3 |
| 01) | | | |
| Kidney Margin (OD04622- | 2.0 | Stomach Margin | 3.1 |
| 03) | | 9060394 | |
| Kidney Cancer (OD04450- | 10.9 | Gastric Cancer 9060397 | 4.7 |
| 01) | | | |
| Kidney Margin (OD04450- | 17.6 | Stomach Margin | 0.0 |
| 03) | | 9060396 | |
| Kidney Cancer 8120607 | 0.8 | Gastric Cancer 064005 | 11.3 |

TABLE AQI
Panel 4.1D

| | Rel. Exp.(%) Ag1533, | | Rel. Exp.(%) Ag1533, |
|---|---|---|---|
| Tissue Name | Run 223794696 | Tissue Name | Run 223794696 |
| Secondary Th1 act | 0.0 | HUVEC IL-1 beta | 11.8 |
| Secondary Th2 act | 20.4 | HUVEC IFN gamma | 11.5 |
| Secondary Tr1 act | 13.1 | HUVEC TNF alpha + IFN | 4.0 |
| | | gamma | |
| Secondary Th1 rest | 25.3 | HUVEC TNF alpha + IL4 | 18.7 |
| Secondary Th2 rest | 8.0 | HUVEC IL-11 | 29.1 |
| Secondary Tr1 rest | 33.9 | Lung Microvascular EC none | 83.5 |
| Primary Th1 act | 0.0 | Lung Microvascular EC | 14.0 |
| | | TNFalpha + IL-1 beta | |
| Primary Th2 act | 25.0 | Microvascular Dermal EC | 8.5 |
| | | none | |
| Primary Tr1 act | 7.9 | Microsvasular Dermal EC | 0.0 |
| | | TNFalpha + IL-1 beta | |
| Primary Th1 rest | 14.5 | Bronchial epithelium | 15.2 |
| | | TNFalpha + IL1beta | |
| Primary Th2 rest | 4.3 | Small airway epithelium none | 0.0 |
| Primary Tr1 rest | 20.7 | Small airway epithelium | 20.9 |
| | | TNFalpha + IL-1 beta | |
| CD45RA CD4 | 21.2 | Coronery artery SMC rest | 0.0 |
| lymphocyte act | | | |
| CD45RO CD4 | 65.5 | Coronery artery SMC | 0.0 |
| lymphocyte act | | TNFalpha + IL-1 beta | |
| CD8 lymphocyte act | 25.9 | Astrocytes rest | 20.3 |
| Secondary CD8 | 19.6 | Astrocytes TNFalpha + IL- | 10.3 |
| lymphocyte rest | | 1 beta | |
| Secondary CD8 | 4.5 | KU-812 (Basophil) rest | 10.9 |
| lymphocyte act | | | |
| CD4 lymphocyte none | 53.2 | KU-812 (Basophil) | 34.2 |
| | | PMA/ionomycin | |
| 2ry Th1/Th2/Tr1_anti- | 19.2 | CCD1106 (Keratinocytes) | 0.0 |
| CD95 CH11 | | none | |
| LAK cells rest | 22.1 | CCD1106 (Keratinocytes) | 8.5 |
| | | TNFalpha + IL-1 beta | |
| LAK cells IL-2 | 51.1 | Liver cirrhosis | 26.6 |
| LAK cells IL-2 + IL-12 | 3.1 | NCI-H292 none | 19.9 |
| LAK cells IL-2 + IFN | 24.0 | NCI-H292 IL-4 | 27.5 |
| gamma | | | |
| LAK cells IL-2 + IL-18 | 14.6 | NCI-H292 IL-9 | 29.3 |
| LAK cells | 0.0 | NCI-H292 IL-13 | 16.2 |
| PMA/ionomycin | | | |
| NK Cells IL-2 rest | 38.7 | NCI-H292 IFN gamma | 44.1 |
| Two Way MLR 3 day | 61.6 | HPAEC none | 8.5 |
| Two Way MLR 5 day | 35.6 | HPAEC TNF alpha + IL-1 | 10.7 |
| | | beta | |
| Two Way MLR 7 day | 9.9 | Lung fibroblast none | 60.7 |
| PBMC rest | 26.2 | Lung fibroblast TNF alpha + | 40.6 |
| | | IL-1 beta | |
| PBMC PWM | 3.6 | Lung fibroblast IL-4 | 8.8 |
| PBMC PHA-L | 4.5 | Lung fibroblast IL-9 | 21.3 |
| Ramos (B cell) none | 0.0 | Lung fibroblast IL-13 | 4.8 |
| Ramos (B cell) | 0.0 | Lung fibroblast IFN gamma | 4.6 |
| ionomycin | | | |
| B lymphocytes PWM | 5.1 | Dermal fibroblast CCD1070 | 0.0 |
| | | rest | |
| B lymphocytes CD40L | 17.0 | Dermal fibroblast CCD1070 | 20.0 |
| and IL-4 | | TNF alpha | |
| EOL-1 dbcAMP | 10.9 | Dermal fibroblast CCD1070 | 4.0 |
| | | IL-1 beta | |
| EOL-1 dbcAMP | 0.0 | Dermal fibroblast IFN gamma | 10.7 |
| PMA/ionomycin | | | |
| Dendritic cells none | 4.1 | Dermal fibroblast IL-4 | 26.6 |
| Dendritic cells LPS | 17.7 | Dermal Fibroblasts rest | 10.4 |
| Dendritic cells anti-CD40 | 14.8 | Neutrophils TNFa + LPS | 10.0 |
| Monocytes rest | 10.7 | Neutrophils rest | 21.3 |
| Monocytes LPS | 16.4 | Colon | 0.0 |
| Macrophages rest | 18.6 | Lung | 4.2 |
| Macrophages LPS | 8.0 | Thymus | 100.0 |
| HUVEC none | 5.4 | Kidney | 81.8 |
| HUVEC starved | 2.6 | | |

TABLE AQJ
Panel 4D

| | Rel. Exp.(%) Ag2862, | | Rel. Exp.(%) Ag2862, |
|---|---|---|---|
| Tissue Name | Run 164299494 | Tissue Name | Run 164299494 |
| Secondary Th1 act | 0.0 | HUVEC IL-1 beta | 0.0 |
| Secondary Th2 act | 10.9 | HUVEC IFN gamma | 4.2 |
| Secondary Tr1 act | 2.6 | HUVEC TNF alpha + IFN | 2.5 |
| | | gamma | |
| Secondary Th1 rest | 4.5 | HUVEC TNF alpha + IL4 | 4.6 |
| Secondary Th2 rest | 13.8 | HUVEC IL-11 | 2.4 |
| Secondary Tr1 rest | 9.8 | Lung Microvascular EC none | 28.9 |
| Primary Th1 act | 0.0 | Lung Microvascular EC | 13.2 |
| | | TNFalpha + IL-1 beta | |
| Primary Th2 act | 0.0 | Microvascular Dermal EC | 11.7 |
| | | none | |
| Primary Tr1 act | 0.0 | Microsvasular Dermal EC | 4.3 |
| | | TNFalpha + IL-1 beta | |
| Primary Th1 rest | 65.5 | Bronchial epithelium | 9.7 |
| | | TNFalpha + IL1beta | |
| Primary Th2 rest | 24.0 | Small airway epithelium none | 1.7 |
| Primary Tr1 rest | 6.8 | Small airway epithelium | 36.1 |
| | | TNFalpha + IL-1 beta | |
| CD45RA CD4 | 12.9 | Coronery artery SMC rest | 11.1 |
| lymphocyte act | | | |
| CD45RO CD4 | 7.4 | Coronery artery SMC | 6.7 |
| lymphocyte act | | TNFalpha + IL-1 beta | |
| CD8 lymphocyte act | 0.0 | Astrocytes rest | 9.4 |
| Secondary CD8 | 13.7 | Astrocytes TNFalpha + IL- | 8.0 |
| lymphocyte rest | | 1 beta | |
| Secondary CD8 | 0.0 | KU-812 (Basophil) rest | 5.9 |
| lymphocyte act | | | |
| CD4 lymphocyte none | 22.1 | KU-812 (Basophil) | 25.9 |
| | | PMA/ionomycin | |
| 2ry Th1/Th2/Tr1_anti- | 20.2 | CCD1106 (Keratinocytes) | 6.5 |
| CD95 CH11 | | none | |
| LAK cells rest | 20.7 | CCD1106 (Keratinocytes) | 2.9 |
| | | TNFalpha + IL-1 beta | |
| LAK cells IL-2 | 53.2 | Liver cirrhosis | 17.8 |
| LAK cells IL-2 + IL-12 | 27.4 | Lupus kidney | 13.9 |
| LAK cells IL-2 + IFN | 73.2 | NCI-H292 none | 23.2 |
| gamma | | | |
| LAK cells IL-2 + IL-18 | 12.6 | NCI-H292 IL-4 | 28.3 |
| LAK cells | 0.0 | NCI-H292 IL-9 | 24.1 |
| PMA/ionomycin | | | |
| NK Cells IL-2 rest | 26.8 | NCI-H292 IL-13 | 2.3 |
| Two Way MLR 3 day | 82.9 | NCI-H292 IFN gamma | 24.1 |
| Two Way MLR 5 day | 12.1 | HPAEC none | 8.0 |
| Two Way MLR 7 day | 0.0 | HPAEC TNF alpha + IL-1 | 6.3 |
| | | beta | |
| PBMC rest | 8.9 | Lung fibroblast none | 9.9 |
| PBMC PWM | 17.4 | Lung fibroblast TNF alpha + | 18.8 |
| | | IL-1 beta | |
| PBMC PHA-L | 7.0 | Lung fibroblast IL-4 | 11.7 |
| Ramos (B cell) none | 0.0 | Lung fibroblast IL-9 | 18.7 |
| Ramos (B cell) | 4.5 | Lung fibroblast IL-13 | 20.0 |
| ionomycin | | | |
| B lymphocytes PWM | 2.5 | Lung fibroblast IFN gamma | 17.3 |
| B lymphocytes CD40L | 12.9 | Dermal fibroblast CCD1070 | 3.0 |
| and IL-4 | | rest | |
| EOL-1 dbcAMP | 4.7 | Dermal fibroblast CCD1070 | 9.9 |
| | | TNF alpha | |
| EOL-1 dbcAMP | 0.0 | Dermal fibroblast CCD1070 | 5.1 |
| PMA/ionomycin | | IL-1 beta | |
| Dendritic cells none | 1.8 | Dermal fibroblast IFN gamma | 1.7 |
| Dendritic cells LPS | 2.8 | Dermal fibroblast IL-4 | 24.5 |
| Dendritic cells anti-CD40 | 0.0 | IBD Colitis 2 | 1.8 |
| Monocytes rest | 6.0 | IBD Crohn's | 2.1 |
| Monocytes LPS | 0.0 | Colon | 14.9 |
| Macrophages rest | 0.0 | Lung | 11.7 |
| Macrophages LPS | 11.9 | Thymus | 82.9 |
| HUVEC none | 0.0 | Kidney | 100.0 |
| HUVEC starved | 3.1 | | |

CNS_neurodegeneration_v1.0 Summary: Ag1533 The CG148698-01 gene shows widespread expression across all regions of the brain, with highest expression in the parietal cortex of a control patient (CT33.5). This gene appears to be upregulated in the temporal cortex of patients with Alzheimer's disease. The temporal cortex is a region that shows severe degeneration in Alzheimer's disease, suggesting the expression of this gene may play a role in the pathogenesis of this disease. Therapeutic modulation of this gene or treatment with an antagonist to the receptor may be of benefit in treating Alzheimer's disease or dementia.

Ag2862 Expression is low/undetected in all samples in this panel (CT>35). (Data not shown.)

General_screening_panel_v1.5 Summary: Ag1533 Highest expression of the CGI48698-01 gene is in the fetal lung (CT=30). Significant levels of expression are also detected in adult lung. This expression profile suggests that the gene product may be involved in the normal homeostasis of the lung. Therefore, therapeutic modulation of the expression or function of this gene may be effective in the treatment of diseases of that affect the lung including asthma, emphysema, and acute respiratory distress syndrome (ARDS).

This gene is also moderately expressed in a variety of metabolic tissues including adipose, adult and fetal heart, adult and fetal skeletal muscle, adrenal, pituitary, thyroid and pancreas. Thus, this gene product may be a small molecule drug target for the treatment of metabolic disease, including obesity and Types 1 and 2 diabetes. Furthermore, this gene is differentially expressed in adult (CT value=37) versus fetal liver (CT values=33.5), and may be used to differentiate between the adult and fetal phenotype in this tissue.

There is moderate expression in some tissues of the central nervous system, including the fetal brain and the spinal cord. Please see CNS_neurodegeneration_v1.0 Summary for discussion of potential utility in the central nervous system.

Overall, the expression of this gene is largely associated with normal tissues. However, significant expression of this gene is seen in cell lines derived ovarian and gastric cancers. Thus therapeutic modulation of this gene, through the use of small molecule drugs, antibodies or protein therapeutics might be of use in the treatment of ovarian or gastric cancer.

Panel 1.2 Summary: Ag1533 The expression of the CG148698-01 gene is highest in a sample derived from an ovarian cancer cell line (CT=29. 1). This particular cell line was derived from a unique form of ovarian cancer, that being ascites. In addition, there appears to be substantial expression of this gene in samples derived from other ovarian cancer cell lines as well-as normal bladder tissue, normal kidney tissue and a cell lined derived from a gastric cancer. Thus, the expression of this gene in these tissues could be used to distinguish these samples from other samples in the panel. Additionally, the expression of this gene could be used to distingush ascites derived samples from other samples in the panel. Furthermore, therapeutic modulation of this gene, through the use of small molecule drugs, antibodies or protein therapeutics might be of benefit in the treatment of ovarian cancer.

This gene is also expressed in a variety of metabolic tissues including Ag1533 is modestly expressed (CT values=31-34) in a variety of metabolic tissues including adult and fetal liver, adult and fetal heart, skeletal muscle, adrenal and pancreas. As is seen from General_screening_panel_v1.5, this suggests a role of the gene product in metabolic function. Thus, this gene product may be a small molecule drug target for the treatment of metabolic disease, including obesity and Types 1 and 2 diabetes.

Panel 1.3D Summary: Ag2617 Expression of the CG148698-01 gene is exclusive to an ovarian cancer cell line (SK-OV-3)(CT=33.1). Expression in this cell line is also detected in Panel 1.2. Interestingly, this cell line was derived from a unique form of ovarian cancer, that being ascites. Thus, the expression of this gene could be used to distinguish samples derived from this cell line from other samples in the panel in addition to distingushing ascites derived samples from other samples in the panel. Moreover, therapeutic modulation of this gene, through the use of small molecule drugs, antibodies or protein therapeutics might be of benefit in the treatment of ovarian cancer.

Panel 2D Summary: Ag1533 The expression of the CG148698-01 gene appears to be highest in a sample derived from normal kidney tissue (CT=31.9). In addition there is substantial expression in samples derived from other samples of normal kidney tissue adjacent to malignant kidney. Moreover, there also appears to be expression associated with tissues, normal or malignant, derived from uterus, prostate, breast, bladder and thyroid. Thus, the expression of this gene could be used to distinguish samples derived from these tissue types when compared to other samples in the panel. Further, therapeutic modulation of this gene, or gene product, through the use of small molecule drugs, antibodies or protein therapeutics might be of benefit in the treatment of cancers of the above listed tissues.

Panel 4.1D Summary: Ag1533 The CG148698-01 transcript is expressed on most tissues in panel 4.1 D regardless of treatment, with highest expression in the thymus(CT=32.8). This transcript encodes a GPCR-like molecule with potential signaling activity and may important in maintaining normal cellular functions in a number of tissues. Therapies designed with the protein encoded for by this transcript could be important in regulating cellular viability or function.

Panel 4D Summary: Ag2862 The CG148698-01 transcript appears to be expressed in this panel regardless of treatment, with highest expression in the kidney (CT=33.2). This result is concordant with the results from Panel 2D. This transcript encodes a GPCR-like molecule with potential signaling activity and may important in maintaining normal cellular functions in a number of tissues. Therapies designed with the protein encoded for by this transcript could be important in regulating cellular viability or function.

AR. CG55956-02: Olfactory Receptor

Expression of gene CG55956-02 was assessed using the primer-probe set Ag2193, described in Table ARA. Results of the RTQ-PCR runs are shown in Tables ARB, ARC and ARD.

TABLE AQA
Probe Name Ag2193

| Primers | Sequences | Length | Start Position | SEQ ID NO |
|---|---|---|---|---|
| Forward | 5′-gccctttagataagtcgtccaa-3′ | 22 | 689 | 338 |
| Probe | TET-5′-agctctgtccactttgactgctcaca-3′-TAMRA | 26 | 711 | 339 |
| Reverse | 5′-catggtccaaagaacaaaagaa-3′ | 22 | 746 | 340 |

TABLE ARB
Panel 1.3D

| | Rel. Exp.(%) Ag2193, | | Rel. Exp.(%) Ag2193, |
|---|---|---|---|
| Tissue Name | Run 165750872 | Tissue Name | Run 165750872 |
| Liver adenocarcinoma | 0.0 | Kidney (fetal) | 0.0 |
| Pancreas | 0.0 | Renal ca. 786-0 | 22.7 |
| Pancreatic ca. CAPAN 2 | 0.0 | Renal ca. A498 | 25.5 |
| Adrenal gland | 0.0 | Renal ca. RXF 393 | 0.0 |
| Thyroid | 0.0 | Renal ca. ACHN | 100.0 |
| Salivary gland | 0.0 | Renal ca. UO-31 | 4.1 |
| Pituitary gland | 0.0 | Renal ca. TK-10 | 21.9 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 1.9 |
| | | HepG2 | |
| Brain (cerebellum) | 3.6 | Lung | 0.0 |
| Brain (hippocampus) | 0.0 | Lung (fetal) | 0.0 |
| Brain (substantia nigra) | 0.0 | Lung ca. (small cell) | 5.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 0.0 | Lung ca. (large | 0.0 |
| | | cell) NCI-H460 | |
| glio/astro U87-MG | 9.0 | Lung ca. (non-sm. cell) | 0.0 |
| | | A549 | |
| glio/astro U-118-MG | 48.0 | Lung ca. (non-s. cell) | 5.0 |
| | | NCI-H23 | |
| Astrocytoma SW1783 | 5.9 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) SW | 0.0 |
| | | 900 | |
| astrocytoma SNB-75 | 2.8 | Lung ca. (squam.) NCI- | 0.0 |
| | | H596 | |
| glioma SNB-19 | 14.1 | Mammary gland | 0.0 |
| glioma U251 | 43.2 | Breast ca.* (pl. ef) | 0.0 |
| | | MCF-7 | |
| glioma SF-295 | 3.7 | Breast ca.* (pl. ef) | 0.0 |
| | | MDA-MB-231 | |
| Heart (fetal) | 0.0 | Breast ca.* (pl. ef) | 0.0 |
| | | T47D | |
| Heart | 0.0 | Breast ca. BT-549 | 6.2 |
| Skeletal muscle (fetal) | 0.0 | Breast ca. MDA-N | 31.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 4.8 |
| Spleen | 0.0 | Ovarian ca. OVCAR-5 | 0.0 |
| Lymph node | 0.0 | Ovarian ca. OVCAR-8 | 8.6 |
| Colorectal | 28.1 | Ovarian ca. IGROV-1 | 0.0 |
| Stomach | 0.0 | Ovarian ca.* (ascites) | 0.0 |
| | | SK-OV-3 | |
| Small intestine | 0.0 | Uterus | 0.0 |
| Colon ca. SW480 | 3.3 | Plancenta | 0.0 |
| Colon ca.* | 0.0 | Prostate | 0.0 |
| SW620 (SW480 met) | | | |
| Colon ca. HT29 | 0.0 | Prostate ca.* (bone | 0.0 |
| | | met) PC-3 | |
| Colon ca. HCT-116 | 0.0 | Testis | 5.2 |
| Colon ca. CaCo-2 | 0.0 | Melanoma Hs688(A).T | 0.0 |
| Colon ca. | 0.0 | Melanoma* (met) | 0.0 |
| tissue (ODO3866) | | Hs688(B).T | |
| Colon ca. HCC-2998 | 4.9 | Melanoma UACC-62 | 27.2 |
| Gastric ca.* (liver met) | 0.0 | Melanoma M14 | 32.8 |
| NCI-N87 | | | |
| Bladder | 0.0 | Melanoma LOX IMVI | 0.0 |
| Trachea | 0.0 | Melanoma* (met) SK- | 11.4 |
| | | MEL-5 | |
| Kidney | 0.0 | Adipose | 4.5 |

TABLE ARC
Panel 2D

| | Rel. Exp.(%) Ag2193, | | Rel. Exp.(%) Ag2193, |
|---|---|---|---|
| Tissue Name | Run 163584683 | Tissue Name | Run 163584683 |
| Normal Colon | 4.9 | Kidney Margin 8120608 | 2.4 |
| CC Well to Mod Diff | 32.5 | Kidney Cancer 8120613 | 2.5 |
| (ODO3866) | | | |
| CC Margin (ODO3866) | 13.2 | Kidney Margin 8120614 | 0.0 |
| CC Gr.2 rectosigmoid | 0.0 | Kidney Cancer 9010320 | 0.0 |
| (ODO3868) | | | |
| CC Margin (ODO3866) | 0.0 | Kidney Margin 9010321 | 0.0 |
| CC Mod Diff (ODO3920) | 2.2 | Normal Uterus | 0.0 |
| CC Margin (ODO3920) | 0.0 | Uterus Cancer 064011 | 0.0 |
| CC Gr.2 ascend colon | 0.0 | Normal Thyroid | 0.0 |
| (ODO3921) | | | |
| CC Margin (ODO3921) | 15.8 | Thyroid Cancer 064010 | 0.0 |
| CC from Partial | 0.0 | Thyroid Cancer | 0.0 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 0.0 | Normal Breast | 0.0 |
| (OD04451-01) | | | |
| Lung Margin (OD04451-02) | 1.7 | Breast Cancer | 8.9 |
| | | (OD04566) | |
| Normal Prostate 6546-1 | 1.6 | Breast Cancer | 0.0 |
| | | (OD04590-01) | |
| Prostate Cancer (OD04410) | 3.8 | Breast Cancer Mets | 0.0 |
| | | (OD04590-03) | |
| Prostate Margin (OD04410) | 0.0 | Breast Cancer | 4.0 |
| | | Metastasis (OD04655- | |
| | | 05) | |
| Prostate Cancer (OD04720- | 0.0 | Breast Cancer 064006 | 0.0 |
| 01) | | | |
| Prostate Margin (OD04720- | 1.6 | Breast Cancer 1024 | 0.0 |
| 02) | | | |
| Normal Lung 061010 | 4.7 | Breast Cancer 9100266 | 0.0 |
| Lung Met to Muscle | 34.6 | Breast Margin 9100265 | 0.0 |
| (ODO4286) | | | |
| Muscle Margin (ODO4286) | 2.5 | Breast Cancer A209073 | 20.7 |
| Lung Malignant Cancer | 0.0 | Breast Margin | 1.6 |
| (OD03126) | | A2090734 | |
| Lung Margin (OD03126) | 0.6 | Normal Liver | 0.0 |
| Lung Cancer (OD04404) | 0.0 | Liver Cancer 064003 | 2.6 |
| Lung Margin (OD04404) | 3.0 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 3.2 |
| Lung Cancer (OD04237-01) | 0.0 | Liver Tissue 6004-N | 15.0 |
| Lung Margin (OD04237-02) | 0.0 | Liver Cancer 6005-T | 0.0 |
| Ocular Mel Met to Liver | 39.0 | Liver Tissue 6005-N | 0.0 |
| (ODO4310) | | | |
| Liver Margin (ODO4310) | 5.9 | Normal Bladder | 0.0 |
| Melanoma Mets to Lung | 11.0 | Bladder Cancer 1023 | 0.0 |
| (OD04321) | | | |
| Lung Margin (OD04321) | 0.0 | Bladder Cancer | 66.9 |
| | | A302173 | |
| Normal Kidney | 4.9 | Bladder Cancer | 0.0 |
| | | (OD04718-01) | |
| Kidney Ca, Nuclear grade 2 | 95.9 | Bladder Normal | 0.0 |
| (OD04338) | | Adjacent (OD04718-03) | |
| Kidney Margin (OD04338) | 23.0 | Normal Ovary | 0.0 |
| Kidney Ca Nuclear grade 1/2 | 100.0 | Ovarian Cancer 064008 | 0.0 |
| (OD04339) | | | |
| Kidney Margin (OD04339) | 37.1 | Ovarian Cancer | 1.5 |
| | | (OD04768-07) | |
| Kidney Ca, Clear cell type | 9.9 | Ovary Margin | 0.0 |
| (OD04340) | | (OD04768-08) | |
| Kidney Margin (OD04340) | 5.8 | Normal Stomach | 0.0 |
| Kidney Ca, Nuclear grade 3 | 0.0 | Gastric Cancer 9060358 | 0.0 |
| (OD04348) | | | |
| Kidney Margin (OD04348) | 15.7 | Stomach Margin | 0.0 |
| | | 9060359 | |
| Kidney Cancer (OD04622- | 0.0 | Gastric Cancer 9060395 | 5.4 |
| 01) | | | |
| Kidney Margin (OD04622- | 2.1 | Stomach Margin | 0.0 |
| 03) | | 9060394 | |
| Kidney Cancer (OD04450- | 66.4 | Gastric Cancer 9060397 | 0.0 |
| 01) | | | |
| Kidney Margin (OD04450- | 7.1 | Stomach Margin | 0.0 |
| 03) | | 9060396 | |
| Kidney Cancer 8120607 | 1.6 | Gastric Cancer 064005 | 46.0 |

TABLE ARD
Panel 4D

| | Rel. Exp.(%) Ag2193, | | Rel. Exp.(%) Ag2193, |
|---|---|---|---|
| Tissue Name | Run 163603073 | Tissue Name | Run 163603073 |
| Secondary Th1 act | 12.1 | HUVEC IL-1 beta | 14.2 |
| Secondary Th2 act | 0.0 | HUVEC IFN gamma | 0.0 |
| Secondary Tr1 act | 0.0 | HUVEC TNF alpha + IFN | 52.9 |
| | | gamma | |
| Secondary Th1 rest | 0.0 | HUVEC TNF alpha + IL4 | 9.2 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1 beta | |
| Primary Th2 act | 5.3 | Microvascular Dermal EC | 0.0 |
| | | none | |
| Primary Tr1 act | 0.0 | Microsvasular Dermal EC | 0.0 |
| | | TNFalpha + IL-1 beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1 beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1 beta | |
| Secondary CD8 | 0.0 | KU-812 (Basophil) rest | 0.0 |
| lymphocyte act | | | |
| CD4 lymphocyte none | 0.0 | KU-812 (Basophil) | 6.8 |
| | | PMA/ionomycin | |
| 2ry Th1/Th2/Tr1_anti- | 7.8 | CCD1106 (Keratinocytes) | 0.0 |
| CD95 CH11 | | none | |
| LAK cells rest | 0.0 | CCD1106 (Keratinocytes) | 7.6 |
| | | TNFalpha + IL-1 beta | |
| LAK cells IL-2 | 7.2 | Liver cirrhosis | 66.0 |
| LAK cells IL-2 + IL-12 | 0.0 | Lupus kidney | 14.9 |
| LAK cells IL-2 + IFN | 0.0 | NCI-H292 none | 68.8 |
| gamma | | | |
| LAK cells IL-2 + IL-18 | 7.1 | NCI-H292 IL-4 | 70.2 |
| LAK cells | 0.0 | NCI-H292 IL-9 | 100.0 |
| PMA/ionomycin | | | |
| NK Cells IL-2 rest | 0.0 | NCI-H292 IL-13 | 31.0 |
| Two Way MLR 3 day | 0.0 | NCI-H292 IFN gamma | 72.2 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 7.9 |
| Ramos (B cell) none | 0.0 | Lung fibroblast IL-9 | 22.4 |
| Ramos (B cell) | 0.0 | Lung fibroblast IL-13 | 7.6 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 13.7 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 24.3 |
| Monocytes rest | 0.0 | IBD Crohn's | 6.7 |
| Monocytes LPS | 0.0 | Colon | 34.9 |
| Macrophages rest | 90.1 | Lung | 12.4 |
| Macrophages LPS | 0.0 | Thymus | 13.7 |
| HUVEC none | 36.9 | Kidney | 0.0 |
| HUVEC starved | 55.9 | | |

Panel 1.3D Summary: Ag2193 The expression of the CG55956-02 gene appears to be highest in a sample derived from a renal cancer cell line (ACHN)(CT=33.2). In addition, there is substantial expression associated with brain cancer cell lines, a melanoma and a breast cancer cell line. Thus, the expression of this gene could be used to distinguish samples derived from the ACHN cell line form other samples in the panel. Moreover, therapeutic modulation of this gene, through the use of small molecule drugs, antibodies or protein therapeutics might be of use in the treatment of melanoma, breast cancer, renal cancer of brain cancer.

Panel 2D Summary: Ag2193 The expression of the CG55956-02 gene is highest in a sample derived from a kidney cancer (CT=32. 1). In addition, there is substantial expression associated with other kidney cancers. Of note is the difference in expression between kidney cancers and their normal adjacent tissues. Thus, the expression of this gene could be used to distinguish kidney cancer samples from other samples in the panel, and in particular, distinguish kidney cancer from normal kidney. Moreover, therapeutic modulation of this gene, through the use of small molecule drugs, antibodies or protein therapeutics might be of use in the treatment of kidney cancer.

Panel 4D Summary: Ag2193 The CG55956-02 gene is expressed at low levels in resting and IL-4, IL-9, and IFN gamma activated-NCI-H292 mucoepidermoid cells, starved and TNF alpha+IFN gamma treated HUVECs, and resting macrophages. The expression of this gene in lung derived cells, endothelial cells and macrophages suggests that this gene may be involved in normal conditions as well as pathological and inflammatory lung disorders including chronic obstructive pulmonary disease, asthma, allergy and emphysema. Small molecules or antibodies that modulate the function of this gene may reduce or eliminate symptoms in chronic obstructive pulmonary disease, asthma, allergy, and emphysema.

AS. CG56103-02: Olfactory Receptor

Expression of gene CG56103-02 was assessed using the primer-probe set Ag2205, described in Table ASA. Results of the RTQ-PCR runs are shown in Tables ASB, and ASC.

TABLE ASA
Probe Name Ag2205

| Primers | Sequences | Length | Start Position | SEQ ID NO |
|---|---|---|---|---|
| Forward | 5′-acctccgtgtctgaattcatc-3′ | 21 | 30 | 341 |
| Probe | TET-5′-ccacctccagctgatgctcttcct-3′-TAMRA | 24 | 74 | 342 |
| Reverse | 5′-acaggtacatcagcaggaacag-3′ | 22 | 99 | 343 |

TABLE ASB
Panel 1.3D

| | Rel. Exp.(%) Ag2205, | | Rel. Exp.(%) Ag2205, |
|---|---|---|---|
| Tissue Name | Run 165974832 | Tissue Name | Run 165974832 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 100.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 19.6 | Lung | 0.0 |
| Brain (hippocampus) | 25.3 | Lung (fetal) | 0.0 |
| Brain (substantia nigra) | 0.0 | Lung ca. (small cell) | 0.0 |
| | | LX-1 | |
| Brain (thalamus) | 15.5 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 18.3 | Lung ca. (large | 0.0 |
| | | cell) NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 46.3 |
| | | HOP-62 | |
| neuro*; met SK-N-AS | 22.7 | Lung ca. (non-s. cl) | 0.0 |
| | | NCI-H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl. ef) | 0.0 |
| | | MCF-7 | |
| glioma SF-295 | 16.2 | Breast ca.* (pl. ef) | 0.0 |
| | | MDA-MB-231 | |
| Heart (Fetal) | 0.0 | Breast ca.* (pl. ef) | 0.0 |
| | | T47D | |
| Heart | 0.0 | Breast ca. BT-549 | 0.0 |
| Skeletal muscle (Fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 17.7 |
| Thymus | 0.0 | Ovarian ca. OVCAR-4 | 2.0 |
| Spleen | 0.0 | Ovarian ca. OVCAR-5 | 0.0 |
| Lymph node | 0.0 | Ovarian ca. OVCAR-8 | 0.0 |
| Colorectal | 5.4 | Ovarian ca. IGROV-1 | 0.0 |
| Stomach | 0.0 | Ovarian ca. (ascites) | 14.7 |
| | | SK-OV-3 | |
| Small intestine | 0.0 | Uterus | 0.0 |
| Colon ca. SW480 | 0.0 | Placenta | 0.0 |
| Colon ca.* SW620 | 0.0 | Prostate | 0.0 |
| (SW480 met) | | | |
| Colon ca. HT29 | 0.0 | Prostate ca.* (bone | 0.0 |
| | | met) PC-3 | |
| Colon ca. HCT-116 | 0.0 | Testis | 1.3 |
| Colon ca. CaCo-2 | 0.0 | Melanoma Hs688(A).T | 0.0 |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 0.0 |
| Gastric ca. (liver met) | 24.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE ASC
Panel 4D

| | Rel. Exp.(%) Ag2205, | | Rel. Exp.(%) Ag2205, |
|---|---|---|---|
| Tissue Name | Run 163623519 | Tissue Name | Run 163623519 |
| Secondary Th1 act | 0.0 | HUVEC IL-1 beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 8.4 |
| Secondary Tr1 rest | 0.0 | Lung Microvascular EC none | 0.0 |
| Primary Th1 act | 0.0 | Lung Microvascular EC | 0.0 |
| | | TNFalpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1 beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 5.5 | Small airway epithelium | 0.0 |
| | | TNFalpha + IL-1 beta | |
| CD45RA CD4 | 0.0 | Coronery artery SMC rest | 0.0 |
| lymphocyte act | | | |
| CD45RO CD4 | 15.5 | Coronery artery SMC | 0.0 |
| lymphocyte act | | TNFalpha + IL-1 beta | |
| CD8 lymphocyte act | 7.0 | Astrocytes rest | 0.0 |
| Secondary CD8 | 0.0 | Astrocytes TNFalpha + IL- | 0.0 |
| lymphocyte rest | | 1 beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1 beta | |
| LAK cells IL-2 | 0.0 | Liver cirrhosis | 100.0 |
| LAK cells IL-2 + IL-12 | 15.1 | Lupus kidney | 0.0 |
| LAK cells IL-2 + IFN | 0.0 | NCI-H292 none | 0.0 |
| gamma | | | |
| LAK cells IL-2 + IL-18 | 5.9 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 9.6 | HPAEC none | 0.0 |
| Two Way MLR 7 day | 14.6 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 8.2 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 8.2 |
| Monocytes rest | 0.0 | IBD Crohn's | 6.9 |
| Monocytes LPS | 0.0 | Colon | 0.0 |
| Macrophages rest | 0.0 | Lung | 54.0 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag2205 Expression of the CG56103-02 gene is low/undetectable across all of the samples on this panel. (Data not shown.)

Panel 1.3D Summary: Ag2205 Significant expression of the CG56103-02 gene is limited to a renal cancer cell line and a lung cancer cell line (CTs=33-34). Thus, expression of this gene could be used to differentiate between these cell lines and other samples on this panel and as a marker to detect the presence of lung and renal cancer.

Panel 2.2 Summary: Ag2205 Expression of the CG56103-02 gene is low/undetectable across all of the samples on this panel. (Data not shown.)

Panel 4D Summary: Ag2205 Low but significant expression of the CG56103-02 gene is detected in a liver cirrhosis sample (CT=33.46). Furthermore, expression of this gene is not detected in normal liver in Panel 1.3D, suggesting that its expression is unique to liver cirrhosis. This gene encodes a putative GPCR; therefore, antibodies or small molecule therapeutics could reduce or inhibit fibrosis that occurs in liver cirrhosis. Antibodies to this putative GPCR could also be used for the diagnosis of liver cirrhosis. In addition, expression in normal lung suggests a possible role in lung homeostasis.

AT. CG55773-02: Olfactory Receptor

Expression of gene CG55773-02 was assessed using the primer-probe set Ag5286, described in Table ATA. Results of the RTQ-PCR runs are shown in Table ATB.

TABLE ATA
Probe Name Ag5286

| Primers | Sequences | Length | Start Position | SEQ ID NO |
|---|---|---|---|---|
| Forward | 5′-ttcctctccatcttgggatc-3′ | 20 | 618 | 344 |
| Probe | TET-5′-tcacactctggtcatcagagctgtgc-3′-TAMRA | 26 | 638 | 345 |
| Reserve | 5′-tagttcgaccagcaccagag-3′ | 20 | 674 | 346 |

TABLE ATB
General_screening_panel_v1.5

| | Rel. Exp.(%) Ag5286, | | Rel. Exp.(%) Ag5286, |
|---|---|---|---|
| Tissue Name | Run 233238991 | Tissue Name | Run 233238991 |
| Adipose | 1.7 | Renal ca. TK-10 | 0.0 |
| Melanoma* | 0.0 | Bladder | 3.5 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* LOXIMVI | 1.6 | Colon ca. SW-948 | 0.0 |
| Melanoma* SK-MEL-5 | 0.5 | Colon ca. SW480 | 0.0 |
| Squamous cell | 2.8 | Colon ca.* (SW480 met) | 0.0 |
| carcinoma SCC-4 | | SW620 | |
| Testis Pool | 5.9 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 11.0 |
| Placenta | 0.0 | Colon cancer tissue | 0.0 |
| Uterus Pool | 1.8 | Colon ca. SW1116 | 0.0 |
| Ovarian ca. OVCAR-3 | 1.0 | Colon ca. Colo-205 | 0.0 |
| Ovarian ca. SK-OV-3 | 8.5 | Colon ca. SW-48 | 0.0 |
| Ovarian ca. OVCAR-4 | 0.5 | Colon Pool | 2.4 |
| Ovarian ca. OVCAR-5 | 1.9 | Small Intestine Pool | 7.3 |
| Ovarian ca. IGROV-1 | 0.0 | Stomach Pool | 0.0 |
| Ovarian ca. OVCAR-8 | 3.3 | Bone Marrow Pool | 1.8 |
| Ovary | 0.0 | Fetal Heart | 0.0 |
| Breast ca. MCF-7 | 0.0 | Heart Pool | 7.3 |
| Breast ca. MDA-MB- | 0.0 | Lymph Node Pool | 5.2 |
| 231 | | | |
| Breast ca. BT 549 | 0.0 | Fetal Skeletal Muscle | 4.9 |
| Breast ca. T47D | 0.0 | Skeletal Muscle Pool | 0.0 |
| Breast ca. MDA-N | 0.0 | Spleen Pool | 0.0 |
| Breast Pool | 1.3 | Thymus Pool | 1.2 |
| Trachea | 0.0 | CNS cancer (glio/astro) | 0.0 |
| | | U87-MG | |
| Lung | 0.0 | CNS cancer (glio/astro) | 0.0 |
| | | U-118-MG | |
| Fetal Lung | 0.0 | CNS cancer (neuro; met) | 0.0 |
| | | SK-N-AS | |
| Lung ca. NCI-N417 | 0.0 | CNS cancer (astro) SF- | 0.0 |
| | | 539 | |
| Lung ca. LX-1 | 2.9 | CNS cancer (astro) SNB- | 5.9 |
| | | 75 | |
| Lung ca. NCI-H146 | 0.0 | CNS cancer (glio) SNB- | 0.0 |
| | | 19 | |
| Lung ca. SHP-77 | 1.4 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 15.3 | Brain (fetal) | 3.9 |
| Lung ca. NCI-H460 | 100.0 | Brain (Hippocampus) | 0.0 |
| | | Pool | |
| Lung ca. HOP-62 | 0.0 | Cerebral Cortex Pool | 3.0 |
| Lung ca. NCI-H522 | 6.0 | Brain (Substantia nigra) | 0.0 |
| | | Pool | |
| Liver | 0.0 | Brain (Thalamus) Pool | 0.0 |
| Fetal Liver | 0.0 | Brain (whole) | 2.0 |
| Liver ca. HepG2 | 0.0 | Spinal Cord Pool | 1.9 |
| Kidney Pool | 0.0 | Adrenal Gland | 0.0 |
| Fetal Kidney | 5.5 | Pituitary gland Pool | 0.0 |
| Renal ca. 786-0 | 0.0 | Salivary Gland | 0.0 |
| Renal ca. A498 | 3.3 | Thyroid (female) | 0.0 |
| Renal ca. ACHN | 0.0 | Pancreatic ca. CAPAN2 | 3.1 |
| Renal ca. UO-31 | 0.0 | Pancreas Pool | 0.0 |

CNS_neurodegeneration_v1.0 Summary: Ag5286 Expression of the CG55773-02 gene is low/undetectable in all samples on this panel (CT>35). (Data not shown.)

General_screening_panel_v1.5 Summary: Ag5286 Expression of the CG55773-02 gene is restricted to two lung cancer cell lines (CTs=31-34). Thus, expression of this gene could be used to differentiate between these samples and other samples on this panel. Furthermore, this expression profile suggests that expression of this gene could potentially be used as a marker to detect the presence of lung cancer

Panel 4.1D Summary: Ag5286 Expression of the CG55773-02 gene is low/undetectable in all samples on this panel (CT>35). (Data not shown.)

AU. CG50285-02 and CG50285-01/SC88066237_A: Olfactory Receptor

Expression of gene CG50285-02 was assessed using the primer-probe set Ag2539, described in Table AUA. Results of the RTQ-PCR runs are shown in Tables AUB, and AUC.

TABLE AUA
Probe Name Ag2539

| | | | | SEQ ID |
|---|---|---|---|---|
| Primers | Sequences | Length | Start Position | NO |
| Forward | 5′-cacctccattcccctatgtact-3′ | 22 | 137 | 347 |
| Probe | TET-5′-tccttagtaacttggccttgttgaca-3′-TAMRA | 27 | 162 | 348 |
| Reverse | 5′-ggactgtagtcgacgtaaagca-3′ | 22 | 191 | 349 |

TABLE AUB
Panel 1.3D

| | Rel. Exp.(%) Ag2539, | | Rel. Exp.(%) Ag2539, |
|---|---|---|---|
| Tissue Name | Run 166177198 | Tissue Name | Run 166177198 |
| Liver adenocarcinoma | 0.0 | Kidney (fetal) | 0.0 |
| Pancreas | 13.6 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 11.9 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 7.6 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 13.7 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 0.0 | Lung ca. (large | 0.0 |
| | | cell) NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 7.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl. ef) | 0.0 |
| | | MCF-7 | |
| glioma SF-295 | 0.0 | Breast ca.* (pl. ef) | 0.0 |
| | | MDA-MB-231 | |
| Heart (Fetal) | 0.0 | Breast ca.* (pl. ef) | 0.0 |
| | | T47D | |
| Heart | 0.0 | Breast ca. BT-549 | 0.0 |
| Skeletal muscle (Fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 11.4 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 28.3 |
| Colorectal | 21.2 | Ovarian ca. IGROV-1 | 0.0 |
| Stomach | 0.0 | Ovarian ca. (ascites) | 7.0 |
| | | SK-OV-3 | |
| Small intestine | 0.0 | Uterus | 0.0 |
| Colon ca. SW480 | 0.0 | Placenta | 0.0 |
| Colon ca.* SW620 | 0.0 | Prostate | 0.0 |
| (SW480 met) | | | |
| Colon ca. HT29 | 0.0 | Prostate ca.* (bone | 12.2 |
| | | met) PC-3 | |
| Colon ca. HCT-116 | 0.0 | Testis | 100.0 |
| Colon ca. CaCo-2 | 0.0 | Melanoma Hs688(A).T | 0.0 |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 2.1 |
| | | MEL-5 | |
| Kidney | 11.4 | Adipose | 0.0 |

TABLE AUC
Panel 4D

| | Rel. Exp.(%) Ag2539, | | Rel. Exp.(%) Ag2539, |
|---|---|---|---|
| Tissue Name | Run 164295847 | Tissue Name | Run 164295847 |
| Secondary Th1 act | 0.0 | HUVEC IL-1 beta | 0.0 |
| Secondary Th2 act | 0.0 | HUVEC IFN gamma | 0.0 |
| Secondary Tr1 act | 3.3 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1 beta | |
| Primary Th1 rest | 0.0 | Bronchial epithelium | 0.0 |
| | | TNFalpha + IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 3.0 |
| lymphocyte act | | TNFalpha + IL-1 beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1 beta | |
| Secondary CD8 | 0.0 | KU-812 (Basophil) rest | 32.5 |
| lymphocyte act | | | |
| CD4 lymphocyte none | 0.0 | KU-812 (Basophil) | 100.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1 beta | |
| LAK cells IL-2 | 0.0 | Liver cirrhosis | 16.0 |
| LAK cells IL-2 + IL-12 | 2.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 2.9 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 4.1 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 2.3 | Dermal fibroblast IL-4 | 0.7 |
| Dendritic cells anti-CD40 | 5.1 | IBD Colitis 2 | 15.4 |
| Monocytes rest | 0.0 | IBD Crohn's | 0.0 |
| Monocytes LPS | 0.0 | Colon | 0.0 |
| Macrophages rest | 3.3 | Lung | 2.6 |
| Macrophages LPS | 0.0 | Thymus | 12.2 |
| HUVEC none | 0.0 | Kidney | 0.0 |
| HUVEC starved | 0.0 | | |

CNS_neurodegeneration_v1.0 Summary: Ag2539 Expression of the CG50285-02 gene is low/undetected (CT>34.5) for all the samples in this panel (Data not shown.)

Panel 1.3D Summary: Ag2539 Expression of the CG50285-02 gene is restricted to the i 5 testis (CT=34.2) Thus, expression of this gene could be used as a marker for testis tissue. The expression of the gene at significant levels in testis only suggests that the CG50285-02 gene product may be involved in fertility. Therefore, therapeutic modulation of the function or expression of the protein encoded by the CG50285-02 gene may be useful in treating disease states where fertility is compromised.

Panel 2.2 Summary: Ag2539 Expression of the the CG50285-02 gene is low/undetected (CT>35) for all the samples in this panel (Data not shown.)

Panel 4D Summary: Ag2539 The CG50285-02 transcript is expressed in the PMA and ionomycin treated basophil cell line KU-812 and to a lesser extent in untreated KU-812 cells. This gene encodes a putative GPCR and it is known that GPCR-type receptors are important in multiple physiological responses mediated by basophils (ref. 1). Therefore, antibody or small molecule therapies designed with the protein encoded for by this gene could block or inhibit inflammation or tissue damage due to basophil activation in response to asthma, allergies, hypersensitivity reactions, psoriasis, and viral infections.

### REFERENCES

1. Heinemann A., Hartnell A., Stubbs V. E., Murakami K., Soler D., LaRosa G., Askenase P. W., Williams T. J., Sabroe I. (2000) Basophil responses to chemokines are regulated by both sequential and cooperative receptor signaling. J. Immunol. 165: 7224-7233.

To investigate human basophil responses to chemokines, we have developed a sensitive assay that uses flow cytometry to measure leukocyte shape change as a marker of cell responsiveness. PBMC were isolated from the blood of volunteers. Basophils were identified as a single population of cells that stained positive for IL-3Ralpha (CDw123) and negative for HLA-DR, and their increase in forward scatter (as a result of cell shape change) in response to chemokines was measured. Shape change responses of basophils to chemokines were highly reproducible, with a rank order of potency: monocyte chemoattractant protein (MCP) 4 (peak at /=eotaxin-2=eotaxin-3>/=eotaxin>MCP-1=MCP-3>macrophage-inflammatory protein-1alpha>RANTES=MCP-2=IL-8. The CCR4-selective ligand macrophage-derived chemokine did not elicit a response at concentrations up to 10 nM. Blocking mAbs to CCR2 and CCR3 demonstrated that responses to higher concentrations (>10 nM) of MCP-1 were mediated by CCR3 rather than CCR2, whereas MCP-4 exhibited a biphasic response consistent with sequential activation of CCR3 at lower concentrations and CCR2 at 10 nM MCP-4 and above. In contrast, responses to MCP-3 were blocked only in the presence of both mAbs, but not after pretreatment with either anti-CCR2 or anti-CCR3 mAb alone. These patterns of receptor usage were different from those seen for eosinophils and monocytes. We suggest that cooperation between CCRs might be a mechanism for preferential recruitment of basophils, as occurs in tissue hypersensitivity responses in vivo.

PMID: 11120855

AV. CG55766-01: GPCR

Expression of gene CG55766-01 was assessed using the primer-probe set Ag2182, described in Table AVA. Results of the RTQ-PCR runs are shown in Tables AVB and AVC.

TABLE AVA
Probe Name Ag2182

| Primers | Sequcnces | Length | Start Position | SEQ ID NO |
|---|---|---|---|---|
| Forward | 5′-aagctgtgtggttgaattcatc-3′ | 22 | 55 | 350 |
| Probe | TET-5′-tctaactatcctgagctccaggggca-3′-TAMRA | 26 | 89 | 351 |
| Reverse | 5′-taaataaccaggaaagccacaa-3′ | 22 | 120 | 352 |

TABLE AVB
Panel 2D

| | Rel. Exp.(%) Ag2182, | | Rel. Exp.(%) Ag2182, |
|---|---|---|---|
| Tissue Name | Run 163582696 | Tissue Name | Run 163582696 |
| Normal Colon | 0.0 | Kidney Margin 8120608 | 0.0 |
| CC Well to Mod Diff | 0.0 | Kidney Cancer 8120613 | 0.0 |
| (ODO3866) | | | |
| CC Margin (ODO3866) | 9.1 | Kidney Margin 8120614 | 0.0 |
| CC Gr.2 rectosigmoid | 0.0 | Kidney Cancer 9010320 | 0.0 |
| (ODO3868) | | | |
| CC Margin (ODO3868) | 0.0 | Kidney Margin 9010321 | 0.0 |
| CC Mod Diff (ODO3920) | 0.0 | Normal Uterus | 0.0 |
| CC Margin (ODO3920) | 0.0 | Uterus Cancer 064011 | 0.0 |
| CC Gr.2 ascend colon | 0.0 | Normal Thyroid | 0.0 |
| (ODO3921) | | | |
| CC Margin (ODO3921) | 21.5 | Thyroid Cancer 064010 | 0.0 |
| CC from Partial | 0.0 | Thyroid Cancer | 0.0 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 0.0 | Normal Breast | 63.7 |
| (OD04451-01) | | | |
| Lung Margin (OD04451-02) | 0.0 | Breast Cancer | 0.0 |
| | | (OD04566) | |
| Normal Prostate 6546-1 | 0.0 | Breast Cancer | 0.0 |
| | | (OD04590-01) | |
| Prostate Cancer (OD04410) | 11.3 | Breast Cancer Mets | 0.0 |
| | | (OD04590-03) | |
| Prostate Margin (OD04410) | 21.0 | Breast Cancer | 0.0 |
| | | Metastasis (OD04655- | |
| | | 05) | |
| Prostate Cancer (OD04720- | 94.6 | Breast Cancer 064006 | 8.2 |
| 01) | | | |
| Prostate Margin (OD04720- | 100.0 | Breast Cancer 1024 | 34.4 |
| 02) | | | |
| Normal Lung 061010 | 16.2 | Breast Cancer 9100266 | 0.0 |
| Lung Met to Muscle | 14.1 | Breast Margin 9100265 | 0.0 |
| (ODO4286) | | | |
| Muscle Margin (ODO4286) | 0.0 | Breast Cancer A209073 | 11.8 |
| Lung Malignant Cancer | 0.0 | Breast Margin | 47.6 |
| (OD03126) | | A2090734 | |
| Lung Margin (OD03126) | 27.9 | Normal Liver | 0.0 |
| Lung Cancer (OD04404) | 0.0 | Liver Cancer 064003 | 1.5 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 33.0 | Liver Cancer 6004-T | 0.0 |
| Lung Cancer (OD04237-01) | 0.0 | Liver Tissue 6004-N | 0.0 |
| Lung Margin (OD04237-02) | 0.0 | Liver Cancer 6005-T | 0.0 |
| Ocular Mel Met to Liver | 0.0 | Liver Tissue 6005-N | 0.0 |
| (ODO4310) | | | |
| Liver Margin (ODO4310) | 0.0 | Normal Bladder | 13.6 |
| Melanoma Mets to Lung | 0.0 | Bladder Cancer 1023 | 4.8 |
| (OD04321) | | | |
| Lung Margin (OD04321) | 34.2 | Bladder Cancer | 31.6 |
| | | A302173 | |
| Normal Kidney | 17.2 | Bladder Cancer | 0.0 |
| | | (OD04718-01) | |
| Kidney Ca, Nuclear grade 2 | 0.0 | Bladder Normal | 0.0 |
| (OD04338) | | Adjacent (OD04718-03) | |
| Kidney Margin (OD04338) | 23.0 | Normal Ovary | 0.0 |
| Kidney Ca Nuclear grade 1/2 | 0.0 | Ovarian Cancer 064008 | 15.3 |
| (OD04339) | | | |
| Kidney Margin (OD04339) | 0.0 | Ovarian Cancer | 14.5 |
| | | (OD04768-07) | |
| Kidney Ca, Clear cell type | 0.0 | Ovary Margin | 0.0 |
| (OD04340) | | (OD04768-08) | |
| Kidney Margin (OD04340) | 32.1 | Normal Stomach | 4.2 |
| Kidney Ca, Nuclear grade 3 | 4.5 | Gastric Cancer 9060358 | 0.0 |
| (OD04348) | | | |
| Kidney Margin (OD04348) | 57.0 | Stomach Margin | 0.0 |
| | | 9060359 | |
| Kidney Cancer (OD04622- | 0.0 | Gastric Cancer 9060395 | 0.0 |
| 01) | | | |
| Kidney Margin (OD04622- | 0.0 | Stomach Margin | 3.7 |
| 03) | | 9060394 | |
| Kidney Cancer (OD04450- | 0.0 | Gastric Cancer 9060397 | 0.0 |
| 01) | | | |
| Kidney Margin (OD04450- | 0.0 | Stomach Margin | 0.0 |
| 03) | | 9060396 | |
| Kidney Cancer 8120607 | 0.0 | Gastric Cancer 064005 | 7.9 |

TABLE AVC
Panel 4D

| | Rel. Exp.(%) Ag2182, | | Rel. Exp.(%) Ag2182, |
|---|---|---|---|
| Tissue Name | Run 163578421 | Tissue Name | Run 163578421 |
| Secondary Th1 act | 0.0 | HUVEC IL-1 beta | 0.0 |
| Secondary Th2 act | 0.0 | HUVEC IFN gamma | 1.3 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1 beta | |
| Primary Th1 rest | 0.0 | Bronchial epithelium | 2.5 |
| | | TNFalpha + IL1beta | |
| Primary Th2 rest | 0.0 | Small airway epithelium none | 3.7 |
| Primary Tr1 rest | 0.0 | Small airway epithelium | 44.1 |
| | | TNFalpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1 beta | |
| CD8 lymphocyte act | 0.0 | Astrocytes rest | 1.7 |
| Secondary CD8 | 0.0 | Astrocytes TNFalpha + IL- | 3.0 |
| lymphocyte rest | | 1 beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 4.2 |
| CD95 CH11 | | none | |
| LAK cells rest | 0.0 | CCD1106 (Keratinocytes) | 1.8 |
| | | TNFalpha + IL-1 beta | |
| LAK cells IL-2 | 0.0 | Liver cirrhosis | 6.0 |
| LAK cells IL-2 + IL-12 | 0.0 | Lupus kidney | 0.8 |
| LAK cells IL-2 + IFN | 0.0 | NCI-H292 none | 77.4 |
| gamma | | | |
| LAK cells IL-2 + IL-18 | 0.0 | NCI-H292 IL-4 | 100.0 |
| LAK cells | 0.0 | NCI-H292 IL-9 | 62.4 |
| PMA/ionomycin | | | |
| NK Cells IL-2 rest | 0.0 | NCI-H292 IL-13 | 35.4 |
| Two Way MLR 3 day | 0.0 | NCI-H292 IFN gamma | 22.4 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 3.1 |
| PBMC PWM | 0.0 | Lung fibroblast TNF alpha + | 0.9 |
| | | IL-1 beta | |
| PBMC PHA-L | 0.0 | Lung fibroblast IL-4 | 1.7 |
| Ramos (B cell) none | 2.6 | Lung fibroblast IL-9 | 3.0 |
| Ramos (B cell) | 0.0 | Lung fibroblast IL-13 | 1.4 |
| ionomycin | | | |
| B lymphocytes PWM | 0.0 | Lung fibroblast IFN gamma | 0.8 |
| B lymphocytes CD40L | 0.0 | Dermal fibroblast CCD1070 | 0.6 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 1.8 |
| Monocytes rest | 0.0 | IBD Crohn's | 0.0 |
| Monocytes LPS | 0.0 | Colon | 1.4 |
| Macrophages rest | 0.0 | Lung | 1.9 |
| Macrophages LPS | 0.0 | Thymus | 0.6 |
| HUVEC none | 0.0 | Kidney | 0.0 |
| HUVEC starved | 1.4 | | |

CNS_neurodegeneration_v1.0 Summary: Ag2182 Expression of the CG55766-01 gene is low/undetectable in all samples in this panel (CTs>35). (Data not shown.)

Panel 1.3D Summary: Ag2182 Expression of the CG55766-01 gene is low/undetectable in all samples in this panel (CTs>35). (Data not shown.)

Panel 2.2 Summary: Ag2182 Expression of the CG55766-01 gene is low/undetectable in all samples in this panel (CTs>35). (Data not shown.)

Panel 2D Summary: Ag2182 The expression of the CG55766-01 gene appears to be highest in normal prostate (CT=333 .5). In addition, there appears to be substantial expression in prostate cancer adjacent to normal prostate and in normal breast and kidney tissue. Of note was the differential expression between many of the normal tissues when compared to their malignant counterparts. Thus, expression of this gene could be used to distinguish between these samples and other samples on this panel and in particular distinguish between normal and cancer. Moreover, therapeutic modulation of this gene, through the use of small molecule drugs, antibodies or protein therapeutics might be of benefit for the treatment of breast cancer, kidney cancer or prostate cancer.

Panel 3D Summary: Ag2182 Expression of the CG55766-01 gene is low/undetectable in all samples in this panel (CTs>35). (Data not shown.)

Panel 4D Summary: Ag2182 The CG55766-01 gene is expressed at a moderate level (CT=28=31) in resting and IL-4, IL-9, or IL-13 and IFN gamma activated NCI-H292 mucoepidermoid cells. Moderate expression of this gene is also detected in the TNFalpha +IL-1beta stimulated small airway epithelial cells, while low but significant levels of expression (CT 33-35) are detected in IL-9 treated lung fibroblasts, untreated lung fibroblasts, TNFalpha +IL-1beta treated bronchial epithelial cells, and untreated Ramos B cells. The expression of this gene in lung derived cells and B cells suggests that this gene may be involved in normal conditions as well as pathological and inflammatory lung disorders including chronic obstructive pulmonary disease, asthma, allergy and emphysema. Therefore, small molecules or antibodies that modulate the function of this gene product may be useful therapeutics for the reduction or elimination of the symptoms in these diseases.

### EQUIVALENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS: 2, 4, 6, 8, 10 ,12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130;
(b) a variant of a mature form of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130; and
(d) a variant of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130, wherein one or more amino acid residues in said variant differs from the amino acid sequence of said mature form, provided that said variant differs in no more than 15% of amino acid residues from said amino acid sequence.

2 The polypeptide of claim 1, wherein said polypeptide comprises the amino acid sequence of a naturally-occurring allelic variant of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130;
(b) a variant of a mature form of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130;
(d) a variant of an amino acid sequence selected from the group consisting SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 and 127, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a GPCRX-associated disorder, said method comprising administering to a subject in which such treatment or prevention is desired the polypeptide of claim 1 in an amount sufficient to treat or prevent said GPCRX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a GPCRX-associated disorder, said method comprising administering to a subject in which such treatment or prevention is desired the nucleic acid of claim 5 in an amount sufficient to treat or prevent said GPCRX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a GPCRX-associated disorder, said method comprising administering to a subject in which such treatment or prevention is desired the antibody of claim 15 in an amount sufficient to treat or prevent said GPCRX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130, or a biologically active fragment thereof.

49. A method of treating a pathological state in a mammal, the method comprising administering to the mammal the antibody of claim 15 in an amount sufficient to alleviate the pathological state.

50. A method for identifying a compound that interacts with an olfactory receptor polypeptide, the method comprising:
a) providing a polypeptide of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130, or a peptide fragment or a variant thereof;
b) contacting said polypeptide with a candidate compound; and
c) detecting a complex, if present, between said polypeptide and said candidate compound
wherein the presence of a complex indicates that the candidate compound interacts with an olfactory receptor polypeptide.

51. A method for identifying a compound that interacts with an olfactory receptor polypeptide, the method comprising:
a) providing a eukaryotic host cell containing a recombinant nucleic acid encoding a polypeptide comprising the amino acid sequence of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130;
b) culturing said cell under conditions allowing for the expression of said polypeptide
c) contacting said culture with a candidate compound; and
d) detecting a second messenger metabolite, if present, in said culture
wherein an alteration in levels of said second messenger metabolite in the presence of the candidate compound as compared to the levels of said second messenger metabolite in the absence of said candidate compound indicates that the candidate compound interacts with an olfactory receptor polypeptide.

52. A method for identifying a compound that interacts with an olfactory receptor polypeptide, the method comprising:
a) providing an olfactory epithelial cell transfected with an adenovirus containing a nucleic acid encoding a polypeptide comprising the amino acid sequence of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 and 130;
b) contacting said olfactory epithelial cell with a candidate compound; and
c) detecting a response, if present, in said cell
wherein an alteration in response in the presence of the candidate compound as compared to the response in the absence of said candidate compound indicates that the candidate compound interacts with an olfactory receptor polypeptide.
